(19) United States
(12) Patent Application Publication
(10) Pub. No.: US 2024/0269309 A1
(43) Pub. Date: Aug. 15, 2024

(54) CAMPTOTHECIN ANALOGUES, CONJUGATES AND METHODS OF USE

(71) Applicant: ZYMEWORKS INC., Vancouver (CA)

(72) Inventors: Mark Edmund Petersen, Vancouver (CA); Michael Brant, Vancouver (CA); Raffaele Colombo, Vancouver (CA); james R. Rich, Vancouver (CA); Manuel Michel Auguste Lasalle, Vancouver (CA); Stuart Barnscher, Vancouver (CA); Samir Das, Vancouver (CA)

(21) Appl. No.: 18/562,049

(22) PCT Filed: May 27, 2022
(86) PCT No.: PCT/CA2022/050864

§ 371 Date: Nov. 17, 2023

Related U.S. Application Data
(60) Provisional application No. 63/194,138, filed on May 27, 2021.
(60) Provisional application No. 63/203,667, filed on Jul. 27, 2021.
(60) Provisional application No. 63/290,587, filed on Dec. 16, 2021.

(51) Int. Cl. A61K 47/68; A61K 31/4745; A61P 35/00; C07D 491/22

(52) U.S. Cl. CPC A61K 47/68037; A61K 31/4745; A61K 47/6851; A61K 47/6889; A61P 35/00; C07D 491/22

(57) ABSTRACT

Camptothecin analogues of Formula (I) and conjugates comprising the camptothecin analogues are described. The camptothecin analogues and conjugates may be used as therapeutic agents, particularly in the treatment of cancer, an autoimmune disease or a viral infection.

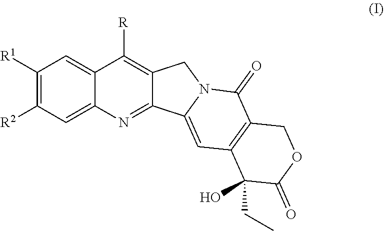

### FIELD

The present disclosure relates to the field of therapeutics and, in particular, to camptothecin analogues, conjugates comprising the camptothecin analogues, and their use in therapy.

### BACKGROUND

Camptothecin is a natural product that inhibits topoisomerase I and has broad spectrum anti-tumor activity. Camptothecin, however, is poorly soluble making it unsuitable for clinical development. As such, considerable effort has been directed towards identifying analogues or derivatives of camptothecin with properties more suitable for therapeutic use. Two derivatives, irinotecan and topotecan, have been approved for treatment of cancer. Irinotecan is a prodrug, which is converted in vivo into SN-38, a more potent analogue. A third derivative, belotecan, has been approved in Korea.

Camptothecin analogues have also been developed as payloads for antibody-drug conjugates (ADCs). Two such ADCs have been approved for treatment of cancer. Trastuzumab deruxtecan (Enhertu™) in which the camptothecin analogue, deruxtecan (Dxd), is conjugated to the anti-HER2 antibody, trastuzumab, via a cleavable tetrapeptide-based linker, and sacituzumab govitecan (Trodelvy™) in which the camptothecin analogue, SN-38, is conjugated to the anti-Trop-2 antibody, sacituzumab, via a hydrolysable, pH-sensitive linker.

Other camptothecin analogues and derivatives, as well as ADCs comprising them have been described. See, for example, International (PCT) Publication Nos. WO 2019/195665; WO 2019/236954; WO 2020/200880 and WO 2020/219287.

This background information is provided for the purpose of making known information believed by the applicant to be of possible relevance to the present disclosure. No admission is necessarily intended, nor should be construed, that any of the preceding information constitutes prior art against the claimed invention.

### SUMMARY

Described herein are camptothecin analogue compounds, conjugates comprising the compounds and methods of treatment using the compounds and conjugates. In one aspect, the present disclosure relates to a compound having Formula (I):

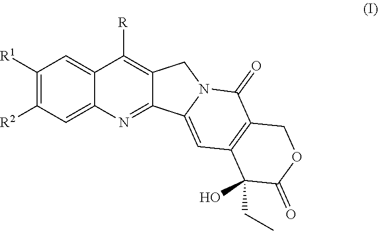

wherein:
   R¹ is selected from: —H, —CH₃, —CHF₂, —CF₃, —F, —Br, —Cl, —OH, —OCH₃, —OCF₃ and —NH₂, and
   R² is selected from: —H, —CH₃, —CF₃, —F, —Br, —Cl, —OH, —OCH₃ and —OCF₃, and wherein:
   when R¹ is —NH₂, then R is R³ or R⁴, and when R¹ is other than —NH₂, then R is R⁴;
   R³ is selected from: —H, —C₁-C₆ alkyl, —C₃-C₈ cycloalkyl, —(C₁-C₆ alkyl)-O—R⁵,

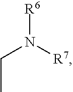

—CO₂R⁸, -aryl, -heteroaryl and —(C₁-C₆ alkyl)-aryl;
R⁴ is selected from:

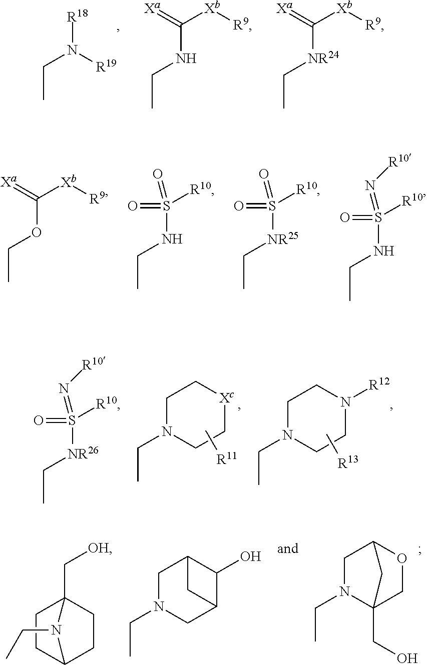

R⁵ is selected from: —H, —C₁-C₆ alkyl, —C₃-C₈ cycloalkyl, -aryl, -heteroaryl, -aryl and —(C₁-C₆ alkyl)-aryl;
R⁶ and R⁷ are each independently selected from: —H, —C₁-C₆ alkyl, —C₃-C₈ cycloalkyl, —(C₁-C₆ alkyl)-O—R⁵, —C₃-C₈ heterocycloalkyl and —C(O)R¹⁷;
R⁸ is selected from: —H, —C₁-C₆ alkyl, —C₃-C₈ cycloalkyl and —C₃-C₈ heterocycloalkyl;
each R⁹ is independently selected from: —H, —C₁-C₆ alkyl, —C₃-C₈ cycloalkyl, -aryl, -heteroaryl and —(C₁-C₆ alkyl)-aryl;
each R¹⁰ is independently selected from: —C₁-C₆ alkyl, —C₃-C₈ cycloalkyl, —NR¹⁴R^{14′}, -aryl, -heteroaryl and —(C₁-C₆ alkyl)-aryl;
R^{10′} is selected from: —H, —C₁-C₆ alkyl, —C₃-C₈ cycloalkyl, -aryl, -heteroaryl, and —(C₁-C₆ alkyl)-aryl;
R¹¹ is selected from: —H and —C₁-C₆ alkyl;
R¹² is selected from: —H, —C₁-C₆ alkyl, —CO₂R⁸, -aryl, -heteroaryl, —(C₁-C₆ alkyl)-aryl, —S(O)₂R¹⁶ and

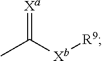

R¹³ is selected from: —H and —C₁-C₆ alkyl;
R¹⁴ and R^{14′} are each independently selected from: —H, C₁-C₆ alkyl, —C₃-C₈ cycloalkyl and —C₃-C₈ heterocycloalkyl;
R¹⁶ is selected from: —C₁-C₆ alkyl, —C₃-C₈ cycloalkyl, -aryl, -heteroaryl and —(C₁-C₆ alkyl)-aryl;
R¹⁷ is selected from: —C₁-C₆ alkyl, —C₃-C₈ cycloalkyl, —C₃-C₈ heterocycloalkyl, —(C₁-C₆ alkyl)-C₃-C₈ heterocycloalkyl, -aryl, -heteroaryl and —(C₁-C₆ alkyl)-aryl;
R¹⁸ and R¹⁹ taken together with the N atom to which they are bonded form a 4-, 5-, 6- or 7-membered ring having 0 to 3 substituents selected from: halogen, —C₁-C₆ alkyl, —C₃-C₈ cycloalkyl and —(C₁-C₆ alkyl)-O—R⁵;
R²⁴, R²⁵ and R²⁶ are each —C₁-C₆ alkyl;
X^{a} and X^{b} are each independently selected from: NH, O and S, and
X^{c} is selected from; O, S and S(O)₂,
with the proviso that the compound is other than (S)-9-amino-11-butyl-4-ethyl-4-hydroxy-1,12-dihydro-14H-pyrano[3′,4′:6,7]indolizino[1,2-b]quinoline-3,14(4H)-dione.

In certain embodiments, in compounds of Formula (I), R¹ is NH₂, and R² is other than —H.

Another aspect of the present disclosure relates to a pharmaceutical composition comprising a compound having Formula (I), and a pharmaceutically acceptable carrier or diluent.

Another aspect of the present disclosure relates to a method of inhibiting the proliferation of cancer cells comprising contacting the cells with an effective amount of a compound according having Formula (I). Another aspect relates to a method of killing cancer cells comprising contacting the cells with an effective amount of a compound having Formula (I).

Another aspect of the present disclosure relates to a method of treating cancer in a subject in need thereof comprising administering to the subject an effective amount of a compound having Formula (I). Another aspect relates to a method of treating an autoimmune disease in a subject in need thereof comprising administering to the subject an effective amount of a compound having Formula (I). Another aspect relates to a method of treating a viral infection in a subject in need thereof comprising administering to the subject an effective amount of a compound having Formula (I).

Another aspect of the present disclosure relates to a compound having Formula (I) for use in therapy. Another aspect relates to a compound of Formula (I) for use in the treatment of cancer, an autoimmune disease or a viral infection.

Another aspect of the present disclosure relates to a use of a compound having Formula (I) in the manufacture of a medicament for the treatment of cancer, an autoimmune disease or a viral infection.

Another aspect of the present disclosure relates to a conjugate having Formula (X):

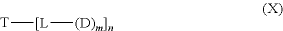

wherein:
T is a targeting moiety;
L is a linker;
D is a camptothecin analogue as described herein;
m is an integer between 1 and 4, and
n is an integer between 1 and 10.

Another aspect of the present disclosure relates to a conjugate having Formula (X):

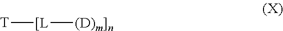

wherein:
T is a targeting moiety;
L is a linker;
m is an integer between 1 and 4;
n is an integer between 1 and 10, and
D is a compound of Formula (IV):

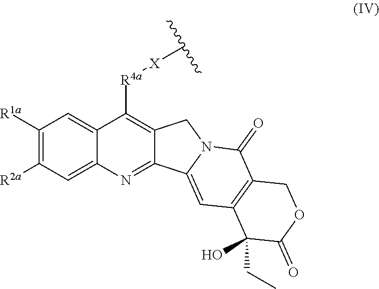

wherein:
   R^{1a} is selected from: —H, —CH₃, —CHF₂, —CF₃, —F, —Br, —Cl, —OH, —OCH₃, —OCF₃ and —NH₂;
   R^{2a} is selected from: —H, —CH₃, —CF₃, —F, —Br, —Cl, —OH, —OCH₃ and —OCF₃;
   X is —O—, —S— or —NH—, and R^{4a} is selected from:

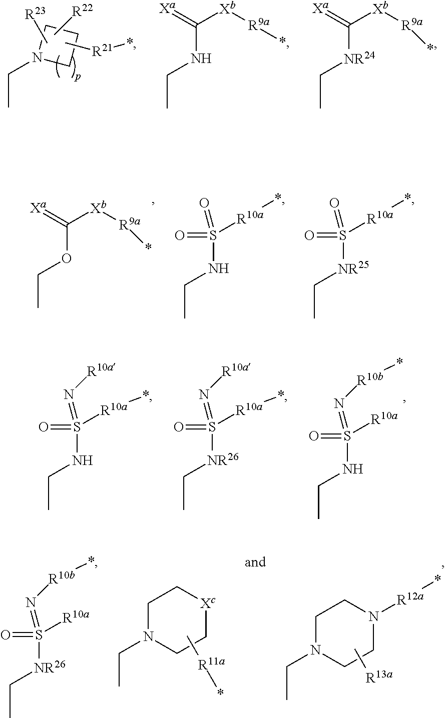

wherein * is the point of attachment to X, and wherein p is 1, 2, 3 or 4; or
X is O, and R^{4a}—X— is selected from:

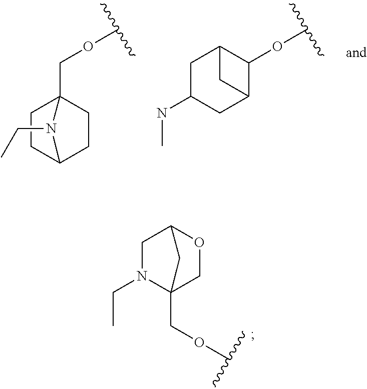 [SMILES: *OCC12CCC(CC1)N2CC ; *OC1C2CC(NC)CC1C2 ; *OCC12COC(CN1CC)C2]

R^{5a} is selected from: —C₁-C₆ alkyl, —C₃-C₈ cycloalkyl, -aryl, -heteroaryl and —(C₁-C₆ alkyl)-aryl;
R^{8a} is selected from: —C₁-C₆ alkyl, —C₃-C₈ cycloalkyl and —C₃-C₈ heterocycloalkyl;
each R^{9a} is independently selected from: —C₁-C₆ alkyl, —C₃-C₈ cycloalkyl, -aryl, -heteroaryl and —(C₁-C₆ alkyl)-aryl; or R^{9a} is absent and X^{b}═X;
each R^{10a} is independently selected from: —C₁-C₆ alkyl, —C₃-C₈ cycloalkyl, -aryl, -heteroaryl, —(C₁-C₆ alkyl)-aryl and;

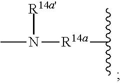

each R^{10a′} is independently selected from: —H, —C₁-C₆ alkyl, —C₃-C₈ cycloalkyl, -aryl, -heteroaryl and —(C₁-C₆ alkyl)-aryl;
each R^{10b} is independently selected from: —C₁-C₆ alkyl, —C₃-C₈ cycloalkyl, -aryl, -heteroaryl and —(C₁-C₆ alkyl)-aryl;
R^{11a} is absent or is —C₁-C₆ alkyl;
R^{12a} is selected from: —C₁-C₆ alkyl, —CO₂R^{8a}, -aryl, -heteroaryl, —(C₁-C₆ alkyl)-aryl, —S(O)₂R^{16a} and

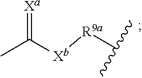

R^{13a} is selected from: —H and —C₁-C₆ alkyl;
R^{14a} is selected from: —C₁-C₆ alkyl, —C₃-C₈ cycloalkyl and —C₃-C₈ heterocycloalkyl;
R^{14a′} is selected from: H, —C₁-C₆ alkyl, —C₃-C₈ cycloalkyl and —C₃-C₈ heterocycloalkyl;
R^{16a} is selected from: —C₁-C₆ alkyl, —C₃-C₈ cycloalkyl, -aryl, -heteroaryl and —(C₁-C₆ alkyl)-aryl;
R²¹ is selected from: —C₁-C₆ alkyl, —C₃-C₈ cycloalkyl and —(C₁-C₆ alkyl)-O—R^{5a};
R²² and R²³ are each independently selected from: —H, -halogen, —C₁-C₆ alkyl and —C₃-C₈ cycloalkyl;
R²⁴, R²⁵ and R²⁶ are each —C₁-C₆ alkyl;
X^{a} and X^{b} are each independently selected from: NH, O and S;
X^{c} is selected from: O, S and S(O)₂, and
[symbol] denotes the point of attachment to linker, L.

Another aspect of the present disclosure relates to a conjugate having Formula (X):

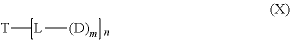

wherein:
T is a targeting moiety;
L is a linker;
m is an integer between 1 and 4;
n is an integer between 1 and 10, and
D is a compound of Formula (V):

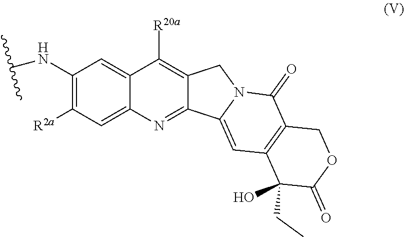

wherein:
   R^{2a} is selected from: —CH₃, —CF₃, —F, —Br, —Cl, —OH, —OCH₃ and —OCF₃;
   R^{20a} is selected from: —H, —C₁-C₆ alkyl, —C₃-C₈ cycloalkyl, —(C₁-C₆ alkyl)-O—R⁵,

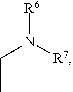

φ₂R⁸, -aryl, -heteroaryl, —(C₁-C₆ alkyl)-aryl

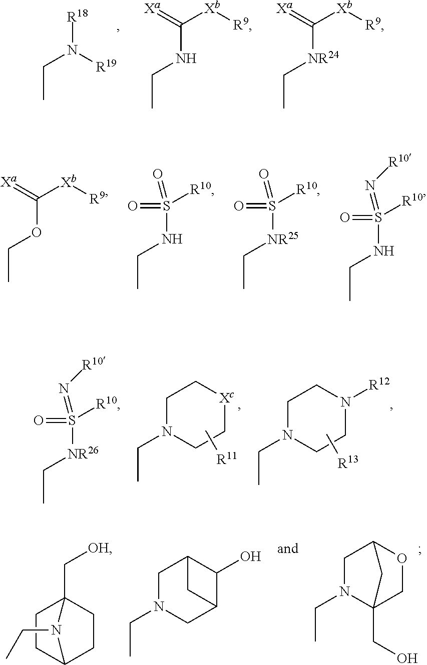

R⁵ is selected from: —H, —C₁-C₆ alkyl, —C₃-C₈ cycloalkyl, -aryl, -heteroaryl and —(C₁-C₆ alkyl)-aryl;
R⁶ and R⁷ are each independently selected from: —H, —C₁-C₆ alkyl, —C₃-C₈ cycloalkyl, —(C₁-C₆ alkyl)-O—R⁵, —C₃-C₈ heterocycloalkyl and —C(O)R¹⁷;
R⁸ is selected from: —H, —C₁-C₆ alkyl, —C₃-C₈ cycloalkyl and —C₃-C₈ heterocycloalkyl;
each R⁹ is independently selected from: —H, —C₁-C₆ alkyl, —C₃-C₈ cycloalkyl, -aryl, -heteroaryl and —(C₁-C₆ alkyl)-aryl;
each R^{10a} is independently selected from: —C₁-C₆ alkyl, —C₃-C₈ cycloalkyl, -aryl, -heteroaryl, —(C₁-C₆ alkyl)-aryl and

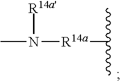

R^{10a′} is selected from: —H, —C₁-C₆ alkyl, —C₃-C₈ cycloalkyl, -aryl, -heteroaryl and —(C₁-C₆ alkyl)-aryl;
R¹¹ is selected from: —H and —C₁-C₆ alkyl;
R¹² is selected from: —H, —C₁-C₆ alkyl, —CO₂R⁸, -aryl, -heteroaryl, —(C₁-C₆ alkyl)-aryl, —S(O)₂R¹⁶ and

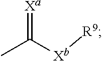

R¹³ is selected from: —H and —C₁-C₆ alkyl;
R¹⁴ and R^{14′} are each independently selected from: —H, C₁-C₆ alkyl, —C₃-C₈ cycloalkyl and —C₃-C₈ heterocycloalkyl;
R¹⁶ is selected from: —C₁-C₆ alkyl, —C₃-C₈ cycloalkyl, -aryl, -heteroaryl and —(C₁-C₆ alkyl)-aryl;
R¹⁷ is selected from: —C₁-C₆ alkyl, —C₃-C₈ cycloalkyl, —C₃-C₈ heterocycloalkyl, —(C₁-C₆ alkyl)-C₃-C₈ heterocycloalkyl, -aryl, -heteroaryl and —(C₁-C₆ alkyl)-aryl; R¹⁸ and R¹⁹ taken together with the N atom to which they are bonded form a 4-, 5-, 6- or 7-membered ring having 0 to 3 substituents selected from: halogen, —C₁-C₆ alkyl, —C₃-C₈cycloalkyl and —(C₁-C₆ alkyl)-O—R⁵;
R²⁴, R²⁵ and R²⁶ are each —C₁-C₆ alkyl;
X^{a} and X^{b} are each independently selected from: NH, O and S; X^{c} is selected from: O, S and S(O)₂, and
[symbol] denotes the point of attachment to linker, L.

Another aspect of the present disclosure relates to a conjugate having Formula (X):

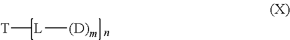

wherein:
T is a targeting moiety;
L is a linker;
m is an integer between 1 and 4;
n is an integer between 1 and 10, and
   D is a compound of Formula (VI):

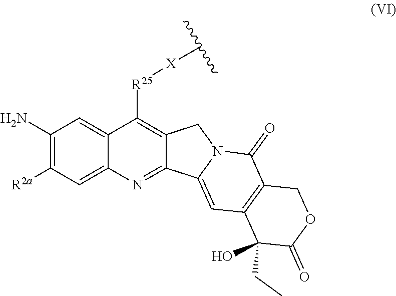

wherein:
   R^{2a} is selected from: —H, —CH₃, —CF₃, —F, —Br, —Cl, —OH, —OCH₃ and —OCF₃;
   X is —O—, —S— or —NH—, and R²⁵ is selected from: —C₁-C₆ alkyl, —(C₁-C₆ alkyl)-O—R^{5a}, —CO₂R^{8a}, —C(O)—, -aryl, -heteroaryl, —(C₁-C₆ alkyl)-aryl,

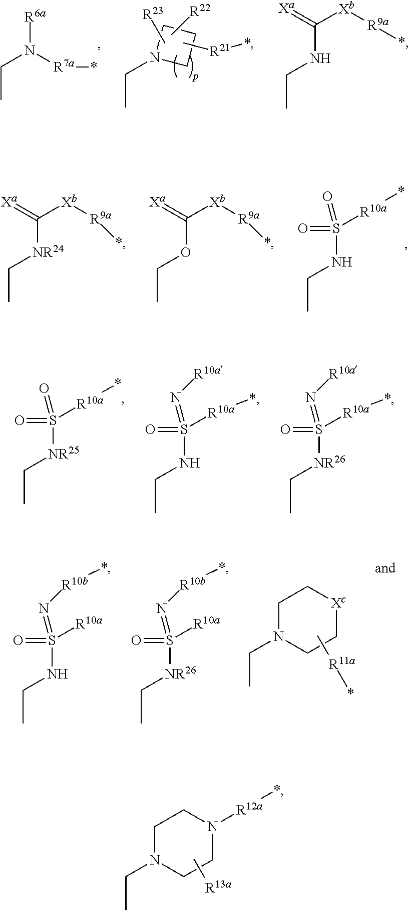

wherein * is the point of attachment to X, and wherein p is 1, 2, 3 or 4; or
X is O, and R²⁵—X— is selected from:

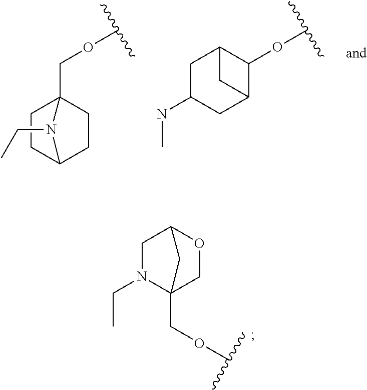 [SMILES: *OCC12CCC(CC1)N2CC ; *OC1C2CC(NC)CC1C2 ; *OCC12COC(CN1CC)C2]

R^{5a} is selected from: —C₁-C₆ alkyl, —C₃-C₈ cycloalkyl, -aryl, -heteroaryl and —(C₁-C₆ alkyl)-aryl;
R^{6a} is selected from: —H, —C₁-C₆ alkyl, —C₃-C₈ cycloalkyl and —C₃-C₈ heterocycloalkyl;
R^{7a} is selected from: —C₁-C₆ alkyl, —C₃-C₈ cycloalkyl, —(C₁-C₆ alkyl)-O—R^{5a}, —C₃-C₈ heterocycloalkyl and —C(O)R^{17a};
R^{8a} is selected from: —C₁-C₆ alkyl, —C₃-C₈ cycloalkyl and —C₃-C₈ heterocycloalkyl;
each R^{9a} is independently selected from: —C₁-C₆ alkyl, —C₃-C₈ cycloalkyl, -aryl, -heteroaryl and —(C₁-C₆ alkyl)-aryl; or R^{9a} is absent and X^{b}═X;
each R^{10a} is independently selected from: —C₁-C₆ alkyl, —C₃-C₈ cycloalkyl, -aryl, -heteroaryl, —(C₁-C₆ alkyl)-aryl and

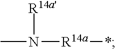

each R^{10a′} is independently selected from: —H, —C₁-C₆ alkyl, —C₃-C₈ cycloalkyl, -aryl, -heteroaryl and —(C₁-C₆ alkyl)-aryl;
each R^{10b} is independently selected from: —C₁-C₆ alkyl, —C₃-C₈ cycloalkyl, -aryl, -heteroaryl and —(C₁-C₆ alkyl)-aryl;
R^{11a} is absent or is —C₁-C₆ alkyl;
R^{12a} is selected from: —C₁-C₆ alkyl, —CO₂R^{8a}, -aryl, -heteroaryl, —(C₁-C₆ alkyl)-aryl, —S(O)₂R^{16a} and

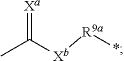

R^{13a} is selected from: —H and —C₁-C₆ alkyl;
R^{14a} is selected from: —C₁-C₆ alkyl, —C₃-C₈ cycloalkyl and —C₃-C₈ heterocycloalkyl;
R^{14a′} is selected from: H, —C₁-C₆ alkyl, —C₃-C₈ cycloalkyl and —C₃-C₈ heterocycloalkyl;
R^{16a} is selected from: —C₁-C₆ alkyl, —C₃-C₈ cycloalkyl, -aryl, -heteroaryl and —(C₁-C₆ alkyl)-aryl;
R^{17a} is selected from: —C₁-C₆ alkyl, —C₃-C₈ cycloalkyl, —C₃-C₈ heterocycloalkyl, —(C₁-C₆ alkyl)-C₃-C₈ heterocycloalkyl, -aryl, -heteroaryl and —(C₁-C₆ alkyl)-aryl;
R²¹ is selected from: —C₁-C₆ alkyl, —C₃-C₈ cycloalkyl and —(C₁-C₆ alkyl)-O—R^{5a};
R²² and R²³ are each independently selected from: —H, -halogen, —C₁-C₆ alkyl and —C₃-C₈ cycloalkyl;
R²⁴, R²⁵ and R²⁶ are each —C₁-C₆ alkyl;
X^{a} and X^{b} are each independently selected from: NH, O and S;
X^{c} is selected from: O, S and S(O)₂, and
[symbol] denotes the point of attachment to linker, L.

Another aspect of the present disclosure relates to a pharmaceutical composition comprising a conjugate of Formula (X), and a pharmaceutically acceptable carrier or diluent.

Another aspect of the present disclosure relates to a method of inhibiting the proliferation of cancer cells comprising contacting the cells with an effective amount of a conjugate having Formula (X). Another aspect relates to a method of killing cancer cells comprising contacting the cells with an effective amount of a conjugate having Formula (X).

Another aspect of the present disclosure relates to a method of treating cancer in a subject in need thereof comprising administering to the subject an effective amount of a conjugate of Formula (X). Another aspect relates to a method of treating an autoimmune disease in a subject in need thereof comprising administering to the subject an effective amount of a conjugate of Formula (X). Another aspect relates to a method of treating a viral infection in a subject in need thereof comprising administering to the subject an effective amount of a conjugate of Formula (X).

Another aspect of the present disclosure relates to a conjugate having Formula (X) for use in therapy. Another aspect relates to a conjugate having Formula (X) for use in the treatment of cancer, an autoimmune disease or a viral infection.

Another aspect of the present disclosure relates to a use of a conjugate having Formula (X) in the manufacture of a medicament for the treatment of cancer, an autoimmune disease or a viral infection.

### BRIEF DESCRIPTION OF THE DRAWINGS

FIG. 1 presents schematics of general procedures that may be used in the preparation of intermediates for the synthesis of camptothecin analogues and conjugates described herein, (A) Synthetic Scheme I: General Procedure 1; (B) Synthetic Scheme II: General Procedure 2; (C) Synthetic Scheme III: General Procedure 3; (D) Synthetic Scheme IV: General Procedure 4; (E) Synthetic Scheme V: General Procedure 5; (F) Synthetic Scheme VI: General Procedure 7, (G) Synthetic Scheme VII: General Procedure 8.

FIG. 2 shows the bystander killing effect of conjugates comprising camptothecin analogues described herein conjugated to trastuzumab at DAR 8 on HER2-negative MDA-MB-468 cancer cells, (A) at 1 nM concentration, and (B) 0.1 nM concentration.

FIG. 3 shows the anti-tumor activity of conjugates comprising camptothecin analogues described herein conjugated to trastuzumab at DAR 8 in a JIMT-1 xenograft model of breast cancer expressing HER2 (mid).

FIG. 4 shows exemplary drug-linker (DL) structures comprising camptothecin analogues of Formula (I) with a C7 linkage (Table 4).

FIG. 5 shows exemplary drug-linker (DL) structures comprising camptothecin analogues of Formula (I) with a C10 linkage (Table 5).

FIG. 6 shows exemplary drug-linker (DL) structures comprising camptothecin analogues of Formula (I) with either a C7 or C10 linkage (Table 6).

FIG. 7 shows exemplary conjugate (DC) structures comprising camptothecin analogues of Formula (I) with a C7 linkage (Table 7).

FIG. 8 shows exemplary conjugate (DC) structures comprising camptothecin analogues of Formula (I) with a C10 linkage (Table 8).

FIG. 9 shows exemplary conjugate (DC) structures comprising camptothecin analogues of Formula (I) with either a C7 or C10 linkage (Table 9).

FIG. 10A-C shows the in vivo anti-tumor activities of an anti-FRα antibody v30384 (A) conjugated to the camptothecin analogues Compound 139 and Compound 141 at DAR 8 in an OV90 xenograft model, (B) conjugated to the camptothecin analogues Compound 140 and Compound 141 at DAR 8 in an OV90 xenograft model and (C) conjugated to the camptothecin analogues Compound 139, Compound 140, Compound 141 and Compound 148 at DAR 8 in a H2110 xenograft model.

### DETAILED DESCRIPTION

The present disclosure relates to camptothecin analogues and conjugates comprising the camptothecin analogues. Camptothecin analogues and conjugates are shown to have cytotoxic activity, for example against cancer cells. Certain embodiments of the present disclosure thus relate to the use of the camptothecin analogues and conjugates as therapeutic agents, particularly in the treatment of cancer.

### Definitions

Unless defined otherwise, all technical and scientific terms used herein have the same meaning as commonly understood by one of ordinary skill in the art.

As used herein, the term “about” refers to an approximately +/−10% variation from a given value. It is to be understood that such a variation is always included in any given value provided herein, whether or not it is specifically referred to.

The use of the word “a” or “an” when used herein in conjunction with the term “comprising” may mean “one,” but it is also consistent with the meaning of “one or more,” “at least one” and “one or more than one.”

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a composition, use or method, denotes that additional elements and/or method steps may be present, but that these additions do not materially affect the manner in which the recited composition, method or use functions. The term “consisting of” when used herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to.

The term “acyl,” as used herein, refers to the group —C(O)R, where R is hydrogen, alkyl, aryl, heteroaryl, cycloalkyl or heterocycloalkyl.

The term “acyloxy” refers to the group —OC(O)R, where R is alkyl.

The term “alkoxy,” as used herein, refers to the group —OR, where R is alkyl, aryl, heteroaryl, cycloalkyl or cycloheteroalkyl.

The term “alkyl,” as used herein, refers to a straight chain or branched saturated hydrocarbon group containing the specified number of carbon atoms. Examples of alkyl include, but are not limited to, methyl, ethyl, n-propyl, isopropyl, n-butyl, sec-butyl, isobutyl, t-butyl, pentyl, isopentyl, t-pentyl, neo-pentyl, 1-methylbutyl, 2-methylbutyl, n-hexyl, and the like.

The term “alkylaminoaryl,” as used herein, refers to an alkyl group as defined herein substituted with one aminoaryl group as defined herein.

The term “alkylheterocycloalkyl,” as used herein, refers to an alkyl group as defined herein substituted with one heterocycloalkyl group as defined herein.

The term “alkylthio,” as used herein, refers to the group —SR, where R is an alkyl group.

The term “amido,” as used herein, refers to the group —C(O)NRR′, where R and R′ are independently hydrogen, alkyl, aryl, heteroaryl, cycloalkyl or heterocycloalkyl.

The term “amino,” as used herein, refers to the group —NRR′, where R and R′ are independently hydrogen, alkyl, aryl, heteroaryl, cycloalkyl or heterocycloalkyl.

The term “aminoalkyl,” as used herein, refers to an alkyl group as defined herein substituted with one or more amino groups, for example, one, two or three amino groups.

The term “aminoaryl,” as used herein, refers to an aryl group as defined herein substituted with one amino group.

The term “aryl,” as used herein, refers to a 6- to 12-membered mono- or bicyclic hydrocarbon ring system in which at least one ring aromatic. Examples of aryl include, but are not limited to, phenyl, naphthalenyl, 1,2,3,4-tetrahydro-naphthalenyl, 5,6,7,8-tetrahydro-naphthalenyl, indanyl, and the like.

The term “carboxy,” as used herein, refers to the group —C(O)OR, where R is H, alkyl, aryl, heteroaryl, cycloalkyl or cycloheteroalkyl.

The term “cyano,” as used herein, refers to the group —CN.

The term “cycloalkyl,” as used herein, refers to a mono- or bicyclic saturated hydrocarbon containing the specified number of carbon atoms. Examples of cycloalkyl include, but are not limited to, cyclopropyl, cyclobutyl, cyclopentyl, cyclohexyl, cycloheptane, bicyclo [2.2.1]heptane, bicyclo [3.1.1] heptane, and the like.

The term “haloalkyl,” as used herein, refers to an alkyl group as defined herein substituted with one or more halogen atoms.

The terms “halogen” and “halo,” as used herein refer to fluorine (F), bromine (Br), chlorine (Cl) and iodine (I).

The term “heteroaryl,” as used herein, refers to a 6- to 12-membered mono- or bicyclic ring system in which at least one ring atom is a heteroatom and at least one ring is aromatic.

Examples of heteroatoms include, but are not limited to, O, S and N. Examples of heteroaryl include, but are not limited to: pyridyl, benzofuranyl, pyrazinyl, pyridazinyl, pyrimidinyl, triazinyl, quinolinyl, benzoxazolyl, benzothiazolyl, isoquinolinyl, quinazolinyl, quinoxalinyl, pyrrolyl, indolyl, and the like.

The term “heterocycloalkyl,” as used herein, refers to a mono- or bicyclic non-aromatic ring system containing the specified number of atoms and in which at least one ring atom is a heteroatom, for example, O, S or N. A heterocyclyl substituent can be attached via any of its available ring atoms, for example, a ring carbon, or a ring nitrogen. Examples of heterocycloalkyl include, but are not limited to, aziridinyl, azetidinyl, piperidinyl, morpholinyl, piperazinyl, pyrrolidinyl, and the like.

The terms “hydroxy” and “hydroxyl,” as used herein, refer to the group —OH.

The term “hydroxyalkyl,” as used herein, refers to an alkyl group as defined herein substituted with one or more hydroxy groups.

The term “nitro,” as used herein, refers to the group —NO₂.

The term “sulfonyl,” as used herein, refers to the group —S(O)₂R, where R is H, alkyl or aryl.

The term “sulfonamido,” as used herein, refers to the group —NH—S(O)₂R, where R is H, alkyl or aryl.

The terms “thio” and “thiol,” as used herein, refer to the group —SH.

Unless specifically stated as being “unsubstituted,” any alkyl, cycloalkyl, heterocycloalkyl, aryl or heteroaryl group referred to herein is understood to be “optionally substituted,” i.e. each such reference includes both unsubstituted and substituted versions of these groups. For example, reference to a “—C₁-C₆ alkyl” includes both unsubstituted —C₁-C₆ alkyl and —C₁-C₆ alkyl substituted with one or more substituents. Examples of substituents include, but are not limited to, halogen, acyl, acyloxy, alkoxy, carboxy, hydroxy, amino, amido, nitro, cyano, azido, alkylthio, thio, sulfonyl, sulfonamido, alkyl, cycloalkyl, heterocycloalkyl, aryl or heteroaryl.

In certain embodiments, each alkyl, cycloalkyl, heterocycloalkyl, aryl or heteroaryl group referred to herein is optionally substituted with one or more substituents selected from: halogen, acyl, acyloxy, alkoxy, carboxy, hydroxy, amino, amido, nitro, cyano, azido, alkylthio, thio, sulfonyl and sulfonamido.

A chemical group described herein that is “substituted” may include one substituent or a plurality of substituents up to the full valence of substitution for that group. For example, a methyl group may include 1, 2, or 3 substituents, and a phenyl group may include 1, 2, 3, 4, or 5 substituents. When a group is substituted with more than one substituent, the substituents may be the same or they may be different.

The terms “subject” and “patient” as used herein refer to an animal in need of treatment. An animal in need of treatment may be a human or a non-human animal, such as a mammal, bird or fish. In certain embodiments, the subject or patient is a mammal. In some embodiments, the subject or patient is a human.

An “effective amount” of a compound or conjugate described herein in respect of a particular result to be achieved is an amount sufficient to achieve the desired result. For example, an “effective amount” of a compound when referred to in respect of the killing of cancer cells, refers to an amount of compound sufficient to produce a killing effect.

It is to be understood that the positive recitation of a feature in one embodiment, serves as a basis for excluding the feature in an alternative embodiment. In particular, where a list of options is presented for a given embodiment or claim, it is to be understood that one or more option may be deleted from the list and the shortened list may form an alternative embodiment, whether or not such an alternative embodiment is specifically referred to.

It is contemplated that any embodiment discussed herein can be implemented with respect to any method, use or composition disclosed herein, and vice versa.

### Camptothecin Analogues

In one aspect, the camptothecin analogue compounds of the present disclosure are compounds having Formula (I):

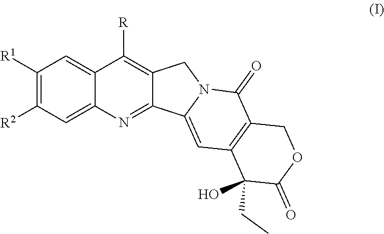

wherein:
   R¹ is selected from: —H, —CH₃, —CHF₂, —CF₃, —F, —Br, —Cl, —OH, —OCH₃, —OCF₃ and —NH₂, and
   R² is selected from: —H, —CH₃, —CF₃, —F, —Br, —Cl, —OH, —OCH₃ and —OCF₃, and wherein:
   when R¹ is —NH₂, then R is R³ or R⁴, and when R¹ is other than —NH₂, then R is R⁴;
   R³ is selected from: —H, —C₁-C₆ alkyl, —C₃-C₈ cycloalkyl, —(C₁-C₆ alkyl)-O—R⁵,

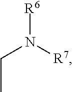

CO₂R⁸, -aryl, -heteroaryl and —(C₁-C₆ alkyl)-aryl;
R⁴ is selected from:

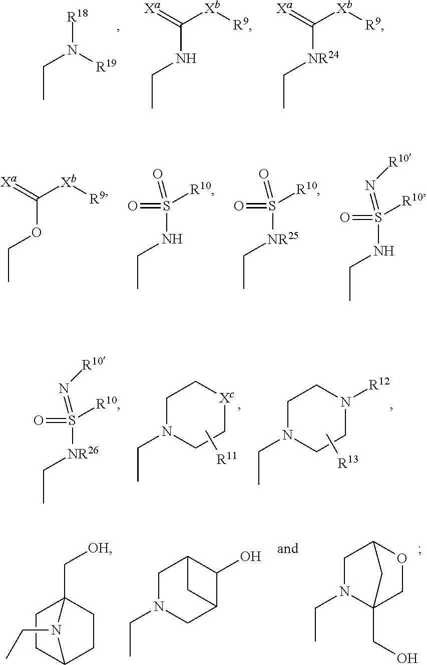

R⁵ is selected from: —H, —C₁-C₆ alkyl, —C₃-C₈ cycloalkyl, -aryl, -heteroaryl, -aryl and —(C₁-C₆ alkyl)-aryl;
R⁶ and R⁷ are each independently selected from: —H, —C₁-C₆ alkyl, —C₃-C₈ cycloalkyl, —(C₁-C₆ alkyl)-O—R⁵, —C₃-C₈ heterocycloalkyl and —C(O)R¹⁷;
R⁸ is selected from: —H, —C₁-C₆ alkyl, —C₃-C₈ cycloalkyl and —C₃-C₈ heterocycloalkyl;
each R⁹ is independently selected from: —H, —C₁-C₆ alkyl, —C₃-C₈ cycloalkyl, -aryl, -heteroaryl and —(C₁-C₆ alkyl)-aryl;
each R¹⁰ is independently selected from: —C₁-C₆ alkyl, —C₃-C₈ cycloalkyl, —NR¹⁴R^{14′}, -aryl, -heteroaryl and —(C₁-C₆ alkyl)-aryl;
each R^{10′} is independently selected from: —H, —C₁-C₆ alkyl, —C₃-C₈ cycloalkyl, -aryl, -heteroaryl, and —(C₁-C₆ alkyl)-aryl;
   R¹¹ is selected from: —H and —C₁-C₆ alkyl;
   R¹² is selected from: —H, —C₁-C₆ alkyl, —CO₂R⁸, -aryl, -heteroaryl, —(C₁-C₆ alkyl)-aryl, —S(O)₂R¹⁶ and

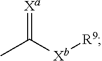

R¹³ is selected from: —H and —C₁-C₆ alkyl;
R¹⁴ and R^{14′} are each independently selected from: —H, C₁-C₆ alkyl, —C₃-C₈ cycloalkyl and —C₃-C₈ heterocycloalkyl;
R¹⁶ is selected from: —C₁-C₆ alkyl, —C₃-C₈ cycloalkyl, -aryl, -heteroaryl and —(C₁-C₆ alkyl)-aryl;
R¹⁷ is selected from: —C₁-C₆ alkyl, —C₃-C₈ cycloalkyl, —C₃-C₈ heterocycloalkyl, —(C₁-C₆ alkyl)-C₃-C₈ heterocycloalkyl, -aryl, -heteroaryl and —(C₁-C₆ alkyl)-aryl;
R¹⁸ and R¹⁹ taken together with the N atom to which they are bonded form a 4-, 5-, 6- or 7-membered ring having 0 to 3 substituents selected from: halogen, —C₁-C₆ alkyl, —C₃-C₈ cycloalkyl and —(C₁-C₆ alkyl)-O—R⁵;
R²⁴, R²⁵ and R²⁶ are each —C₁-C₆ alkyl;
X^{a} and X^{b} are each independently selected from: NH, O and S, and
X^{c} is selected from; O, S and S(O)₂,
with the proviso that the compound is other than (S)-9-amino-11-butyl-4-ethyl-4-hydroxy-1,12-dihydro-14H-pyrano[3′,4′:6,7]indolizino[1,2-b]quinoline-3,14(4H)-dione:

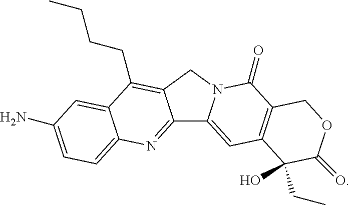 [SMILES: CCCCc1c2c(nc3ccc(N)cc13)-c1cc3c(c(=O)n1C2)COC(=O)[C@]3(O)CC]

In some embodiments, the camptothecin analogues are compounds of Formula (I), with the proviso that when R¹ is NH₂, R² is other than H.

In some embodiments, in compounds of Formula (I), R¹ is selected from: —CH₃, —CF₃, —OCH₃, —OCF₃ and NH₂.

In some embodiments, in compounds of Formula (I), R¹ is NH₂.

In some embodiments, in compounds of Formula (I), R¹ is selected from: —H, —CH₃, —CF₃, —F, —Br, —Cl, —OH, —OCH₃ and —OCF₃.

In some embodiments, in compounds of Formula (I), R¹ is selected from: —CH₃, —CF₃, —OCH₃ and —OCF₃.

In some embodiments, in compounds of Formula (I), R² is selected from: —H, —CH₃, —CF₃, —F, —Cl, —OCH₃ and —OCF₃.

In some embodiments, in compounds of Formula (I), R² is selected from: —CH₃, —CF₃, —F, —Cl, —OCH₃ and —OCF₃.

In some embodiments, in compounds of Formula (I), R² is selected from: —H, —F, —Br and —Cl.

In some embodiments, in compounds of Formula (I), R² is selected from: —F, —Br and —Cl.

In some embodiments, in compounds of Formula (I), R³ is selected from: —H, unsubstituted —C₁-C₆ alkyl, —C₁-C₆ haloalkyl, —C₁-C₆ hydroxyalkyl, —C₃-C₈ cycloalkyl, —(C₁-C₆ alkyl)-O—R⁵,

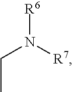

—CO₂R⁸, unsubstituted -aryl, -aminoaryl, -heteroaryl and —(C₁-C₆ alkyl)-aminoaryl.

In some embodiments, in compounds of Formula (I), R⁴ is selected from:

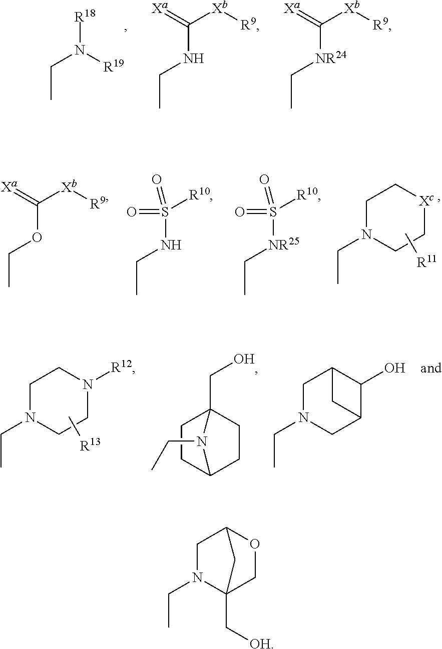

In some embodiments, in compounds of Formula (I), R⁵ is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aminoaryl.

In some embodiments, in compounds of Formula (I), R⁶ and R⁷ are each independently selected from: —H, unsubstituted —C₁-C₆ alkyl, —C₁-C₆ haloalkyl, —C₁-C₆ hydroxyalkyl, —C₃-C₈ cycloalkyl, —(C₁-C₆ alkyl)-O—R⁵, —C₃-C₈ heterocycloalkyl and —C(O)R¹⁷.

In some embodiments, in compounds of Formula (I), R⁸ is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (I), each R⁹ is independently selected from: —C₁-C₆ alkyl, —C₃-C₈ cycloalkyl, -aryl and —(C₁-C₆ alkyl)-aryl.

In some embodiments, in compounds of Formula (I), each R⁹ is independently selected from: —C₁-C₆ alkyl and —(C₁-C₆ alkyl)-aryl.

In some embodiments, in compounds of Formula (I), each R⁹ is independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aminoaryl.

In some embodiments, in compounds of Formula (I), each R¹⁰ is independently selected from: —C₁-C₆ alkyl, —C₃-C₈ cycloalkyl, —NR¹⁴R^{14′}, -aryl and —(C₁-C₆ alkyl)-aryl.

In some embodiments, in compounds of Formula (I), each R¹⁰ is independently selected from: —C₁-C₆ alkyl, —NR¹⁴R^{14′}, -aryl and —(C₁-C₆ alkyl)-aryl.

In some embodiments, in compounds of Formula (I), each R¹⁰ is independently selected from: unsubstituted —C₁-C₆ alkyl, —C₁-C₆ haloalkyl, —C₁-C₆ hydroxyalkyl, —C₁-C₆ aminoalkyl, —C₃-C₈ cycloalkyl, —NR¹⁴R^{14′}, unsubstituted -aryl, -aminoaryl, -heteroaryl and —(C₁-C₆ alkyl)-aryl.

In some embodiments, in compounds of Formula (I), each R^{10′} is independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aryl.

In some embodiments, in compounds of Formula (I), R¹¹ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (I), R¹² is selected from: —H, —C₁-C₆ alkyl, —CO₂R⁸, -aryl, —(C₁-C₆ alkyl)-aryl and —S(O)₂R¹⁶.

In some embodiments, in compounds of Formula (I), R¹² is selected from: —H, unsubstituted —C₁-C₆ alkyl, —C₁-C₆ haloalkyl, —C₁-C₆ hydroxyalkyl, —C₁-C₆ aminoalkyl, —CO₂R⁸, unsubstituted -aryl, -aminoaryl, -heteroaryl, —(C₁-C₆ alkyl)-aminoaryl, —S(O)₂R¹⁶ and

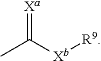

In some embodiments, in compounds of Formula (I), R¹³ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (I), R¹⁴ and R^{14′} are each independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (I), R¹⁶ is selected from: -aryl, -heteroaryl and —(C₁-C₆ alkyl)-aryl.

In some embodiments, in compounds of Formula (I), R¹⁶ is selected from: unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted aryl, -aminoaryl, -heteroaryl and —(C₁-C₆ alkyl)-aminoaryl.

In some embodiments, in compounds of Formula (I), R¹⁷ is selected from: unsubstituted C₁-C₆ alkyl, —C₁-C₆ hydroxyalkyl, —C₃-C₈ cycloalkyl, —C₃-C₈ heterocycloalkyl, —(C₁-C₆ alkyl)-C₃-C₈ heterocycloalkyl, unsubstituted aryl, -hydroxyaryl, -aminoaryl, -heteroaryl and —(C₁-C₆ alkyl)-aminoaryl.

In some embodiments, in compounds of Formula (I), R¹⁸ and R¹⁹ taken together with the N atom to which they are bonded form a 4-, 5-, 6- or 7-membered ring having 0 to 3 substituents selected from: halogen, unsubstituted C₁-C₆ alkyl, —C₁-C₆ haloalkyl, —C₁-C₆ hydroxyalkyl, —C₁-C₆ aminoalkyl, —C₃-C₈ cycloalkyl and —(C₁-C₆ alkyl)-O—R⁵.

In some embodiments, in compounds of Formula (I), X^{a} and X^{b} are each independently selected from: NH and O.

Combinations of any of the foregoing embodiments for compounds of Formula (I) are also contemplated and each combination forms a separate embodiment for the purposes of the present disclosure.

In certain embodiments, the compound of Formula (I) has Formula (Ia):

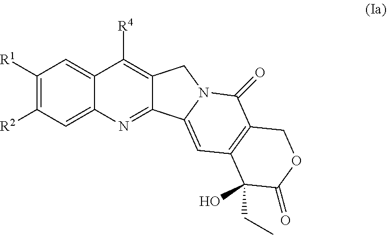

wherein: R¹, R², R⁴, R⁵, R⁸, R⁹, R¹⁰, R^{10′}, R¹¹, R¹², R¹³, R¹⁴, R^{14′}, R¹⁶, R¹⁸, R¹⁹, X^{a}, X^{b} and X^{c} are as defined for Formula (I).

In some embodiments, in compounds of Formula (Ia), R¹ is selected from: —CH₃, —CF₃, —OCH₃, —OCF₃ and —NH₂.

In some embodiments, in compounds of Formula (Ia), R² is selected from: —H, —CH₃, —CF₃, —F, —Cl, —OCH₃ and —OCF₃.

In some embodiments, in compounds of Formula (Ia), R² is selected from: —H, —F and —Cl.

In some embodiments, in compounds of Formula (Ia), R⁴ is selected from:

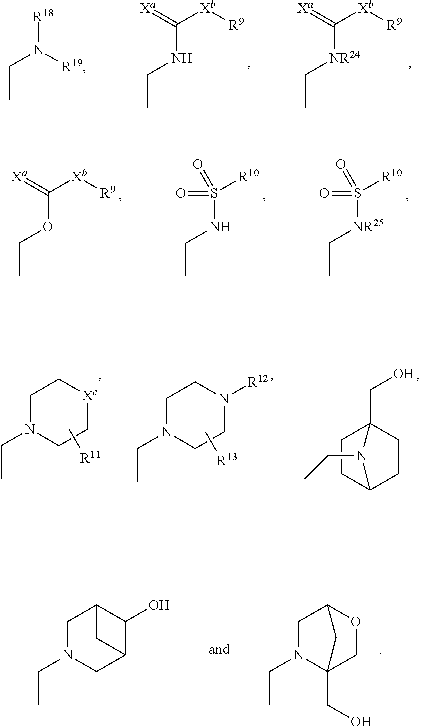

In some embodiments, in compounds of Formula (Ia), R⁵ is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aminoaryl.

In some embodiments, in compounds of Formula (Ia), R⁸ is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (Ia), each R⁹ is independently selected from: —C₁-C₆ alkyl, —C₃-C₈ cycloalkyl, -aryl and —(C₁-C₆ alkyl)-aryl.

In some embodiments, in compounds of Formula (Ia), each R⁹ is independently selected from: —C₁-C₆ alkyl and —(C₁-C₆ alkyl)-aryl.

In some embodiments, in compounds of Formula (Ia), each R⁹ is independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aminoaryl.

In some embodiments, in compounds of Formula (Ia), each R¹⁰ is independently selected from: —C₁-C₆ alkyl, —C₃-C₈ cycloalkyl, —NR¹⁴R^{14′}, -aryl and —(C₁-C₆ alkyl)-aryl.

In some embodiments, in compounds of Formula (Ia), each R¹⁰ is independently selected from: —C₁-C₆ alkyl, —NR¹⁴R^{14′}, -aryl and —(C₁-C₆ alkyl)-aryl.

In some embodiments, in compounds of Formula (Ia), each R¹⁰ is independently selected from: unsubstituted —C₁-C₆ alkyl, —C₁-C₆ haloalkyl, —C₁-C₆ hydroxyalkyl, —C₁-C₆ aminoalkyl, —C₃-C₈ cycloalkyl, —NR¹⁴R^{14′}, unsubstituted -aryl, -aminoaryl, -heteroaryl and —(C₁-C₆ alkyl)-aryl.

In some embodiments, in compounds of Formula (Ia), each R^{10′} is independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aryl.

In some embodiments, in compounds of Formula (Ia), R¹¹ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (Ia), R¹² is selected from: —H, —C₁-C₆ alkyl, —CO₂R⁸, -aryl and —(C₁-C₆ alkyl)-aryl and —S(O)₂R¹⁶.

In some embodiments, in compounds of Formula (Ia), R¹² is selected from: —H, unsubstituted —C₁-C₆ alkyl, —C₁-C₆ haloalkyl, —C₁-C₆ hydroxyalkyl, —C₁-C₆ aminoalkyl, —CO₂R⁸, unsubstituted -aryl, -aminoaryl, -heteroaryl, —(C₁-C₆ alkyl)-aminoaryl, —S(O)₂R¹⁶ and

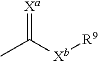

In some embodiments, in compounds of Formula (Ia), R¹³ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (Ia), R¹⁴ and R^{14′} are each independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (Ia), R¹⁶ is selected from: -aryl, -heteroaryl and —(C₁-C₆ alkyl)-aryl.

In some embodiments, in compounds of Formula (Ia), R¹⁶ is selected from: unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aminoaryl.

In some embodiments, in compounds of Formula (Ia), R¹⁷ is selected from: unsubstituted C₁-C₆ alkyl, —C₁-C₆ hydroxyalkyl, —C₃-C₈ cycloalkyl, —C₃-C₈ heterocycloalkyl, —(C₁-C₆ alkyl)-C₃-C₈ heterocycloalkyl, unsubstituted aryl, -hydroxyaryl, -aminoaryl, -heteroaryl and —(C₁-C₆ alkyl)-aminoaryl.

In some embodiments, in compounds of Formula (Ia), R¹⁸ and R¹⁹ taken together with the N atom to which they are bonded form a 4-, 5-, 6- or 7-membered ring having 0 to 3 substituents selected from: halogen, unsubstituted C₁-C₆ alkyl, —C₁-C₆ haloalkyl, —C₁-C₆ hydroxyalkyl, —C₁-C₆ aminoalkyl, —C₃-C₈ cycloalkyl and —(C₁-C₆ alkyl)-O—R⁵.

In some embodiments, in compounds of Formula (Ia), X^{a} and X^{b} are each independently selected from: NH and O.

Combinations of any of the foregoing embodiments for compounds of Formula (Ia) are also contemplated and each combination forms a separate embodiment for the purposes of the present disclosure.

In certain embodiments, the compound of Formula (I) has Formula (II):

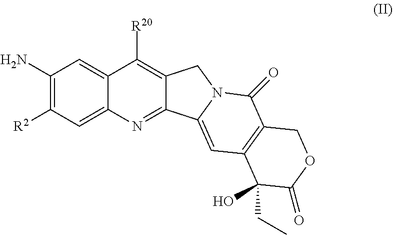

wherein:
   R² is selected from: —H, —CH₃, —CF₃, —F, —Br, —Cl, —OH, —OCH₃ and —OCF₃;
   R²⁰ is selected from: —H, —C₁-C₆ alkyl, —C₃-C₈ cycloalkyl, —(C₁-C₆ alkyl)-O—R⁵,

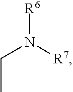

—CO₂R⁸, -aryl, -heteroaryl, —(C₁-C₆ alkyl)-aryl,

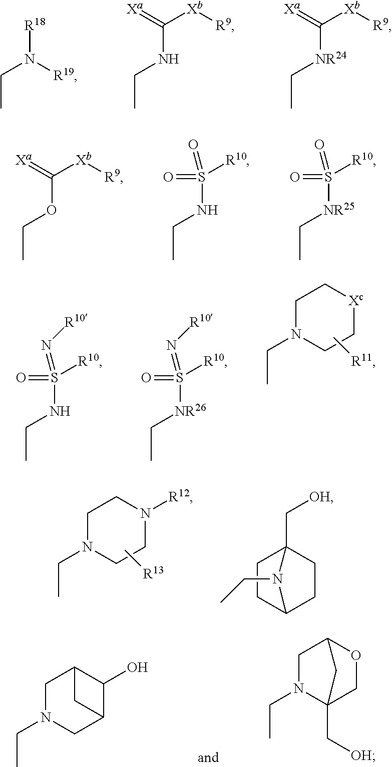

R⁵ is selected from: —H, —C₁-C₆ alkyl, —C₃-C₈ cycloalkyl, -aryl, -heteroaryl and —(C₁-C₆ alkyl)-aryl;
R⁶ and R⁷ are each independently selected from: —H, —C₁-C₆ alkyl, —C₃-C₈ cycloalkyl, —(C₁-C₆ alkyl)-O—R⁵, —C₃-C₈ heterocycloalkyl and —C(O)R¹⁷;
R⁸ is selected from: —H, —C₁-C₆ alkyl, —C₃-C₈ cycloalkyl and —C₃-C₈ heterocycloalkyl;
each R⁹ is independently selected from: —H, —C₁-C₆ alkyl, —C₃-C₈ cycloalkyl, -aryl, -heteroaryl and —(C₁-C₆ alkyl)-aryl;
each R¹⁰ is independently selected from: —C₁-C₆ alkyl, —C₃-C₈ cycloalkyl, —NR¹⁴R^{14′}, -aryl, -heteroaryl and —(C₁-C₆ alkyl)-aryl;
each R^{10′} is independently selected from: —H, —C₁-C₆ alkyl, —C₃-C₈ cycloalkyl, -aryl, -heteroaryl, and —(C₁-C₆ alkyl)-aryl;
R¹¹ is selected from: —H and —C₁-C₆ alkyl;
R¹² is selected from: —H, —C₁-C₆ alkyl, —CO₂R⁸, -aryl, -heteroaryl, —(C₁-C₆ alkyl)-aryl, —S(O)₂R¹⁶ and

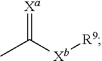

R¹³ is selected from: —H and —C₁-C₆ alkyl;
R¹⁴ and R^{14′} are each independently selected from: —H, C₁-C₆ alkyl, —C₃-C₈ cycloalkyl and —C₃-C₈ heterocycloalkyl;
R¹⁶ is selected from: —C₁-C₆ alkyl, —C₃-C₈ cycloalkyl, -aryl, -heteroaryl and —(C₁-C₆ alkyl)-aryl;
R¹⁷ is selected from: —C₁-C₆ alkyl, —C₃-C₈ cycloalkyl, —C₃-C₈ heterocycloalkyl, —(C₁-C₆ alkyl)-C₃-C₈ heterocycloalkyl, -aryl, -heteroaryl and —(C₁-C₆ alkyl)-aryl;
R¹⁸ and R¹⁹ taken together with the N atom to which they are bonded form a 4-, 5-, 6-, or 7-membered ring having 0 to 3 substituents selected from: halogen, —C₁-C₆ alkyl, —C₃-C₈ cycloalkyl and —(C₁-C₆ alkyl)-O—R⁵;
R²⁴, R²⁵ and R²⁶ are each —C₁-C₆ alkyl;
X^{a} and X^{b} are each independently selected from: NH, O and S, and
X^{c} is selected from: O, S and S(O)₂,
with the proviso that the compound is other than (S)-9-amino-11-butyl-4-ethyl-4-hydroxy-1,12-dihydro-14H-pyrano[3′,4′:6,7]indolizino[1,2-b]quinoline-3,14(4H)-dione.

In some embodiments, in compounds of Formula (II), R² is selected from: —CH₃, —CF₃, —F, —Br, —Cl, —OH, —OCH₃ and —OCF₃.

In some embodiments, in compounds of Formula (II), R² is selected from: —CH₃, —CF₃, —F, —Cl, —OCH₃ and —OCF₃.

In some embodiments, in compounds of Formula (II), R² is selected from F and Cl.

In some embodiments, in compounds of Formula (II), R²⁰ is selected from: —H, —C₁-C₆ alkyl, —(C₁-C₆ alkyl)-O—R⁵,

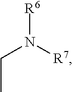

—(C₁-C₆ alkyl)-aryl,

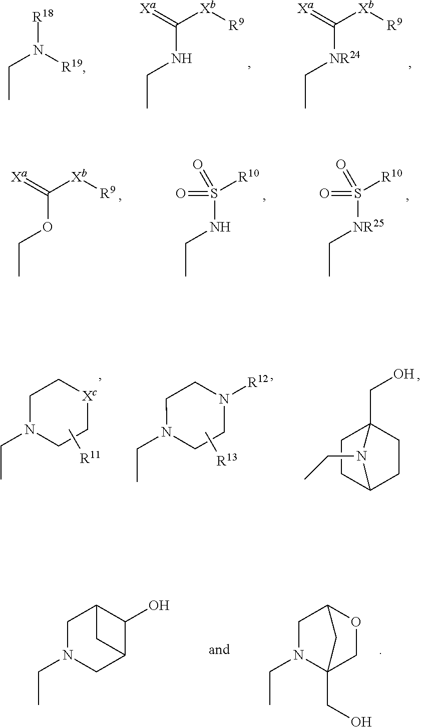

In some embodiments, in compounds of Formula (II), R²⁰ is selected from: —H, —C₁-C₆ alkyl —(C₁-C₆ alkyl)-O—R⁵

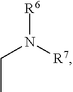

—(C₁-C₆ alkyl)-aryl,

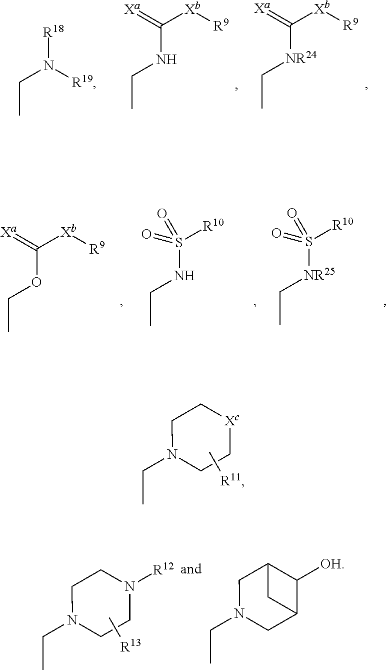

In some embodiments, in compounds of Formula (II), R²⁰ is selected from: —H, —C₁-C₆ alkyl, —(C₁-C₆ alkyl)-O—R⁵,

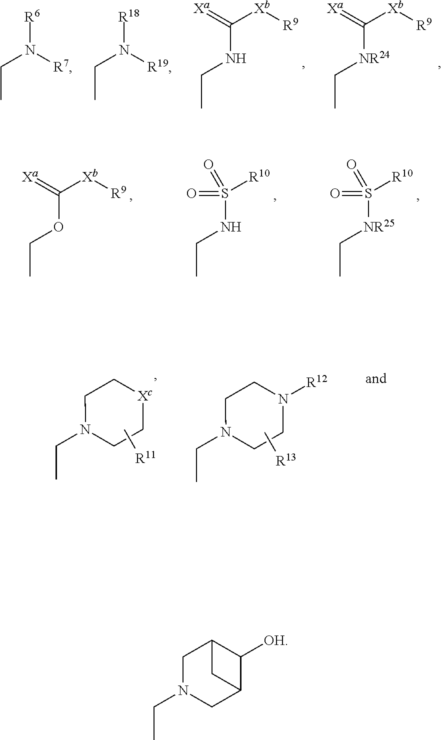

In some embodiments, in compounds of Formula (II), R²⁰ is selected from: —H, unsubstituted —C₁-C₆ alkyl, —C₁-C₆ haloalkyl, —C₁-C₆ hydroxyalkyl, —C₃-C₈ cycloalkyl, —(C₁-C₆ alkyl)-O—R⁵,

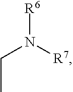

—CO₂R⁸, unsubstituted aryl, -aminoaryl, -heteroaryl, —(C₁-C₆ alkyl)-aminoaryl,

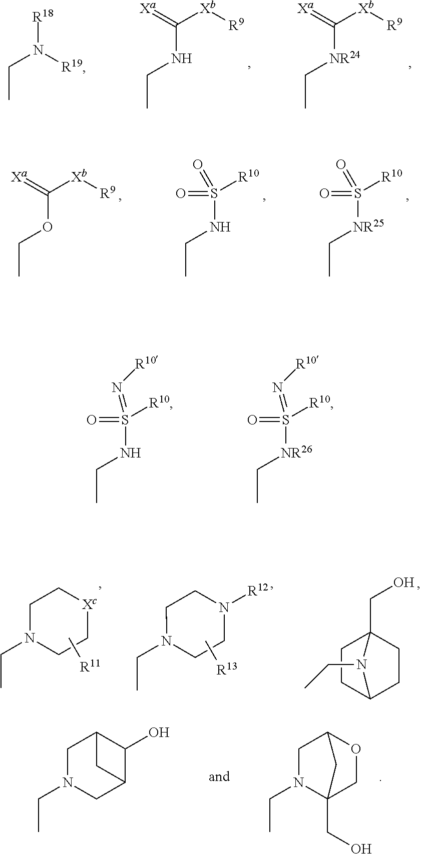

In some embodiments, in compounds of Formula (II), R² is selected from: —CH₃, —CF₃, —F, —Br, —Cl, —OH, —OCH₃ and —OCF₃, and R²⁰ is selected from: —H, —C₁-C₆ alkyl, —(C₁-C₆ alkyl)-O—R⁵,

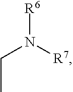

—(C₁-C₆ alkyl)-aryl,

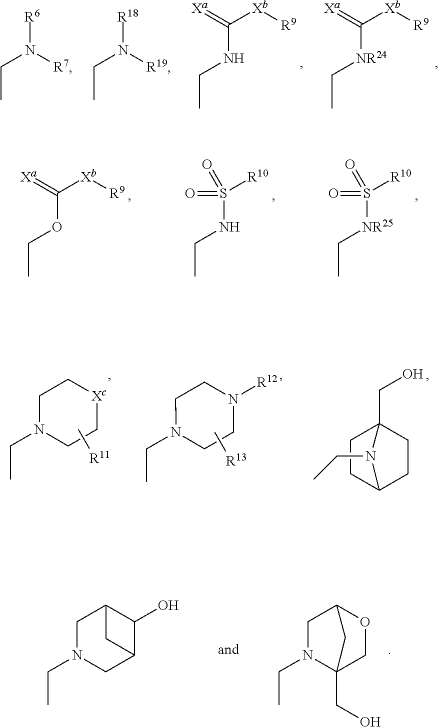

In some embodiments, in compounds of Formula (II), R² is selected from: —CH₃, —CF₃, —F, —Br, —Cl, —OH, —OCH₃ and —OCF₃, and R²⁰ is selected from: —H, —C₁-C₆ alkyl, —(C₁-C₆ alkyl)-O—R⁵,

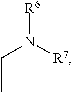

—(C₁-C₆ alkyl)-aryl,

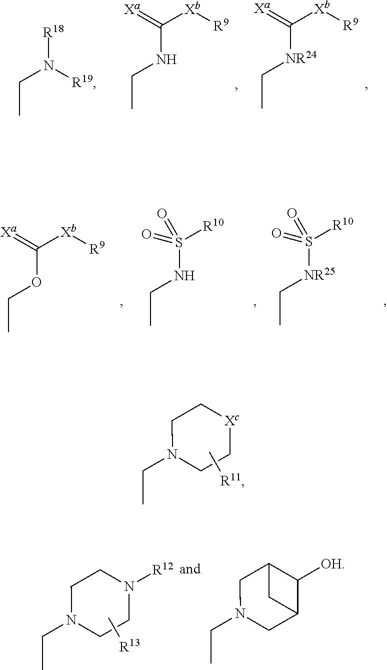

In some embodiments, in compounds of Formula (II), R² is selected from: —CH₃, —CF₃, —F, —Br, —Cl, —OH, —OCH₃ and —OCF₃, and R²⁰ is selected from: —H, —C₁-C₆ alkyl, —(C₁-C₆ alkyl)-O—R⁵,

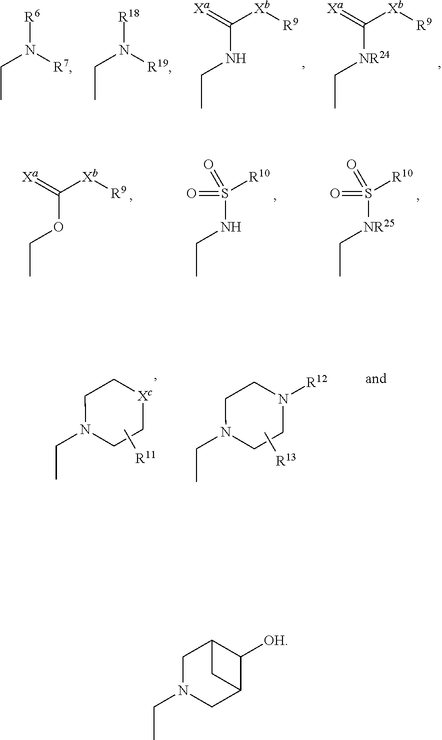

In some embodiments, in compounds of Formula (II), R⁵ is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aminoaryl.

In some embodiments, in compounds of Formula (II), R⁶ and R⁷ are each independently selected from: —H, —C₁-C₆ alkyl, —C₃-C₈ cycloalkyl and —C(O)R¹⁷.

In some embodiments, in compounds of Formula (II), R⁶ is H, and R⁷ is selected from: —H, —C₁-C₆ alkyl, —C₃-C₈ cycloalkyl, —(C₁-C₆ alkyl)-O—R⁵, —C₃-C₈ heterocycloalkyl and —C(O)R¹⁷.

In some embodiments, in compounds of Formula (II), R⁶ is H, and R⁷ is selected from: —H, —C₁-C₆ alkyl, —C₃-C₈ cycloalkyl and —C(O)R¹⁷.

In some embodiments, in compounds of Formula (II), R⁶ and R⁷ are each independently selected from: —H, unsubstituted —C₁-C₆ alkyl, —C₁-C₆ haloalkyl, —C₁-C₆ hydroxyalkyl, —C₃-C₈ cycloalkyl, —(C₁-C₆ alkyl)-O—R⁵, —C₃-C₈ heterocycloalkyl and —C(O)R¹⁷.

In some embodiments, in compounds of Formula (II), R⁸ is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (II), each R⁹ is independently selected from: —C₁-C₆ alkyl, —C₃-C₈ cycloalkyl, -aryl and —(C₁-C₆ alkyl)-aryl.

In some embodiments, in compounds of Formula (II), each R⁹ is independently selected from: —C₁-C₆ alkyl and —(C₁-C₆ alkyl)-aryl.

In some embodiments, in compounds of Formula (II), each R⁹ is independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aminoaryl.

In some embodiments, in compounds of Formula (II), each R¹⁰ is independently selected from: —C₁-C₆ alkyl, —C₃-C₈ cycloalkyl, —NR¹⁴R^{14′}, -aryl and —(C₁-C₆ alkyl)-aryl.

In some embodiments, in compounds of Formula (II), each R¹⁰ is independently selected from: —C₁-C₆ alkyl, —NR¹⁴R^{14′}, -aryl and —(C₁-C₆ alkyl)-aryl.

In some embodiments, in compounds of Formula (II), each R¹⁰ is independently selected from: unsubstituted —C₁-C₆ alkyl, —C₁-C₆ haloalkyl, —C₁-C₆ hydroxyalkyl, —C₁-C₆ aminoalkyl, —C₃-C₈ cycloalkyl, —NR¹⁴R^{14′}, unsubstituted -aryl, -aminoaryl, -heteroaryl and —(C₁-C₆ alkyl)-aryl.

In some embodiments, in compounds of Formula (II), each R^{10′} is independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aryl.

In some embodiments, in compounds of Formula (II), R¹¹ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (II), R¹² is selected from: —H, —C₁-C₆ alkyl, —CO₂R⁸, -aryl, —(C₁-C₆ alkyl)-aryl and —S(O)₂R¹⁶.

In some embodiments, in compounds of Formula (II), R¹² is selected from: —H, unsubstituted —C₁-C₆ alkyl, —C₁-C₆ haloalkyl, —C₁-C₆ hydroxyalkyl, —C₁-C₆ aminoalkyl, —CO₂R⁸, unsubstituted -aryl, -aminoaryl, -heteroaryl, —(C₁-C₆ alkyl)-aminoaryl, —S(O)₂R¹⁶ and

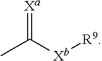

In some embodiments, in compounds of Formula (II), R¹³ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (II), R¹⁴ and R^{14′} are each independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (II), R¹⁶ is selected from: -aryl, -heteroaryl and —(C₁-C₆ alkyl)-aryl.

In some embodiments, in compounds of Formula (II), R¹⁶ is selected from: unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aminoaryl.

In some embodiments, in compounds of Formula (II), R¹⁷ is —C₁-C₆ alkyl.

In some embodiments, in compounds of Formula (II), R¹⁷ is selected from: unsubstituted C₁-C₆ alkyl, —C₁-C₆ hydroxyalkyl, —C₃-C₈ cycloalkyl, —C₃-C₈ heterocycloalkyl, —(C₁-C₆ alkyl)-C₃-C₈ heterocycloalkyl, unsubstituted aryl, -hydroxyaryl, -aminoaryl, -heteroaryl and —(C₁-C₆ alkyl)-aminoaryl.

In some embodiments, in compounds of Formula (II), R¹⁸ and R¹⁹ taken together with the N atom to which they are bonded form a 4-, 5-, 6- or 7-membered ring having 0 to 3 substituents selected from: halogen, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —(C₁-C₆ alkyl)-O—R⁵.

In some embodiments, in compounds of Formula (II), X^{a} and X^{b} are each independently selected from: NH and O.

Combinations of any of the foregoing embodiments for compounds of Formula (II) are also contemplated and each combination forms a separate embodiment for the purposes of the present disclosure.

In certain embodiments, the compound of Formula (I) has Formula (IIa):

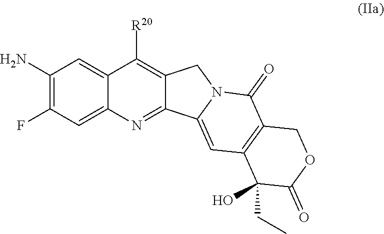

wherein: R²⁰, R⁵, R⁶, R⁷, R⁸, R⁹, R¹⁰, R^{10′}, R¹¹, R¹², R¹³, R¹⁴, R^{14′}, R¹⁶, R¹⁷, R¹⁸, R¹⁹, X^{a}, X^{b} and X^{c} are as defined for Formula (II).

In some embodiments, in compounds of Formula (IIa), R²⁰ is selected from: —H, —C₁-C₆ alkyl, —(C₁-C₆ alkyl)-O—R⁵,

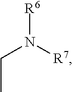

—(C₁-C₆ alkyl)-aryl,

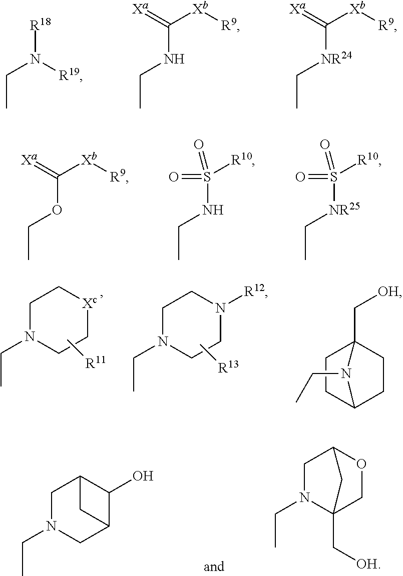

In some embodiments, in compounds of Formula (IIa), R²⁰ is selected from: —H, —C₁-C₆ alkyl, —(C₁-C₆ alkyl)-O—R⁵,

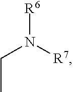

—(C₁-C₆ alkyl)-aryl

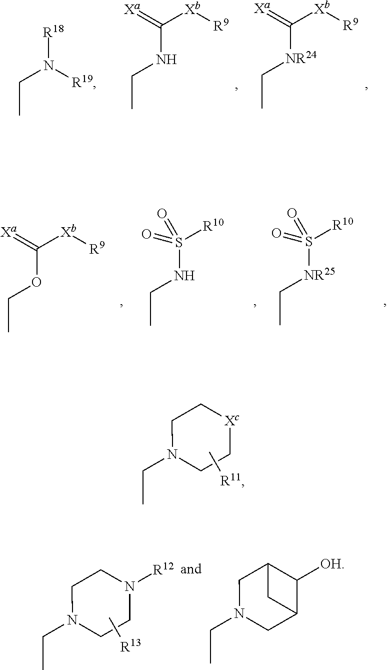

In some embodiments, in compounds of Formula (IIa), R²⁰ is selected from: —H, —C₁-C₆ alkyl, —(C₁-C₆ alkyl)-O—R⁵,

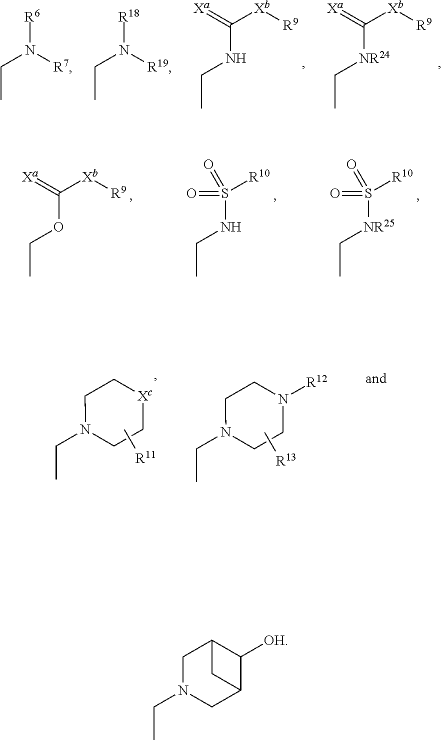

In some embodiments, in compounds of Formula (IIa), R²⁰ is selected from: —H, unsubstituted —C₁-C₆ alkyl, —C₁-C₆ haloalkyl, —C₁-C₆ hydroxyalkyl, —C₃-C₈ cycloalkyl, —(C₁-C₆ alkyl)-O—R⁵,

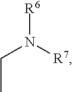

—CO₂R⁸, unsubstituted aryl, -aminoaryl, -heteroaryl, —(C₁-C₆ alkyl)-aminoaryl,

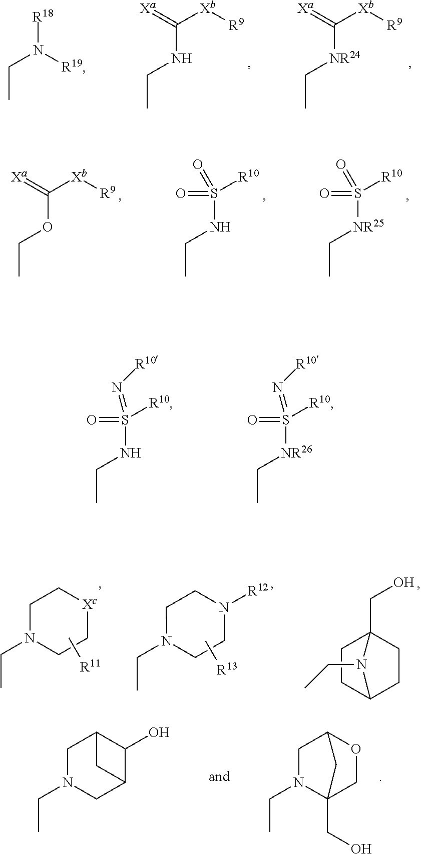

In some embodiments, in compounds of Formula (IIa), R⁵ is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aminoaryl.

In some embodiments, in compounds of Formula (IIa), R⁶ and R⁷ are each independently selected from: —H, —C₁-C₆ alkyl, —C₃-C₈ cycloalkyl and —C(O)R¹⁷.

In some embodiments, in compounds of Formula (IIa), R⁶ is H, and R⁷ is selected from: —H, —C₁-C₆ alkyl, —C₃-C₈ cycloalkyl, —(C₁-C₆ alkyl)-O—R⁵, —C₃-C₈ heterocycloalkyl and —C(O)R¹⁷.

In some embodiments, in compounds of Formula (IIa), R⁶ is H, and R⁷ is selected from: —H, —C₁-C₆ alkyl, —C₃-C₈ cycloalkyl and —C(O)R¹⁷.

In some embodiments, in compounds of Formula (IIa), R⁶ and R⁷ are each independently selected from: —H, unsubstituted —C₁-C₆ alkyl, —C₁-C₆ haloalkyl, —C₁-C₆ hydroxyalkyl, —C₃-C₈ cycloalkyl, —(C₁-C₆ alkyl)-O—R⁵, —C₃-C₈ heterocycloalkyl and —C(O)R¹⁷.

In some embodiments, in compounds of Formula (IIa), R⁸ is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (IIa), each R⁹ is independently selected from: —C₁-C₆ alkyl, —C₃-C₈ cycloalkyl, -aryl and —(C₁-C₆ alkyl)-aryl.

In some embodiments, in compounds of Formula (IIa), each R⁹ is independently selected from: —C₁-C₆ alkyl and —(C₁-C₆ alkyl)-aryl.

In some embodiments, in compounds of Formula (IIa), each R⁹ is independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aminoaryl.

In some embodiments, in compounds of Formula (IIa), each R¹⁰ is independently selected from: —C₁-C₆ alkyl, —C₃-C₈ cycloalkyl, —NR¹⁴R^{14′}, -aryl and —(C₁-C₆ alkyl)-aryl.

In some embodiments, in compounds of Formula (IIa), each R¹⁰ is independently selected from: —C₁-C₆ alkyl, —NR¹⁴R^{14′}, -aryl and —(C₁-C₆ alkyl)-aryl.

In some embodiments, in compounds of Formula (IIa), each R¹⁰ is independently selected from: unsubstituted —C₁-C₆ alkyl, —C₁-C₆ haloalkyl, —C₁-C₆ hydroxyalkyl, —C₁-C₆ aminoalkyl, —C₃-C₈ cycloalkyl, —NR¹⁴R^{14′}, unsubstituted -aryl, -aminoaryl, -heteroaryl and —(C₁-C₆ alkyl)-aryl.

In some embodiments, in compounds of Formula (IIa), each R^{10′} is independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aryl.

In some embodiments, in compounds of Formula (IIa), R¹¹ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (IIa), R¹² is selected from: —H, —C₁-C₆ alkyl, —CO₂R⁸, -aryl, —(C₁-C₆ alkyl)-aryl and —S(O)₂R¹⁶.

In some embodiments, in compounds of Formula (IIa), R¹² is selected from: —H, unsubstituted —C₁-C₆ alkyl, —C₁-C₆ haloalkyl, —C₁-C₆ hydroxyalkyl, —C₁-C₆ aminoalkyl, —CO₂R⁸, unsubstituted -aryl, -aminoaryl, -heteroaryl, —(C₁-C₆ alkyl)-aminoaryl, —S(O)₂R¹⁶ and

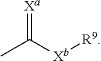

In some embodiments, in compounds of Formula (IIa), R¹³ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (IIa), R¹⁴ and R^{14′} are each independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (IIa), R¹⁶ is selected from: -aryl, -heteroaryl and —(C₁-C₆ alkyl)-aryl.

In some embodiments, in compounds of Formula (IIa), R¹⁶ is selected from: unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aminoaryl.

In some embodiments, in compounds of Formula (IIa), R¹⁷ is —C₁-C₆ alkyl.

In some embodiments, in compounds of Formula (IIa), R¹⁷ is selected from: unsubstituted C₁-C₆ alkyl. —C₁-C₆ hydroxyalkyl, —C₃-C₈ cycloalkyl, —C₃-C₈ heterocycloalkyl, —(C₁-C₆ alkyl)-C₃-C₈ heterocycloalkyl, unsubstituted -aryl, -hydroxyaryl, -aminoaryl, -heteroaryl and —(C₁-C₆ alkyl)-aminoaryl.

In some embodiments, in compounds of Formula (IIa), R′⁸ and R¹⁹ taken together with the N atom to which they are bonded form a 4-, 5-, 6- or 7-membered ring having 0 to 3 substituents selected from: halogen, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —(C₁-C₆ alkyl)-O—R⁵.

In some embodiments, in compounds of Formula (IIa), X^{a} and X^{b} are each independently selected from: NH and O.

Combinations of any of the foregoing embodiments for compounds of Formula (IIa) are also contemplated and each combination forms a separate embodiment for the purposes of the present disclosure.

In certain embodiments, the compound of Formula (I) has Formula (III):

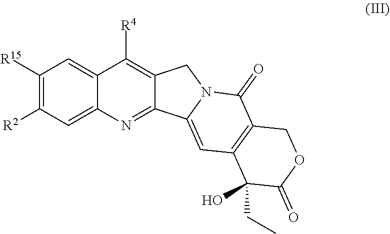

wherein:
   R² is selected from: —H, —CH₃, —CF₃, —F, —Br, —Cl, —OH, —OCH₃ and —OCF₃;
   R¹⁵ is selected from: —H, —CH₃, —CHF₂, —CF₃, —F, —Br, —Cl, —OH, —OCH₃ and —OCF₃;
   R⁴ is selected from:

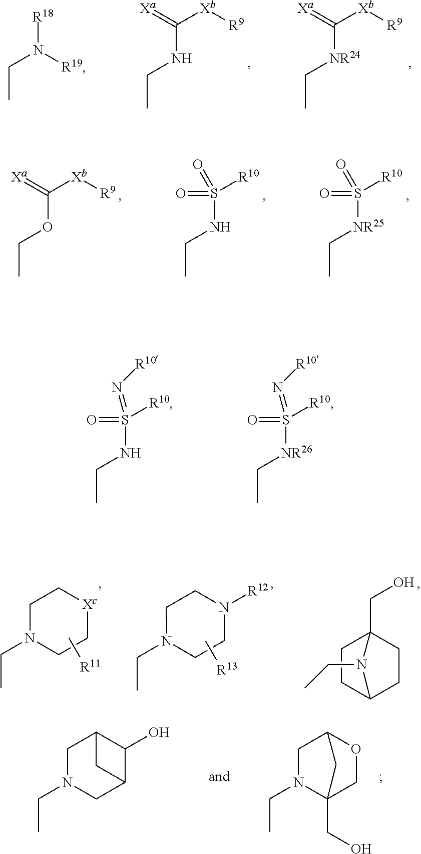

R⁵ is selected from: —H, —C₁-C₆ alkyl, —C₃-C₈ cycloalkyl, -aryl, -heteroaryl and —(C₁-C₆ alkyl)-aryl;
R⁸ is selected from: —H, —C₁-C₆ alkyl, —C₃-C₈ cycloalkyl and —C₃-C₈ heterocycloalkyl;
each R⁹ is independently selected from: —H, —C₁-C₆ alkyl, —C₃-C₈ cycloalkyl, -aryl, -heteroaryl and —(C₁-C₆ alkyl)-aryl;
each R¹⁰ is independently selected from: —C₁-C₆ alkyl, —C₃-C₈ cycloalkyl, —NR¹⁴R^{14′}, -aryl, -heteroaryl and —(C₁-C₆ alkyl)-aryl;
each R^{10′} is independently selected from: —H, —C₁-C₆ alkyl, —C₃-C₈ cycloalkyl, -aryl, -heteroaryl and —(C₁-C₆ alkyl)-aryl;
R¹¹ is selected from: —H and —C₁-C₆ alkyl;
R¹² is selected from: —H, —C₁-C₆ alkyl, —CO₂R⁸, -aryl, -heteroaryl, —(C₁-C₆ alkyl)-aryl, —S(O)₂R¹⁶ and

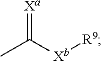

R¹³ is selected from: —H and —C₁-C₆ alkyl;
R¹⁴ and R^{14′} are each independently selected from: —H, C₁-C₆ alkyl, —C₃-C₈ cycloalkyl and —C₃-C₈ heterocycloalkyl;
R¹⁶ is selected from: —C₁-C₆ alkyl, —C₃-C₈ cycloalkyl, -aryl, -heteroaryl and —(C₁-C₆ alkyl)-aryl;
R¹⁸ and R¹⁹ taken together with the N atom to which they are bonded form a 4-, 5-, 6-, or 7-membered ring having 0 to 3 substituents selected from: halogen, —C₁-C₆ alkyl, —C₃-C₈ cycloalkyl and —(C₁-C₆ alkyl)-O—R⁵;
R²⁴, R²⁵ and R²⁶ are each —C₁-C₆ alkyl;
X^{a} and X^{b} are each independently selected from: NH, O and S, and
X^{c} is selected from: O, S and S(O)₂.

In some embodiments, in compounds of Formula (III), R² is selected from: —H, —CH₃, —CF₃, —F, —Cl, —OCH₃ and —OCF₃.

In some embodiments, in compounds of Formula (III), R² is selected from: —H, —F and —Cl.

In some embodiments, in compounds of Formula (III), R¹⁵ is selected from: —CH₃, —CF₃, —OCH₃ and —OCF₃.

In some embodiments, in compounds of Formula (III), R¹⁵ is selected from: —CH₃ and —OCH₃.

In some embodiments, in compounds of Formula (III), R² is selected from: —H, —F and —Cl, and R¹⁵ is selected from: —CH₃, —CF₃, —OCH₃ and —OCF₃.

In some embodiments, in compounds of Formula (III), R² is selected from: —H, —F and —Cl, and R¹⁵ is selected from: —CH₃ and —OCH₃.

In some embodiments, in compounds of Formula (III), R⁴ is selected from

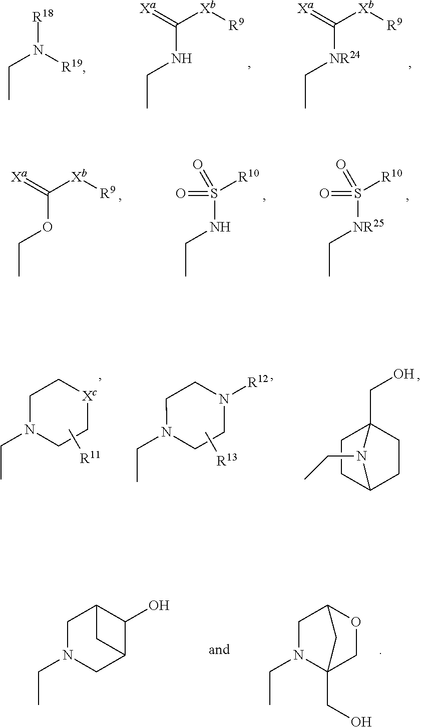

In some embodiments, in compounds of Formula (III), R⁵ is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aminoaryl.

In some embodiments, in compounds of Formula (III), R⁸ is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (III), each R⁹ is independently selected from: —C₁-C₆ alkyl, —C₃-C₈ cycloalkyl, -aryl and —(C₁-C₆ alkyl)-aryl.

In some embodiments, in compounds of Formula (III), each R⁹ is independently selected from: —C₁-C₆ alkyl and —(C₁-C₆ alkyl)-aryl.

In some embodiments, in compounds of Formula (III), each R⁹ is independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aminoaryl.

In some embodiments, in compounds of Formula (III), each R¹⁰ is independently selected from: —C₁-C₆ alkyl, —C₃-C₈ cycloalkyl, —NR¹⁴R^{14′}, -aryl and —(C₁-C₆ alkyl)-aryl.

In some embodiments, in compounds of Formula (III), each R¹⁰ is independently selected from: —C₁-C₆ alkyl, —NR¹⁴R^{14′}, -aryl and —(C₁-C₆ alkyl)-aryl.

In some embodiments, in compounds of Formula (III), each R¹⁰ is independently selected from: unsubstituted —C₁-C₆ alkyl, —C₁-C₆ haloalkyl, —C₁-C₆ hydroxyalkyl, —C₁-C₆ aminoalkyl, —C₃-C₈ cycloalkyl, —NR¹⁴R^{14′}, unsubstituted -aryl, -aminoaryl, -heteroaryl and —(C₁-C₆ alkyl)-aryl.

In some embodiments, in compounds of Formula (III), each R^{0′} is independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aryl.

In some embodiments, in compounds of Formula (III), R¹¹ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (III), R¹² is selected from: —H, —C₁-C₆ alkyl, —CO₂R⁸, -aryl, —(C₁-C₆ alkyl)-aryl and —S(O)₂R¹⁶.

In some embodiments, in compounds of Formula (III), R¹² is selected from: —H, unsubstituted —C₁-C₆ alkyl, —C₁-C₆ haloalkyl, —C₁-C₆ hydroxyalkyl, —C₁-C₆ aminoalkyl, —CO₂R⁸, unsubstituted -aryl, -aminoaryl, -heteroaryl, —(C₁-C₆ alkyl)-aminoaryl, —S(O)₂R¹⁶ and

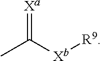

In some embodiments, in compounds of Formula (III), R¹³ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (III), R¹⁴ and R^{14′} are each independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (III), R¹⁶ is selected from: -aryl, -heteroaryl and —(C₁-C₆ alkyl)-aryl.

In some embodiments, in compounds of Formula (III), R¹⁶ is selected from: unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aminoaryl.

In some embodiments, in compounds of Formula (III), R¹⁸ and R¹⁹ taken together with the N atom to which they are bonded form a 4-, 5-, 6- or 7-membered ring having 0 to 3 substituents selected from: halogen, unsubstituted C₁-C₆ alkyl, —C₁-C₆ haloalkyl, —C₁-C₆ hydroxyalkyl, —C₁-C₆ aminoalkyl, —C₃-C₈ cycloalkyl and —(C₁-C₆ alkyl)-O—R⁵.

In some embodiments, in compounds of Formula (III), X^{a} and X^{b} are each independently selected from: NH and O.

Combinations of any of the foregoing embodiments for compounds of Formula (III) are also contemplated and each combination forms a separate embodiment for the purposes of the present disclosure.

In certain embodiments, the compound of Formula (I) has Formula (IIIa) or (IIIb):

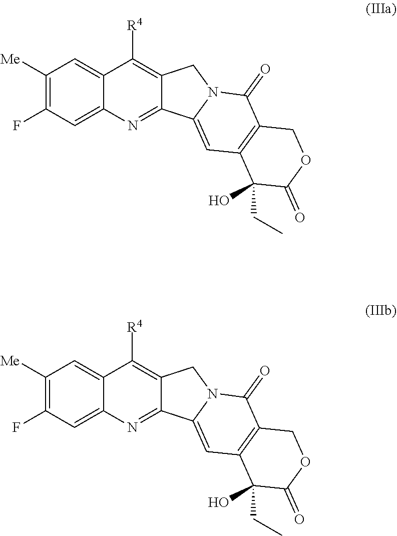

wherein: R⁴, R⁵, R⁸, R⁹, R¹⁰, R¹⁰, R¹¹, R¹², R¹³, R¹⁴, R^{14′}, R¹⁶, R¹⁸, R¹⁹, X^{a}, X^{b} and X^{c} are as defined in Formula (III).

In some embodiments, in compounds of Formula (IIIa) or Formula (IIIb), R⁴ is selected from:

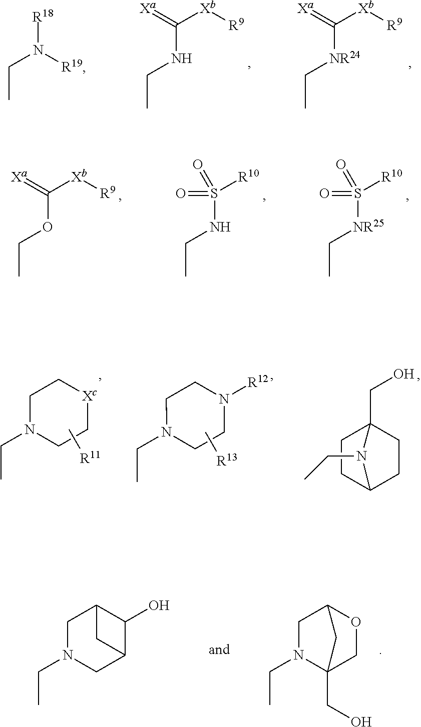

In some embodiments, in compounds of Formula (IIIa) or Formula (IIIb), R⁵ is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aminoaryl.

In some embodiments, in compounds of Formula (IIIa) or Formula (IIIb), R⁸ is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (IIIa) or Formula (IIIb), each R⁹ is independently selected from: —C₁-C₆ alkyl, —C₃-C₈ cycloalkyl, -aryl and —(C₁-C₆ alkyl)-aryl.

In some embodiments, in compounds of Formula (IIIa) or Formula (IIIb), each R⁹ is independently selected from: —C₁-C₆ alkyl and —(C₁-C₆ alkyl)-aryl.

In some embodiments, in compounds of Formula (IIIa) or Formula (IIIb), each R⁹ is independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aminoaryl.

In some embodiments, in compounds of Formula (IIIa) or Formula (IIIb), each R¹⁰ is independently selected from: —C₁-C₆ alkyl, —C₃-C₈ cycloalkyl, —NR¹⁴R^{14′}, -aryl and —(C₁-C₆ alkyl)-aryl.

In some embodiments, in compounds of Formula (IIIa) or Formula (IIIb), each R¹⁰ is independently selected from: —C₁-C₆ alkyl, —NR¹⁴R^{14′}, -aryl and —(C₁-C₆ alkyl)-aryl.

In some embodiments, in compounds of Formula (IIIa) or Formula (IIIb), each R¹⁰ is independently selected from: unsubstituted —C₁-C₆ alkyl, —C₁-C₆ haloalkyl, —C₁-C₆ hydroxyalkyl, —C₁-C₆ aminoalkyl, —C₃-C₈ cycloalkyl, —NR¹⁴R^{14′} unsubstituted -aryl, -aminoaryl, -heteroaryl and —(C₁-C₆ alkyl)-aryl.

In some embodiments, in compounds of Formula (IIIa) or Formula (IIIb), each R^{10′} is independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aryl.

In some embodiments, in compounds of Formula (IIIa) or Formula (IIIb), R¹¹ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (IIIa) or Formula (IIIb), R¹² is selected from: —H, —C₁-C₆ alkyl, —CO₂R⁸, -aryl, —(C₁-C₆ alkyl)-aryl and —S(O)₂R¹⁶.

In some embodiments, in compounds of Formula (IIIa) or Formula (IIIb), R¹² is selected from: —H, unsubstituted —C₁-C₆ alkyl, —C₁-C₆ haloalkyl, —C₁-C₆ hydroxyalkyl, —C₁-C₆ aminoalkyl, —CO₂R⁸, unsubstituted -aryl, -aminoaryl, -heteroaryl, —(C₁-C₆ alkyl)-aminoaryl, —S(O)₂R¹⁶ and

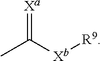

In some embodiments, in compounds of Formula (IIIa) or Formula (IIIb), R¹³ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (IIIa) or Formula (IIIb), R¹⁴ and R^{14′} are each independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (IIIa) or Formula (IIIb), R¹⁶ is selected from: -aryl, -heteroaryl and —(C₁-C₆ alkyl)-aryl.

In some embodiments, in compounds of Formula (IIIa) or Formula (IIIb), R¹⁶ is selected from: unsubstituted —C₁-C₆ alkyl, —C₁-C₆ haloalkyl, —C₁-C₆ hydroxyalkyl, —C₁-C₆ aminoalkyl, —C₃-C₈cycloalkyl, unsubstituted -aryl, -aminoaryl, -heteroaryl and —(C₁-C₆ alkyl)-aminoaryl.

In some embodiments, in compounds of Formula (IIIa) or Formula (IIIb), R¹⁸ and R¹⁹ taken together with the N atom to which they are bonded form a 4-, 5-, 6- or 7-membered ring having 0 to 3 substituents selected from: halogen, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —(C₁-C₆ alkyl)-O—R⁵.

In some embodiments, in compounds of Formula (IIIa) or Formula (IIIb), X^{a} and X^{b} are each independently selected from: NH and O.

Combinations of any of the foregoing embodiments for each of compounds of Formula (IIIa) and Formula (IIIb) are also contemplated and each combination forms a separate embodiment for the purposes of the present disclosure.

As described above, certain compounds of Formulae (I), (Ia), (II), (IIa), (III), (IIIa) or (IIIb) may include one or more free amino, hydroxy, carbonyl (for example, keto or aldehyde) or carboxylic acid groups. Also encompassed by the present disclosure are protected versions of the compounds of Formulae (I), (Ia), (II), (IIa), (III), (IIIa) or (IIIb) in which an otherwise free amino, hydroxy, carbonyl (for example, keto or aldehyde) or carboxylic acid group is protected with an appropriate protecting group. The term “protecting group” refers to a chemical group that, when attached to a potentially reactive functional group, masks, reduces or prevents the reactivity of the functional group. Typically, a protecting group can be selectively removed as desired during the course of a synthesis.

Protecting groups are well-known in the art and various examples are described, for example, in “Protective Groups in Organic Chemistry” (Greene, W. & Wuts, P. G. M., 2006, John Wiley & Sons). Examples of amino protecting groups include, but are not limited to, formyl, acetyl, trifluoroacetyl, benzyl (Bn), benzoyl (Bz), benzyloxycarbonyl (CBZ), tert-butoxycarbonyl (Boc), trimethylsilyl (TMS), 2-trimethylsilyl-ethanesulfonyl (TES), trityl, substituted trityl, tosyl, phthalimide, alloxycarbonyl (Alloc) and 9-fluorenylmethyloxycarbonyl (FMOC). Examples of hydroxy protecting groups include, but are not limited to, acetyl, benzyl (Bn), t-butyl, benzoyl (Bz), β-methoxyethoxymethyl ether (MEM), dimethoxytrityl (DMT), methoxymethyl ether (MOM), methoxytrityl [(4-methoxyphenyl)diphenylmethyl] (MMT), p-methoxybenzyl ether (PMB), p-methoxyphenyl ether (PMP), methylthiomethyl ether, pivaloyl (Piv), tetrahydropyranyl (THP), tetrahydrofuran (THF), trityl, trimethylsilyl (TMS), tert-butyldimethylsilyl (TBDMS or TBS), tri-iso-propylsilyloxymethyl (TOM), and triisopropylsilyl (TIPS). Examples of carbonyl protecting groups include, but are not limited to, acetals, hemi-acetals and ketals. Examples of carboxylic acid protecting groups include, but are not limited to, methyl esters, benzyl esters, tert-butyl esters, silyl esters, orthoesters and oxazoline.

Certain embodiments of the present disclosure relate to protected compounds of Formula (II) or (IIa) in which the free amino group at C10 is protected. Some embodiments relate to protected compounds of Formula (II) or (IIa) in which the free amino group at C10 is protected with a formyl, acetyl, trifluoroacetyl, benzyl (Bn), benzoyl (Bz), benzyloxycarbonyl (CBZ), tert-butoxycarbonyl (Boc), trityl, substituted trityl, tosyl, phthalimide, alloxycarbonyl (Alloc) or 9-fluorenylmethyloxycarbonyl (FMOC) group. Some embodiments relate to protected versions of compounds of Formula (II) or (IIa) in which the free amino group at C10 is protected with an acetyl group.

In certain embodiments, each alkyl, cycloalkyl, heterocycloalkyl, aryl and heteroaryl group as defined in any one of Formulae (I), (Ia), (II), (IIa), (III), (IIIa) or (IIIb) is optionally substituted with one or more substituents selected from: halogen, acyl, acyloxy, alkoxy, carboxy, hydroxy, amino, amido, nitro, cyano, azido, alkylthio, thio, sulfonyl, sulfonamido, alkyl, cycloalkyl, heterocycloalkyl, aryl and heteroaryl. In some embodiments, each alkyl, cycloalkyl, heterocycloalkyl, aryl and heteroaryl group as defined in any one of Formulae (I), (Ia), (II), (IIa), (III), (IIIa) or (IIIb) is optionally substituted with one or more substituents selected from: halogen, acyl, acyloxy, alkoxy, carboxy, hydroxy, amino, amido, nitro, cyano, azido, alkylthio, thio, sulfonyl and sulfonamido.

In certain embodiments of the present disclosure, the camptothecin analogue is a compound having Formula (I) or a protected version thereof and is selected from the compounds shown in Table 1.

TABLE 1
Exemplary Camptothecin Analogues of Formula (I)

| | Compound | |
|---|---|---|
| Structure | Name | Number |
| 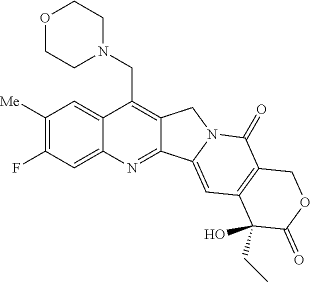 [SMILES: CC[C@@]1(O)C(=O)OCc2c1cc1n(c2=O)Cc2c-1nc1cc(F)c(C)cc1c2CN1CCOCC1] | (S)-4-ethyl-8-fluoro-4-hydroxy-9-methyl- 11-(morpholinomethyl)-1,12-dihydro-14H- pyrano[3′,4′:6,7]indolizino[1,2- b]quinoline-3,14(4H)-dione | Compound 100 |
| 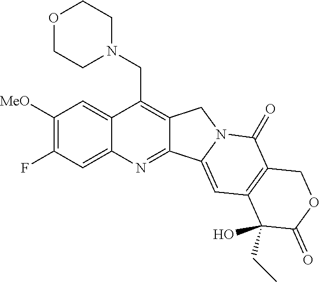 [SMILES: CC[C@@]1(O)C(=O)OCc2c1cc1n(c2=O)Cc2c-1nc1cc(F)c(OC)cc1c2CN1CCOCC1] | (S)-4-ethyl-8-fluoro-4-hydroxy-9-methoxy- 11-(morpholinomethyl)-1,12-dihydro-14H- pyrano[3′,4′:6,7]indolizino[1,2- b]quinoline-3,14(4H)-dione | Compound 101 |
| 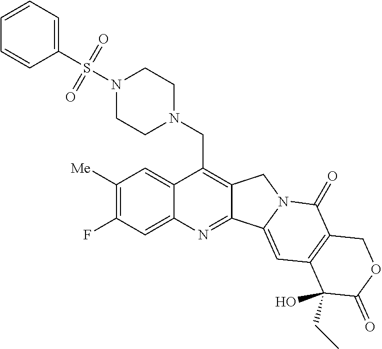 [SMILES: CC[C@@]1(O)C(=O)OCc2c1cc1n(c2=O)Cc2c-1nc1cc(F)c(C)cc1c2CN1CCN(S(=O)(=O)c2ccccc2)CC1] | (S)-4-ethyl-8-fluoro-4-hydroxy-9-methyl- 11-((4-(phenylsulfonyl)piperazin-1- yl)methyl)-1,12-dihydro-14H- pyrano[3′,4′:6,7]indolizino[1,2- b]quinoline-3,14(4H)-dione | Compound 102 |
| 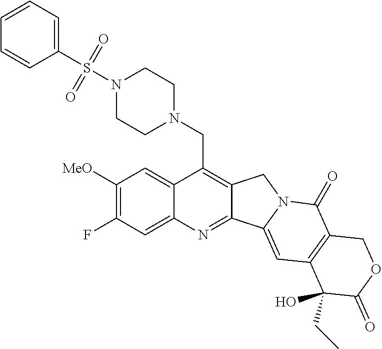 [SMILES: CC[C@@]1(O)C(=O)OCc2c1cc1n(c2=O)Cc2c-1nc1cc(F)c(OC)cc1c2CN1CCN(S(=O)(=O)c2ccccc2)CC1] | (S)-4-ethyl-8-fluoro-4-hydroxy-9-methoxy- 11-((4-(phenylsulfonyl)piperazin-1- yl)methyl)-1,12-dihydro-14H- pyrano[3′,4′:6,7]indolizino[1,2- b]quinoline-3,14(4H)-dione | Compound 103 |
| 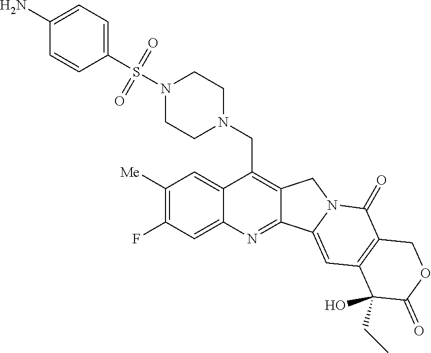 [SMILES: CC[C@@]1(O)C(=O)OCc2c1cc1n(c2=O)Cc2c-1nc1cc(F)c(C)cc1c2CN1CCN(S(=O)(=O)c2ccc(N)cc2)CC1] | (S)-11-((4-((4- aminophenyl)sulfonyl)piperazin-1- yl)methyl)-4-ethyl-8-fluoro-4-hydroxy-9- methyl-1,12-dihydro-14H- pyrano[3′,4′:6,7]indolizino[1,2- b]quinoline-3,14(4H)-dione | Compound 104 |
| 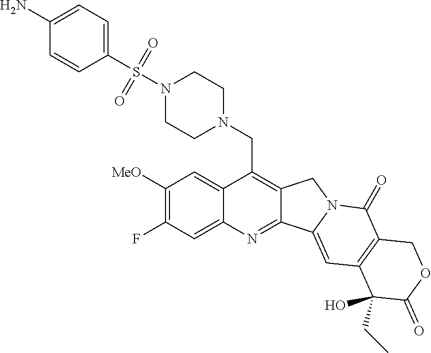 [SMILES: CC[C@@]1(O)C(=O)OCc2c1cc1n(c2=O)Cc2c-1nc1cc(F)c(OC)cc1c2CN1CCN(S(=O)(=O)c2ccc(N)cc2)CC1] | (S)-11-((4-((4- aminophenyl)sulfonyl)piperazin-1- yl)methyl)-4-ethyl-8-fluoro-4-hydroxy-9- methoxy-1,12-dihydro-14H- pyrano[3′,4′:6,7]indolizino[1,2- b]quinoline-3,14(4H)-dione | Compound 105 |
| 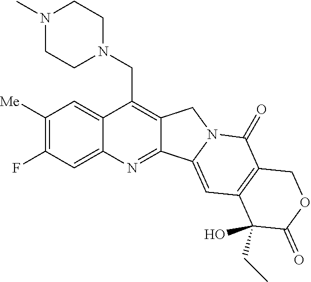 [SMILES: CC[C@@]1(O)C(=O)OCc2c1cc1n(c2=O)Cc2c-1nc1cc(F)c(C)cc1c2CN1CCN(C)CC1] | (S)-4-ethyl-8-fluoro-4-hydroxy-9-methyl- 11-((4-methylpiperazin-1-yl)methyl)-1,12- dihydro-14H- pyrano[3′,4′:6,7]indolizino[1,2- b]quinoline-3,14(4H)-dione | Compound 106 |
| 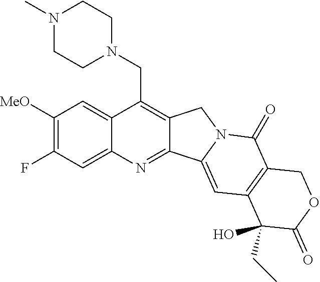 [SMILES: CC[C@@]1(O)C(=O)OCc2c1cc1n(c2=O)Cc2c-1nc1cc(F)c(OC)cc1c2CN1CCN(C)CC1] | (S)-4-ethyl-8-fluoro-4-hydroxy-9-methoxy- 11-((4-methylpiperazin-1-yl)methyl)-1,12- dihydro-14H- pyrano[3′,4′:6,7]indolizino[1,2- b]quinoline-3,14(4H)-dione | Compound 107 |
| 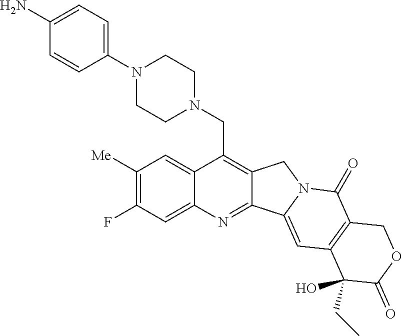 [SMILES: CC[C@@]1(O)C(=O)OCc2c1cc1n(c2=O)Cc2c-1nc1cc(F)c(C)cc1c2CN1CCN(c2ccc(N)cc2)CC1] | (S)-11-((4-(4-aminophenyl)piperazin-1- yl)methyl)-4-ethyl-8-fluoro-4-hydroxy-9- methyl-1,12-dihydro-14H- pyrano[3′,4′:6,7]indolizino[1,2- b]quinoline-3,14(4H)-dione | Compound 108 |
| 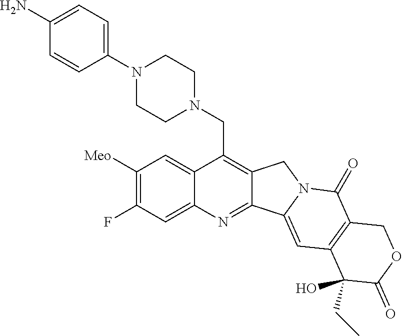 | (S)-11-((4-(4-aminophenyl)piperazin-1- yl)methyl)-4-ethyl-8-fluoro-4-hydroxy-9- methoxy-1,12-dihydro-14H- pyrano[3′,4′:6,7]indolizino[1,2- b]quinoline-3,14(4H)-dione | Compound 109 |
| 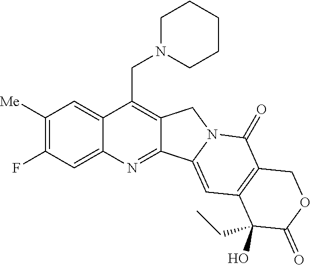 [SMILES: CC[C@@]1(O)C(=O)OCc2c1cc1n(c2=O)Cc2c-1nc1cc(F)c(C)cc1c2CN1CCCCC1] | (S)-4-ethyl-8-fluoro-4-hydroxy-9-methyl- 11-(piperidin-1-ylmethyl)-1,12-dihydro- 14H-pyrano[3′,4′:6,7]indolizino[1,2- b]quinoline-3,14(4H)-dione | Compound 110 |
| 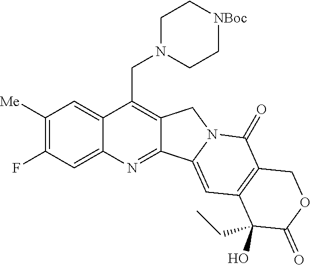 [SMILES: CC[C@@]1(O)C(=O)OCc2c1cc1n(c2=O)Cc2c-1nc1cc(F)c(C)cc1c2CN1CCN(C(=O)OC(C)(C)C)CC1] | tert-butyl (S)-4-((4-ethyl-8-fluoro-4- hydroxy-9-methyl-3,14-dioxo-3,4,12,14- tetrahydro-1H- pyrano[3′,4′:6,7]indolizino[1,2-b]quinolin- 11-yl)methyl)piperazine-1-carboxylate | Compound 111 |
| 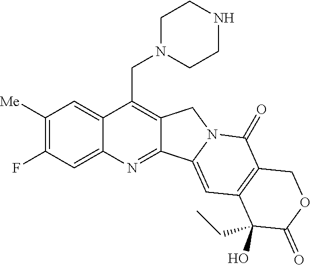 [SMILES: CC[C@@]1(O)C(=O)OCc2c1cc1n(c2=O)Cc2c-1nc1cc(F)c(C)cc1c2CN1CCNCC1] | (S)-4-ethyl-8-fluoro-4-hydroxy-9-methyl- 11-(piperazin-1-ylmethyl)-1,12-dihydro- 14H-pyrano[3′,4′:6,7]indolizino[1,2- b]quinoline-3,14(4H)-dione | Compound 112 |
| 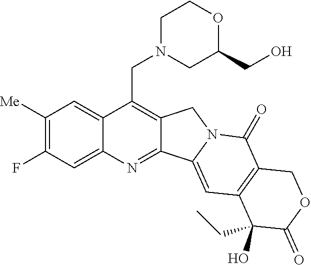 [SMILES: CC[C@@]1(O)C(=O)OCc2c1cc1n(c2=O)Cc2c-1nc1cc(F)c(C)cc1c2CN1CCO[C@@H](CO)C1] | (S)-4-ethyl-8-fluoro-4-hydroxy-11-(((R)-2- (hydroxymethyl)morpholino)methyl)-9- methyl-1,12-dihydro-14H- pyrano[3′,4′:6,7]indolizino[1,2- b]quinoline-3,14(4H)-dione | Compound 113 |
| 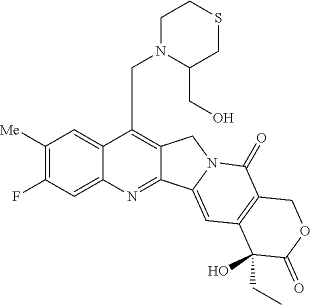 [SMILES: CC[C@@]1(O)C(=O)OCc2c1cc1n(c2=O)Cc2c-1nc1cc(F)c(C)cc1c2CN1CCSCC1CO] | (4S)-4-ethyl-8-fluoro-4-hydroxy-11-((3- (hydroxymethyl)thiomorpholino)methyl)- 9-methyl-1,12-dihydro-14H- pyrano[3′,4′:6,7]indolizino[1,2- b]quinoline-3,14(4H)-dione | Compound 114 |
| 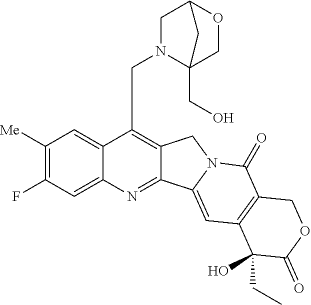 [SMILES: CC[C@@]1(O)C(=O)OCc2c1cc1n(c2=O)Cc2c-1nc1cc(F)c(C)cc1c2CN1CC2CC1(CO)CO2] | (4S)-4-ethyl-8-fluoro-4-hydroxy-11-((4- (hydroxymethyl)-2-oxa-5- azabicyclo[2.2.1]heptan-5-yl)methyl)-9- methyl-1,12-dihydro-14H- pyrano[3′,4′:6,7]indolizino[1,2- b]quinoline-3,14(4H)-dione | Compound 115 |
| 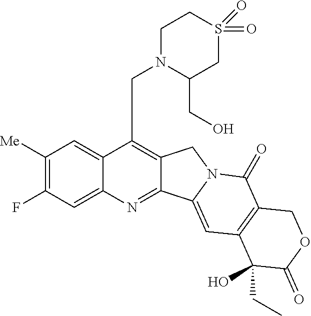 [SMILES: CC[C@@]1(O)C(=O)OCc2c1cc1n(c2=O)Cc2c-1nc1cc(F)c(C)cc1c2CN1CCS(=O)(=O)CC1CO] | (4S)-4-ethyl-8-fluoro-4-hydroxy-11-((3- (hydroxymethyl)-1,1- dioxidothiomorpholino)methyl)-9-methyl- 1,12-dihydro-14H- pyrano[3′,4′:6,7]indolizino[1,2- b]quinoline-3,14(4H)-dione | Compound 116 |
| 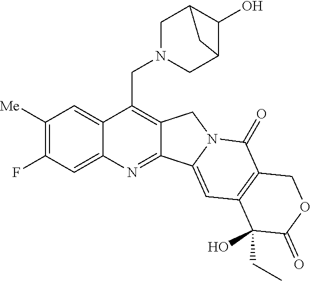 [SMILES: CC[C@@]1(O)C(=O)OCc2c1cc1n(c2=O)Cc2c-1nc1cc(F)c(C)cc1c2CN1CC2CC(C1)C2O] | (4S)-4-ethyl-8-fluoro-4-hydroxy-11-((6- hydroxy-3-azabicyclo[3.1.1]heptan-3- yl)methyl)-9-methyl-1,12-dihydro-14H- pyrano[3′,4′:6,7]indolizino[1,2- b]quinoline-3,14(4H)-dione | Compound 117 |
| 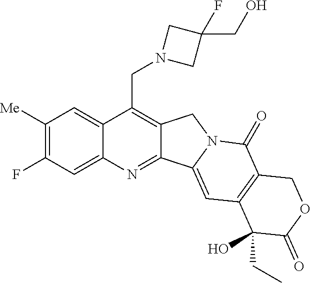 [SMILES: CC[C@@]1(O)C(=O)OCc2c1cc1n(c2=O)Cc2c-1nc1cc(F)c(C)cc1c2CN1CC(F)(CO)C1] | (S)-4-ethyl-8-fluoro-11-((3-fluoro-3- (hydroxymethyl)azetidin-1-yl)methyl)-4- hydroxy-9-methyl-1,12-dihydro-14H- pyrano[3′,4′:6,7]indolizino[1,2- b]quinoline-3,14(4H)-dione | Compound 118 |
| 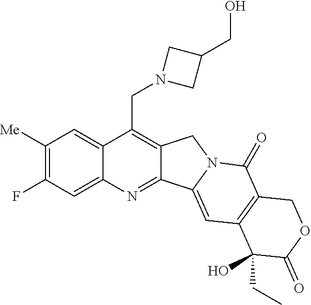 [SMILES: CC[C@@]1(O)C(=O)OCc2c1cc1n(c2=O)Cc2c-1nc1cc(F)c(C)cc1c2CN1CC(CO)C1] | (S)-4-ethyl-8-fluoro-4-hydroxy-11-((3- (hydroxymethyl)azetidin-1-yl)methyl)-9- methyl-1,12-dihydro-14H- pyrano[3′,4′:6,7]indolizino[1,2- b]quinoline-3,14(4H)-dione | Compound 119 |
| 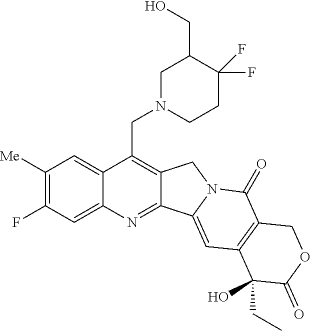 [SMILES: CC[C@@]1(O)C(=O)OCc2c1cc1n(c2=O)Cc2c-1nc1cc(F)c(C)cc1c2CN1CCC(F)(F)C(CO)C1] | (4S)-11-((4,4-difluoro-3- (hydroxymethyl)piperidin-1-yl)methyl)-4- ethyl-8-fluoro-4-hydroxy-9-methyl-1,12- dihydro-14H- pyrano[3′,4′:6,7]indolizino[1,2- b]quinoline-3,14(4H)-dione | Compound 120 |
| 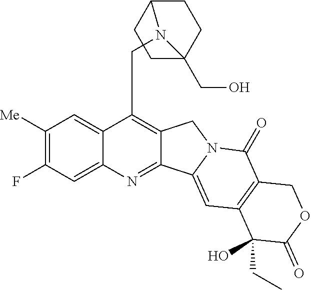 [SMILES: CC[C@@]1(O)C(=O)OCc2c1cc1n(c2=O)Cc2c-1nc1cc(F)c(C)cc1c2CN1C2CCC1(CO)CC2] | (4S)-11-((4,4-difluoro-3- (hydroxymethyl)piperidin-1-yl)methyl)-4- ethyl-8-fluoro-4-hydroxy-9-methyl-1,12- dihydro-14H- pyrano[3′,4′:6,7]indolizino[1,2- b]quinoline-3,14(4H)-dione | Compound 121 |
| 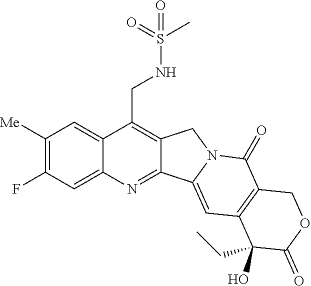 [SMILES: CC[C@@]1(O)C(=O)OCc2c1cc1n(c2=O)Cc2c-1nc1cc(F)c(C)cc1c2CNS(C)(=O)=O] | (S)-N-((4-ethyl-8-fluoro-4-hydroxy-9- methyl-3,14-dioxo-3,4,12,14-tetrahydro- 1H-pyrano[3′,4′:6,7]indolizino[1,2- b]quinolin-11- yl)methyl)methanesulfonamide | Compound 122 |
| 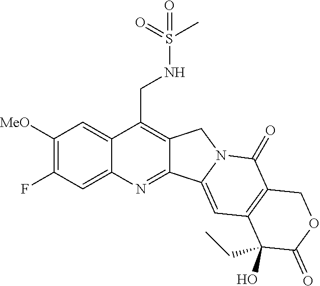 [SMILES: CC[C@@]1(O)C(=O)OCc2c1cc1n(c2=O)Cc2c-1nc1cc(F)c(OC)cc1c2CNS(C)(=O)=O] | (S)-N-((4-ethyl-8-fluoro-4-hydroxy-9- methoxy-3,14-dioxo-3,4,12,14-tetrahydro- 1H-pyrano[3′,4′:6,7]indolizino[1,2- b]quinolin-11- yl)methyl)methanesulfonamide | Compound 123 |
| 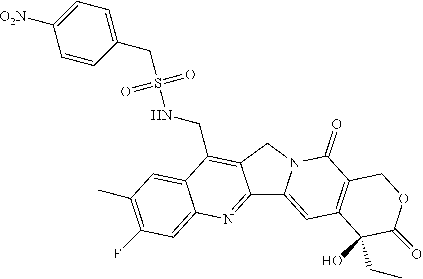 [SMILES: CC[C@@]1(O)C(=O)OCc2c1cc1n(c2=O)Cc2c-1nc1cc(F)c(C)cc1c2CNS(=O)(=O)Cc1ccc([N+](=O)[O-])cc1] | (S)-N-((4-ethyl-8-fluoro-4-hydroxy-9- methyl-3,14-dioxo-3,4,12,14-tetrahydro- 1H-pyrano[3′,4′:6,7]indolizino[1,2- b]quinolin-11-yl)methyl)-1-(4- nitrophenyl)methanesulfonamide | Compound 124 |
| 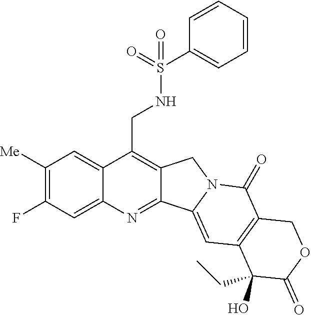 [SMILES: CC[C@@]1(O)C(=O)OCc2c1cc1n(c2=O)Cc2c-1nc1cc(F)c(C)cc1c2CNS(=O)(=O)c1ccccc1] | (S)-N-((4-ethyl-8-fluoro-4-hydroxy-9- methyl-3,14-dioxo-3,4,12,14-tetrahydro- 1H-pyrano[3′,4′:6,7]indolizino[1,2- b]quinolin-11- yl)methyl)benzenesulfonamide | Compound 125 |
| 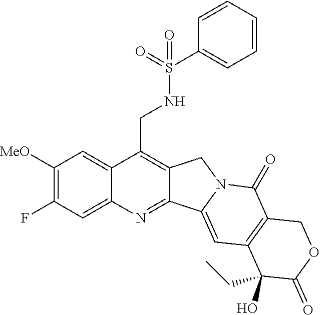 [SMILES: CC[C@@]1(O)C(=O)OCc2c1cc1n(c2=O)Cc2c-1nc1cc(F)c(OC)cc1c2CNS(=O)(=O)c1ccccc1] | (S)-N-((4-ethyl-8-fluoro-4-hydroxy-9- methoxy-3,14-dioxo-3,4,12,14-tetrahydro- 1H-pyrano[3′,4′:6,7]indolizino[1,2- b]quinolin-11- yl)methyl)benzenesulfonamide | Compound 126 |
| 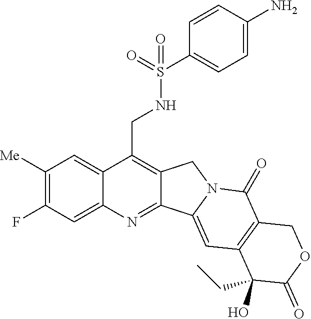 [SMILES: CC[C@@]1(O)C(=O)OCc2c1cc1n(c2=O)Cc2c-1nc1cc(F)c(C)cc1c2CNS(=O)(=O)c1ccc(N)cc1] | (S)-4-amino-N-((4-ethyl-8-fluoro-4- hydroxy-9-methyl-3,14-dioxo-3,4,12,14- tetrahydro-1H- pyrano[3′,4′:6,7]indolizino[1,2-b]quinolin- 11-yl)methyl)benzenesulfonamide | Compound 127 |
| 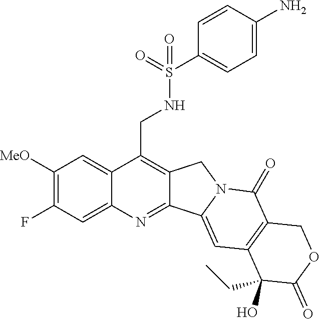 [SMILES: CC[C@@]1(O)C(=O)OCc2c1cc1n(c2=O)Cc2c-1nc1cc(F)c(OC)cc1c2CNS(=O)(=O)c1ccc(N)cc1] | (S)-4-amino-N-((4-ethyl-8-fluoro-4- hydroxy-9-methoxy-3,14-dioxo-3,4,12,14- tetrahydro-1H- pyrano[3′,4′:6,7]indolizino[1,2-b]quinolin- 11-yl)methyl)benzenesulfonamide | Compound 128 |
| 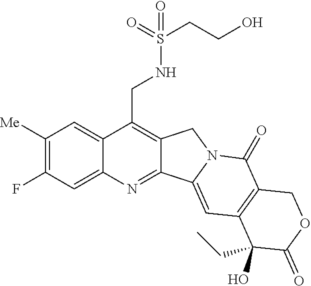 [SMILES: CC[C@@]1(O)C(=O)OCc2c1cc1n(c2=O)Cc2c-1nc1cc(F)c(C)cc1c2CNS(=O)(=O)CCO] | (S)-N-((4-ethyl-8-fluoro-4-hydroxy-9- methyl-3,14-dioxo-3,4,12,14-tetrahydro- 1H-pyrano[3′,4′:6,7]indolizino[1,2- b]quinolin-11-yl)methyl)-2- hydroxyethane-1-sulfonamide | Compound 129 |
| 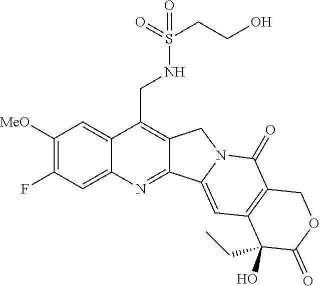 [SMILES: CC[C@@]1(O)C(=O)OCc2c1cc1n(c2=O)Cc2c-1nc1cc(F)c(OC)cc1c2CNS(=O)(=O)CCO] | (S)-N-((4-ethyl-8-fluoro-4-hydroxy-9- methoxy-3,14-dioxo-3,4,12,14-tetrahydro- 1H-pyrano[3′,4′:6,7]indolizino[1,2- b]quinolin-11-yl)methyl)-2- hydroxyethane-1-sulfonamide | Compound 130 |
| 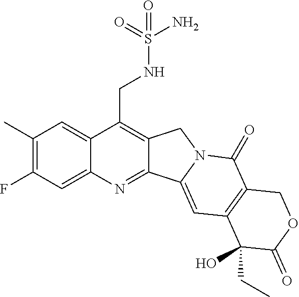 [SMILES: CC[C@@]1(O)C(=O)OCc2c1cc1n(c2=O)Cc2c-1nc1cc(F)c(C)cc1c2CNS(N)(=O)=O] | (S)-((4-ethyl-8-fluoro-4-hydroxy-9-methyl- 3,14-dioxo-3,4,12,14-tetrahydro-1H- pyrano[3′,4′:6,7]indolizino[1,2-b]quinolin- 11-yl)methyl)sulfamide | Compound 131 |
| 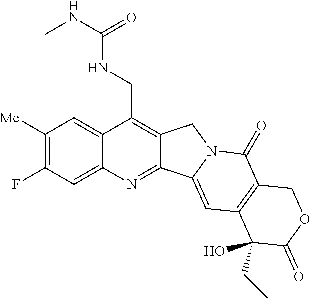 [SMILES: CC[C@@]1(O)C(=O)OCc2c1cc1n(c2=O)Cc2c-1nc1cc(F)c(C)cc1c2CNC(=O)NC] | (S)-1-((4-ethyl-8-fluoro-4-hydroxy-9- methyl-3,14-dioxo-3,4,12,14-tetrahydro- 1H-pyrano[3′,4′:6,7]indolizino[1,2- b]quinolin-11-yl)methyl)-3-methylurea | Compound 132 |
| 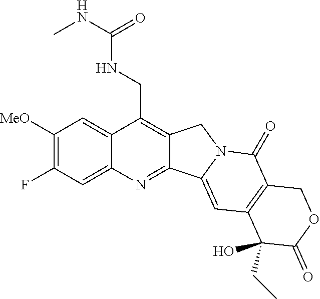 [SMILES: CC[C@@]1(O)C(=O)OCc2c1cc1n(c2=O)Cc2c-1nc1cc(F)c(OC)cc1c2CNC(=O)NC] | (S)-1-((4-ethyl-8-fluoro-4-hydroxy-9- methoxy-3,14-dioxo-3,4,12,14-tetrahydro- 1H-pyrano[3′,4′:6,7]indolizino[1,2- b]quinolin-11-yl)methyl)-3-methylurea | Compound 133 |
| 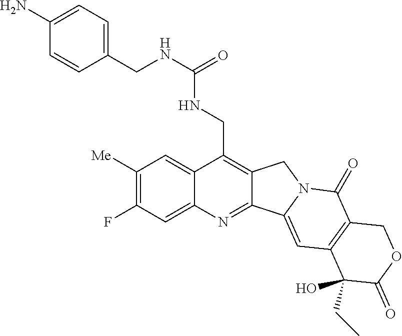 [SMILES: CC[C@@]1(O)C(=O)OCc2c1cc1n(c2=O)Cc2c-1nc1cc(F)c(C)cc1c2CNC(=O)NCc1ccc(N)cc1] | (S)-1-(4-aminobenzyl)-3-((4-ethyl-8- fluoro-4-hydroxy-9-methyl-3,14-dioxo- 3,4,12,14-tetrahydro-1H- pyrano[3′,4′:6,7]indolizino[1,2-b]quinolin- 11-yl)methyl)urea | Compound 134 |
| 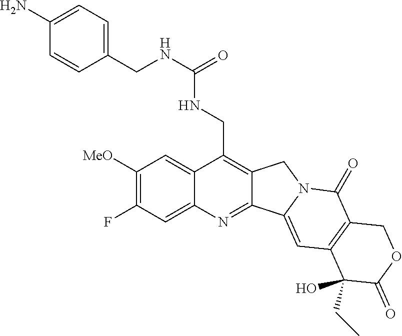 [SMILES: CC[C@@]1(O)C(=O)OCc2c1cc1n(c2=O)Cc2c-1nc1cc(F)c(OC)cc1c2CNC(=O)NCc1ccc(N)cc1] | (S)-1-(4-aminobenzyl)-3-((4-ethyl-8- fluoro-4-hydroxy-9-methoxy-3,14-dioxo- 3,4,12,14-tetrahydro-1H- pyrano[3′,4′:6,7]indolizino[1,2-b]quinolin- 11-yl)methyl)urea | Compound 135 |
| 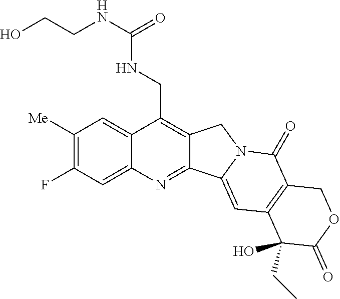 [SMILES: CC[C@@]1(O)C(=O)OCc2c1cc1n(c2=O)Cc2c-1nc1cc(F)c(C)cc1c2CNC(=O)NCCO] | (S)-1-((4-ethyl-8-fluoro-4-hydroxy-9- methyl-3,14-dioxo-3,4,12,14-tetrahydro- 1H-pyrano[3′,4′:6,7]indolizino[1,2- b]quinolin-11-yl)methyl)-3-(2- hydroxyethyl)urea | Compound 136 |
| 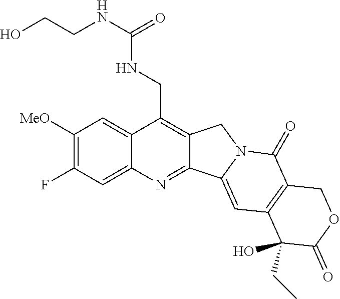 [SMILES: CC[C@@]1(O)C(=O)OCc2c1cc1n(c2=O)Cc2c-1nc1cc(F)c(OC)cc1c2CNC(=O)NCCO] | (S)-1-((4-ethyl-8-fluoro-4-hydroxy-9- methoxy-3,14-dioxo-3,4,12,14-tetrahydro- 1H-pyrano[3′,4′:6,7]indolizino[1,2- b]quinolin-11-yl)methyl)-3-(2- hydroxyethyl)urea | Compound 137 |
| 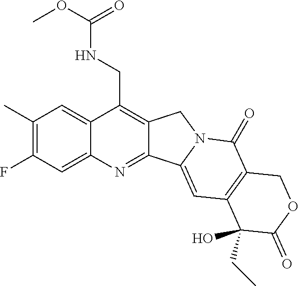 [SMILES: CC[C@@]1(O)C(=O)OCc2c1cc1n(c2=O)Cc2c-1nc1cc(F)c(C)cc1c2CNC(=O)OC] | methyl (S)-((4-ethyl-8-fluoro-4-hydroxy-9- methyl-3,14-dioxo-3,4,12,14-tetrahydro- 1H-pyrano[3′,4′:6,7]indolizino[1,2- b]quinolin-11-yl)methyl)carbamate | Compound 138 |
| 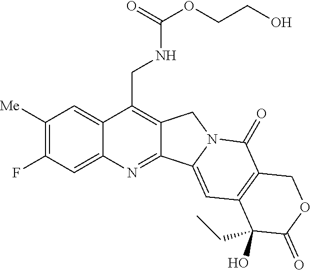 [SMILES: CC[C@@]1(O)C(=O)OCc2c1cc1n(c2=O)Cc2c-1nc1cc(F)c(C)cc1c2CNC(=O)OCCO] | 2-hydroxyethyl (S)-((4-ethyl-8-fluoro-4- hydroxy-9-methyl-3,14-dioxo-3,4,12,14- tetrahydro-1H- pyrano[3′,4′:6,7]indolizino[1,2-b]quinolin- 11-yl)methyl)carbamate | Compound 139 |
| 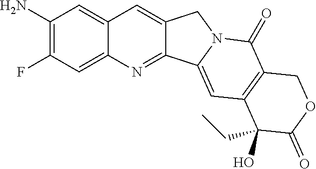 [SMILES: CC[C@@]1(O)C(=O)OCc2c1cc1n(c2=O)Cc2cc3cc(N)c(F)cc3nc2-1] | (S)-9-amino-4-ethyl-8-fluoro-4-hydroxy- 1,12-dihydro-14H- pyrano[3′,4′:6,7]indolizino[1,2- b]quinoline-3,14(4H)-dione | Compound 140 |
| 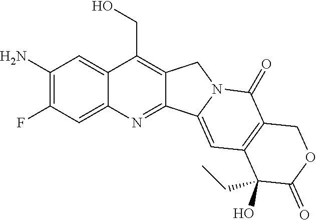 [SMILES: CC[C@@]1(O)C(=O)OCc2c1cc1n(c2=O)Cc2c-1nc1cc(F)c(N)cc1c2CO] | (S)-9-amino-4-ethyl-8-fluoro-4-hydroxy- 11-(hydroxymethyl)-1,12-dihydro-14H- pyrano[3′,4′:6,7]indolizino[1,2- b]quinoline-3,14(4H)-dione | Compound 141 |
| 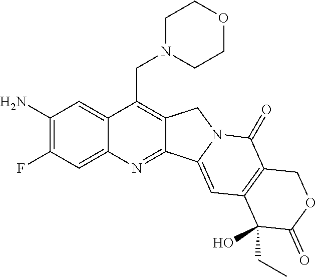 [SMILES: CC[C@@]1(O)C(=O)OCc2c1cc1n(c2=O)Cc2c-1nc1cc(F)c(N)cc1c2CN1CCOCC1] | (S)-9-amino-4-ethyl-8-fluoro-4-hydroxy- 11-(morpholinomethyl)-1,12-dihydro-14H- pyrano[3′,4′:6,7]indolizino[1,2- b]quinoline-3,14(4H)-dione | Compound 142 |
| 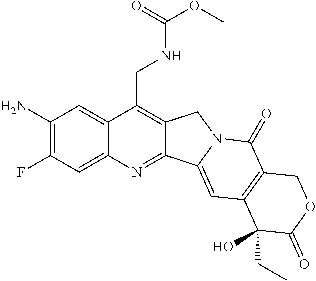 [SMILES: CC[C@@]1(O)C(=O)OCc2c1cc1n(c2=O)Cc2c-1nc1cc(F)c(N)cc1c2CNC(=O)OC] | methyl (S)-((9-amino-4-ethyl-8-fluoro-4- hydroxy-3,14-dioxo-3,4,12,14-tetrahydro- 1H-pyrano[3′,4′:6,7]indolizino[1,2- b]quinolin-11-yl)methyl)carbamate | Compound 143 |
| 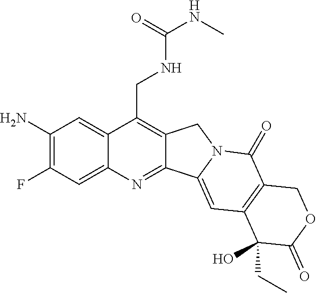 [SMILES: CC[C@@]1(O)C(=O)OCc2c1cc1n(c2=O)Cc2c-1nc1cc(F)c(N)cc1c2CNC(=O)NC] | (S)-1-((9-amino-4-ethyl-8-fluoro-4- hydroxy-3,14-dioxo-3,4,12,14-tetrahydro- 1H-pyrano[3′,4′:6,7]indolizino[1,2- b]quinolin-11-yl)methyl)-3-methylurea | Compound 144 |
| 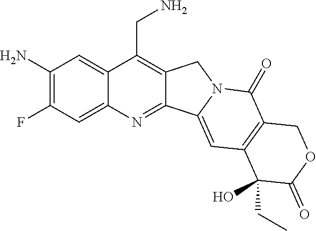 [SMILES: CC[C@@]1(O)C(=O)OCc2c1cc1n(c2=O)Cc2c-1nc1cc(F)c(N)cc1c2CN] | (S)-9-amino-11-(aminomethyl)-4-ethyl-8- fluoro-4-hydroxy-1,12-dihydro-14H- pyrano[3′,4′:6,7]indolizino[1,2- b]quinoline-3,14(4H)-dione | Compound 145 |
| 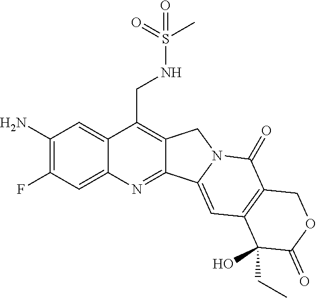 [SMILES: CC[C@@]1(O)C(=O)OCc2c1cc1n(c2=O)Cc2c-1nc1cc(F)c(N)cc1c2CNS(C)(=O)=O] | (S)-N-((9-amino-4-ethyl-8-fluoro-4- hydroxy-3,14-dioxo-3,4,12,14-tetrahydro- 1H-pyrano[3′,4′:6,7]indolizino[1,2- b]quinolin-11- yl)methyl)methanesulfonamide | Compound 146 |
| 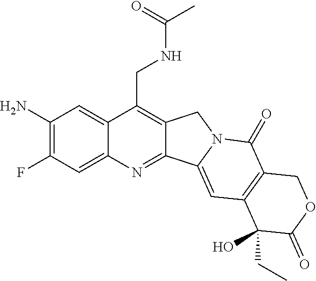 [SMILES: CC[C@@]1(O)C(=O)OCc2c1cc1n(c2=O)Cc2c-1nc1cc(F)c(N)cc1c2CNC(C)=O] | (S)-N-((9-amino-4-ethyl-8-fluoro-4- hydroxy-3,14-dioxo-3,4,12,14-tetrahydro- 1H-pyrano[3′,4′:6,7]indolizino[1,2- b]quinolin-11-yl)methyl)acetamide | Compound 147 |
| 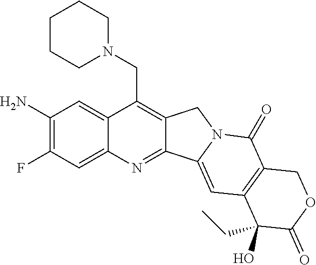 [SMILES: CC[C@@]1(O)C(=O)OCc2c1cc1n(c2=O)Cc2c-1nc1cc(F)c(N)cc1c2CN1CCCCC1] | (S)-9-amino-4-ethyl-8-fluoro-4-hydroxy- 11-(piperidin-1-ylmethyl)-1,12-dihydro- 14H-pyrano[3′,4′:6,7]indolizino[1,2- b]quinoline-3,14(4H)-dione | Compound 148 |
| 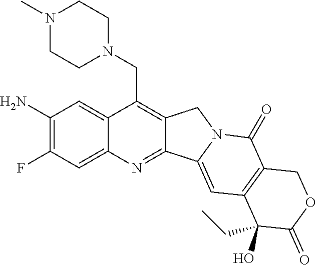 [SMILES: CC[C@@]1(O)C(=O)OCc2c1cc1n(c2=O)Cc2c-1nc1cc(F)c(N)cc1c2CN1CCN(C)CC1] | (S)-9-amino-4-ethyl-8-fluoro-4-hydroxy- 11-((4-methylpiperazin-1-yl)methyl)-1,12- dihydro-14HI- pyrano[3′,4′:6,7]indolizino[1,2- b]quinoline-3,14(4H)-dione | Compound 149 |
| 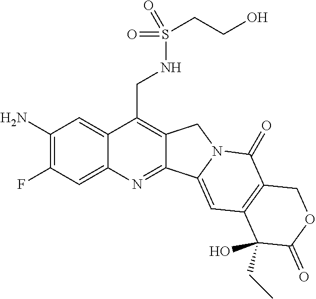 [SMILES: CC[C@@]1(O)C(=O)OCc2c1cc1n(c2=O)Cc2c-1nc1cc(F)c(N)cc1c2CNS(=O)(=O)CCO] | (S)-N-((9-amino-4-ethyl-8-fluoro-4- hydroxy-3,14-dioxo-3,4,12,14-tetrahydro- 1H-pyrano[3′,4′:6,7]indolizino[1,2- b]quinolin-11-yl)methyl)-2- hydroxyethane-1-sulfonamide | Compound 150 |
| 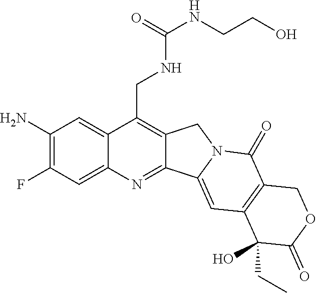 [SMILES: CC[C@@]1(O)C(=O)OCc2c1cc1n(c2=O)Cc2c-1nc1cc(F)c(N)cc1c2CNC(=O)NCCO] | (S)-1-((9-amino-4-ethyl-8-fluoro-4- hydroxy-3,14-dioxo-3,4,12,14-tetrahydro- 1H-pyrano[3′,4′:6,7]indolizino[1,2- b]quinolin-11-yl)methyl)-3-(2- hydroxyethyl)urea | Compound 151 |
| 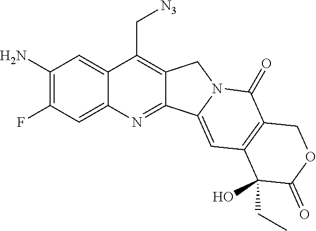 [SMILES: CC[C@@]1(O)C(=O)OCc2c1cc1n(c2=O)Cc2c-1nc1cc(F)c(N)cc1c2CN=[N+]=[N-]] | (S)-9-amino-11-(azidomethyl)-4-ethyl-8- fluoro-4-hydroxy-1,12-dihydro-14H- pyrano[3′,4′:6,7]indolizino[1,2- b]quinoline-3,14(4H)-dione | Compound 152 |
| 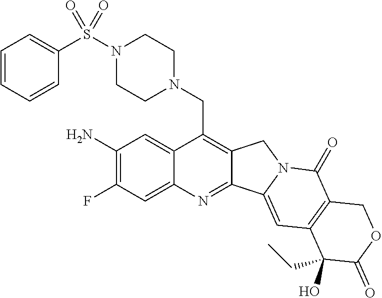 [SMILES: CC[C@@]1(O)C(=O)OCc2c1cc1n(c2=O)Cc2c-1nc1cc(F)c(N)cc1c2CN1CCN(S(=O)(=O)c2ccccc2)CC1] | (S)-9-amino-4-ethyl-8-fluoro-4-hydroxy- 11-((4-(phenylsulfonyl)piperazin-1- yl)methyl)-1,12-dihydro-14H- pyrano[3′,4′:6,7]indolizino[1,2- b]quinoline-3,14(4H)-dione | Compound 153 |
| 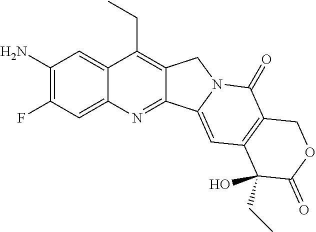 [SMILES: CCc1c2c(nc3cc(F)c(N)cc13)-c1cc3c(c(=O)n1C2)COC(=O)[C@]3(O)CC] | (S)-9-amino-4,11-diethyl-8-fluoro-4- hydroxy-1,12-dihydro-14H- pyrano[3′,4′:6,7]indolizino[1,2- b]quinoline-3,14(4H)-dione | Compound 154 |
| 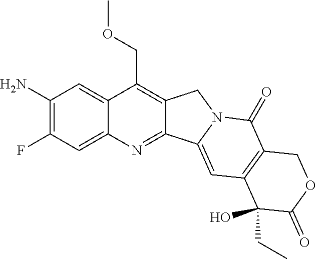 [SMILES: CC[C@@]1(O)C(=O)OCc2c1cc1n(c2=O)Cc2c-1nc1cc(F)c(N)cc1c2COC] | (S)-9-amino-4-ethyl-8-fluoro-4-hydroxy- 11-(methoxymethyl)-1,12-dihydro-14H- pyrano[3′,4′:6,7]indolizino[1,2- b]quinoline-3,14(4H)-dione | Compound 155 |
| 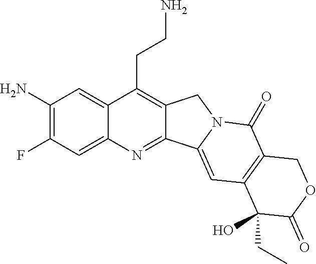 [SMILES: CC[C@@]1(O)C(=O)OCc2c1cc1n(c2=O)Cc2c-1nc1cc(F)c(N)cc1c2CCN] | (S)-9-amino-11-(2-aminoethyl)-4-ethyl-8- fluoro-4-hydroxy-1,12-dihydro-14H- pyrano[3′,4′:6,7]indolizino[1,2- b]quinoline-3,14(4H)-dione | Compound 156 |
| 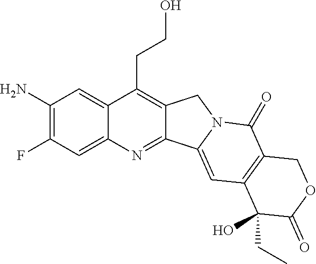 [SMILES: CC[C@@]1(O)C(=O)OCc2c1cc1n(c2=O)Cc2c-1nc1cc(F)c(N)cc1c2CCO] | (S)-9-amino-4-ethyl-8-fluoro-4-hydroxy- 11-(2-hydroxyethyl)-1,12-dihydro-14H- pyrano[3′,4′:6,7]indolizino[1,2- b]quinoline-3,14(4H)-dione | Compound 157 |
| 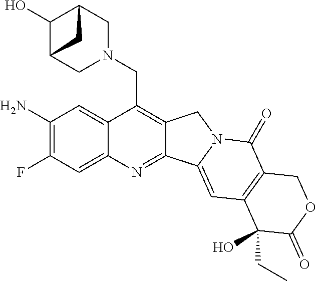 [SMILES: CC[C@@]1(O)C(=O)OCc2c1cc1n(c2=O)Cc2c-1nc1cc(F)c(N)cc1c2CN1C[C@H]2C[C@@H](C1)C2O] | (4S)-9-amino-4-ethyl-8-fluoro-4-hydroxy- 11-(((1R,5S)-6-hydroxy-3- azabicyclo[3.1.1]heptan-3-yl)methyl)-1,12- dihydro-14H- pyrano[3′,4′:6,7]indolizino[1,2- b]quinoline-3,14(4H)-dione | Compound 158 |
| 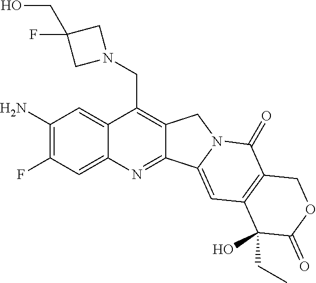 [SMILES: CC[C@@]1(O)C(=O)OCc2c1cc1n(c2=O)Cc2c-1nc1cc(F)c(N)cc1c2CN1CC(F)(CO)C1] | (S)-9-amino-4-ethyl-8-fluoro-11-((3- fluoro-3-(hydroxymethyl)azetidin-1- yl)methyl)-4-hydroxy-1,12-dihydro-14H- pyrano[3′,4′:6,7]indolizino[1,2- b]quinoline-3,14(4H)-dione | Compound 159 |
| 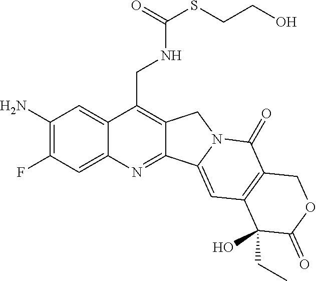 [SMILES: CC[C@@]1(O)C(=O)OCc2c1cc1n(c2=O)Cc2c-1nc1cc(F)c(N)cc1c2CNC(=O)SCCO] | S-(2-hydroxyethyl) (S)-((9-amino-4-ethyl- 8-fluoro-4-hydroxy-3,14-dioxo-3,4,12,14- tetrahydro-1H- pyrano[3′,4′:6,7]indolizino[1,2-b]quinolin- 11-yl)methyl)carbamothioate | Compound 160 |
| 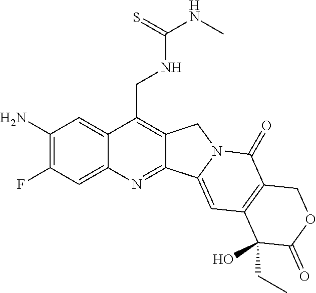 [SMILES: CC[C@@]1(O)C(=O)OCc2c1cc1n(c2=O)Cc2c-1nc1cc(F)c(N)cc1c2CNC(=S)NC] | (S)-1-((9-amino-4-ethyl-8-fluoro-4- hydroxy-3,14-dioxo-3,4,12,14-tetrahydro- 1H-pyrano[3′,4′:6,7]indolizino[1,2- b]quinolin-11-yl)methyl)-3-methylthiourea | Compound 161 |
| 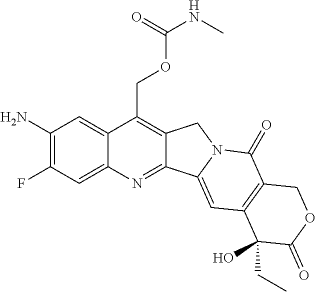 [SMILES: CC[C@@]1(O)C(=O)OCc2c1cc1n(c2=O)Cc2c-1nc1cc(F)c(N)cc1c2COC(=O)NC] | (S)-(9-amino-4-ethyl-8-fluoro-4-hydroxy- 3,14-dioxo-3,4,12,14-tetrahydro-1H- pyrano[3′,4′:6,7]indolizino[1,2-b]quinolin- 11-yl)methyl methylcarbamate | Compound 162 |
| 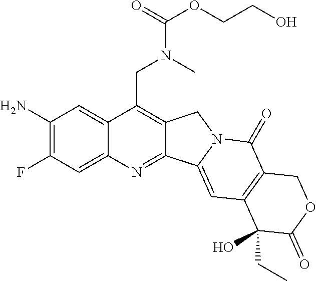 [SMILES: CC[C@@]1(O)C(=O)OCc2c1cc1n(c2=O)Cc2c-1nc1cc(F)c(N)cc1c2CN(C)C(=O)OCCO] | 2-hydroxyethyl (S)-((9-amino-4-ethyl-8- fluoro-4-hydroxy-3,14-dioxo-3,4,12,14- tetrahydro-1H- pyrano[3′,4′:6,7]indolizino[1,2-b]quinolin- 11-yl)methyl)(methyl)carbamate | Compound 163 |
| 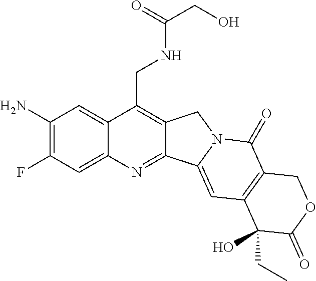 [SMILES: CC[C@@]1(O)C(=O)OCc2c1cc1n(c2=O)Cc2c-1nc1cc(F)c(N)cc1c2CNC(=O)CO] | (S)-N-((9-amino-4-ethyl-8-fluoro-4- hydroxy-3,14-dioxo-3,4,12,14-tetrahydro- 1H-pyrano[3′,4′:6,7]indolizino[1,2- b]quinolin-11-yl)methyl)-2- hydroxyacetamide | Compound 164 |
| 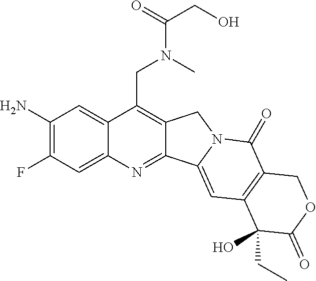 [SMILES: CC[C@@]1(O)C(=O)OCc2c1cc1n(c2=O)Cc2c-1nc1cc(F)c(N)cc1c2CN(C)C(=O)CO] | (S)-N-((9-amino-4-ethyl-8-fluoro-4- hydroxy-3,14-dioxo-3,4,12,14-tetrahydro- 1H-pyrano[3′,4′:6,7]indolizino[1,2- b]quinolin-11-yl)methyl)-2-hydroxy-N- methylacetamide | Compound 165 |
| 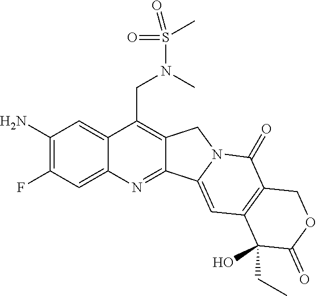 [SMILES: CC[C@@]1(O)C(=O)OCc2c1cc1n(c2=O)Cc2c-1nc1cc(F)c(N)cc1c2CN(C)S(C)(=O)=O] | (S)-N-((9-amino-4-ethyl-8-fluoro-4- hydroxy-3,14-dioxo-3,4,12,14-tetrahydro- 1H-pyrano[3′,4′:6,7]indolizino[1,2- b]quinolin-11-yl)methyl)-N- methylmethanesulfonamide | Compound 166 |
| 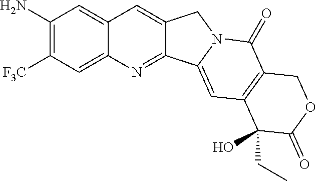 [SMILES: CC[C@@]1(O)C(=O)OCc2c1cc1n(c2=O)Cc2cc3cc(N)c(C(F)(F)F)cc3nc2-1] | (S)-9-amino-4-ethyl-4-hydroxy-8- (trifluoromethyl)-1,12-dihydro-14H- pyrano[3′,4′:6,7]indolizino[1,2- b]quinoline-3,14(4H)-dione | Compound 167 |
| 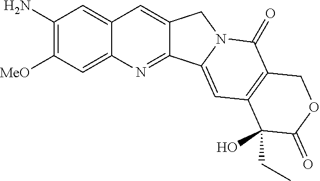 [SMILES: CC[C@@]1(O)C(=O)OCc2c1cc1n(c2=O)Cc2cc3cc(N)c(OC)cc3nc2-1] | (S)-9-amino-4-ethyl-4-hydroxy-8- methoxy-1,12-dihydro-14H- pyrano[3′,4′:6,7]indolizino[1,2- b]quinoline-3,14(4H)-dione | Compound 168 |
| 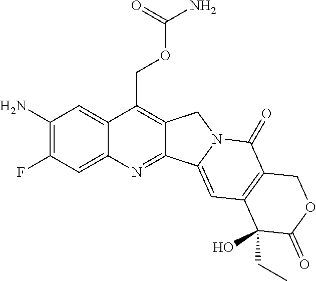 [SMILES: CC[C@@]1(O)C(=O)OCc2c1cc1n(c2=O)Cc2c-1nc1cc(F)c(N)cc1c2COC(N)=O] | (S)-(9-amino-4-ethyl-8-fluoro-4-hydroxy- 3,14-dioxo-3,4,12,14-tetrahydro-1H- pyrano[3′,4′:6,7]indolizino[1,2-b]quinolin- 11-yl)methyl carbamate | Compound 169 |
| 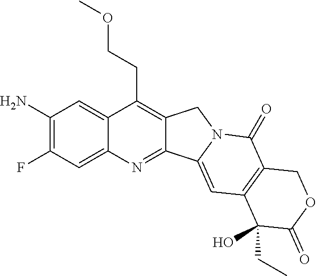 [SMILES: CC[C@@]1(O)C(=O)OCc2c1cc1n(c2=O)Cc2c-1nc1cc(F)c(N)cc1c2CCOC] | (S)-9-amino-4-ethyl-8-fluoro-4-hydroxy- 11-(2-methoxyethyl)-1,12-dihydro-14H- pyrano[3′,4′:6,7]indolizino[1,2- b]quinoline-3,14(4H)-dione | Compound 170 |
| 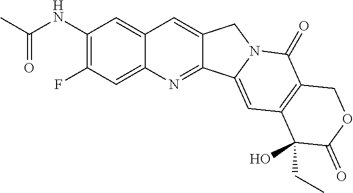 [SMILES: CC[C@@]1(O)C(=O)OCc2c1cc1n(c2=O)Cc2cc3cc(NC(C)=O)c(F)cc3nc2-1] | (S)-N-(4-ethyl-8-fluoro-4-hydroxy-3,14- dioxo-3,4,12,14-tetrahydro-1H- pyrano[3′,4′:6,7]indolizino[1,2-b]quinolin- 9-yl)acetamide | Compound 171 |

In certain embodiments, the camptothecin analogue is a compound having Formula (II) or a protected version thereof and is selected from the compounds shown in Table 2.

TABLE 2
Exemplary Camptothecin Analogues of Formula (II)

| | Compound | |
|---|---|---|
| Structure | Name | Number |
| 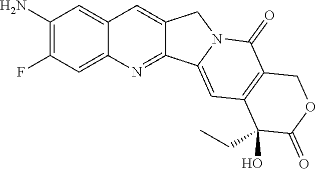 [SMILES: CC[C@@]1(O)C(=O)OCc2c1cc1n(c2=O)Cc2cc3cc(N)c(F)cc3nc2-1] | (S)-9-amino-4-ethyl-8-fluoro- 4-hydroxy-1,12-dihydro-14H- pyrano[3′,4′:6,7]indolizino- [1,2-b]quinoline-3,14(4H)- dione | Compound 140 |
| 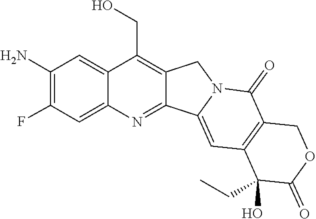 [SMILES: CC[C@@]1(O)C(=O)OCc2c1cc1n(c2=O)Cc2c-1nc1cc(F)c(N)cc1c2CO] | (S)-9-amino-4-ethyl-8-fluoro- 4-hydroxy-11-(hydroxymeth- yl)-1,12-dihydro-14H-pyrano- [3′,4′:6,7]indolizino[1,2-b]- quinoline-3,14(4H)-dione | Compound 141 |
| 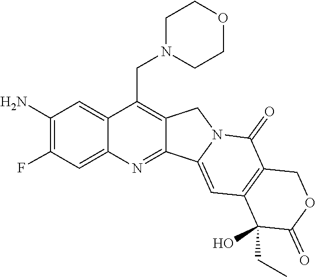 [SMILES: CC[C@@]1(O)C(=O)OCc2c1cc1n(c2=O)Cc2c-1nc1cc(F)c(N)cc1c2CN1CCOCC1] | (S)-9-amino-4-ethyl-8-fluoro- 4-hydroxy-11-(morpholino- methyl)-1,12-dihydro-14H- pyrano[3′,4′:6,7]indolizino[1,2- b]quinoline-3,14(4H)-dione | Compound 142 |
| 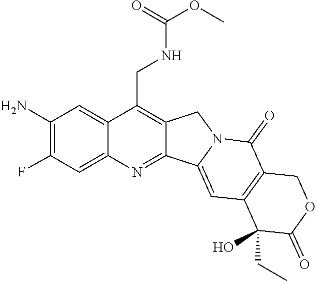 [SMILES: CC[C@@]1(O)C(=O)OCc2c1cc1n(c2=O)Cc2c-1nc1cc(F)c(N)cc1c2CNC(=O)OC] | methyl (S)-((9-amino-4-ethyl- 8-fluoro-4-hydroxy-3,14- dioxo-3,4,12,14-tetrahydro-1H- pyrano[3′,4′:6,7]indolizino[1,2- b]quinolin-11-yl)methyl)- carbamate | Compound 143 |
| 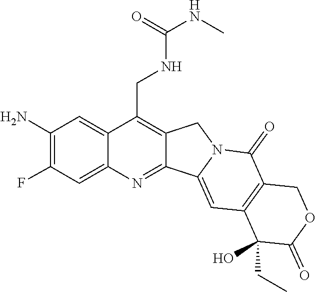 [SMILES: CC[C@@]1(O)C(=O)OCc2c1cc1n(c2=O)Cc2c-1nc1cc(F)c(N)cc1c2CNC(=O)NC] | (S)-1-((9-amino-4-ethyl-8- fluoro-4-hydroxy-3,14-dioxo- 3,4,12,14-tetrahydro-1H- pyrano[3′,4′:6,7]indolizino- [1,2-b]quinolin-11-yl)methyl)- 3-methylurea | Compound 144 |
| 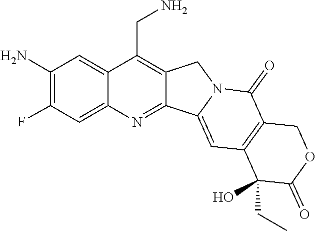 [SMILES: CC[C@@]1(O)C(=O)OCc2c1cc1n(c2=O)Cc2c-1nc1cc(F)c(N)cc1c2CN] | (S)-9-amino-11-(aminometh- yl)-4-ethyl-8-fluoro-4-hydroxy- 1,12-dihydro-14H-pyrano- [3′,4′:6,7]indolizino[1,2-b]- quinoline-3,14(4H)-dione | Compound 145 |
| 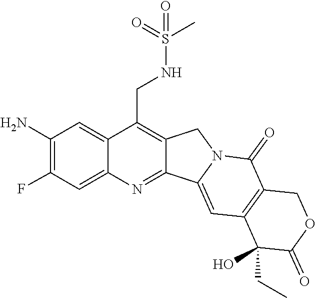 [SMILES: CC[C@@]1(O)C(=O)OCc2c1cc1n(c2=O)Cc2c-1nc1cc(F)c(N)cc1c2CNS(C)(=O)=O] | (S)-N-((9-amino-4-ethyl-8- fluoro-4-hydroxy-3,14-dioxo- 3,4,12,14-tetrahydro-1H- pyrano[3′,4′:6,7]indolizino- [1,2-b]quinolin-11-yl)methyl)- methanesulfonamide | Compound 146 |
| 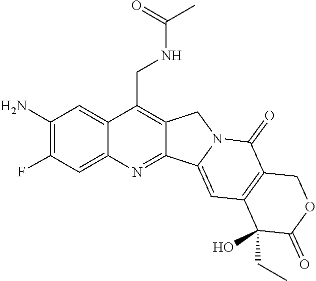 [SMILES: CC[C@@]1(O)C(=O)OCc2c1cc1n(c2=O)Cc2c-1nc1cc(F)c(N)cc1c2CNC(C)=O] | (S)-N-((9-amino-4-ethyl-8- fluoro-4-hydroxy-3,14-dioxo- 3,4,12,14-tetrahydro-1H- pyrano[3′,4′:6,7]indolizino- [1,2-b]quinolin-11-yl)methyl)- acetamide | Compound 147 |
| 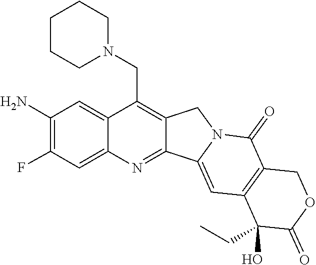 [SMILES: CC[C@@]1(O)C(=O)OCc2c1cc1n(c2=O)Cc2c-1nc1cc(F)c(N)cc1c2CN1CCCCC1] | (S)-9-amino-4-ethyl-8-fluoro- 4-hydroxy-11-(piperidin-1- ylmethyl)-1,12-dihydro-14H- pyrano[3′,4′:6,7]indolizino[1,2- b]quinoline-3,14(4H)-dione | Compound 148 |
| 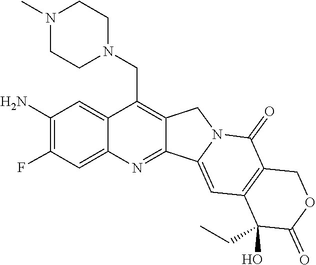 [SMILES: CC[C@@]1(O)C(=O)OCc2c1cc1n(c2=O)Cc2c-1nc1cc(F)c(N)cc1c2CN1CCN(C)CC1] | (S)-9-amino-4-ethyl-8-fluoro- 4-hydroxy-11-((4-methylpiper- azin-1-yl)methyl)-1,12- dihydro-14H-pyrano[3′,4′:6,7]- indolizino[1,2-b]quinoline-3, 14(4H)-dione | Compound 149 |
| 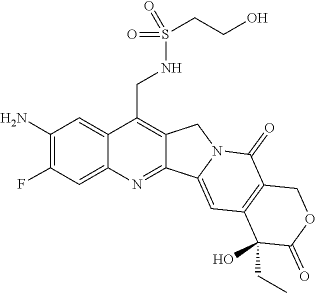 [SMILES: CC[C@@]1(O)C(=O)OCc2c1cc1n(c2=O)Cc2c-1nc1cc(F)c(N)cc1c2CNS(=O)(=O)CCO] | (S)-N-((9-amino-4-ethyl-8- fluoro-4-hydroxy-3,14-dioxo- 3,4,12,14-tetrahydro-1H- pyrano[3′,4′:6,7]indolizino- [1,2-b]quinolin-11-yl)methyl)-2- hydroxyethane-1-sulfonamide | Compound 150 |
| 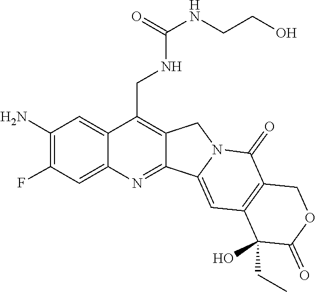 [SMILES: CC[C@@]1(O)C(=O)OCc2c1cc1n(c2=O)Cc2c-1nc1cc(F)c(N)cc1c2CNC(=O)NCCO] | (S)-1-((9-amino-4-ethyl-8- fluoro-4-hydroxy-3,14-dioxo- 3,4,12,14-tetrahydro-1H- pyrano[3′,4′:6,7]indolizino- [1,2-b]quinolin-11-yl)methyl)- 3-(2-hydroxyethyl)urea | Compound 151 |
| 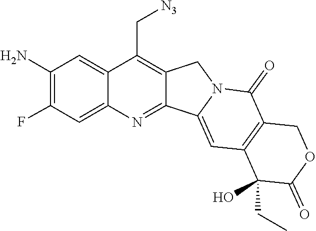 [SMILES: CC[C@@]1(O)C(=O)OCc2c1cc1n(c2=O)Cc2c-1nc1cc(F)c(N)cc1c2CN=[N+]=[N-]] | (S)-9-amino-11-(azidomethyl)- 4-ethyl-8-fluoro-4-hydroxy- 1,12-dihydro-14H-pyrano[3′,4′: 6,7]indolizino[1,2-b]quinoline- 3,14(4H)-dione | Compound 152 |
| 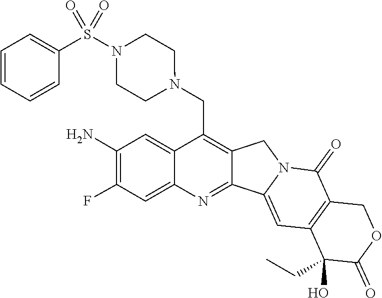 [SMILES: CC[C@@]1(O)C(=O)OCc2c1cc1n(c2=O)Cc2c-1nc1cc(F)c(N)cc1c2CN1CCN(S(=O)(=O)c2ccccc2)CC1] | (S)-9-amino-4-ethyl-8-fluoro- 4-hydroxy-11-((4-(phenylsul- fonyl)piperazin-1-yl)methyl)- 1,12-dihydro-14H-pyrano[3′,4′: 6,7]indolizino[1,2-b]quinoline- 3,14(4H)-dion | Compound 153 |
| 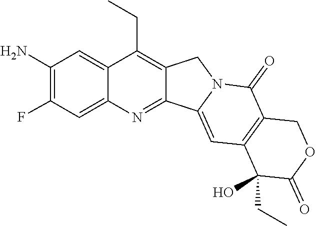 [SMILES: CCc1c2c(nc3cc(F)c(N)cc13)-c1cc3c(c(=O)n1C2)COC(=O)[C@]3(O)CC] | (S)-9-amino-4,11-diethyl-8- fluoro-4-hydroxy-1,12-dihydro- 14H-pyrano[3′,4′:6,7]indoli- zino[1,2-b]quinoline-3,14(4H)- dione | Compound 154 |
| 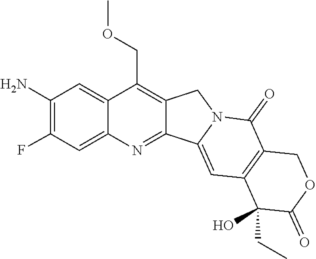 [SMILES: CC[C@@]1(O)C(=O)OCc2c1cc1n(c2=O)Cc2c-1nc1cc(F)c(N)cc1c2COC] | (S)-9-amino-4-ethyl-8-fluoro- 4-hydroxy-11-(methoxymeth- yl)-1,12-dihydro-14H-pyrano- [3′,4′:6,7]indolizino[1,2-b]- quinoline-3,14(4H)-dione | Compound 155 |
| 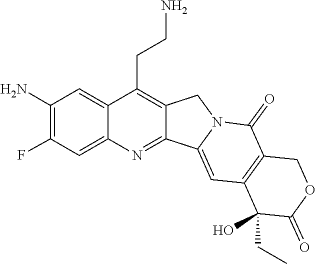 [SMILES: CC[C@@]1(O)C(=O)OCc2c1cc1n(c2=O)Cc2c-1nc1cc(F)c(N)cc1c2CCN] | (S)-9-amino-11-(2-aminoeth- yl)-4-ethyl-8-fluoro-4-hydroxy- 1,12-dihydro-14H-pyrano[3′,4′: 6,7]indolizino[1,2-b]quinoline- 3,14(4H)-dione | Compound 156 |
| 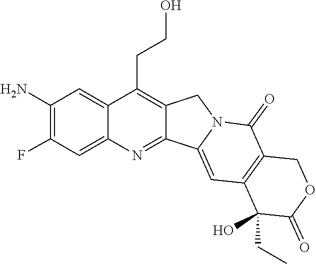 [SMILES: CC[C@@]1(O)C(=O)OCc2c1cc1n(c2=O)Cc2c-1nc1cc(F)c(N)cc1c2CCO] | (S)-9-amino-4-ethyl-8-fluoro- 4-hydroxy-11-(2-hydroxyeth- yl)-1,12-dihydro-14H-pyrano- [3′,4′:6,7]indolizino[1,2-b]- quinoline-3,14(4H)-dione | Compound 157 |
| 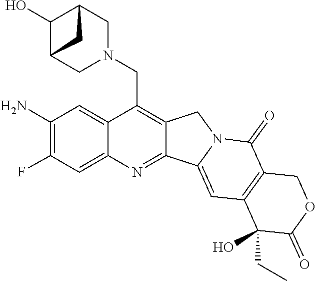 [SMILES: CC[C@@]1(O)C(=O)OCc2c1cc1n(c2=O)Cc2c-1nc1cc(F)c(N)cc1c2CN1C[C@H]2C[C@@H](C1)C2O] | (4S)-9-amino-4-ethyl-8-fluoro- 4-hydroxy-11-(((1R,5S)-6- hydroxy-3-azabicyclo[3.1.1]- heptan-3-yl)methyl)-1,12- dihydro-14H-pyrano[3′,4′:6,7]- indolizino[1,2-b]quinoline- 3,14(4H)-dione | Compound 158 |
| 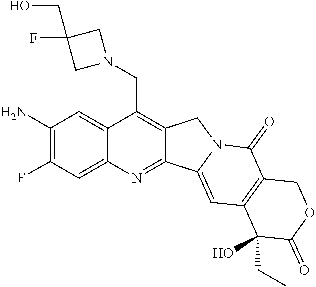 [SMILES: CC[C@@]1(O)C(=O)OCc2c1cc1n(c2=O)Cc2c-1nc1cc(F)c(N)cc1c2CN1CC(F)(CO)C1] | (S)-9-amino-4-ethyl-8-fluoro- 11-((3-fluoro-3-(hydroxy- methyl)azetidin-1-yl)methyl)- 4-hydroxy-1,12-dihydro-14H- pyrano[3′,4′:6,7]indolizino[1,2- b]quinoline-3,14(4H)-dione | Compound 159 |
| 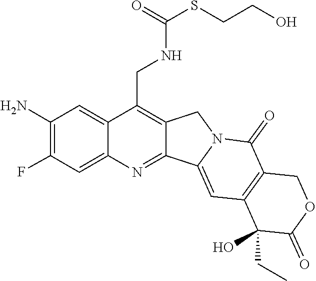 [SMILES: CC[C@@]1(O)C(=O)OCc2c1cc1n(c2=O)Cc2c-1nc1cc(F)c(N)cc1c2CNC(=O)SCCO] | S-(2-hydroxyethyl) (S)-((9- amino-4-ethyl-8-fluoro-4- hydroxy-3,14-dioxo-3,4,12,14- tetrahydro-1H-pyrano[3′,4′:6,7]- indolizino[1,2-b]quinolin-11- yl)methyl)carbamothioate | Compound 160 |
| 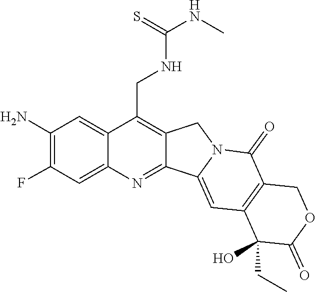 [SMILES: CC[C@@]1(O)C(=O)OCc2c1cc1n(c2=O)Cc2c-1nc1cc(F)c(N)cc1c2CNC(=S)NC] | (S)-1-((9-amino-4-ethyl-8- fluoro-4-hydroxy-3,14-dioxo- 3,4,12,14-tetrahydro-1H- pyrano[3′,4′:6,7]indolizino- [1,2-b]quinolin-11-yl)methyl)- 3-methylthiourea | Compound 161 |
| 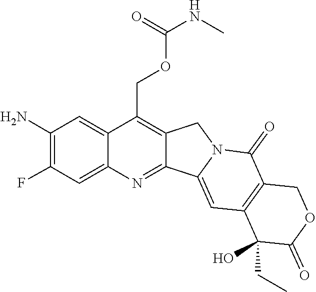 [SMILES: CC[C@@]1(O)C(=O)OCc2c1cc1n(c2=O)Cc2c-1nc1cc(F)c(N)cc1c2COC(=O)NC] | (S)-(9-amino-4-ethyl-8-fluoro- 4-hydroxy-3,14-dioxo-3,4,12, 14-tetrahydro-1H-pyrano- [3′,4′:6,7]indolizino[1,2-b]- quinolin-11-yl)methyl methyl- carbamate | Compound 162 |
| 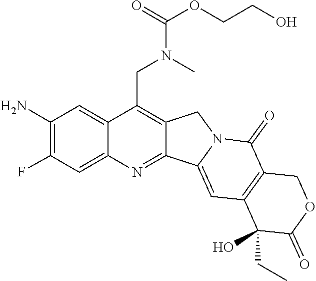 [SMILES: CC[C@@]1(O)C(=O)OCc2c1cc1n(c2=O)Cc2c-1nc1cc(F)c(N)cc1c2CN(C)C(=O)OCCO] | 2-hydroxyethyl (S)-((9-amino- 4-ethyl-8-fluoro-4-hydroxy- 3,14-dioxo-3,4,12,14-tetra- hydro-1H-pyrano[3′,4′:6,7]- indolizino[1,2-b]quinolin-11- yl)methyl)(methyl)carbamate | Compound 163 |
| 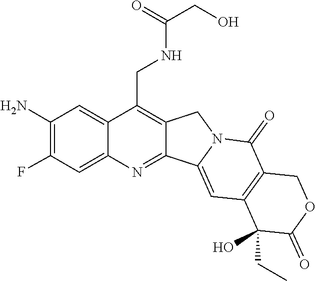 [SMILES: CC[C@@]1(O)C(=O)OCc2c1cc1n(c2=O)Cc2c-1nc1cc(F)c(N)cc1c2CNC(=O)CO] | (S)-N-((9-amino-4-ethyl-8- fluoro-4-hydroxy-3,14-dioxo- 3,4,12,14-tetrahydro-1H- pyrano[3′,4′:6,7]indolizino- [1,2-b]quinolin-11-yl)methyl)- 2-hydroxyacetamide | Compound 164 |
| 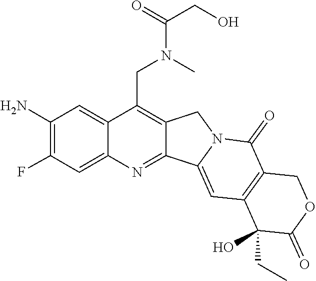 [SMILES: CC[C@@]1(O)C(=O)OCc2c1cc1n(c2=O)Cc2c-1nc1cc(F)c(N)cc1c2CN(C)C(=O)CO] | (S)-N-((9-amino-4-ethyl-8- fluoro-4-hydroxy-3,14-dioxo- 3,4,12,14-tetrahydro-1H- pyrano[3′,4′:6,7]indolizino- [1,2-b]quinolin-11-yl)methyl)- 2-hydroxy-N-methylacetamide | Compound 165 |
| 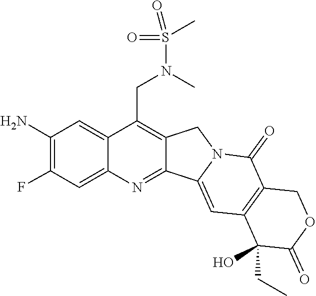 [SMILES: CC[C@@]1(O)C(=O)OCc2c1cc1n(c2=O)Cc2c-1nc1cc(F)c(N)cc1c2CN(C)S(C)(=O)=O] | (S)-N-((9-amino-4-ethyl-8- fluoro-4-hydroxy-3,14-dioxo- 3,4,12,14-tetrahydro-1H- pyrano[3′,4′:6,7]indolizino- [1,2-b]quinolin-11-yl)methyl)- N-methylmethanesulfonamide | Compound 166 |
| 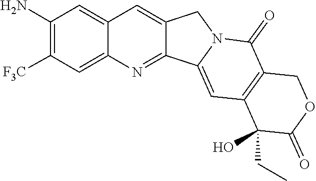 [SMILES: CC[C@@]1(O)C(=O)OCc2c1cc1n(c2=O)Cc2cc3cc(N)c(C(F)(F)F)cc3nc2-1] | (S)-9-amino-4-ethyl-4- hydroxy-8-(trifluoromethyl)- 1,12-dihydro-14H-pyrano[3′,4′: 6,7]indolizino[1,2-b]quinoline- 3,14(4H)-dione | Compound 167 |
| 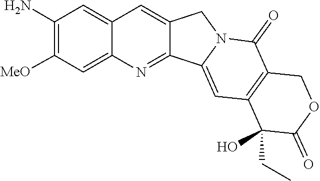 [SMILES: CC[C@@]1(O)C(=O)OCc2c1cc1n(c2=O)Cc2cc3cc(N)c(OC)cc3nc2-1] | (S)-9-amino-4-ethyl-4- hydroxy-8-methoxy-1,12- dihydro-14H-pyrano[3′,4′:6,7]- indolizino[1,2-b]quinoline- 3,14(4H)-dione | Compound 168 |
| 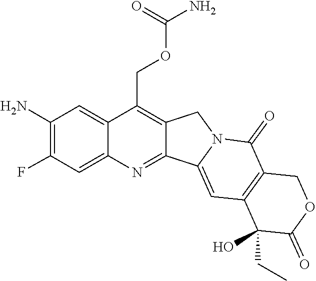 [SMILES: CC[C@@]1(O)C(=O)OCc2c1cc1n(c2=O)Cc2c-1nc1cc(F)c(N)cc1c2COC(N)=O] | (S)-(9-amino-4-ethyl-8-fluoro- 4-hydroxy-3,14-dioxo-3,4,12, 14-tetrahydro-1H-pyrano[3′,4′: 6,7]indolizino[1,2-b]quinolin- 11-yl)methyl carbamate | Compound 169 |
| 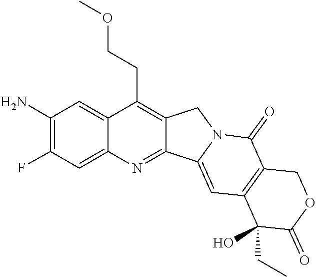 [SMILES: CC[C@@]1(O)C(=O)OCc2c1cc1n(c2=O)Cc2c-1nc1cc(F)c(N)cc1c2CCOC] | (S)-9-amino-4-ethyl-8-fluoro- 4-hydroxy-11-(2-methoxyeth- yl)-1,12-dihydro-14H-pyrano- [3′,4′:6,7]indolizino[1,2-b]- quinoline-3,14(4H)-dione | Compound 170 |
| 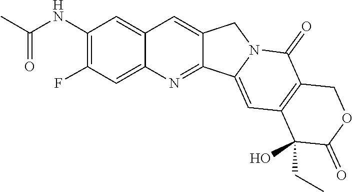 [SMILES: CC[C@@]1(O)C(=O)OCc2c1cc1n(c2=O)Cc2cc3cc(NC(C)=O)c(F)cc3nc2-1] | (S)-N-(4-ethyl-8-fluoro-4- hydroxy-3,14-dioxo-3,4,12,14- tetrahydro-1H-pyrano[3′,4′: 6,7]indolizino[1,2-b]quinolin- 9-yl)acetamide | Compound 171 |

In certain embodiments, the camptothecin analogue is a compound having Formula (III) or a protected version thereof and is selected from the compounds shown in Table 3.

TABLE 3
Exemplary Camptothecin Analogues of Formula (III)

| | Compound | |
|---|---|---|
| Structure | Name | Number |
| 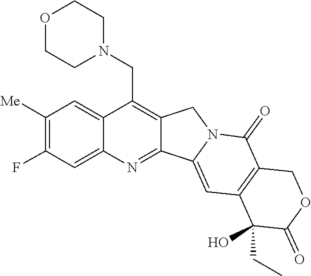 [SMILES: CC[C@@]1(O)C(=O)OCc2c1cc1n(c2=O)Cc2c-1nc1cc(F)c(C)cc1c2CN1CCOCC1] | (S)-4-ethyl-8-fluoro-4-hydroxy- 9-methyl-11-(morpholinometh- yl)-1,12-dihydro-14H-pyrano- [3′,4′:6,7]indolizino[1,2-b]quin- oline-3,14(4H)-dione | Compound 100 |
| 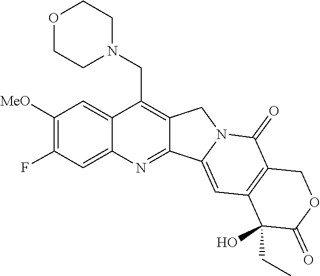 [SMILES: CC[C@@]1(O)C(=O)OCc2c1cc1n(c2=O)Cc2c-1nc1cc(F)c(OC)cc1c2CN1CCOCC1] | (S)-4-ethyl-8-fluoro-4-hydroxy- 9-methoxy-11-morpholinometh- yl)-1,12-dihydro-14H-pyrano- [3′,4′:6,7]indolizino[1,2-b]quin- oline-3,14(4H)-dione | Compound 101 |
| 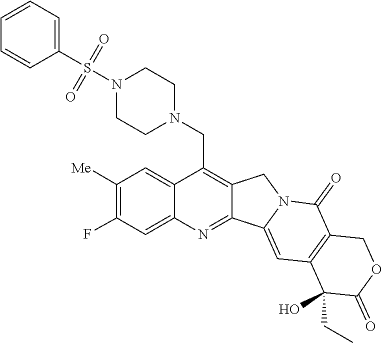 [SMILES: CC[C@@]1(O)C(=O)OCc2c1cc1n(c2=O)Cc2c-1nc1cc(F)c(C)cc1c2CN1CCN(S(=O)(=O)c2ccccc2)CC1] | (S)-4-ethyl-8-fluoro-4-hydroxy- 9-methyl-11-((4-(phenylsulfon- yl)piperazin-1-yl)methyl)-1,12- dihydro-14H-pyrano[3′,4′:6,7]- indolizino[1,2-b]quinoline- 3,14(4H)-dione | Compound 102 |
| 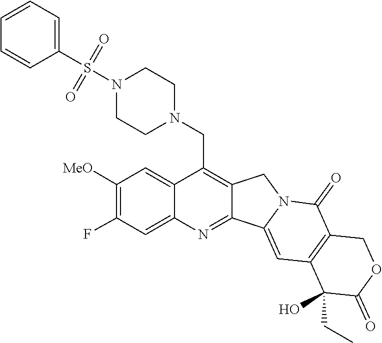 [SMILES: CC[C@@]1(O)C(=O)OCc2c1cc1n(c2=O)Cc2c-1nc1cc(F)c(OC)cc1c2CN1CCN(S(=O)(=O)c2ccccc2)CC1] | (S)-4-ethyl-8-fluoro-4-hydroxy- 9-methoxy-11-((4-(phenylsulfon- yl)piperazin-1-yl)methyl)-1,12- dihydro-14H-pyrano[3′,4′:6,7]- indolizino[1,2-b]quinoline- 3,14(4H)-dione | Compound 103 |
| 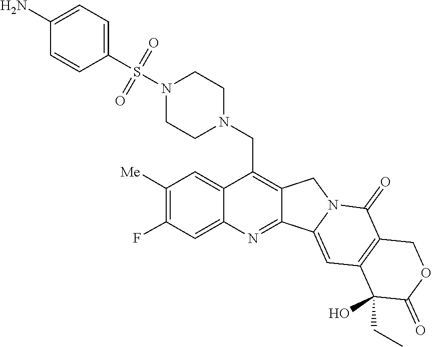 [SMILES: CC[C@@]1(O)C(=O)OCc2c1cc1n(c2=O)Cc2c-1nc1cc(F)c(C)cc1c2CN1CCN(S(=O)(=O)c2ccc(N)cc2)CC1] | (S)-11-((4-((4-aminophenyl)sul- fonyl)piperazin-1-yl)methyl)-4- ethyl-8-fluoro-4-hydroxy-9- methyl-1,12-dihydro-14H- pyrano[3′,4′:6,7]indolizino[1,2- b]quinoline-3,14(4H)-dione | Compound 104 |
| 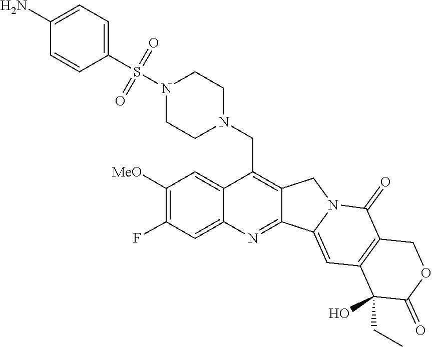 [SMILES: CC[C@@]1(O)C(=O)OCc2c1cc1n(c2=O)Cc2c-1nc1cc(F)c(OC)cc1c2CN1CCN(S(=O)(=O)c2ccc(N)cc2)CC1] | (S)-11-((4-((4-aminophenyl)- sulfonyl)piperazin-1-yl)methyl)- 4-ethyl-8-fluoro-4-hydroxy-9- methoxy-1,12-dihydro-14H- pyrano[3′,4′:6,7]indolizino[1,2- b]quinoline-3,14(4H)-dione | Compound 105 |
| 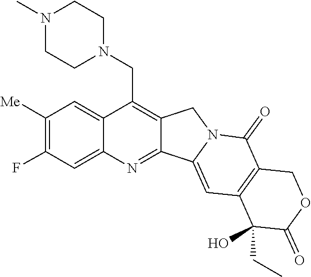 [SMILES: CC[C@@]1(O)C(=O)OCc2c1cc1n(c2=O)Cc2c-1nc1cc(F)c(C)cc1c2CN1CCN(C)CC1] | (S)-4-ethyl-8-fluoro-4-hydroxy- 9-methyl-11-((4-methylpiper- azin-1-yl)methyl)-1,12-dihydro- 14H-pyrano[3′4′:6,7]indolizino- [1,2-b]quinoline-3,14(4H)-dione | Compound 106 |
| 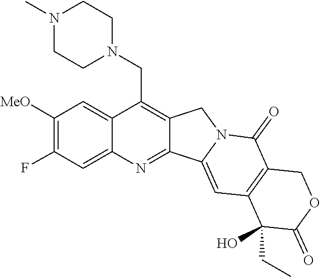 [SMILES: CC[C@@]1(O)C(=O)OCc2c1cc1n(c2=O)Cc2c-1nc1cc(F)c(OC)cc1c2CN1CCN(C)CC1] | (S)-4-ethyl-8-fluoro-4-hydroxy- 9-methoxy-11-((4-methylpiper- azin-1-yl)methyl)-1,12-dihydro- 14H-pyrano[3′,4′:6,7]indolizino- [1,2-b]quinoline-3,14(4H)-dione | Compound 107 |
| 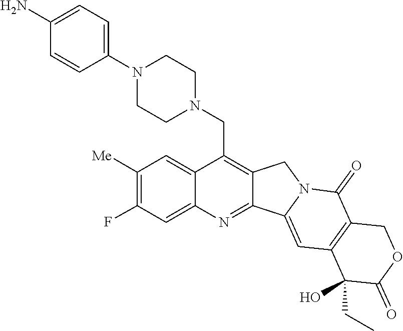 [SMILES: CC[C@@]1(O)C(=O)OCc2c1cc1n(c2=O)Cc2c-1nc1cc(F)c(C)cc1c2CN1CCN(c2ccc(N)cc2)CC1] | (S)-11-((4-(4-aminophenyl)- piperazin-1-yl)methyl)-4-ethyl- 8-fluoro-4-hydroxy-9-methyl- 1,12-dihydro-14H-pyrano- [3′,4′:6,7]indolizino[1,2-b]- quinoline-3,14(4H)-dione | Compound 108 |
| 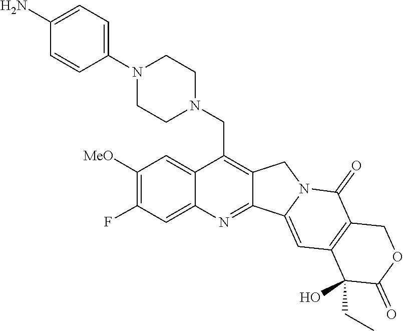 [SMILES: CC[C@@]1(O)C(=O)OCc2c1cc1n(c2=O)Cc2c-1nc1cc(F)c(OC)cc1c2CN1CCN(c2ccc(N)cc2)CC1] | (S)-11-((4-(4-aminophenyl)- piperazin-1-yl)methyl)-4-ethyl- 8-fluoro-4-hydroxy-9-methoxy- 1,12-dihydro-14H-pyrano- [3′4′:6,7]indolizino[1,2-b]- quinoline-3,14(4H)-dione | Compound 109 |
| 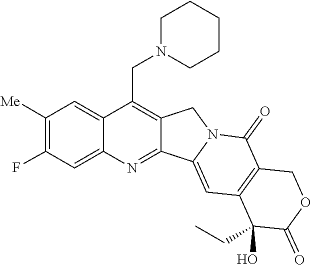 [SMILES: CC[C@@]1(O)C(=O)OCc2c1cc1n(c2=O)Cc2c-1nc1cc(F)c(C)cc1c2CN1CCCCC1] | (S)-4-ethyl-8-fluoro-4-hydroxy- 9-methyl-11-(piperidin-1-yl- methyl)-1,12-dihydro-14H- pyrano[3′,4′:6,7]indolizino[1,2- b]quinoline-3,14(4H)-dione | Compound 110 |
| 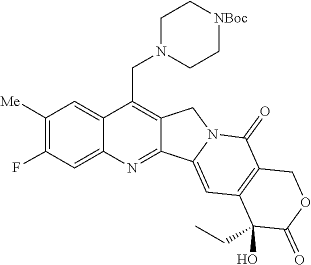 [SMILES: CC[C@@]1(O)C(=O)OCc2c1cc1n(c2=O)Cc2c-1nc1cc(F)c(C)cc1c2CN1CCN(C(=O)OC(C)(C)C)CC1] | tert-butyl (S)-4-((4-ethyl-8- fluoro-4-hydroxy-9-methyl- 3,14-dioxo-3,4,12,14-tetrahydro- 1H-pyrano[3′,4′:6,7]indolizino- [1,2-b]quinolin-11-yl)methyl)- piperazine-1-carboxylate | Compound 111 |
| 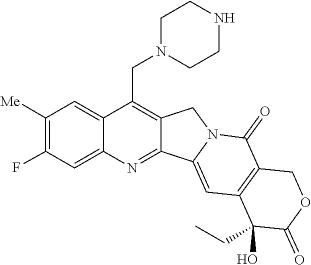 [SMILES: CC[C@@]1(O)C(=O)OCc2c1cc1n(c2=O)Cc2c-1nc1cc(F)c(C)cc1c2CN1CCNCC1] | (S)-4-ethyl-8-fluoro-4-hydroxy- 9-methyl-11-(piperazin-1-yl- methyl)-1,12-dihydro-14H- pyrano[3′,4′:6,7]indolizino[1,2- b]quinoline-3,14(4H)-dione | Compound 112 |
| 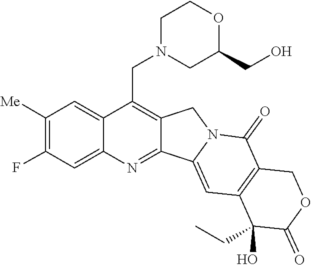 [SMILES: CC[C@@]1(O)C(=O)OCc2c1cc1n(c2=O)Cc2c-1nc1cc(F)c(C)cc1c2CN1CCO[C@@H](CO)C1] | (S)-4-ethyl-8-fluoro-4-hydroxy- 11-(((R)-2-(hydroxymethyl)- morpholino)methyl)-9-methyl- 1,12-dihydro-14H-pyrano- [3′,4′:6,7]indolizino[1,2-b]quin- oline-3,14(4H)-dione | Compound 113 |
| 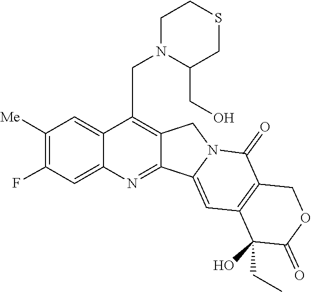 [SMILES: CC[C@@]1(O)C(=O)OCc2c1cc1n(c2=O)Cc2c-1nc1cc(F)c(C)cc1c2CN1CCSCC1CO] | (4S)-4-ethyl-8-fluoro-4-hydroxy- 11-((3-(hydroxymethyl)thio- morpholino)methyl)-9-methyl- 1,12-dihydro-14H-pyrano[3′,4′: 6,7]indolizino[1,2-b]quinoline-3,14(4H)-dione | Compound 114 |
| 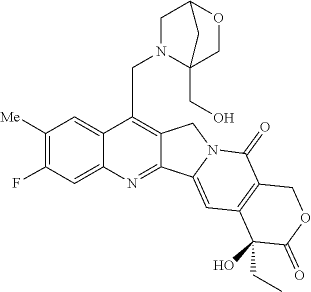 [SMILES: CC[C@@]1(O)C(=O)OCc2c1cc1n(c2=O)Cc2c-1nc1cc(F)c(C)cc1c2CN1CC2CC1(CO)CO2] | (4S)-4-ethyl-8-fluoro-4-hydroxy- 11-((4-(hydroxymethyl)-2-oxa- 5-azabicyclo[2.2.1]heptan-5-yl)- methyl)-9-methyl-1,12-dihydro- 14H-pyrano[3′,4′:6,7]indolizino- [1,2-b]quinoline-3,14(4H)-dione | Compound 115 |
| 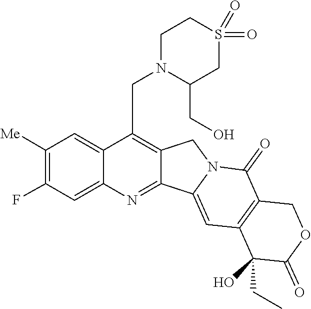 [SMILES: CC[C@@]1(O)C(=O)OCc2c1cc1n(c2=O)Cc2c-1nc1cc(F)c(C)cc1c2CN1CCS(=O)(=O)CC1CO] | (4S)-4-ethyl-8-fluoro-4-hydroxy- 11-((3-(hydroxymethyl)-1,1- dioxidothiomorpholino)methyl)- 9-methyl-1,12-dihydro-14H- pyrano[3′,4′:6,7]indolizino[1,2- b]quinoline-3,14(4H)-dione | Compound 116 |
| 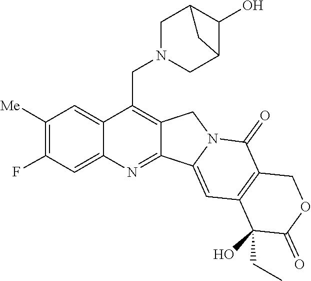 [SMILES: CC[C@@]1(O)C(=O)OCc2c1cc1n(c2=O)Cc2c-1nc1cc(F)c(C)cc1c2CN1CC2CC(C1)C2O] | (4S)-4-ethyl-8-fluoro-4-hydroxy- 11-((6-hydroxy-3-azabicyclo- [3.1.1]heptan-3-yl)methyl)-9- methyl-1,12-dihydro-14H- pyrano[3′,4′:6,7]indolizino[1,2- b]quinoline-3,14(4H)-dione | Compound 117 |
| 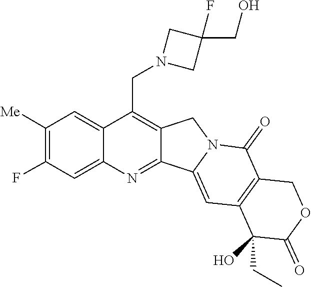 [SMILES: CC[C@@]1(O)C(=O)OCc2c1cc1n(c2=O)Cc2c-1nc1cc(F)c(C)cc1c2CN1CC(F)(CO)C1] | (S)-4-ethyl-8-fluoro-11-((3- fluoro-3-(hydroxymethyl)azeti- din-1-yl)methyl)-4-hydroxy-9- methyl-1,12-dihydro-14H- pyrano[3′,4′:6,7]indolizino[1,2- b]quinoline-3,14(4H)-dione | Compound 118 |
| 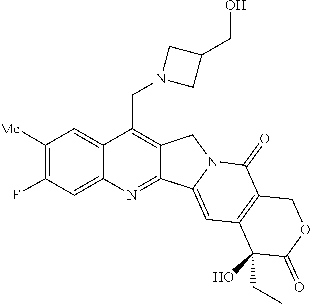 [SMILES: CC[C@@]1(O)C(=O)OCc2c1cc1n(c2=O)Cc2c-1nc1cc(F)c(C)cc1c2CN1CC(CO)C1] | (S)-4-ethyl-8-fluoro-4-hydroxy- 11-((3-(hydroxymethyl)azetidin- 1-yl)methyl)-9-methyl-1,12- dihydro-14H-pyrano[3′,4′:6,7]- indolizino[1,2-b]quinoline- 3,14(4H)-dione | Compound 119 |
| 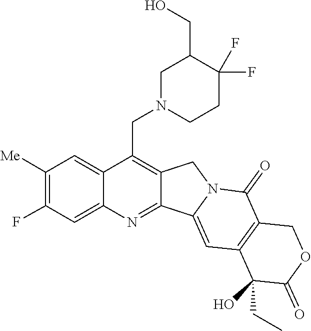 [SMILES: CC[C@@]1(O)C(=O)OCc2c1cc1n(c2=O)Cc2c-1nc1cc(F)c(C)cc1c2CN1CCC(F)(F)C(CO)C1] | (4S)-11-((4,4-difluoro-3- (hydroxymethyl)piperidin-1-yl)- methyl)-4-ethyl-8-fluoro-4- hydroxy-9-methyl-1,12-dihydro- 14H-pyrano[3′,4′:6,7]indolizino- [1,2-b]quinoline-3,14(4H)-dione | Compound 120 |
| 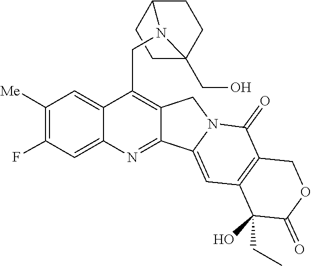 [SMILES: CC[C@@]1(O)C(=O)OCc2c1cc1n(c2=O)Cc2c-1nc1cc(F)c(C)cc1c2CN1C2CCC1(CO)CC2] | (4S)-11-((4,4-difluoro-3- (hydroxymethyl)piperidin-1-yl)- methyl)-4-ethyl-8-fluoro-4- hydroxy-9-methyl-1,12-dihydro- 14H-pyrano[3′,4′:6,7]indolizino- [1,2-b]quinoline-3,14(4H)-dione | Compound 121 |
| 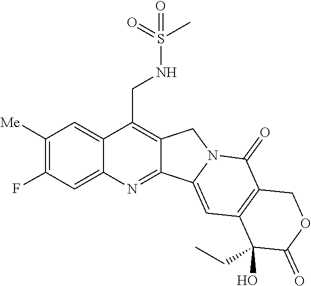 [SMILES: CC[C@@]1(O)C(=O)OCc2c1cc1n(c2=O)Cc2c-1nc1cc(F)c(C)cc1c2CNS(C)(=O)=O] | (S)-N-((4-ethyl-8-fluoro-4- hydroxy-9-methyl-3,14-dioxo- 3,4,12,14-tetrahydro-1H-pyrano- [3′,4′:6,7]indolizino[1,2-b]quin- olin-11-yl)methyl)methane- sulfonamide | Compound 122 |
| 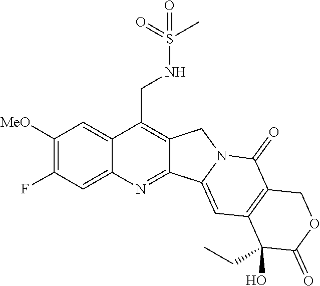 [SMILES: CC[C@@]1(O)C(=O)OCc2c1cc1n(c2=O)Cc2c-1nc1cc(F)c(OC)cc1c2CNS(C)(=O)=O] | (S)-N-((4-ethyl-8-fluoro-4- hydroxy-9-methoxy-3,14-dioxo- 3,4,12,14-tetrahydro-1H-pyrano- [3′,4′:6,7]indolizino[1,2-b]quin- olin-11-yl)methyl)methane- sulfonamide | Compound 123 |
| 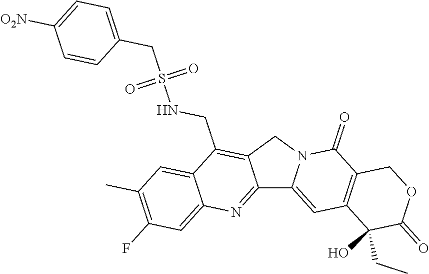 [SMILES: CC[C@@]1(O)C(=O)OCc2c1cc1n(c2=O)Cc2c-1nc1cc(F)c(C)cc1c2CNS(=O)(=O)Cc1ccc([N+](=O)[O-])cc1] | (S)-N-((4-ethyl-8-fluoro-4- hydroxy-9-methyl-3,14-dioxo- 3,4,12,14-tetrahydro-1H-pyrano- [3′,4′:6,7]indolizino[1,2-b]quin- olin-11-yl)methyl)-1-(4-nitro- phenyl)methanesulfonamide | Compound 124 |
| 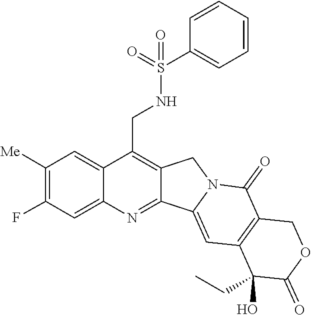 [SMILES: CC[C@@]1(O)C(=O)OCc2c1cc1n(c2=O)Cc2c-1nc1cc(F)c(C)cc1c2CNS(=O)(=O)c1ccccc1] | (S)-N-((4-ethyl-8-fluoro-4- hydroxy-9-methyl-3,14-dioxo- 3,4,12,14-tetrahydro-1H-pyrano- [3′,4′:6,7]indolizino[1,2-b]quin- olin-11-yl)methyl)benzene- sulfonamide | Compound 125 |
| 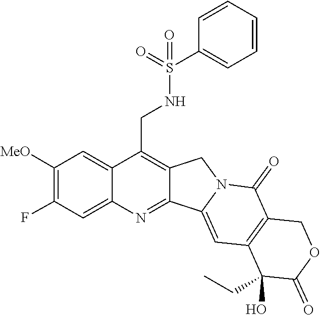 [SMILES: CC[C@@]1(O)C(=O)OCc2c1cc1n(c2=O)Cc2c-1nc1cc(F)c(OC)cc1c2CNS(=O)(=O)c1ccccc1] | (S)-N-((4-ethyl-8-fluoro-4- hydroxy-9-methoxy-3,14-dioxo- 3,4,12,14-tetrahydro-1H-pyrano- [3′,4′:6,7]indolizino[1,2-b]quin- olin-11-yl)methyl)benzene- sulfonamide | Compound 126 |
| 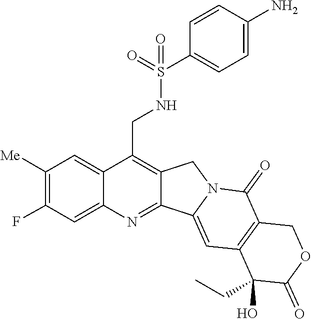 [SMILES: CC[C@@]1(O)C(=O)OCc2c1cc1n(c2=O)Cc2c-1nc1cc(F)c(C)cc1c2CNS(=O)(=O)c1ccc(N)cc1] | (S)-4-amino-N-((4-ethyl-8- fluoro-4-hydroxy-9-methyl-3,14- dioxo-3,4,12,14-tetrahydro-1H- pyrano[3′,4′:6,7]indolizino[1,2- b]quinolin-11-yl)methyl)- benzenesulfonamide | Compound 127 |
| 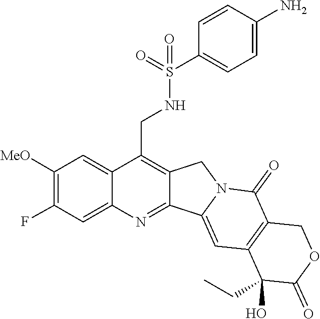 [SMILES: CC[C@@]1(O)C(=O)OCc2c1cc1n(c2=O)Cc2c-1nc1cc(F)c(OC)cc1c2CNS(=O)(=O)c1ccc(N)cc1] | (S)-4-amino-N-((4-ethyl-8- fluoro-4-hydroxy-9-methoxy- 3,14-dioxo-3,4,12,14-tetrahydro- 1H-pyrano[3′,4′:6,7]indolizino- [1,2-b]quinolin-11-yl)methyl)- benzenesulfonamide | Compound 128 |
| 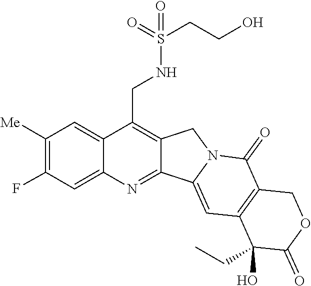 [SMILES: CC[C@@]1(O)C(=O)OCc2c1cc1n(c2=O)Cc2c-1nc1cc(F)c(C)cc1c2CNS(=O)(=O)CCO] | (S)-N-((4-ethyl-8-fluoro-4- hydroxy-9-methyl-3,14-dioxo- 3,4,12,14-tetrahydro-1H- pyrano[3′,4′:6,7]indolizino[1,2- b]quinolin-11-yl)methyl)-2- hydroxyethane-1-sulfonamide | Compound 129 |
| 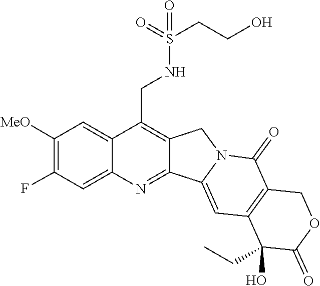 [SMILES: CC[C@@]1(O)C(=O)OCc2c1cc1n(c2=O)Cc2c-1nc1cc(F)c(OC)cc1c2CNS(=O)(=O)CCO] | (S)-N-((4-ethyl-8-fluoro-4- hydroxy-9-methoxy-3,14-dioxo- 3,4,12,14-tetrahydro-1H-pyrano- [3′,4′:6,7]indolizino[1,2-b]quin- olin-11-yl)methyl)-2-hydroxy- ethane-1-sulfonamide | Compound 130 |
| 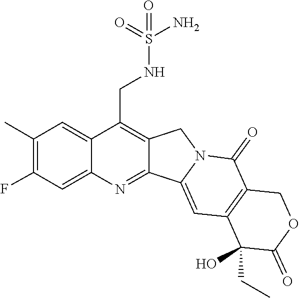 [SMILES: CC[C@@]1(O)C(=O)OCc2c1cc1n(c2=O)Cc2c-1nc1cc(F)c(C)cc1c2CNS(N)(=O)=O] | (S)-((4-ethyl-8-fluoro-4-hydroxy- 9-methyl-3,14-dioxo-3,4,12,14- tetrahydro-1H-pyrano[3′,4′:6,7]- indolizino[1,2-b]quinolin-11-yl)- methyl)sulfamide | Compound 131 |
| 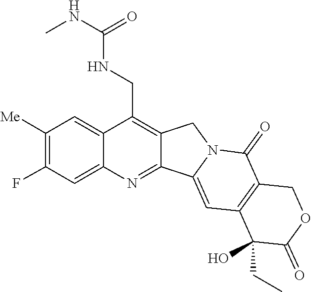 [SMILES: CC[C@@]1(O)C(=O)OCc2c1cc1n(c2=O)Cc2c-1nc1cc(F)c(C)cc1c2CNC(=O)NC] | (S)-1-((4-ethyl-8-fluoro-4- hydroxy-9-methyl-3,14-dioxo- 3,4,12,14-tetrahydro-1H-pyrano- [3′,4′:6,7]indolizino[1,2-b]quin- olin-11-yl)methyl)-3-methylurea | Compound 132 |
| 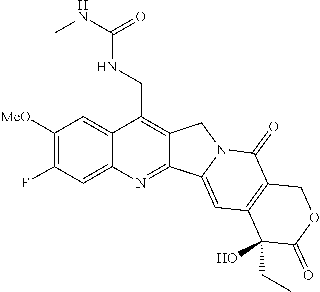 [SMILES: CC[C@@]1(O)C(=O)OCc2c1cc1n(c2=O)Cc2c-1nc1cc(F)c(OC)cc1c2CNC(=O)NC] | (S)-1-((4-ethyl-8-fluoro-4- hydroxy-9-methoxy-3,14-dioxo- 3,4,12,14-tetrahydro-1H-pyrano- [3′,4′:6,7]indolizino[1,2-b]quin- olin-11-yl)methyl)-3-methylurea | Compound 133 |
| 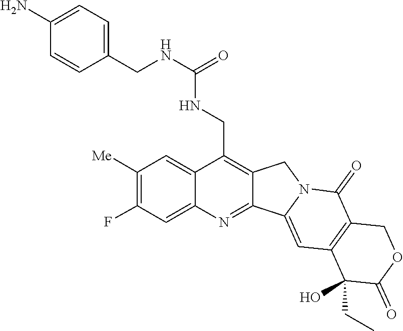 [SMILES: CC[C@@]1(O)C(=O)OCc2c1cc1n(c2=O)Cc2c-1nc1cc(F)c(C)cc1c2CNC(=O)NCc1ccc(N)cc1] | (S)-1-(4-aminobenzyl)-3-((4- ethyl-8-fluoro-4-hydroxy-9- methyl-3,14-dioxo-3,4,12,14- tetrahydro-1H-pyrano[3′,4′:6,7]- indolizino[1,2-b]quinolin-11- yl)methyl)urea | Compound 134 |
| 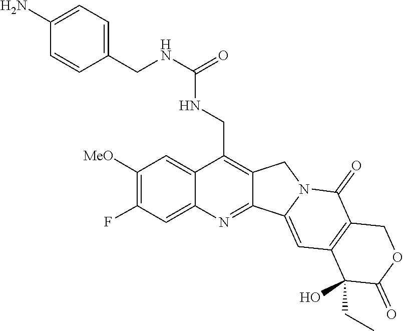 [SMILES: CC[C@@]1(O)C(=O)OCc2c1cc1n(c2=O)Cc2c-1nc1cc(F)c(OC)cc1c2CNC(=O)NCc1ccc(N)cc1] | (S)-1-(4-aminobenzyl)-3-((4- ethyl-8-fluoro-4-hydroxy-9- methoxy-3,14-dioxo-3,4,12,14- tetrahydro-1H-pyrano[3′,4′: 6,7]indolizino[1,2-b]quinolin- 11-yl)methyl)urea | Compound 135 |
| 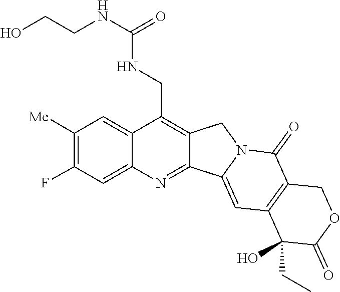 [SMILES: CC[C@@]1(O)C(=O)OCc2c1cc1n(c2=O)Cc2c-1nc1cc(F)c(C)cc1c2CNC(=O)NCCO] | (S)-1-((4-ethyl-8-fluoro-4- hydroxy-9-methyl-3,14-dioxo- 3,4,12,14-tetrahydro-1H-pyrano- [3′,4′:6,7]indolizino[1,2-b]quin- olin-11-yl)methyl)-3-(2-hydroxy- ethyl)urea | Compound 136 |
| 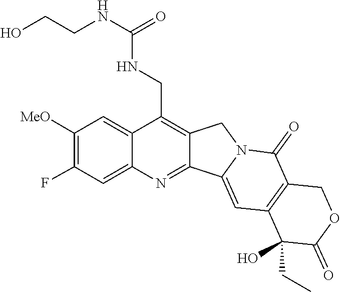 [SMILES: CC[C@@]1(O)C(=O)OCc2c1cc1n(c2=O)Cc2c-1nc1cc(F)c(OC)cc1c2CNC(=O)NCCO] | (S)-1-((4-ethyl-8-fluoro-4- hydroxy-9-methoxy-3,14-dioxo- 3,4,12,14-tetrahydro-1H-pyrano- [3′,4′:6,7]indolizino[1,2-b]quin- olin-11-yl)methyl)-3-(2-hydroxy- ethyl)urea | Compound 137 |
| 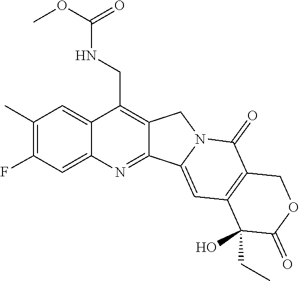 [SMILES: CC[C@@]1(O)C(=O)OCc2c1cc1n(c2=O)Cc2c-1nc1cc(F)c(C)cc1c2CNC(=O)OC] | methyl (S)-((4-ethyl-8-fluoro-4- hydroxy-9-methyl-3,14-dioxo- 3,4,12,14-tetrahydro-1H-pyrano- [3′,4′:6,7]indolizino[1,2-b]quin- olin-11-yl)methyl)carbamate | Compound 138 |
| 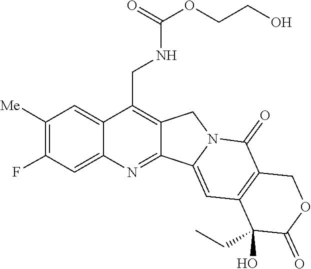 [SMILES: CC[C@@]1(O)C(=O)OCc2c1cc1n(c2=O)Cc2c-1nc1cc(F)c(C)cc1c2CNC(=O)OCCO] | 2-hydroxyethyl (S)-((4-ethyl-8- fluoro-4-hydroxy-9-methyl- 3,14-dioxo-3,4,12,14-tetrahydro- 1H-pyrano[3′,4′:6,7]indolizino- [1,2-b]quinolin-11-yl)methyl)- carbamate | Compound 139 |

It is to be understood that reference to compounds of Formula (I) throughout the remainder of this disclosure, includes in various embodiments, compounds of Formula (Ia), Formula (II), Formula (IIa), Formula (III), Formula (IIIa) and Formula (IIIb), to the same extent as if embodiments reciting each of these Formulae individually were specifically recited.

In certain embodiments, compounds of Formula (I) may possess a sufficiently acidic group, a sufficiently basic group, or both functional groups, and accordingly react with a number of organic and inorganic bases, or organic and inorganic acids, to form pharmaceutically acceptable salts. The term “pharmaceutically acceptable salt” as used herein, refers to a salt of a compound of Formula (I), which is substantially non-toxic to living organisms. Typical pharmaceutically acceptable salts include those salts prepared by reaction of a compound of Formula (I) with a pharmaceutically acceptable mineral or organic acid or an organic or inorganic base. Such salts are known as acid addition and base addition salts.

Acids commonly employed to form acid addition salts are inorganic acids including, but are not limited to, hydrochloric acid, hydrobromic acid, hydroiodic acid, sulfuric acid, phosphoric acid, and organic acids including, but not limited to, p-toluenesulfonic acid, methanesulfonic acid, oxalic acid, p-bromophenylsulfonic acid, carbonic acid, succinic acid, citric acid, benzoic acid and acetic acid. Examples of pharmaceutically acceptable salts include, but are not limited to, sulfates, pyrosulfates, bisulfates, sulfites, phosphates, monohydrogenphosphates, dihydrogenphosphates, metaphosphates, pyrophosphates, bromides, iodides, acetates, propionates, decanoates, caprylates, acrylates, formates, hydrochlorides, dihydrochlorides, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, hydroxybenzoates, methoxybenzoates, phthalates, xylenesulfonates, phenylacetates, phenylpropionates, phenylbutyrates, citrates, lactates, gamma-hydroxybutyrates, glycolates, tartrates, methanesulfonates, propanesulfonates, naphthalene-1-sulfonates, napththalene-2-sulfonates and mandelates. Pharmaceutically acceptable acid addition salts of particular interest are those formed with mineral acids such as hydrochloric acid and hydrobromic acid, and those formed with organic acids such as maleic acid and methanesulfonic acid.

Salts of amine groups may also comprise quaternary ammonium salts in which the amino nitrogen carries a suitable organic group such as an alkyl, lower alkenyl, lower alkynyl or aralkyl moiety.

Base addition salts include those derived from inorganic bases, such as ammonium or alkali or alkaline earth metal hydroxides, carbonates, bicarbonates, and the like. Bases useful in preparing pharmaceutically acceptable salts include, but are not limited to, sodium hydroxide, potassium hydroxide, ammonium hydroxide, potassium carbonate, sodium carbonate, sodium bicarbonate, potassium bicarbonate, calcium hydroxide and calcium carbonate.

One skilled in the art will understand that the particular counterion forming a part of a pharmaceutically acceptable salt is usually not of a critical nature, so long as the salt as a whole is pharmacologically acceptable and as long as the counterion does not contribute undesired qualities to the salt as a whole.

Certain embodiments relate to pharmaceutically acceptable solvates of a compound of Formula (I). One skilled in the art will appreciate that certain compounds of Formula (I) may combine with solvents such as water, methanol, ethanol or acetonitrile to form pharmaceutically acceptable solvates such as the corresponding hydrate, methanolate, ethanolate or acetonitrilate.

Other examples of solvents that may be used to prepare solvates include isopropanol, dimethyl sulfoxide, ethyl acetate, acetic acid, ethanolamine and acetone, as well as miscible formulations of solvate mixtures as would be known by the skilled artisan.

### Preparation of Camptothecin Analogues

Camptothecin analogues of Formula (I) may be prepared by standard synthetic organic chemistry methods from commercially available starting materials and reagents. See, also, Li, et al., 2019, ACS Med. Chem. Lett., 10(10): 1386-1392 and U.S. Patent Application Publication No.

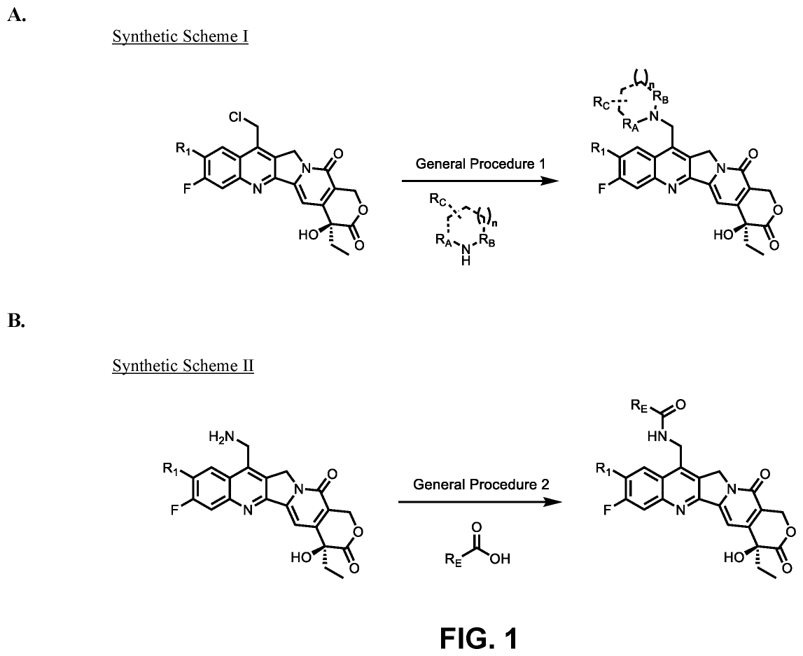

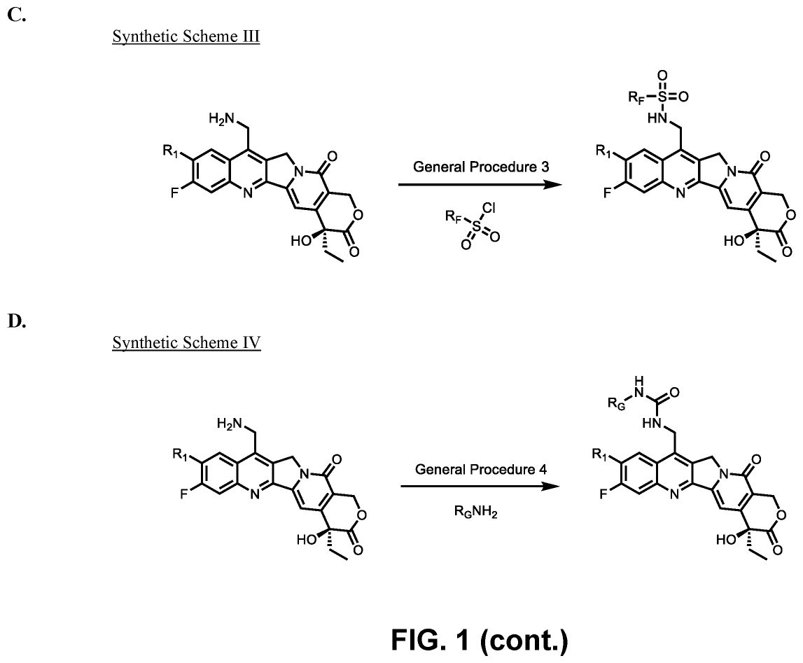

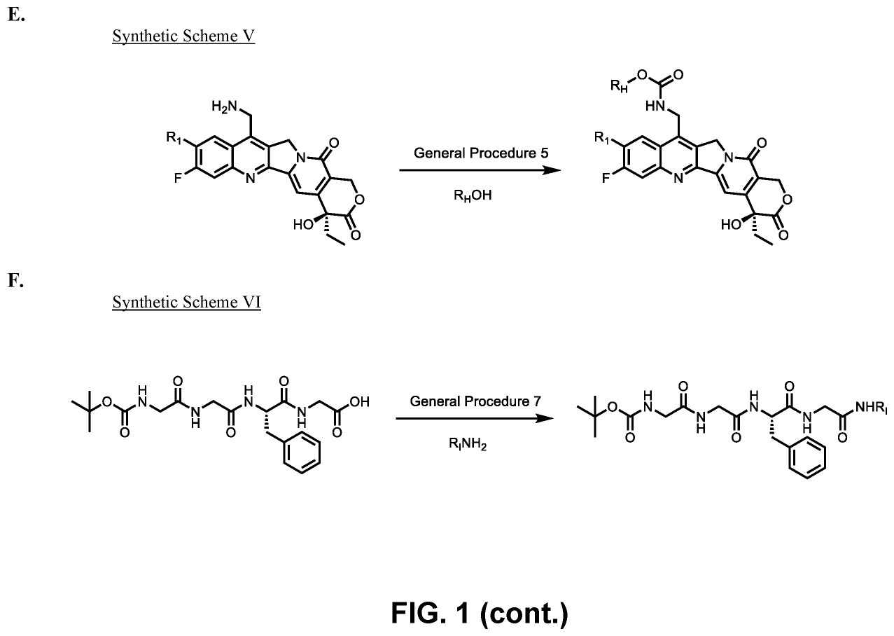

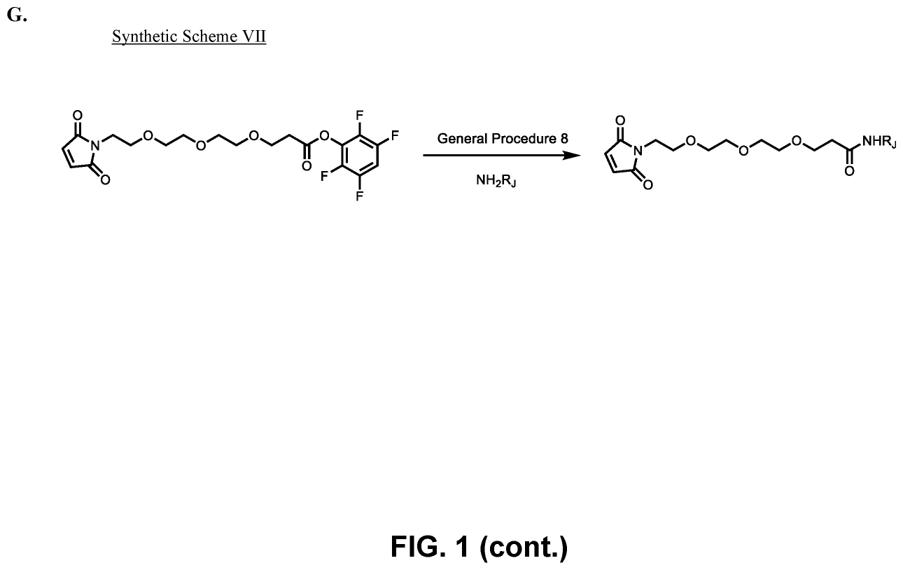

US 2004/0266803. Representative examples of suitable synthetic routes are described in detail in the Examples provided herein (see also FIG. 1). One skilled in the art will recognize that alternative methods may be employed to synthesize camptothecin analogues of Formula (I), and that the approaches described herein are therefore not intended to be exhaustive.

### Conjugates

Certain embodiments of the present disclosure relate to conjugates of compounds of Formula (I) comprising one or more compounds of Formula (I) conjugated to a targeting moiety via one or more linkers.

The conjugates of the present disclosure may comprise one or multiple compounds of Formula (I) conjugated to the targeting moiety. For example, multiple compounds of Formula (I) may be conjugated to the targeting moiety by attaching the compound at multiple different sites on the targeting moiety. Alternatively, or in addition, multiple compounds of Formula (I) may be conjugated to the targeting moiety by employing one or more multivalent linkers each allowing for attachment of multiple compounds to a single site on the targeting moiety.

Accordingly, certain embodiments of the present disclosure relate to conjugates of Formula (X):

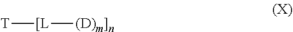

wherein:
T is a targeting moiety;
L is a linker;
D is a camptothecin analogue as described herein;
m is an integer between 1 and 4, and
n is an integer between 1 and 10.

In certain embodiments, in conjugates of Formula (X), m is between 1 and 2. In some embodiments, m is 1.

In some embodiments, in conjugates of Formula (X), n is between 1 and 8, for example, between 2 and 8, or between 2 and 6. In some embodiments, n is between 2 and 4.

As noted above and reflected by parameters m and n in Formula (X), a targeting moiety, “T,” can be conjugated to more than one compound of Formula (I), “D.” Those skilled in the art will appreciate that, while any particular targeting moiety T is conjugated to an integer number of compounds D, analysis of a preparation of the conjugate to determine the ratio of compound D to targeting moiety T may give a non-integer result, reflecting a statistical average. This ratio of compound D to targeting moiety T may generally be referred to as the drug-to-antibody ratio, or “DAR.” Accordingly, conjugate preparations having non-integer DARs are intended to be encompassed by Formula (X). One skilled in the art will appreciate that the term “DAR” may be employed to define conjugates comprising targeting moieties other than antibodies.

### Camptothecin Analogue, D

In accordance with the present disclosure, conjugates of Formula (X) comprise a camptothecin analogue as the drug moiety, D, where the camptothecin analogue is a compound of Formula (I).

In certain embodiments, in the conjugates of Formula (X), D is a compound of Formula (Ia), Formula (II), Formula (IIa), Formula (III), Formula (IIIa) or Formula (IIIb). In certain embodiments, in the conjugates of Formula (X), D is a compound selected from the compounds shown in Tables 1-3.

Certain embodiments of the present disclosure relate to conjugates having Formula (X), in which D is a compound of Formula (IV):

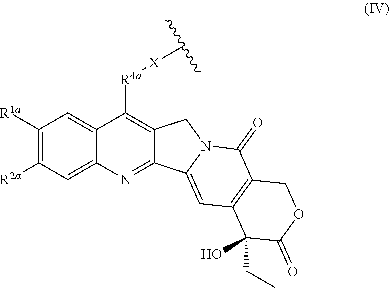

wherein:
   R^{1a} is selected from: —H, —CH₃, —CHF₂, —CF₃, —F, —Br, —Cl, —OH, —OCH₃, —OCF₃ and —NH₂;
   R^{2a} is selected from: —H, —CH₃, —CF₃, —F, —Br, —Cl, —OH, —OCH₃ and —OCF₃;
   X is —O—, —S— or —NH—, and R^{4a} is selected from:

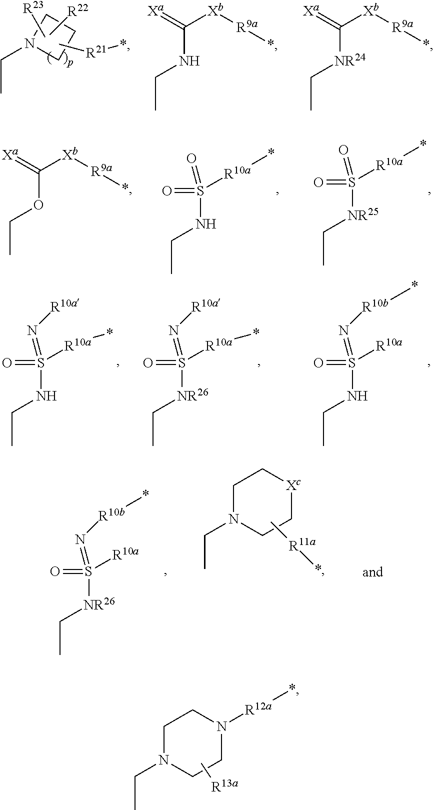

wherein * is the point of attachment to X, and wherein p is 1, 2, 3 or 4; or
X is 0, and R^{4a}—X is selected from:

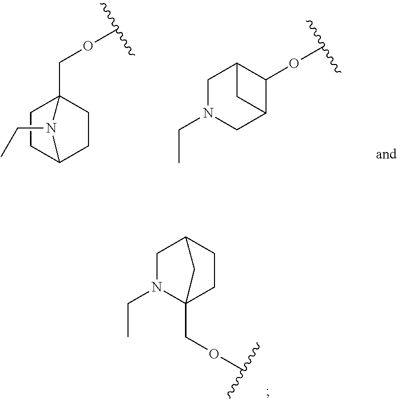 [SMILES: *OCC12CCC(CC1)N2CC ; *OC1C2CC1CN(CC)C2 ; *OCC12CCC(CN1CC)C2]

R^{5a} is selected from: —C₁-C₆ alkyl, —C₃-C₈ cycloakyl, -aryl, -heteroaryl an —C₁-C₆ alkyl)-aryl;
R^{8a} is selected from: —C₁-C₆ alkyl, —C₃-C₈ cycloalkyl and —C₃-C₈ heterocycloalkyl;
each R^{9a} is independently selected from: —C₁-C₆ alkyl, —C₃-C₈ cycloalkyl, -aryl, -heteroaryl and —(C₁-C₆ alkyl)-aryl; or R^{9a} is absent and X^{b}═X;
each R^{10a} is independently selected from: —C₁-C₆ alkyl, —C₃-C₈ cycloalkyl, -aryl, -heteroaryl, —(C₁-C₆ alkyl)-aryl and

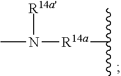

each R^{10a′} is independently selected from: —H, —C₁-C₆ alkyl, —C₁-C₈ cycloalkyl, -aryl, -heteroaryl and —(C₁-C₆ alkyl)-aryl;
each R^{10b} is independently selected from: —C₁-C₆ alkyl, —C₃-C₈ cycloalkyl, -aryl, -heteroaryl and —(C₁-C₆ alkyl)-aryl;
R^{11a} is absent or is —C₁-C₆ alkyl;
R^{12a} is selected from: —C₁-C₆ alkyl, —CO₂R^{5a}, -aryl, -heteroaryl, —(C₁-C₆ alkyl)-aryl, —S(O)₂R^{16a} and

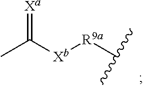

R^{13a} is selected from: —H and —C₁-C₆ alkyl;
R^{14a} is selected from: —C₁-C₆ alkyl, —C₃-C₈ cycloalkyl and —C₃-C₈ heterocycloalkyl;
R^{14′} is selected from: H, —C₁-C₆ alkyl, —C₃-C₈ cycloalkyl and —C₃-C₈ heterocycloalkyl;
R^{16a} is selected from: —C₁-C₆ alkyl, —C₃-C₈ cycloalkyl, -aryl, -heteroaryl and —(C₁-C₆ alkyl)-aryl;
R²¹ is selected from: —C₁-C₆ alkyl, —C₃-C₈ cycloalkyl and —(C₁-C₆ alkyl)-O—R^{5a};
R²² and R²³ are each independently selected from: —H, -halogen, —C₁-C₆ alkyl and —C₃-C₈ cycloalkyl;
R²⁴, R²⁵ and R²⁶ are each —C₁-C₆ alkyl;
X^{a} and X^{b} are each independently selected from: NH, O and S;
X^{c} is selected from: O, S and S(O)₂, and
[symbol] denotes the point of attachment to linker, L.

In some embodiments, in compounds of Formula (IV), R^{1a} is selected from: —CH₃, —CF₃, —OCH₃, —OCF₃ and —NH₂.

In some embodiments, in compounds of Formula (IV), R^{1a} is selected from: —CH₃, —CF₃, —OCH₃ and —OCF₃.

In some embodiments, in compounds of Formula (IV), R^{1a} is selected from: —CH₃, —OCH₃ and NH₂.

In some embodiments, in compounds of Formula (IV), R^{1a} is selected from: —CH₃ and —OCH₃.

In some embodiments, in compounds of Formula (IV), R^{2a} is selected from: —H, —CH₃, —CF₃, —F, —Cl, —OCH₃ and —OCF₃.

In some embodiments, in compounds of Formula (IV), R^{2a} is selected from: —H, —F and —Cl.

In some embodiments, in compounds of Formula (IV), R^{2a} is —F.

In some embodiments, in compounds of Formula (IV), X is —O—, —S— or —NH—, and R^{4a} is selected from:

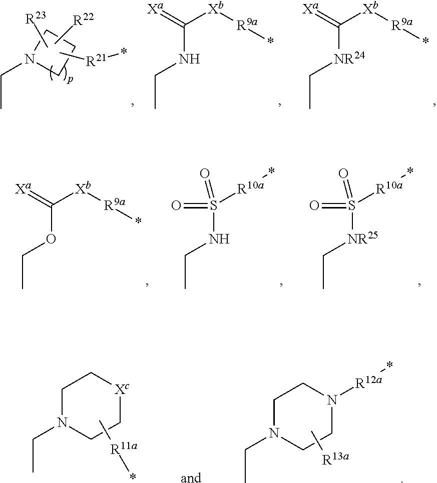

In some embodiments, in compounds of Formula (IV), X is —O— or —NH—.

In some embodiments, in compounds of Formula (IV), each R^{9a} is independently selected from: —C₁-C₆ alkyl, —C₃-C₈ cycloalkyl, -aryl and —(C₁-C₆ alkyl)-aryl.

In some embodiments, in compounds of Formula (IV), each R^{9a} is independently selected from: —C₁-C₆ alkyl and —(C₁-C₆ alkyl)-aryl.

In some embodiments, in compounds of Formula (IV), each R^{10a} is independently selected from: —C₁-C₆ alkyl, —C₃-C₈ cycloalkyl, -aryl, —(C₁-C₆ alkyl)-aryl and

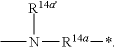

In some embodiments, in compounds of Formula (IV), each R^{10a} is independently selected from: —C₁-C₆ alkyl, -aryl, —(C₁-C₆ alkyl)-aryl and

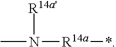

In some embodiments, in compounds of Formula (IV), R^{12a} is selected from: —C₁-C₆ alkyl, -aryl, —(C₁-C₆ alkyl)-aryl and —S(O)₂R¹⁶.

In some embodiments, in compounds of Formula (IV), R^{13a} is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (IV), R^{14a′} is selected from: H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (IV), R^{16a} is selected from: -aryl, -heteroaryl and —(C₁-C₆ alkyl)-aryl.

In some embodiments, in compounds of Formula (IV), R²² and R²³ are each independently selected from: —H, -halogen, unsubstituted —C₁-C₆ alkyl, —C₁-C₆ haloalkyl, —C₁-C₆ aminoalkyl, —C₁-C₆ hydroxyalkyl and —C₃-C₈ cycloalkyl.

In some embodiments, in compounds of Formula (IV), X^{a} and X^{b} are each independently selected from: NH and O.

Combinations of any of the foregoing embodiments for compounds of Formula (IV) are also contemplated and each combination forms a separate embodiment for the purposes of the present disclosure.

Certain embodiments of the present disclosure relate to conjugates having Formula (X), in which D is a compound of Formula (V):

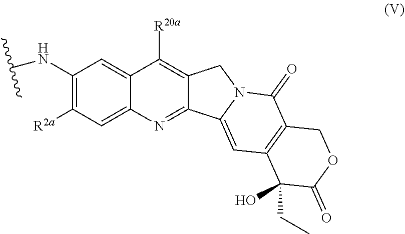

wherein:
   R^{2a} is selected from: —CH₃, —CF₃, —F, —Br, —Cl, —OH, —OCH₃ and —OCF₃;
   R^{20a} is selected from: —H, —C₁-C₆ alkyl, —C₃-C₈ cycloalkyl, —(C₁-C₆ alkyl)-O—R⁵,

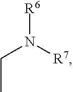

—CO₂R⁸, -aryl, -heteroaryl, —(C₁-C₆ alkyl)-aryl,

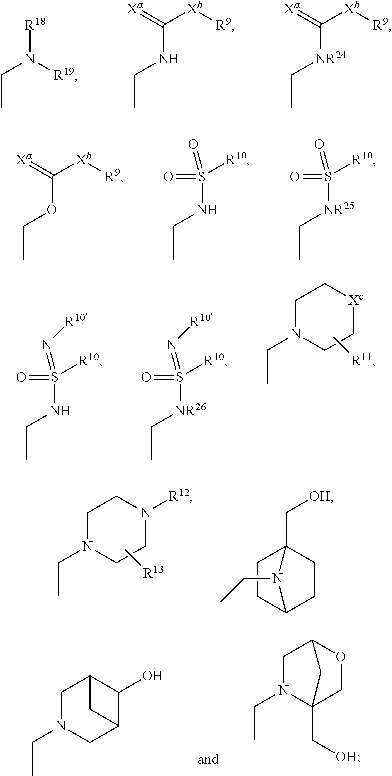

R⁵ is selected from: —H, —C₁-C₆ alkyl, —C₃-C₈ cycloalkyl, -aryl, -heteroaryl and —(C₁-C₆ alkyl)-aryl;
R⁶ and R⁷ are each independently selected from: —H, —C₁-C₆ alkyl, —C₃-C₈ cycloalkyl, —(C₁-C₆ alkyl)-O—R⁵, —C₃-C₈ heterocycloalkyl and —C(O)R′⁷;
R⁸ is selected from: —H, —C₁-C₆ alkyl, —C₃-C₈ cycloalkyl and —C₃-C₈ heterocycloalkyl;
each R⁹ is independently selected from: —H, —C₁-C₆ alkyl, —C₃-C₈ cycloalkyl, -aryl, -heteroaryl and —(C₁-C₆ alkyl)-aryl;
each R¹⁰ is independently selected from: —C₁-C₆ alkyl, —C₃-C₈ cycloalkyl, -aryl, -heteroaryl, —(C₁-C₆ alkyl)-aryl and

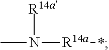

each R^{10′} is independently selected from: —H, —C₁-C₆ alkyl, —C₃-C₈ cycloalkyl, -aryl, -heteroaryl and —(C₁-C₆ alkyl)-aryl;
R¹¹ is selected from: —H and —C₁-C₆ alkyl;
R¹² is selected from: —H, —C₁-C₆ alkyl, —CO₂R⁸, -aryl, -heteroaryl, —(C₁-C₆ alkyl)-aryl, —S(O)₂R¹⁶ and

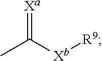

R¹³ is selected from: —H and —C₁-C₆ alkyl;
R¹⁴ and R^{14′} are each independently selected from: —H, C₁-C₆ alkyl, —C₃-C₈ cycloalkyl and —C₃-C₈ heterocycloalkyl;
R¹⁶ is selected from: —C₁-C₆ alkyl, —C₃-C₈ cycloalkyl, -aryl, -heteroaryl and —(C₁-C₆ alkyl)-aryl;
R¹⁷ is selected from: —C₁-C₆ alkyl, —C₃-C₈ cycloalkyl, —C₃-C₈ heterocycloalkyl, —(C₁-C₆ alkyl)-C₃-C₈ heterocycloalkyl, -aryl, -heteroaryl and —(C₁-C₆ alkyl)-aryl;
R¹⁸ and R¹⁹ taken together with the N atom to which they are bonded form a 4-, 5-, 6-, or 7-membered ring having 0 to 3 substituents selected from: halogen, —C₁-C₆ alkyl, —C₃-C₈cycloalkyl and —(C₁-C₆ alkyl)-O—R⁵;
R²⁴, R²⁵ and R²⁶ are each —C₁-C₆ alkyl;
X^{a} and X^{b} are each independently selected from: NH, O and S;
X^{c} is selected from: O, S and S(O)₂, and
[symbol] denotes the point of attachment to linker, L.

In some embodiments, in compounds of Formula (V), R^{2a} is selected from: —CH₃, —CF₃, —F, —Cl, —OCH₃ and —OCF₃.

In some embodiments, in compounds of Formula (V), R^{2a} is selected from: —CF₃, —F, —Cl and —OCH₃.

In some embodiments, in compounds of Formula (V), R^{2a} is F.

In some embodiments, in compounds of Formula (V), R^{20a} is selected from: —H, —C₁-C₆ alkyl, —C₃-C₈ cycloalkyl, —(C₁-C₆ alkyl)-O—R⁵,

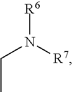

—CO₂R⁸, -aryl, -heteroaryl, —(C₁-C₆ alkyl)-aryl,

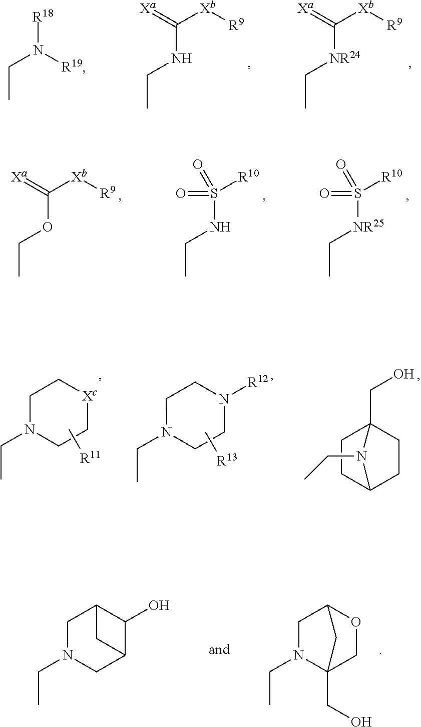

In some embodiments, in compounds of Formula (V), R^{20a} is selected from: —H, —C₁-C₆ alkyl, —(C₁-C₆ alkyl)-O—R⁵,

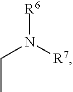

—(C₁-C₆ alkyl)-aryl,

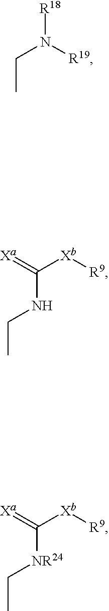

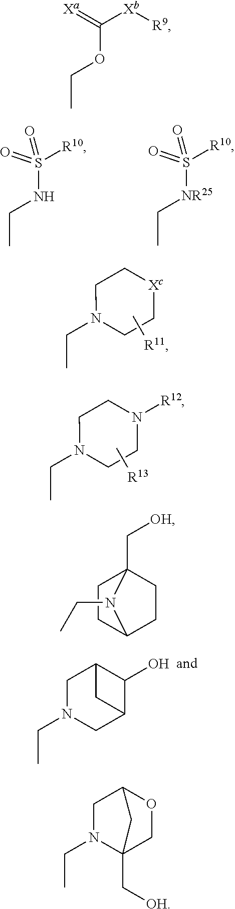

In some embodiments, in compounds of Formula (V), R^{20a} is selected from: —H, —C₁-C₆ alkyl, —(C₁-C₆ alkyl)-O—R⁵,

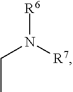

—(C₁-C₆ alkyl)-aryl,

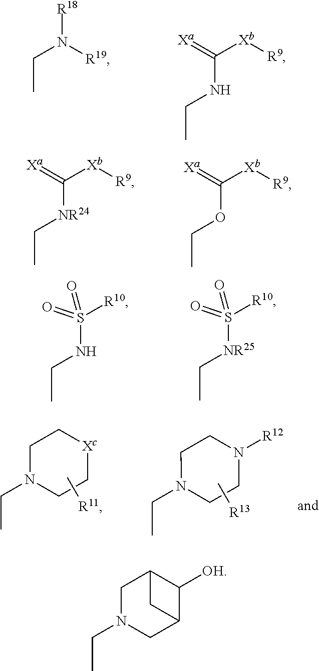

In some embodiments, in compounds of Formula (V), R^{20a} is selected from: —H, —C₁-C₆ alkyl, —(C₁-C₆ alkyl)-O—R⁵,

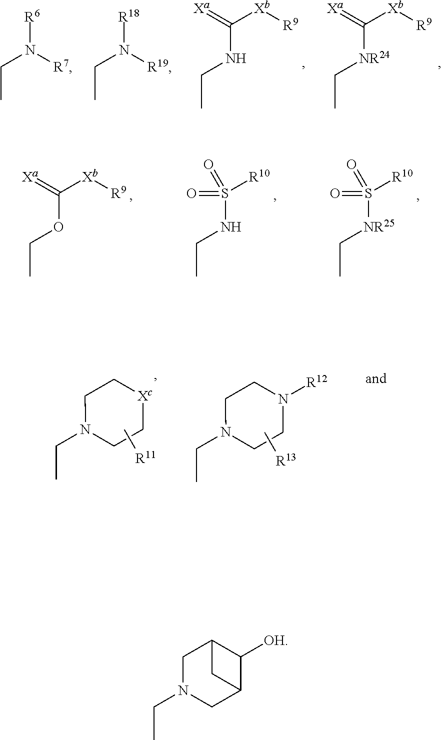

In some embodiments, in compounds of Formula (V), R^{20a} is selected from: —H, unsubstituted —C₁-C₆ alkyl, —C₁-C₆ haloalkyl, —C₁-C₆ hydroxyalkyl, —C₃-C₈ cycloalkyl, —(C₁-C₆ alkyl)-O—R⁵,

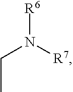

—CO₂R⁸, unsubstituted -aryl, -aminoaryl, -heteroaryl, —(C₁-C₆ alkyl)-aminoaryl,

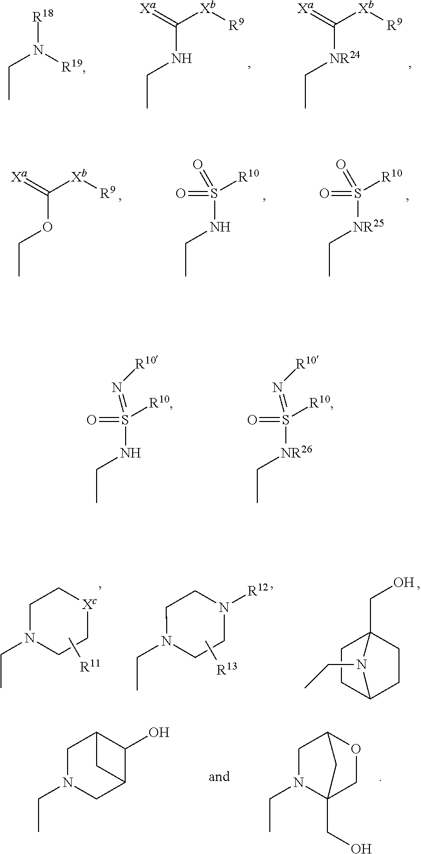

In some embodiments, in compounds of Formula (V), R⁶ and R⁷ are each independently selected from: —H, —C₁-C₆ alkyl, —C₃-C₈ cycloalkyl and —C(O)R¹⁷.

In some embodiments, in compounds of Formula (V), R⁶ is H, and R⁷ is selected from: —H, —C₁-C₆ alkyl, —C₃-C₈ cycloalkyl, —(C₁-C₆ alkyl)-O—R⁵, —C₃-C₈ heterocycloalkyl and —C(O)R¹⁷.

In some embodiments, in compounds of Formula (V), R⁶ is H, and R⁷ is selected from: —H, —C₁-C₆ alkyl, —C₃-C₈ cycloalkyl and —C(O)R¹⁷.

In some embodiments, in compounds of Formula (V), R⁶ and R⁷ are each independently selected from: —H, unsubstituted —C₁-C₆ alkyl, —C₁-C₆ haloalkyl, —C₁-C₆ hydroxyalkyl, —C₃-C₈ cycloalkyl, —(C₁-C₆ alkyl)-O—R⁵, —C₃-C₈ heterocycloalkyl and —C(O)R¹⁷.

In some embodiments, in compounds of Formula (V), R⁸ is selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (V), each R⁹ is independently selected from: —C₁-C₆ alkyl, —C₃-C₈ cycloalkyl, -aryl and —(C₁-C₆ alkyl)-aryl.

In some embodiments, in compounds of Formula (V), each R⁹ is independently selected from: —C₁-C₆ alkyl and —(C₁-C₆ alkyl)-aryl.

In some embodiments, in compounds of Formula (V), each R⁹ is independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aminoaryl.

In some embodiments, in compounds of Formula (V), each R¹⁰ is independently selected from: —C₁-C₆ alkyl, —C₃-C₈ cycloalkyl, —NR¹⁴R^{14′}, -aryl and —(C₁-C₆ alkyl)-aryl.

In some embodiments, in compounds of Formula (V), each R¹⁰ is independently selected from: —C₁-C₆ alkyl, —NR¹⁴R^{14′}, -aryl and —(C₁-C₆ alkyl)-aryl.

In some embodiments, in compounds of Formula (V), R¹¹ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (V), R¹² is selected from: —H, —C₁-C₆ alkyl, -aryl, —(C₁-C₆ alkyl)-aryl and —S(O)₂R¹⁶.

In some embodiments, in compounds of Formula (V), R¹² is selected from: —H, unsubstituted —C₁-C₆ alkyl, —C₁-C₆ haloalkyl, —C₁-C₆ hydroxyalkyl, —C₁-C₆ aminoalkyl, —CO₂R⁸, unsubstituted -aryl, -aminoaryl, -heteroaryl, —(C₁-C₆ alkyl)-aminoaryl, —S(O)₂R¹⁶ and

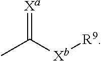

In some embodiments, in compounds of Formula (V), R¹³ is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (V), R¹⁴ and R^{14′} are each independently selected from: —H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (V), R¹⁶ is selected from: -aryl, -heteroaryl and —(C₁-C₆ alkyl)-aryl.

In some embodiments, in compounds of Formula (V), R¹⁶ is selected from: unsubstituted —C₁-C₆ alkyl, —C₁-C₆ haloalkyl, —C₁-C₆ hydroxyalkyl, —C₁-C₆ aminoalkyl, —C₃-C₈ cycloalkyl, unsubstituted -aryl, -aminoaryl, -heteroaryl and —(C₁-C₆ alkyl)-aminoaryl.

In some embodiments, in compounds of Formula (V), R¹⁷ is selected from: unsubstituted —C₁-C₆ alkyl, —C₃-C₈ cycloalkyl, —C₃-C₈ heterocycloalkyl, —(C₁-C₆ alkyl)-C₃-C₈ heterocycloalkyl, unsubstituted -aryl, -hydroxyaryl, -aminoaryl, -heteroaryl and —(C₁-C₆ alkyl)-aminoaryl.

In some embodiments, in compounds of Formula (V), R¹⁸ and R¹⁹ taken together with the N atom to which they are bonded form a 4-, 5-, 6-, or 7-membered ring having 0 to 3 substituents selected from: halogen, unsubstituted —C₁-C₆ alkyl, —C₁-C₆ haloalkyl, —C₁-C₆ aminoalkyl, —C₁-C₆ hydroxyalkyl, —C₃-C₈ cycloalkyl and —(C₁-C₆ alkyl)-O—R⁵.

In some embodiments, in compounds of Formula (V), R¹⁷ is —C₁-C₆ alkyl.

In some embodiments, in compounds of Formula (V), X^{a} and X^{b} are each independently selected from: NH and O.

Combinations of any of the foregoing embodiments for compounds of Formula (V) are also contemplated and each combination forms a separate embodiment for the purposes of the present disclosure.

Certain embodiments of the present disclosure relate to conjugates having Formula (X), in which D is a compound of Formula (VI):

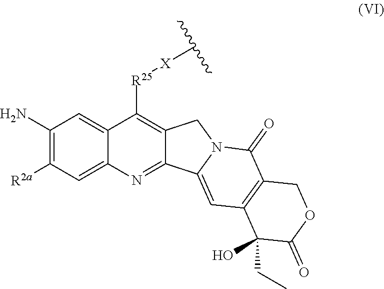

wherein:
   R^{2a} is selected from: —H, —CH₃, —CF₃, —F, —Br, —Cl, —OH, —OCH₃ and —OCF₃;
   X is —O—, —S— or —NH—, and R²⁵ is selected from: —C₁-C₆ alkyl, —(C₁-C₆ alkyl)-O—R^{5a}, —CO₂R^{8a}, —C(O)—, -aryl, -heteroaryl, —(C₁-C₆ alkyl)-aryl,

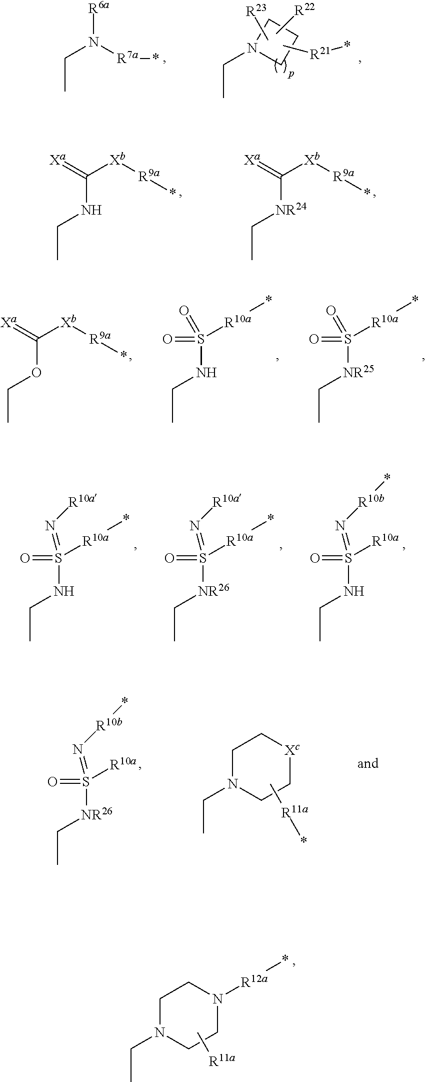

wherein * is the point of attachment to X, and wherein p is 1, 2, 3 or 4; or
X is O, and R²⁵—X— is selected from:

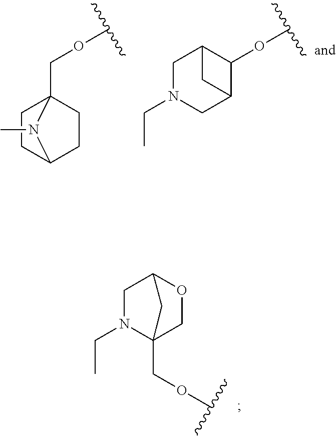 [SMILES: *OCC12CCC(CC1)N2C ; *OC1C2CC1CN(CC)C2 ; *OCC12COC(CN1CC)C2]

R^{5a} is selected from: —C₁-C₆ alkyl, —C₃-C₈ cycloalkyl, -aryl, -heteroaryl and —(C₁-C₆ alkyl)-aryl;
R^{6a} is selected from: —H, —C₁-C₆ alkyl, —C₃-C₈ cycloalkyl and —C₃-C₈ heterocycloalkyl;
R^{7a} is selected from: —C₁-C₆ alkyl, —C₃-C₈ cycloalkyl, —(C₁-C₆ alkyl)-O—R^{5a}, —C₃-C₈ heterocycloalkyl and —C(O)R^{17a};
R^{8a} is selected from: —C₁-C₆ alkyl, —C₃-C₈ cycloalkyl and —C₃-C₈ heterocycloalkyl;
each R^{9a} is independently selected from: —C₁-C₆ alkyl, —C₃-C₈ cycloalkyl, -aryl, -heteroaryl and —(C₁-C₆ alkyl)-aryl; or R^{9a} is absent and X^{b}═X;
each R^{10a} is independently selected from: —C₁-C₆ alkyl, —C₃-C₈ cycloalkyl, -aryl, -heteroaryl, —(C₁-C₆ alkyl)-aryl and

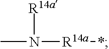

each R^{10a′} is independently selected from: —H, —C₁-C₆ alkyl, —C₃-C₈ cycloalkyl, -aryl, -heteroaryl and —(C₁-C₆ alkyl)-aryl;
each R^{10b} is independently selected from: —C₁-C₆ alkyl, —C₃-C₈ cycloalkyl, -aryl, -heteroaryl and —(C₁-C₆ alkyl)-aryl;
R^{11a} is absent or is —C₁-C₆ alkyl;
R^{12a} is selected from: —C₁-C₆ alkyl, —CO₂R^{8a}, -aryl, -heteroaryl, —(C₁-C₆ alkyl)-aryl, —S(O)₂R^{16a} and

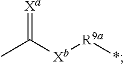

R^{13a} is selected from: —H and —C₁-C₆ alkyl;
R^{14a} is selected from: —C₁-C₆ alkyl, —C₃-C₈ cycloalkyl and —C₃-C₈ heterocycloalkyl;
R^{14a′} is selected from: H, —C₁-C₆ alkyl, —C₃-C₈ cycloalkyl and —C₃-C₈ heterocycloalkyl;
R^{16a} is selected from: —C₁-C₆ alkyl, —C₃-C₈ cycloalkyl, -aryl, -heteroaryl and —(C₁-C₆ alkyl)-aryl;
R^{17a} is selected from: —C₁-C₆ alkyl, —C₃-C₈ cycloalkyl, —C₃-C₈ heterocycloalkyl, —(C₁-C₆ alkyl)-C₃-C₈ heterocycloalkyl, -aryl, -heteroaryl and —(C₁-C₆ alkyl)-aryl;
R²¹ is selected from: —C₁-C₆ alkyl, —C₃-C₈ cycloalkyl and —(C₁-C₆ alkyl)-O—R^{5a};
R²² and R²³ are each independently selected from: —H, -halogen, —C₁-C₆ alkyl and —C₃-C₈ cycloalkyl;
R²⁴, R²⁵ and R²⁶ are each —C₁-C₆ alkyl;
X^{a} and X^{b} are each independently selected from: NH, O and S;
X^{c} is selected from: O, S and S(O)₂, and
[symbol] denotes the point of attachment to linker, L.

In some embodiments, in compounds of Formula (VI), R^{2a} is selected from: —CH₃, —CF₃, —F, —Br, —Cl, —OH, —OCH₃ and —OCF₃.

In some embodiments, in compounds of Formula (VI), R^{2a} is selected from: —CH₃, —CF₃, —F, —Cl, —OCH₃ and —OCF₃.

In some embodiments, in compounds of Formula (VI), R^{2a} is selected from: F and Cl.

In some embodiments, in compounds of Formula (VI), R^{2a} is F.

In some embodiments, in compounds of Formula (VI), X is —O—, —S— or —NH—, and R²⁵ is selected from: —C₁-C₆ alkyl, —(C₁-C₆ alkyl)-O—R^{5a}, —(C₁-C₆ alkyl)-aryl,

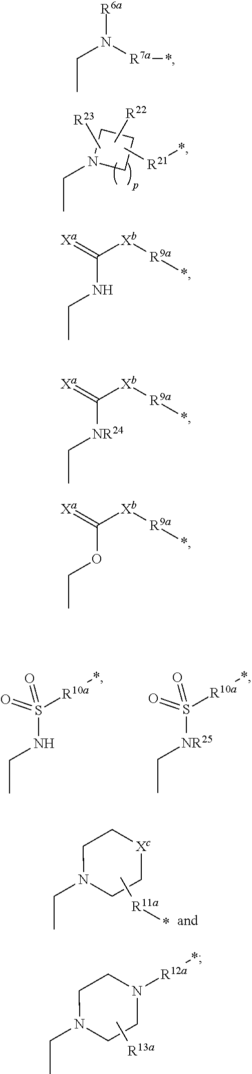

and or X is O, and R²⁵—X— is selected from:

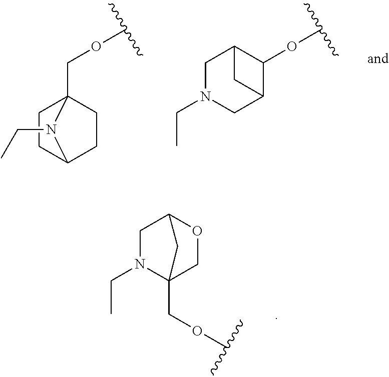 [SMILES: *OCC12CCC(CC1)N2CC ; *OC1C2CC1CN(CC)C2 ; "and" ; *OCC12COC(CN1CC)C2 ; "."]

In some embodiments, in compounds of Formula (VI), X is —O—, —S— or —NH—, and R²⁵ is selected from: —C₁-C₆ alkyl, —(C₁-C₆ alkyl)-O—R^{5a}, —(C₁-C₆ alkyl)-aryl,

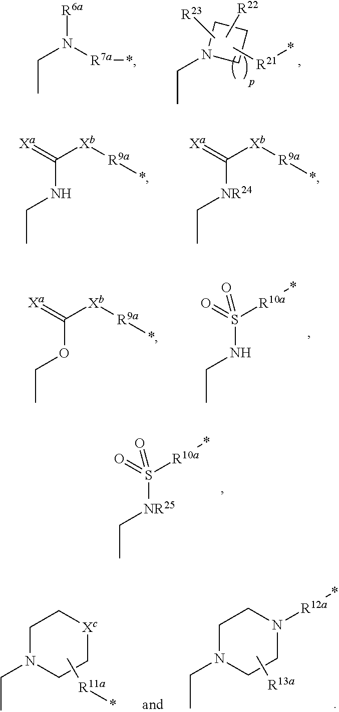

In some embodiments, in compounds of Formula (VI), X is —O—, —S— or —NH—, and R²⁵ is selected from: —C₁-C₆ alkyl, —(C₁-C₆ alkyl)-O—R^{5a},

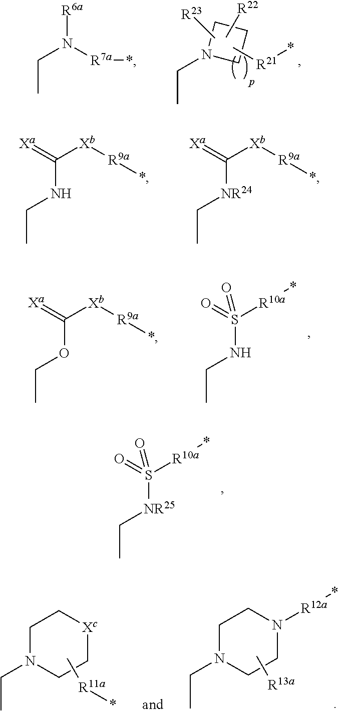

In some embodiments, in compounds of Formula (VI), X is —O—, —S— or —NH—, and R²⁵ is selected from:

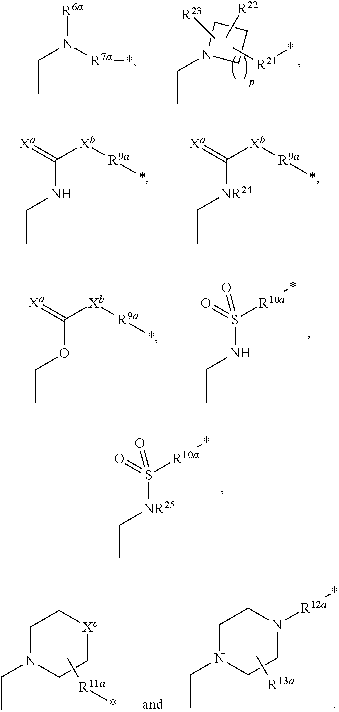

In some embodiments, in compounds of Formula (VI), X is —O— or —NH—.

In some embodiments, in compounds of Formula (VI), R^{6a} is H.

In some embodiments, in compounds of Formula (VI), R^{6a} is selected from: —H, unsubstituted —C₁-C₆ alkyl, —C₁-C₆ haloalkyl, —C₁-C₆ hydroxyalkyl, —C₃-C₈ cycloalkyl and —C₃-C₈ heterocycloalkyl.

In some embodiments, in compounds of Formula (VI), R^{7a} is selected from: —C₁-C₆ alkyl, —C₃-C₈ cycloalkyl and —C(O)R^{17a}.

In some embodiments, in compounds of Formula (VI), each R^{9a} is independently selected from: —C₁-C₆ alkyl, —C₃-C₈ cycloalkyl, -aryl and —(C₁-C₆ alkyl)-aryl.

In some embodiments, in compounds of Formula (VI), each R^{9a} is independently selected from: —C₁-C₆ alkyl and —(C₁-C₆ alkyl)-aryl.

In some embodiments, in compounds of Formula (VI), each R^{10a} is independently selected from: —C₁-C₆ alkyl, —C₃-C₈ cycloalkyl, -aryl, —(C₁-C₆ alkyl)-aryl and

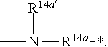

In some embodiments, in compounds of Formula (VI), each R^{10a} is independently selected from: —C₁-C₆ alkyl, -aryl, —(C₁-C₆ alkyl)-aryl and

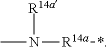

In some embodiments, in compounds of Formula (VI), R^{12a} is selected from: —C₁-C₆ alkyl, -aryl, —(C₁-C₆ alkyl)-aryl and —S(O)₂R^{16a}.

In some embodiments, in compounds of Formula (VI), R^{13a} is selected from: —H, unsubstituted —C₁-C₆ alkyl, —C₁-C₆ haloalkyl, —C₁-C₆ hydroxyalkyl and —C₁-C₆ aminoalkyl.

In some embodiments, in compounds of Formula (VI), R^{14a′} is selected from: H, unsubstituted —C₁-C₆ alkyl, —C₁-C₆ haloalkyl, —C₁-C₆ hydroxyalkyl, —C₁-C₆ aminoalkyl, —C₃-C₈ cycloalkyl and —C₃-C₈ heterocycloalkyl.

In some embodiments, in compounds of Formula (VI), R^{16a} is selected from: -aryl, -heteroaryl and —(C₁-C₆ alkyl)-aryl.

In some embodiments, in compounds of Formula (VI), R^{17a} is —C₁-C₆ alkyl.

In some embodiments, in compounds of Formula (VI), R²² and R²³ are each independently selected from: —H, -halogen, unsubstituted —C₁-C₆ alkyl, —C₁-C₆ haloalkyl, —C₁-C₆ hydroxyalkyl, —C₁-C₆ aminoalkyl and —C₃-C₈ cycloalkyl.

In some embodiments, in compounds of Formula (VI), X^{a} and X^{b} are each independently selected from: NH and O.

Combinations of any of the foregoing embodiments for compounds of Formula (VI) are also contemplated and each combination forms a separate embodiment for the purposes of the present disclosure.

In certain embodiments, each alkyl, cycloalkyl, heterocycloalkyl, aryl and heteroaryl group as defined in any one of Formulae (IV), (V) or (VI) is optionally substituted with one or more substituents selected from: halogen, acyl, acyloxy, alkoxy, carboxy, hydroxy, amino, amido, nitro, cyano, azido, alkylthio, thio, sulfonyl, sulfonamido, alkyl, cycloalkyl, heterocycloalkyl, aryl and heteroaryl. In some embodiments, each alkyl, cycloalkyl, heterocycloalkyl, aryl and heteroaryl group as defined in any one of Formulae (IV), (V) or (VI) is optionally substituted with one or more substituents selected from: halogen, acyl, acyloxy, alkoxy, carboxy, hydroxy, amino, amido, nitro, cyano, azido, alkylthio, thio, sulfonyl and sulfonamido.

### Targeting Moiety, T

The targeting moiety, T, comprised by the conjugates of Formula (X) is a molecule that binds, reactively associates or complexes with a receptor, antigen or other receptive moiety associated with a given target cell population. Typically, the targeting moiety, T, functions to deliver the camptothecin analogue, D, to the particular target cell population with which the targeting moiety, T, reacts. Examples of targeting moieties include, but are not limited to, proteins (such as antibodies, antibody fragments and growth factors), glycoproteins, peptides (such as bombesin and gastrin-releasing peptide), lectins, vitamins (such as folic acid) and nutrient-transport molecules (such as transferrin).

Typically, the targeting moiety, T, will be bonded to linker, L, via a heteroatom of targeting moiety, T, such as a sulfur (for example, from a sulfhydryl group), oxygen (for example, from a carbonyl, carboxyl or hydroxyl group) or nitrogen (for example, from a primary or secondary amino group). These heteroatoms may be naturally present on targeting moiety, T, or may be introduced through engineering and/or expression, or may be introduced via chemical or enzymatic modification using techniques known in the art.

In some embodiments, targeting moiety, T, is an antibody. Accordingly, certain embodiments of the present disclosure relate to antibody-drug conjugates (ADCs) having general Formula (X) in which the targeting moiety, T, is an antibody.

When the conjugate is an ADC, the antibody included as the targeting moiety, T, may be a full-size polyclonal or monoclonal antibody, an antigen-binding antibody fragment (such as Fab, scFab, Fab′, F(ab′)₂, Fv or scFv), a domain antibody (dAb) or an antibody mimetic (such as an affibody, a DARPin, an anticalin, a versabody, a duocalin, a lipocalin or an avimer). The antibody is typically directed to a particular antigen, for example, a disease-associated antigen such as a tumor-associated antigen, an antigen associated with an autoimmune disease or a viral antigen.

In certain embodiments in which the conjugate is an ADC, the targeting moiety, T, is a monoclonal antibody, an antigen-binding antibody fragment (such as Fab, scFab, Fab′, F(ab′)₂, Fv or scFv) or a domain antibody (dAb).

Methods of producing polyclonal and monoclonal antibodies are known in the art. By way of example, monoclonal antibodies may be produced by methods including, but not limited to, the hybridoma technique originally described by Kohler and Milstein (1975, Nature 256:495-497), the human B cell hybridoma technique (Kozbor et al., 1983, Immunology Today 4:72), the EBV-hybridoma technique (Cole et al., 1985, Monoclonal Antibodies and Cancer Therapy, Alan R. Liss, Inc., pp. 77-96), and the Selected Lymphocyte Antibody Method (SLAM) (Babcook, et al., 1996, Proc Natl Acad Sci USA, 93(15):7843-8; McLean et al., 2005, J Immunol., 174(8):4768-4778). Antibodies of various immunoglobulin classes including IgG, IgM, IgE, IgA, and IgD and subclasses thereof, may find application as targeting moieties in various embodiments. In some embodiments, the targeting moiety is an antibody of the IgG class.

In certain embodiments, targeting moiety, T, may be a monoclonal antibody. The monoclonal antibody may be, for example, a non-human monoclonal antibody (such as a mouse antibody), a human monoclonal antibody, a humanized monoclonal antibody or a chimeric antibody (for example, a human-mouse antibody). Human monoclonal antibodies may be made by any of numerous techniques known in the art (see, for example, Teng et al., 1983, Proc. Natl. Acad. Sci. USA 80:7308-7312; Kozbor et al., 1983, Immunology Today 4:72-79; Olsson et al., 1983, Meth. Enzymol. 92:3-16; Huse et al., 1989, Science 246:1275-1281, and U.S. Pat. No. 8,012,714). Chimeric and humanized monoclonal antibodies can be produced by recombinant DNA techniques known in the art, for example using methods described in International Patent Publication Nos. WO 87/02671 and WO 86/01533; European Patent Publication Nos. 0 184 187; 0 171 496 and 0 173 494; U.S. Pat. Nos. 4,816,567 and 5,225,539; Berter et al., 1988, Science 240:1041-1043; Liu et al., 1987, J. Immunol., 139:3521-3526; Sun et al., 1987, Proc. Nat. Acad. Sci. USA, 84:214-218; Wood et al., 1985, Nature, 314:446-449; Shaw et al., 1988, J. Natl. Cancer Inst., 80:1553-1559; Oi et al., 1986, BioTechniques, 4:214; Jones et al., 1986, Nature, 321:552-525, and Beidler et al., 1988, J. Immunol., 141:4053-4060). Antibodies immunospecific for a given target antigen may also be obtained commercially.

In certain embodiments, the antibody included in the conjugate may be a bispecific or multispecific antibody. Methods for making bispecific and multispecific antibodies are known in the art (see, for example, Milstein et al., 1983, Nature, 305:537-539; Traunecker et al., 1991, EMBO J., 10:3655-3659; Suresh et al., 1986, Meth. Enzymol., 121:210; Rodrigues et al., 1993, J. Immunol., 151:6954-6961; Carter et al., 1992, Bio Technology, 10:163-167; Carter et al., 1995, J. Hematotherapy, 4:463-470; Merchant et al., 1998, Nature Biotechnology, 16:677-681, and International (PCT) Publication Nos. WO 94/04690, WO 2012/032080, WO 2012/058768 and WO 2013/063702).

In certain embodiments, targeting moiety, T, comprised by the conjugate is an antibody or antigen-binding antibody fragment that binds to a tumor-associated antigen (TAA). Examples of tumor-associated antigens include, but are not limited to, 5T4, ADAM-9, ALK, AMHRII, ASCT2, Axl, B7-H3, BCMA, C4.4a, CA6, CA9, CanAg, CD123, CD138, CD142, CD166, CD184, CD19, CD20, CD205, CD22, CD248, CD25, CD3, CD30, CD33, CD352, CD37, CD38, CD40L, CD44v6, CD45, CD46, CD48, CD51, CD56, CD7, CD70, CD71, CD74, CD79b, CDH6, CEACAM5, CEACAM6, cKIT, CLDN18.2, CLDN6, CLL-1, c-MET, Cripto, CSP-1, CXCR5, DLK-1, DLL3, DPEP3, Dysadherin, EFNA4, EGFR, EGFRviii, ENPP3, EpCAM, EphA2, EphA3, ETBR, FGFR2, FGFR3, FLT3, FRa, FSH, GCC, GD2, GD3, Globo H, GPC-1, GPC3, gpNMB, HER-2, HER-3, HLA-DR, HSP90, IGF-1R, IL-13R, ILIRAP, IL7R, IL4R, KAAG-1, LAMP-1, Lewis Y antigen, LGALS3BP, LGR5, LH/hCG, LHRH, LIV-1, LRP-1, LRRC15, Ly6E, MAGE, MSLN, MET, MICA, MICB, MT1-MMP, MTX3, MTX5, MUC1, MUC16, NaPi2b, Nectin-4, NOTCH3, OAcGD2, OX001L, p-cadherin, PD-1, PD-L1, phosphatidylserine (PS), polymorphic epithelial mucin (PEM), prolactin receptor (PRLR), PSMA, PTK7, RNF43, ROR1, ROR2, SAIL, SLAMF7, SLC44A4, SLITRK6, SSTR2, STEAP-1, STING, sialyl-Tn, TIM-1, TM4SF1, TNFα, TRA, TROP-2, TAG-72, TA-MUC1, TIM-3, UPK2 and UPK1b.

### Linker, L

The conjugates of Formula (X) include a linker, L, which is a bifunctional or multifunctional moiety capable of linking one or more camptothecin analogues, D, to targeting moiety, T. A bifunctional (or monovalent) linker, L, links a single compound D to a single site on targeting moiety, T, whereas a multifunctional (or polyvalent) linker, L, links more than one compound, D, to a single site on targeting moiety, T. A linker that links one compound, D, to more than one site on targeting moiety, T, may also be considered to be multifunctional in certain embodiments.

Linker, L, includes a functional group capable of reacting with the target group or groups on targeting moiety, T, and at least one functional group capable of reacting with a target group on the camptothecin analogue, D. Suitable functional groups are known in the art and include those described, for example, in Bioconjugate Techniques (G. T. Hermanson, 2013, Academic Press). Groups on targeting moiety, T, and the camptothecin analogue, D, that may serve as target groups for linker attachment include, but are not limited to, thiol, hydroxyl, carboxyl, amine, aldehyde and ketone groups.

Non-limiting examples of functional groups capable of reacting with thiols include maleimide, haloacetamide, haloacetyl, activated esters (such as succinimide esters, 4-nitrophenyl esters, pentafluorophenyl esters and tetrafluorophenyl esters), anhydrides, acid chlorides, sulfonyl chlorides, isocyanates and isothiocyanates. Also useful in this context are “self-stabilizing” maleimides as described in Lyon et al., 2014, Nat. Biotechnol., 32:1059-1062.

Non-limiting examples of functional groups capable of reacting with amines include activated esters (such as N-hydroxysuccinamide (NHS) esters, sulfo-NHS esters, imido esters such as Traut's reagent, tetrafluorophenyl (TFP) esters and sulfodichlorophenyl esters), isothiocyanates, aldehydes and acid anhydrides (such as diethylenetriaminepentaacetic anhydride (DTPA)). Other examples include succinimido-1,1,3,3-tetra-methyluronium tetrafluoroborate (TSTU) and benzotriazol-1-yl-oxytripyrrolidinophosphonium hexafluorophosphate (PyBOP).

Non-limiting examples of functional groups capable of reacting with an electrophilic group such as an aldehyde or ketone carbonyl group include hydrazide, oxime, amino, hydrazine, thiosemicarbazone, hydrazine carboxylate and arylhydrazide.

In certain embodiments in which targeting moiety, T, is an antibody, linker, L, may include a functional group that allows for bridging of two interchain cysteines on the antibody, such as a ThioBridge™ linker (Badescu et al., 2014, Bioconjug. Chem. 25:1124-1136), a dithiomaleimide (DTM) linker (Behrens et al., 2015, Mol. Pharm. 12:3986-3998), a dithioaryl(TCEP)pyridazinedione-based linker (Lee et al., 2016, Chem. Sci., 7:799-802) or a dibromopyridazinedione-based linker (Maruani et al., 2015, Nat. Commun., 6:6645).

Alternatively, targeting moiety, T, may be modified to include a non-natural reactive group, such as an azide, that allows for conjugation to the linker via a complementary reactive group on the linker. For example, conjugation of the linker to the targeting moiety may make use of click chemistry reactions (see, for example, Chio & Bane, 2020, Methods Mol. Biol., 2078:83-97), such as the azide-alkyne cycloaddition (AAC) reaction, which has been used successfully in the development of antibody-drug conjugates. The AAC reaction may be a copper-catalyzed AAC (CuAAC) reaction, which involves coupling of an azide with a linear alkyne, or a strain-promoted AAC (SPAAC) reaction, which involves coupling of an azide with a cyclooctyne.

Linker, L, may be a cleavable or a non-cleavable linker. A cleavable linker is a linker that is susceptible to cleavage under specific conditions, for example, intracellular conditions (such as in an endosome or lysosome) or within the vicinity of a target cell (such as in the tumor microenvironment). Examples include linkers that are protease-sensitive, acid-sensitive or reduction-sensitive. Non-cleavable linkers by contrast, rely on the degradation of the antibody in the cell, which typically results in the release of an amino acid-linker-drug moiety.

Examples of cleavable linkers include, for example, linkers comprising an amino acid sequence that is a cleavage recognition sequence for a protease. Many such cleavage recognition sequences are known in the art. For conjugates that are not intended to be internalized by a cell, for example, an amino acid sequence that is recognized and cleaved by a protease present in the extracellular matrix in the vicinity of a target cell, such as a cancer cell, may be employed. Examples of extracellular tumor-associated proteases include, for example, plasmin, matrix metalloproteases (MMPs), elastase and kallikrein-related peptidases.

For conjugates intended to be internalized by a cell, linker, L, may comprise an amino acid sequence that is recognized and cleaved by an endosomal or lysosomal protease. Examples of such proteases include, for example, cathepsins B, C, D, H, L and S, and legumain.

Cleavage recognition sequences may be, for example, dipeptides, tripeptides or tetrapeptides. Non-limiting examples of dipeptide recognition sequences that may be included in cleavable linkers include, but are not limited to, Ala-(D)Asp, Ala-Lys, Ala-Phe, Asn-Lys, Asn-(D)Lys, Asp-Val, His-Val, Ile-Cit, Ile-Pro, Ile-Val, Leu-Cit, Me₃Lys-Pro, Met-Lys, Met-(D)Lys, NorVal-(D)Asp, Phe-Arg, Phe-Cit, Phe-Lys, PhenylGly-(D)Lys, Pro-(D)Lys, Trp-Cit, Val-Ala, Val-(D)Asp, Val-Cit, Val-Gly, Val-Gln and Val-Lys. Examples of tri- and tetrapeptide cleavage sequences include, but are not limited to, Ala-Ala-Asn, Ala-Val-Cit, (D)Ala-Phe-Lys, Asp-Val-Ala, Asp-Val-Cit, Gly-Cit-Val, Lys-Val-Ala, Lys-Val-Cit, Met-Cit-Val, (D)Phe-Phe-Lys, Asn-Pro-Val, Ala-Leu-Ala-Leu, Gly-Phe-Leu-Gly, Gly-Gly-Phe-Gly and Gly-Phe-Gly-Gly.

Additional examples of cleavable linkers include disulfide-containing linkers such as N-succinimydyl-4-(2-pyridyldithio) butanoate (SPDB) and N-succinimydyl-4-(2-pyridyldithio)-2-sulfo butanoate (sulfo-SPDB). Disulfide-containing linkers may optionally include additional groups to provide steric hindrance adjacent to the disulfide bond in order to improve the extracellular stability of the linker, for example, inclusion of a geminal dimethyl group. Other cleavable linkers include linkers hydrolyzable at a specific pH or within a pH range, such as hydrazone linkers. Linkers comprising combinations of these functionalities may also be useful, for example, linkers comprising both a hydrazone and a disulfide are known in the art.

A further example of a cleavable linker is a linker comprising a β-glucuronide, which is cleavable by β-glucuronidase, an enzyme present in lysosomes and tumor interstitium (see, for example, De Graaf et al., 2002, Curr. Pharm. Des. 8:1391-1403, and International Patent Publication No. WO 2007/011968). β-glucuronide may also function to improve the hydrophilicity of linker, L.

Another example of a linker that is cleaved internally within a cell and improves hydrophilicity is a linker comprising a pyrophosphate diester moiety (see, for example, Kern et al., 2016, J Am Chem Soc., 138:2430-1445).

In certain embodiments, the linker, L, comprised by the conjugate of Formula (X) is a cleavable linker. In some embodiments, linker, L, comprises a cleavage recognition sequence. In some embodiments, linker, L, may comprise an amino acid sequence that is recognized and cleaved by a lysosomal protease.

Cleavable linkers may optionally further comprise one or more additional functionalities such as self-immolative and self-elimination groups, stretchers or hydrophilic moieties.

Self-immolative and self-elimination groups that find use in linkers include, for example, p-aminobenzyl (PAB) and p-aminobenzyloxycarbonyl (PABC) groups, and methylated ethylene diamine (MED). Other examples of self-immolative groups include, but are not limited to, aromatic compounds that are electronically similar to the PAB or PABC group such as heterocyclic derivatives, for example 2-aminoimidazol-5-methanol derivatives as described in U.S. Pat. No. 7,375,078. Other examples include groups that undergo cyclization upon amide bond hydrolysis, such as substituted and unsubstituted 4-aminobutyric acid amides (Rodrigues et al., 1995, Chemistry Biology 2:223-227) and 2-aminophenylpropionic acid amides (Amsberry, et al., 1990, J. Org. Chem. 55:5867-5877). Self-immolative/self-elimination groups are typically attached to an amino or hydroxyl group on the compound, D. Self-immolative/self-elimination groups, alone or in combination are often included in peptide-based linkers, but may also be included in other types of linkers.

Stretchers that find use in linkers for drug conjugates include, for example, alkylene groups and stretchers based on aliphatic acids, diacids, amines or diamines, such as diglycolate, malonate, caproate and caproamide. Other stretchers include, for example, glycine-based stretchers and polyethylene glycol (PEG) or monomethoxy polyethylene glycol (mPEG) stretchers.

PEG and mPEG stretchers can also function as hydrophilic moieties within a linker. For example, PEG or mPEG may be included in a linker either “in-line” or as pendant groups to increase the hydrophilicity of the linker (see, for example, U.S. Patent Application Publication No. US 2016/0310612). Various PEG-containing linkers are commercially available from companies such as Quanta BioDesign, Ltd (Plain City, OH). Other hydrophilic groups that may optionally be incorporated into linker, L, include, for example, 0-glucuronide, sulfonate groups, carboxylate groups and pyrophosphate diesters.

In certain embodiments, conjugates of Formula (X) may comprise a cleavable linker. In some embodiments, conjugates of Formula (X) may comprise a peptide-containing linker. In some embodiments, conjugates of Formula (X) may comprise a protease-cleavable linker.

In some embodiments, in conjugates of Formula (X), linker, L, is a cleavable linker having Formula (XI):

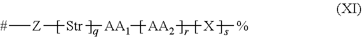

wherein:
Z is a linking group that joins the linker to a target group on targeting moiety, T;
Str is a stretcher;
AA₁ and AA₂ are each independently an amino acid, wherein AA₁-[AA₂]ᵣ forms a
protease cleavage site;
X is a self-immolative group;
q is 0 or 1;
r is 1, 2 or 3;
s is 0, 1 or 2;
# is the point of attachment to targeting moiety, T, and
% is the point of attachment to the camptothecin analogue, D.

In some embodiments, in linkers of Formula (XI), q is 1.

In some embodiments, in linkers of Formula (XI), s is 1. In some embodiments, in linkers of Formula (XI), s is 0.

In some embodiments, in linkers of Formula (XI):
Z is

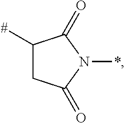

where # is the point of attachment to T, and * is the point of attachment to the remainder of the linker.

In some embodiments, in linkers of Formula (XI), Str is selected from:

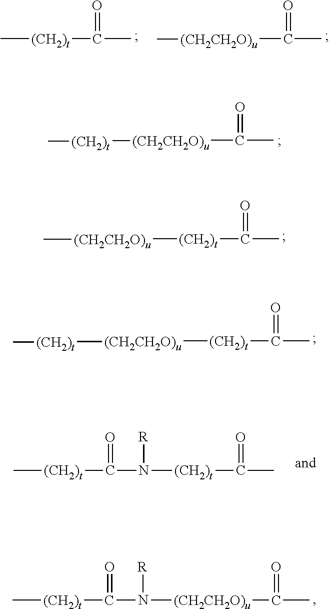

wherein:
R is H or C₁-C₆ alkyl;
t is an integer between 2 and 10, and
u is an integer between 1 and 10.

In some embodiments, in linkers of Formula (XI), Str is selected from:

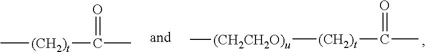

wherein:
R is H or C₁-C₆ alkyl;
t is an integer between 2 and 10, and
u is an integer between 1 and 10.

In some embodiments, in linkers of Formula (XI), AA₁-[AA₂]ᵣ has a sequence selected from: Ala-(D)Asp, Ala-Lys, Ala-Phe, Asn-Lys, Asn-(D)Lys, Asp-Val, His-Val, Ile-Cit, Ile-Pro, Ile-Val, Leu-Cit, Me₃Lys-Pro, Met-Lys, Met-(D)Lys, NorVal-(D)Asp, Phe-Arg, Phe-Cit, Phe-Lys, PhenylGly-(D)Lys, Pro-(D)Lys, Trp-Cit, Val-Ala, Val-(D)Asp, Val-Cit, Val-Gly, Val-Gln and Val-Lys. Examples of tri- and tetrapeptide cleavage sequences include, but are not limited to, Ala-Ala-Asn, Ala-Val-Cit, (D)Ala-Phe-Lys, Asp-Val-Ala, Asp-Val-Cit, Gly-Cit-Val, Lys-Val-Ala, Lys-Val-Cit, Met-Cit-Val, (D)Phe-Phe-Lys, Asn-Pro-Val, Ala-Leu-Ala-Leu, Gly-Phe-Leu-Gly, Gly-Gly-Phe-Gly and Gly-Phe-Gly-Gly.

In some embodiments, in conjugates of Formula (X), m is 1, and linker, L, has Formula (XI).

In certain embodiments, in conjugates of Formula (X), linker, L, is a cleavable linker having Formula (XII):

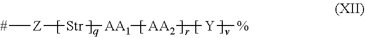

wherein:
Z is a linking group that joins the linker to a target group on targeting moiety, T;
Str is a stretcher;
AA₁ and AA₂ are each independently an amino acid, wherein AA₁-[AA₂]ᵣ forms a protease cleavage site;
Y is —NH—CH₂— or —NH—CH₂—C(O)—;
q is 0 or 1;
r is 1, 2 or 3;
v is 0 or 1;
# is the point of attachment to targeting moiety, T, and
% is the point of attachment to the camptothecin analogue, D.

In some embodiments, in linkers of Formula (XII), q is 1.

In some embodiments, in linkers of Formula (XII), s is 0. In some embodiments, in linkers of Formula (XII), s is 1.

In some embodiments, in linkers of Formula (XII):
Z is

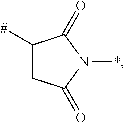

where # is the point of attachment to T, and * is the point of attachment to the remainder of the linker.

In some embodiments, in linkers of Formula (XII), Str is selected from:

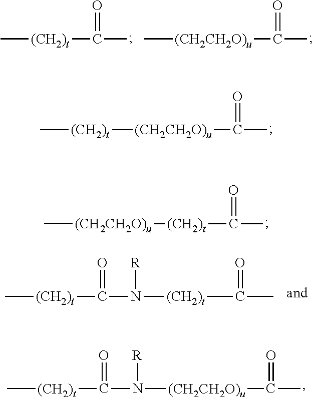

wherein:
R is H or C₁-C₆ alkyl;
t is an integer between 2 and 10, and
u is an integer between 1 and 10.

In some embodiments, in linkers of Formula (XII), Str is selected from:

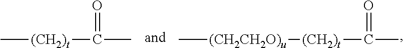

wherein:
R is H or C₁-C₆ alkyl;
t is an integer between 2 and 10, and
u is an integer between 1 and 10.

In some embodiments, in linkers of Formula (XII), AA₁-[AA₂]ᵣ has a sequence selected from: Ala-(D)Asp, Ala-Lys, Ala-Phe, Asn-Lys, Asn-(D)Lys, Asp-Val, His-Val, Ile-Cit, Ile-Pro, Ile-Val, Leu-Cit, Me₃Lys-Pro, Met-Lys, Met-(D)Lys, NorVal-(D)Asp, Phe-Arg, Phe-Cit, Phe-Lys, PhenylGly-(D)Lys, Pro-(D)Lys, Trp-Cit, Val-Ala, Val-(D)Asp, Val-Cit, Val-Gly, Val-Gln and Val-Lys. Examples of tri- and tetrapeptide cleavage sequences include, but are not limited to, Ala-Ala-Asn, Ala-Val-Cit, (D)Ala-Phe-Lys, Asp-Val-Ala, Asp-Val-Cit, Gly-Cit-Val, Lys-Val-Ala, Lys-Val-Cit, Met-Cit-Val, (D)Phe-Phe-Lys, Asn-Pro-Val, Ala-Leu-Ala-Leu, Gly-Phe-Leu-Gly, Gly-Gly-Phe-Gly and Gly-Phe-Gly-Gly.

In some embodiments, in conjugates of Formula (X), m is 1, and linker, L, has Formula (XII).

In some embodiments, conjugates of Formula (X) may comprise a disulfide-containing linker. In some embodiments, in conjugates of Formula (X), linker, L, is a cleavable linker having Formula (XIII):

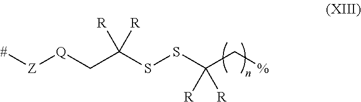

wherein:
Z is a linking group that joins the linker to a target group on targeting moiety, T;
Q is —(CH₂)ₚ— or —(CH₂CH₂O)_{q}—, wherein p and q are each independently an integer between 1 and 10;
each R is independently H or C₁-C₆ alkyl;
n is 1, 2 or 3;
# is the point of attachment to targeting moiety, T, and
% is the point of attachment to the camptothecin analogue, D.

In some embodiments, in conjugates of Formula (X), m is 1, and linker, L, has Formula (XIII).

In some embodiments, conjugates of Formula (X) may comprise a 0-glucuronide-containing linker.

Various non-cleavable linkers are known in the art for linking drugs to targeting moieties and may be useful in the conjugate compositions of the present disclosure in certain embodiments. Examples of non-cleavable linkers include linkers having an N-succinimidyl ester or N-sulfosuccinimidyl ester moiety for reaction with the cell binding agent, as well as a maleimido- or haloacetyl-based moiety for reaction with the drug, or vice versa. An example of such a non-cleavable linker is based on sulfosuccinimidyl-4-[N-maleimidomethyl]cyclohexane-1-carboxylate (sulfo-SMCC). Sulfo-SMCC conjugation typically occurs via a maleimide group which reacts with sulfhydryls (thiols, SH) on compound D, while the sulfo-NHS ester is reactive toward primary amines (as found in lysine and at the N-terminus of proteins or peptides) on targeting moiety T. Other non-limiting examples of such linkers include those based on N-succinimidyl 4-(maleimidomethyl)cyclohexanecarboxylate (SMCC), N-succinimidyl-4-(N-maleimidomethyl)-cyclohexane-1-carboxy-(6-amidocaproate) (“long chain” SMCC or LC-SMCC), κ-maleimidoundecanoic acid N-succinimidyl ester (KMUA), γ-maleimidobutyric acid N-succinimidyl ester (GMBS), ε-maleimidocaproic acid N-hydroxysuccinimide ester (EMCS), m-maleimidobenzoyl-N-hydroxysuccinimide ester (MBS), N-(α-maleimidoacetoxy)-succinimide ester (AMAS), succinimidyl-6-(β-maleimidopropionamido)hexanoate (SMPH), N-succinimidyl 4-(p-maleimidophenyl)-butyrate (SMPB) and N-(p-maleimidophenyl)isocyanate (PMPI). Other examples include those comprising a haloacetyl-based functional group such as N-succinimidyl-4-(iodoacetyl)-aminobenzoate (SIAB), N-succinimidyl iodoacetate (SIA), N-succinimidyl bromoacetate (SBA) and N-succinimidyl 3-(bromoacetamido)propionate (SBAP).

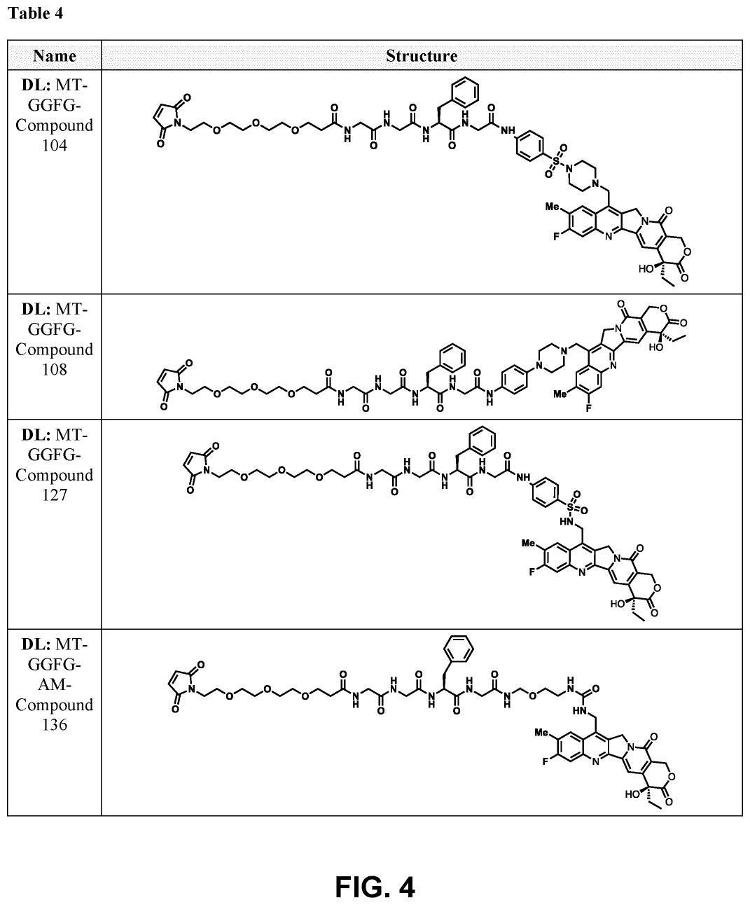

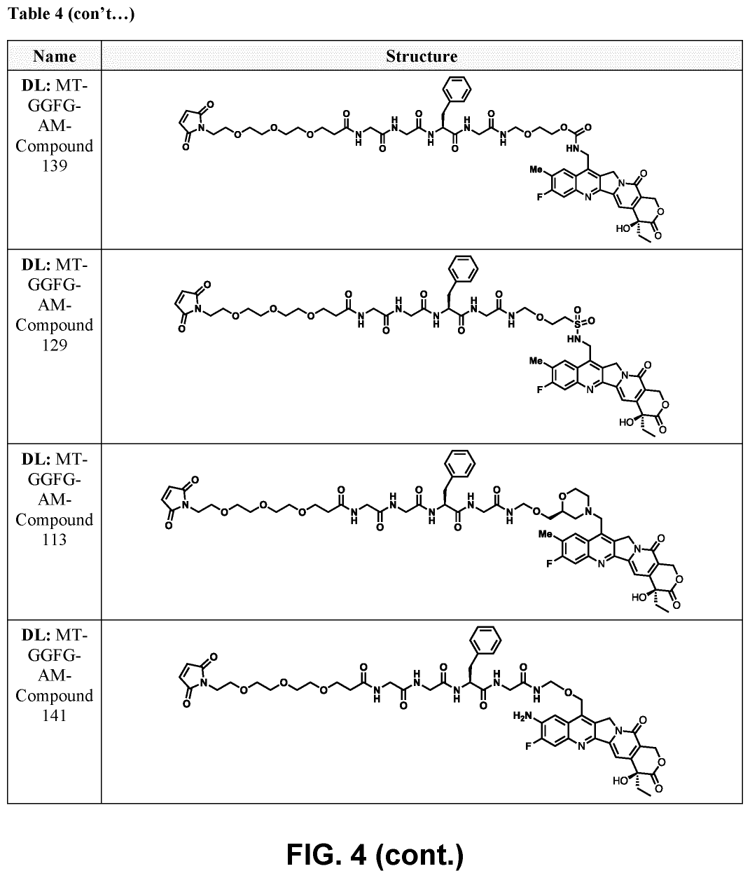

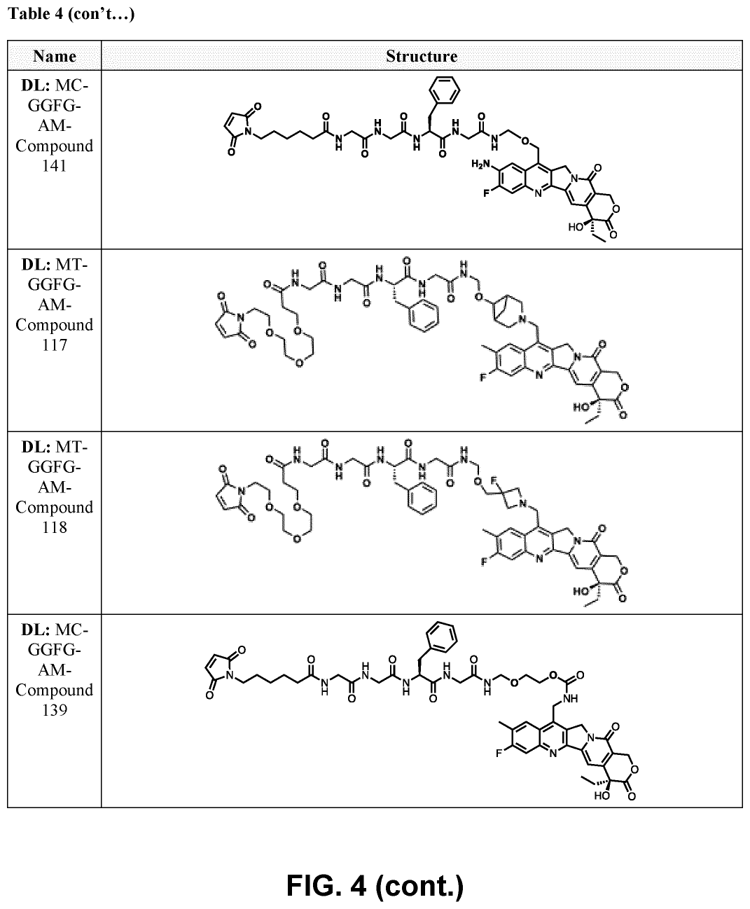

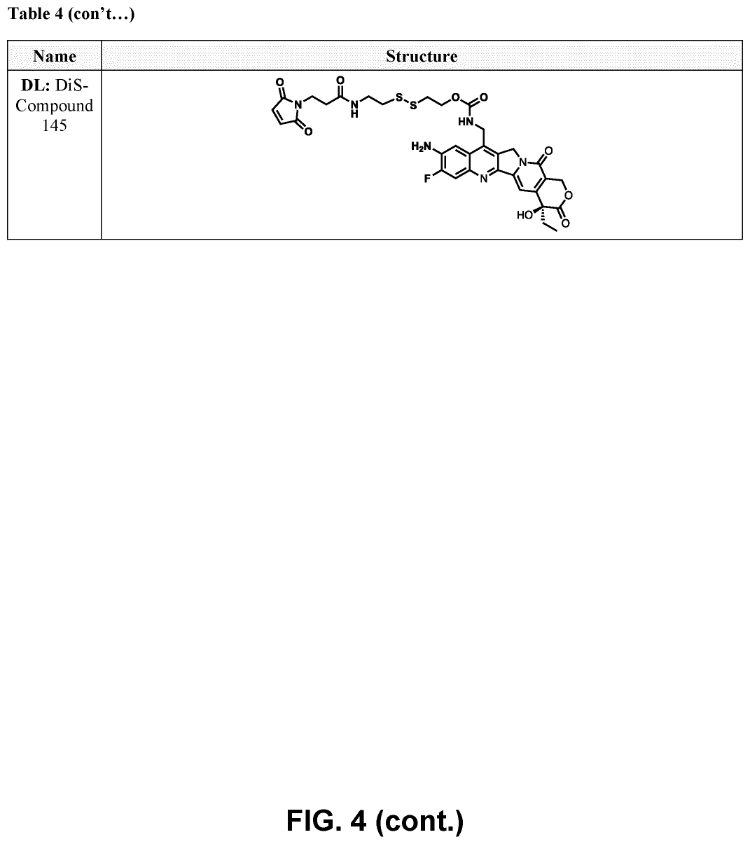

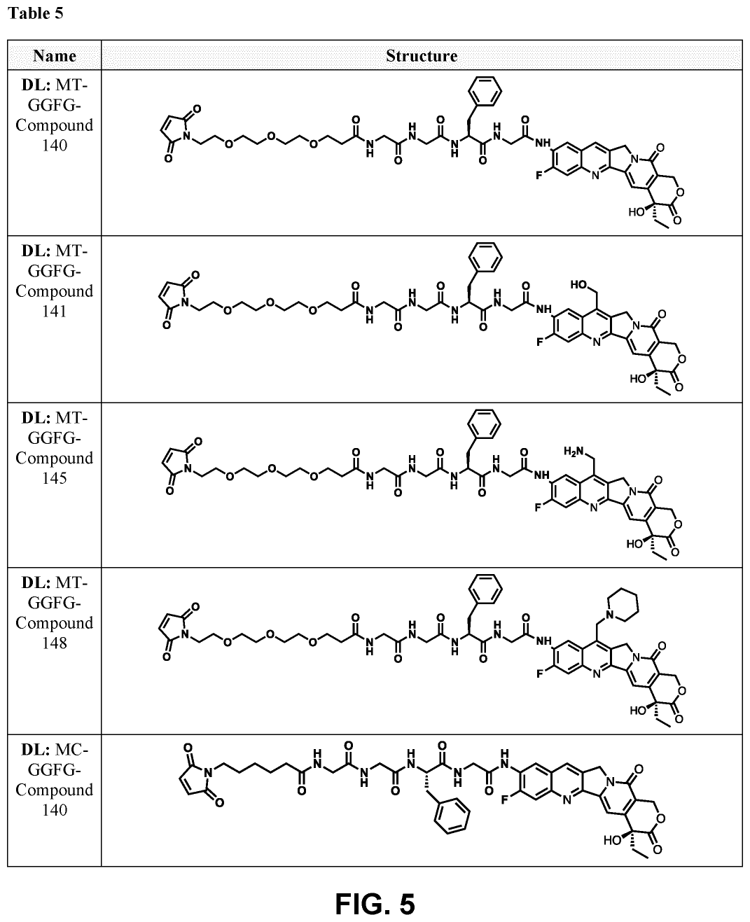

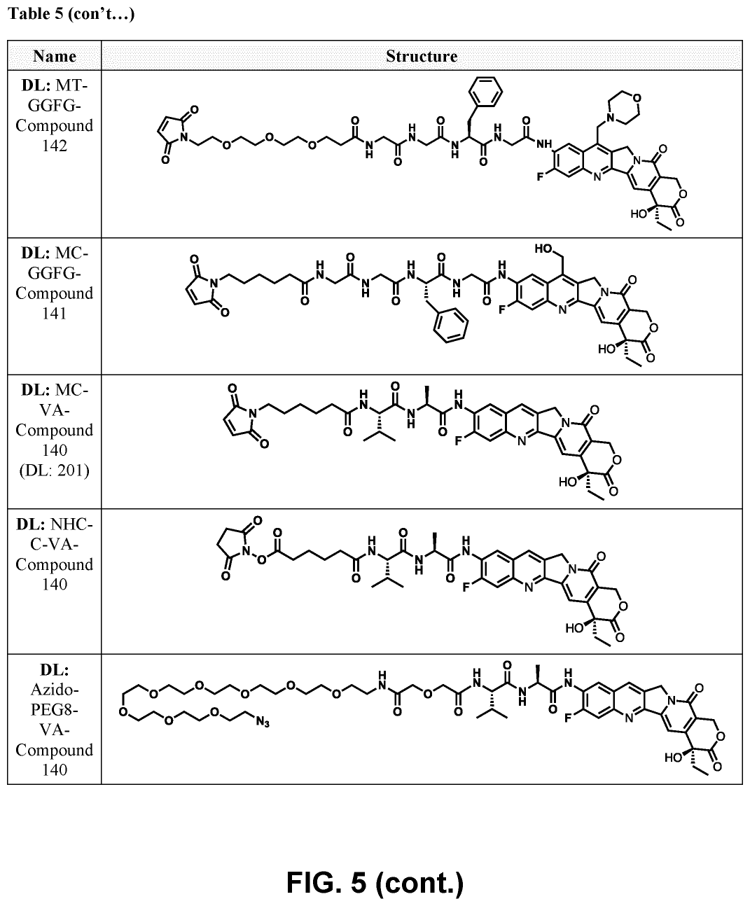

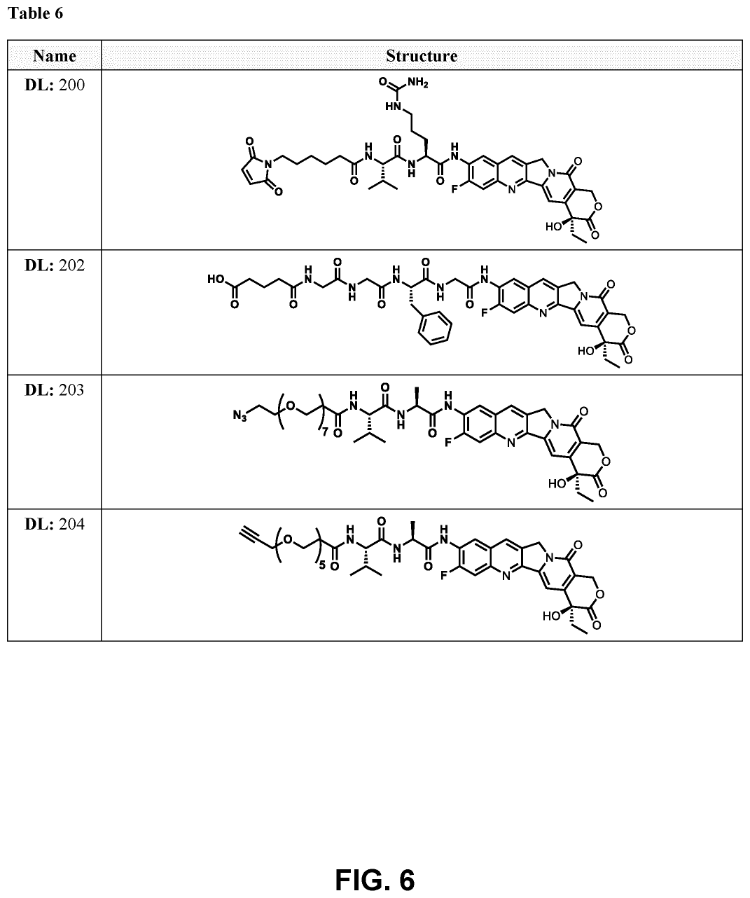

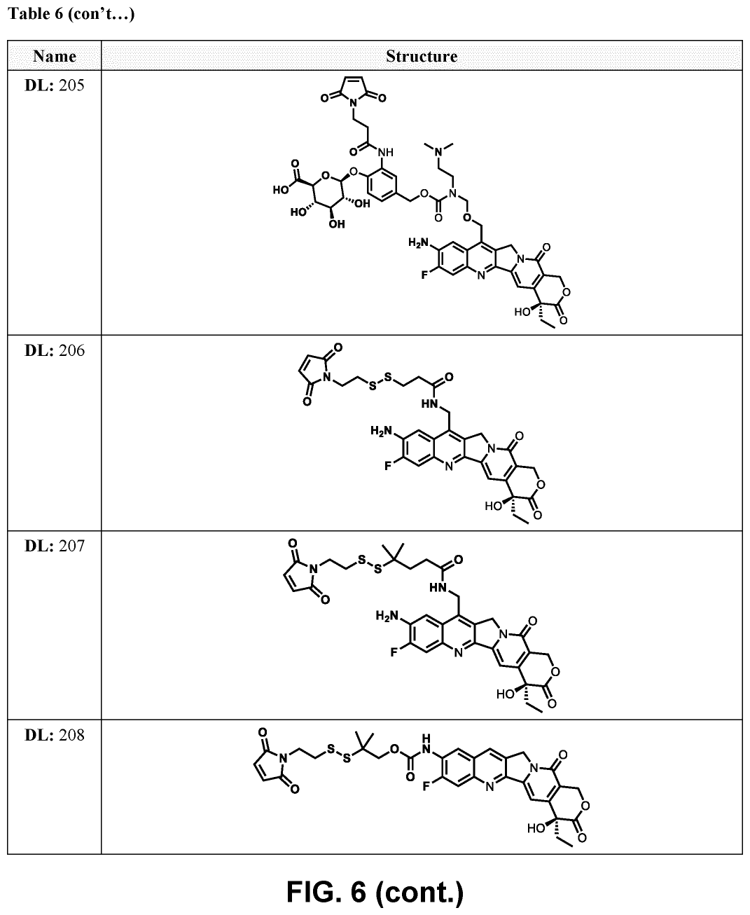

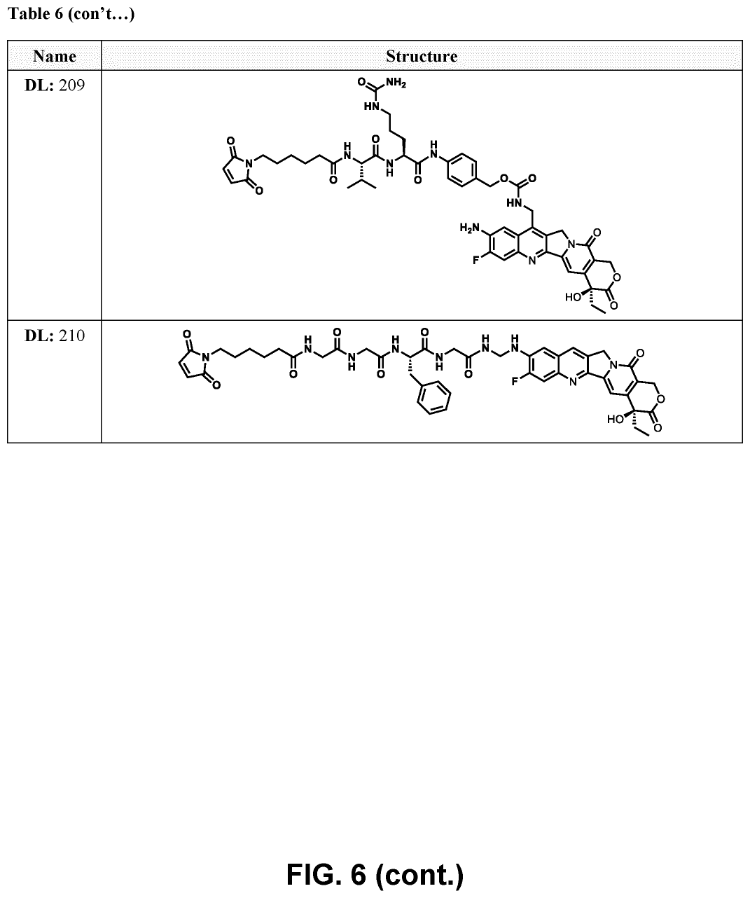

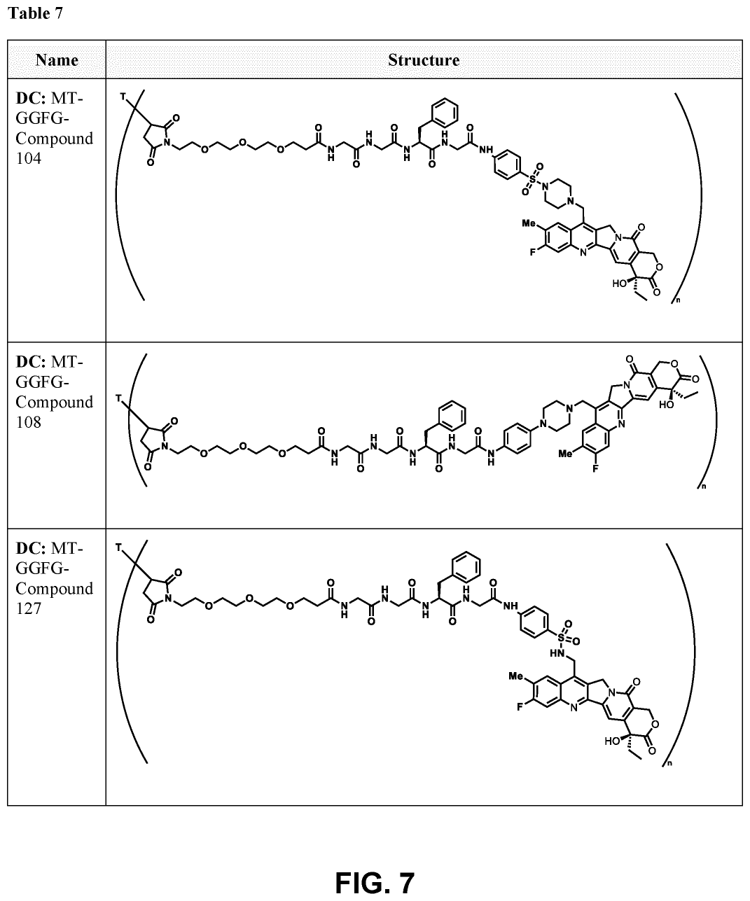

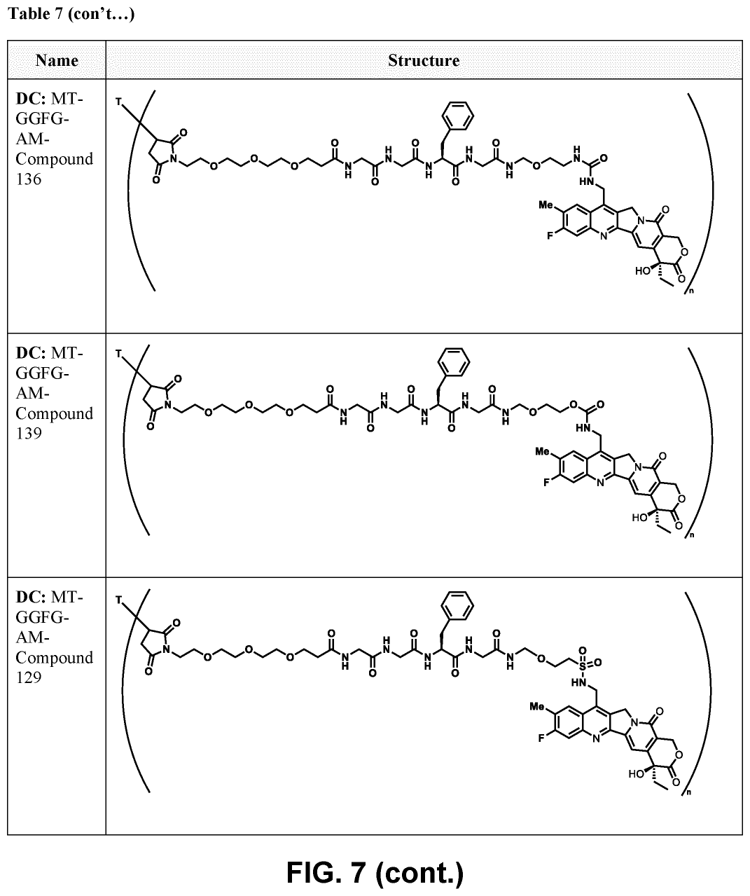

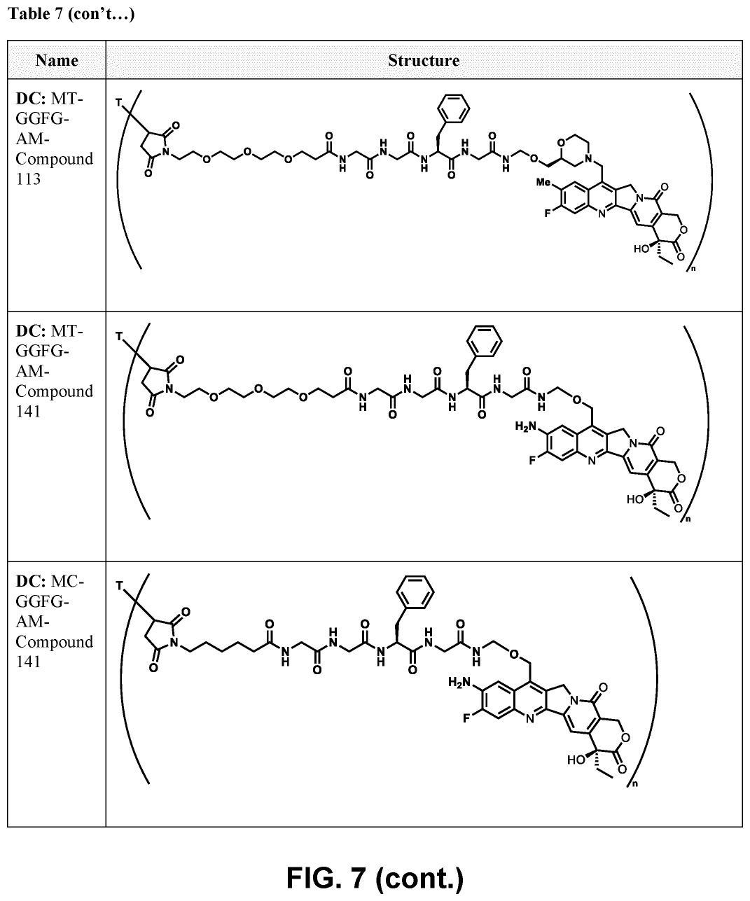

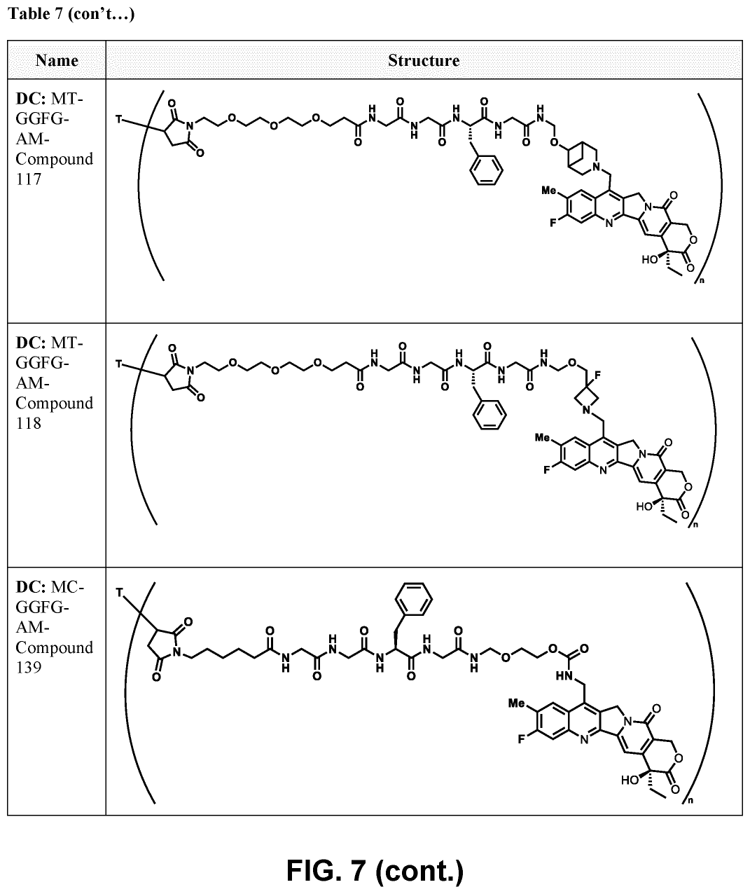

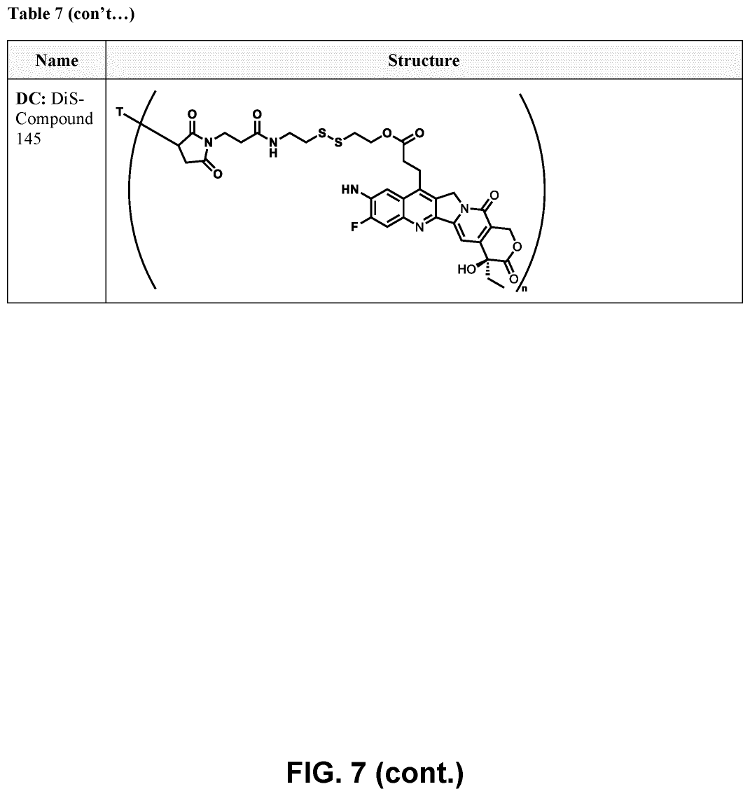

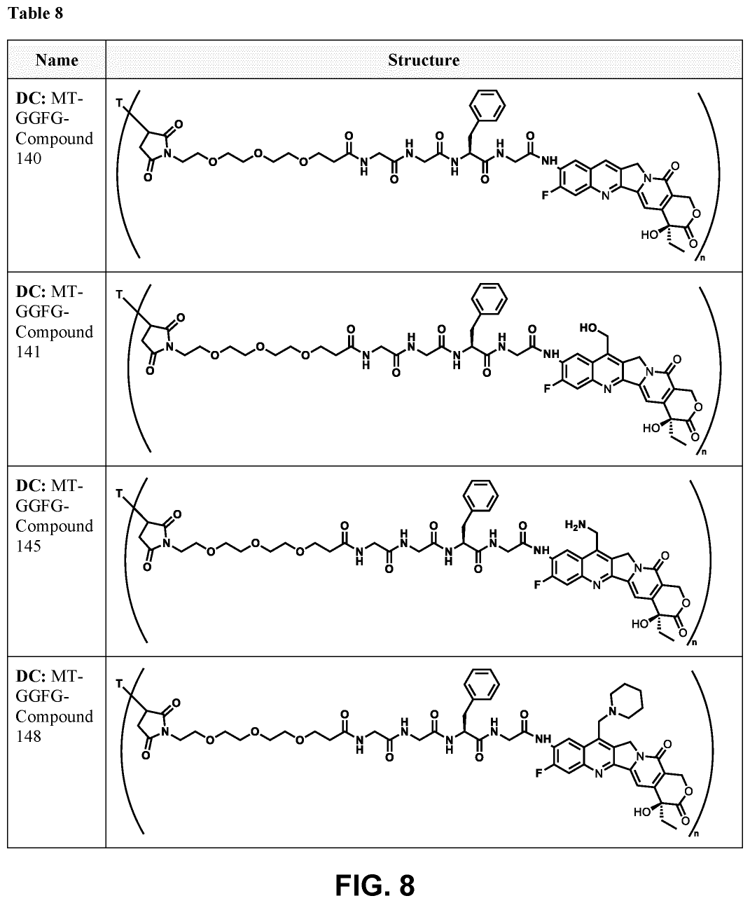

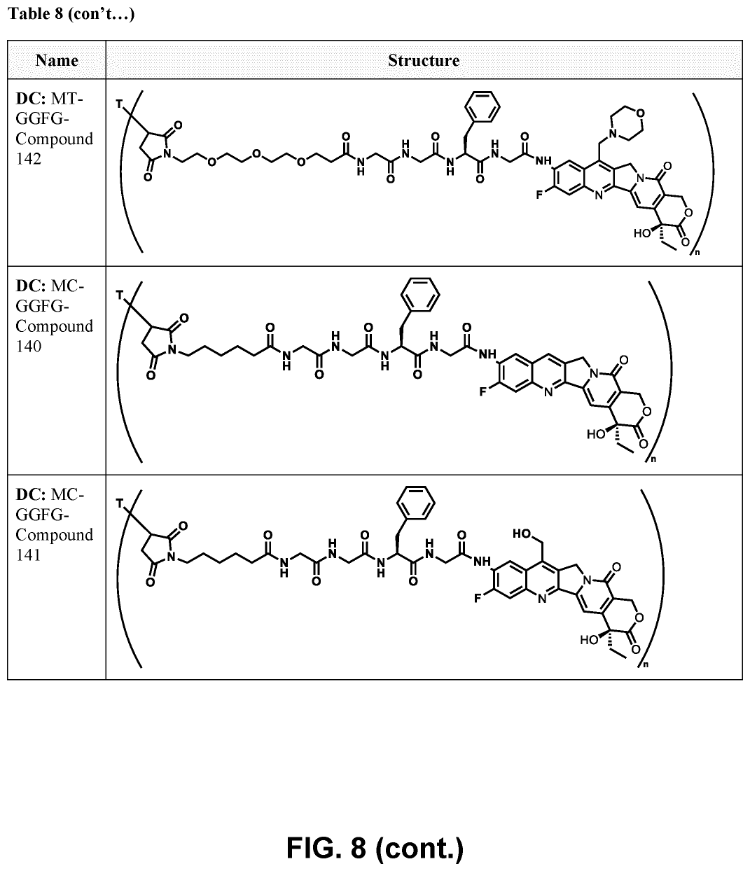

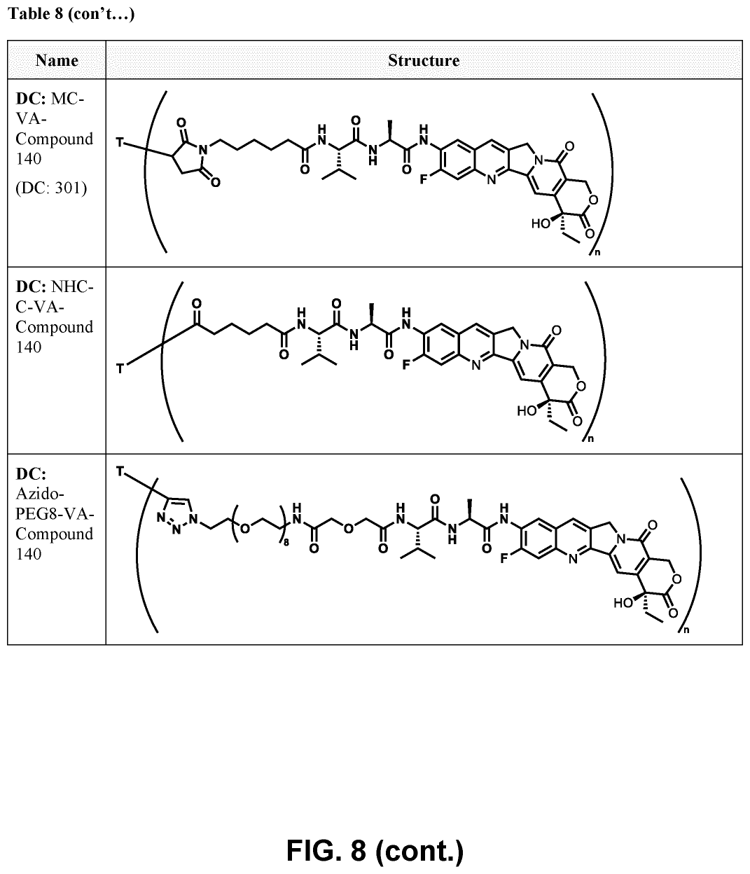

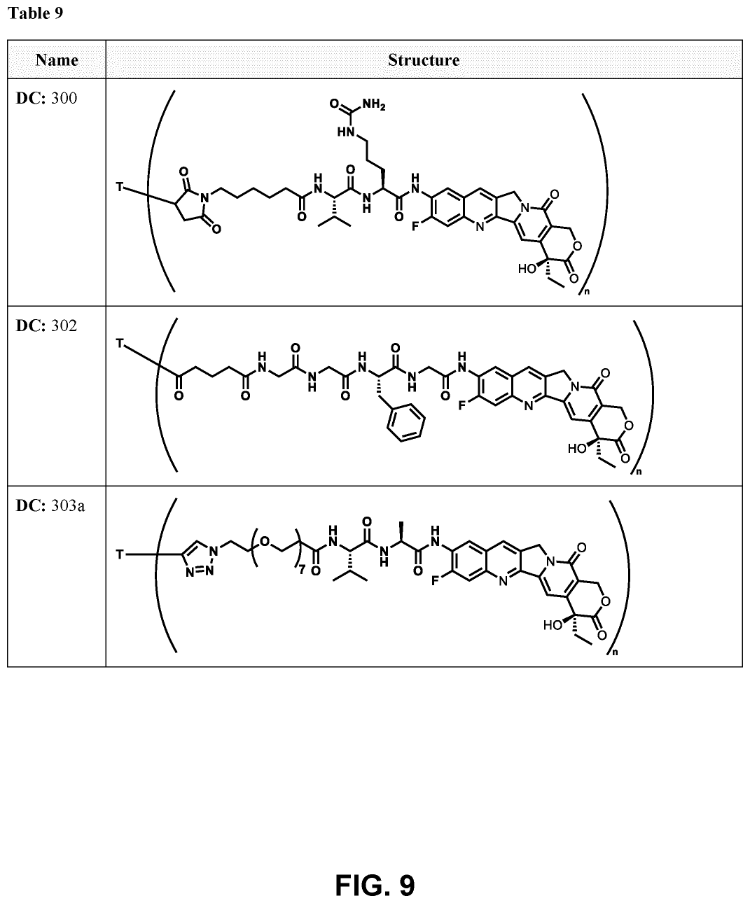

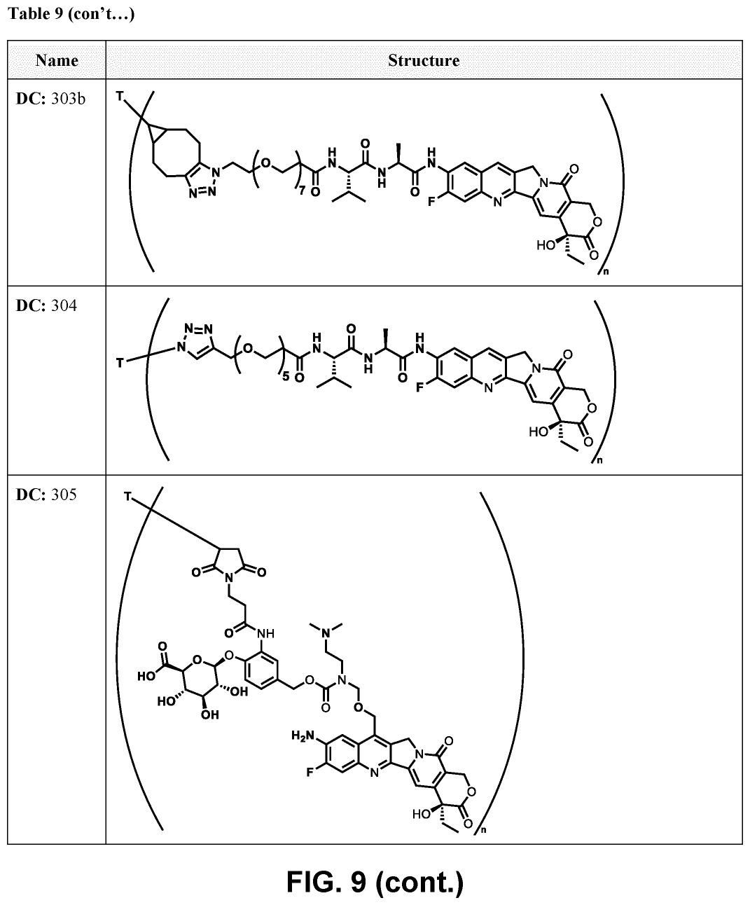

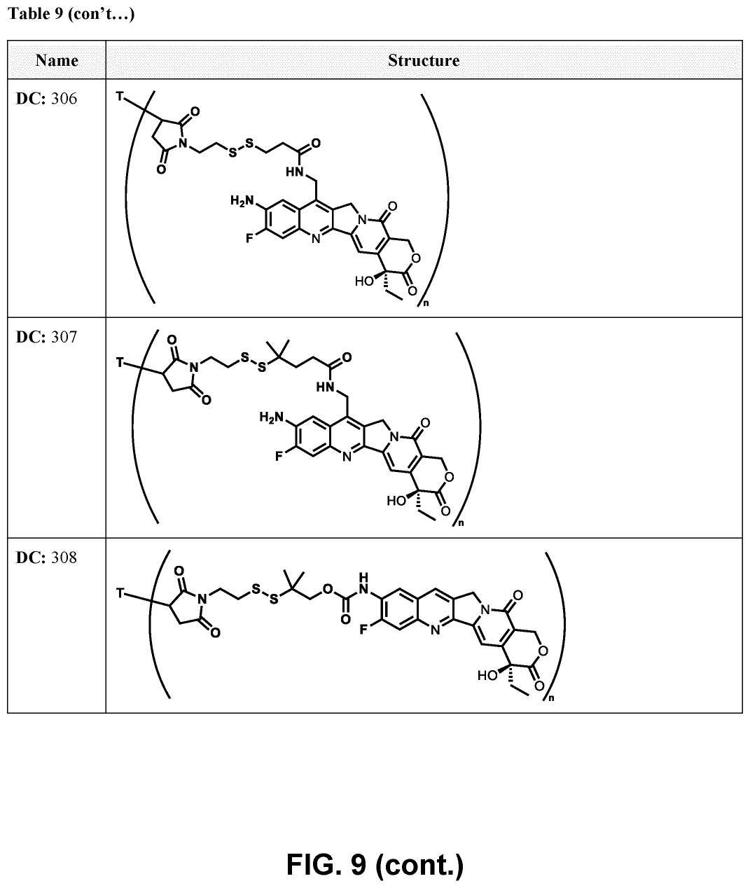

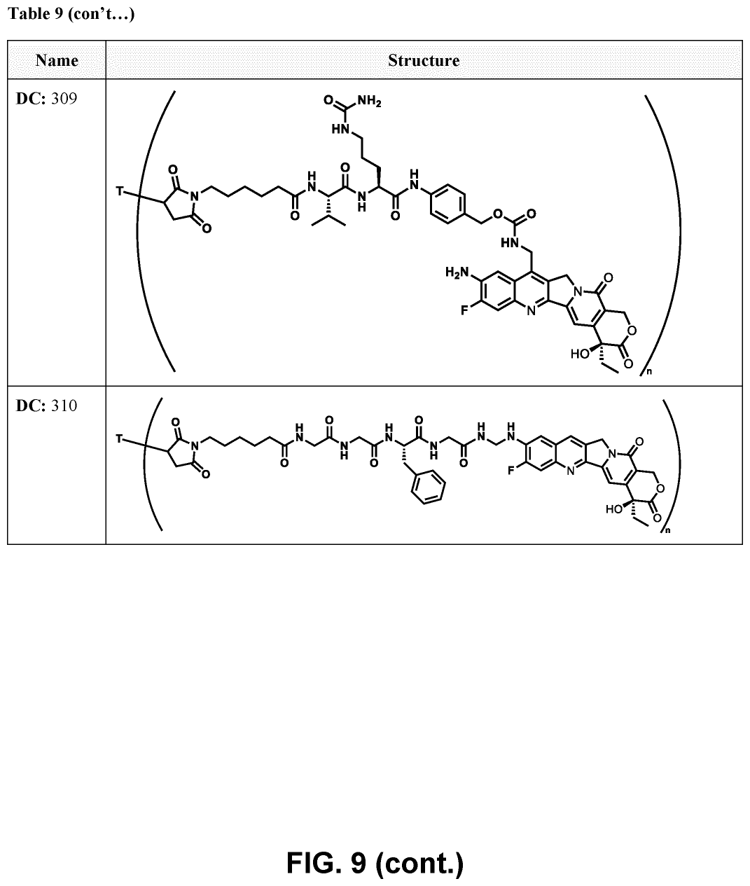

Non-limiting examples of drug-linkers comprising camptothecin analogues of Formula (I) are shown in Table 4 (FIG. 4), Table 5 (FIG. 5) and Table 6 (FIG. 6). Non-limiting examples of conjugates comprising these drug-linkers are shown in Table 7 (FIG. 7), Table 8 (FIG. 8) and Table 9 (FIG. 9). In certain embodiments, the conjugate of Formula (X) comprises a drug-linker selected from the drug-linkers shown in Tables 4, 5 and 6. In certain embodiments, the conjugate of Formula (X) is selected from the conjugates shown in Tables 7, 8 and 9, where T is the targeting moiety and n is an integer between 1 and 10. In some embodiments, the conjugate of Formula (X) is selected from the conjugates shown in Tables 7, 8 and 9, where T is the targeting moiety and n is an integer between 2 and 8. In some embodiments, the conjugate of Formula (X) is selected from the conjugates shown in Tables 7, 8 and 9, where T is an antibody or antigen-binding antibody fragment.

### Preparation

Conjugates of Formula (X) may be prepared by standard methods known in the art (see, for example, Bioconjugate Techniques (G. T. Hermanson, 2013, Academic Press)). Various linkers and linker components are commercially available or may be prepared using standard synthetic organic chemistry techniques (see, for example, March's Advanced Organic Chemistry (Smith & March, 2006, Sixth Ed., Wiley); Toki et al., (2002) J. Org. Chem. 67:1866-1872; Frisch et al., (1997) Bioconj. Chem. 7:180-186; Bioconjugate Techniques (G. T. Hermanson, 2013, Academic Press)). In addition, various antibody drug conjugation services are available commercially from companies such as Lonza Inc. (Allendale, NJ), Abzena PLC (Cambridge, UK), ADC Biotechnology (St. Asaph, UK), Baxter BioPharma Solutions (Baxter Healthcare Corporation, Deerfield, IL) and Piramel Pharma Solutions (Grangemouth, UK).

Typically, preparation of the conjugates comprises first preparing a drug-linker, D-L, comprising one or more camptothecin analogues of Formula (I) and linker L, and then conjugating the drug-linker, D-L, to an appropriate group on targeting moiety, T. Ligation of linker, L, to targeting moiety, T, and subsequent ligation of the targeting moiety-linker, T-L, to one or more camptothecin analogues of Formula (I), D, however is an alternative approach that may be employed in some embodiments.

Suitable groups on compounds of Formula (I), D, for attachment of linker, L, in either of the above approaches include, but are not limited to, thiol groups, amine groups, carboxylic acid groups and hydroxyl groups. In some embodiments of the present disclosure, linker, L, is attached to a compound of Formula (I), D, via a hydroxyl or amine group on the compound.

Suitable groups on targeting moiety, T, for attachment of linker, L, in either of the above approaches include sulfhydryl groups (for example, on the side-chain of cysteine residues), amino groups (for example, on the side-chain of lysine residues), carboxylic acid groups (for example, on the side-chains of aspartate or glutamate residues), and carbohydrate groups.

For example, targeting moiety T may comprise one or more naturally occurring sulfhydryl groups allowing targeting moiety, T, to bond to linker, L, via the sulfur atom of a sulfhydryl group. Alternatively, targeting moiety, T, may comprise one or more lysine residues that can be chemically modified to introduce one or more sulfhydryl groups. Reagents that can be used to modify lysine residues include, but are not limited to, N-succinimidyl S-acetylthioacetate (SATA), N-succinimidyl-3-(2-pyridyldithio)propionate (“SPDP”) and 2-iminothiolane hydrochloride (Traut's Reagent). Alternatively, targeting moiety, T, may comprise one or more carbohydrate groups that can be chemically modified to include one or more sulfhydryl groups.

Carbohydrate groups on targeting moiety, T, may also be oxidized to provide an aldehyde (—CHO) group (see, for example, Laguzza et al., 1989, J. Med. Chem. 32(3):548-55), which could subsequently be reacted with linker, L, for example, via a hydrazine or hydroxylamine group on linker, L.

Targeting moiety, T, may also be modified to include additional cysteine residues (see, for example, U.S. Pat. Nos. 7,521,541; 8,455,622 and 9,000,130) or non-natural amino acids that provide reactive handles, such as selenomethionine, p-acetylphenylalanine, formylglycine or p-azidomethyl-L-phenylalanine (see, for example, Hofer et al., 2009, Biochemistry, 48:12047-12057; Axup et al., 2012, PNAS, 109:16101-16106; Wu et al., 2009, PNAS, 106:3000-3005; Zimmerman et al., 2014, Bioconj. Chem., 25:351-361), to allow for site-specific conjugation.

Alternatively, targeting moiety, T, may be modified to include a non-natural reactive group, such as an azide, that allows for conjugation to the linker via a complementary reactive group on the linker, for example, for example, by click chemistry (see, for example, Chio & Bane, 2020, Methods Mol. Biol., 2078:83-97).

Other protocols for the modification of proteins for the attachment or association of linker, L, are known in the art and include those described in Coligan et al., Current Protocols in Protein Science, vol. 2, John Wiley & Sons (2002).

In those embodiments in which targeting moiety, T, is an antibody, several different reactive groups on the antibody may function as a conjugation site, including F-amino groups on lysine residues, pendant carbohydrate moieties, side-chain carboxylic acid groups on aspartate or glutamate residues, cysteine-cysteine disulfide groups and cysteine thiol groups. The amino acids used for conjugation may be part of the natural sequence of the antibody, or they may be introduced by site-specific engineering techniques known in the art, as noted above.

Alternatively, antibody-drug conjugates may be prepared using the enzyme transglutaminase, for example, bacterial transglutaminase (BTG) from Streptomyces mobaraensis (see, for example, Jeger et al., 2010, Angew. Chem. Int. Ed., 49:9995-9997). BTG forms an amide bond between the side chain carboxamide of a glutamine (the amine acceptor, typically on the antibody) and an alkyleneamino group (the amine donor, typically on the drug-linker), which can be, for example, the F-amino group of a lysine or a 5-amino-n-pentyl group. Antibodies may also be modified to include a glutamine containing peptide, or “tag,” which allows BTG conjugation to be used to conjugate the antibody to a drug-linker (see, for example, U.S. Patent Application Publication No. US 2013/0230543 and International (PCT) Publication No. WO 2016/144608).

A similar conjugation approach utilizes the enzyme sortase A. In this approach, the antibody is typically modified to include the sortase A recognition motif (LPXTG, where X is any natural amino acid) and the drug-linker is designed to include an oligoglycine motif (typically GGG) to allow for sortase A-mediated transpeptidation (see, for example, Beerli, et al., 2015, PLos One, 10:e0131177; Chen et al., 2016, Nature: Scientific Reports, 6:31899).

Once conjugation is complete, the average number of compounds of Formula (I) conjugated to targeting moiety, T, (i.e. the “drug-to-antibody ratio” or DAR) may be determined by standard techniques such as UV/VIS spectroscopic analysis, ELISA-based techniques, chromatography techniques such as hydrophobic interaction chromatography (HIC), UV-MALDI mass spectrometry (MS) and MALDI-TOF MS. In addition, distribution of drug-linked forms (for example, the fraction of targeting moiety, T, containing zero, one, two, three, etc. compounds of Formula (I), D) may also optionally be analyzed. Various techniques are known in the art to measure DAR distribution, including MS (with or without an accompanying chromatographic separation step), hydrophobic interaction chromatography, reverse-phase HPLC or iso-electric focusing gel electrophoresis (IEF) (see, for example, Wakankar et al., 2011, mAbs, 3:161-172).

### Pharmaceutical Compositions

Compounds of Formula (I) and conjugates comprising compounds of Formula (I) are typically formulated for therapeutic use. Certain embodiments of the present disclosure thus relate to pharmaceutical compositions comprising a compound of Formula (I) or a conjugate thereof, such as conjugate having Formula (X), and a pharmaceutically acceptable carrier, diluent, or excipient. Such pharmaceutical compositions may be prepared by known procedures using well-known and readily available ingredients.

Pharmaceutical compositions may be formulated for administration to a subject by, for example, oral (including, for example, buccal or sublingual), topical, parenteral, rectal or vaginal routes, or by inhalation or spray. The term parenteral as used herein includes subcutaneous injection, and intradermal, intra-articular, intravenous, intramuscular, intravascular, intrasternal, intrathecal injection or infusion. The pharmaceutical composition will typically be formulated in a format suitable for administration to the subject, for example, as a syrup, elixir, tablet, troche, lozenge, hard or soft capsule, pill, suppository, oily or aqueous suspension, dispersible powder or granule, emulsion, injectable or solution. Pharmaceutical compositions may be provided as unit dosage formulations.

Compositions intended for oral use may be prepared in either solid or fluid unit dosage forms. Fluid unit dosage forms may be prepared according to procedures known in the art for the manufacture of pharmaceutical compositions and such compositions may contain one or more agents such as sweetening agents, flavouring agents, colouring agents and preserving agents in order to provide pharmaceutically elegant and palatable preparations. An elixir may be prepared by using a hydroalcoholic (for example, ethanol) carrier with suitable sweeteners such as sugar and/or saccharin, together with an aromatic flavoring agent. Suspensions may be prepared with an aqueous carrier and a suspending agent such as acacia, tragacanth, methylcellulose and the like.

Solid formulations, such as tablets, contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents for example, corn starch, or alginic acid; binding agents, for example starch, gelatin or acacia, and/or lubricating agents, for example magnesium stearate, stearic acid or talc, as well as other conventional ingredients such as dicalcium phosphate, magnesium aluminum silicate, calcium sulfate, starch, lactose, methylcellulose, and functionally similar materials. The tablets may be uncoated or they may be coated by known techniques, for example, in order to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules in which the active ingredient is mixed with an inert solid diluent, for example, calcium carbonate, calcium phosphate or kaolin, or as soft gelatin capsules in which the active ingredient is mixed with water or an oil medium, for example peanut oil, liquid paraffin or olive oil. Soft gelatin capsules are typically prepared by machine encapsulation of a slurry of the active ingredient with an acceptable vegetable oil, light liquid petrolatum or other inert oil.

Aqueous suspensions contain the active ingredient in admixture with excipients suitable for the manufacture of aqueous suspensions. Such excipients include suspending agents, for example sodium carboxylmethylcellulose, methyl cellulose, hydropropylmethylcellulose, sodium alginate, polyvinylpyrrolidone, gum tragacanth and gum acacia, and dispersing or wetting agents. Dispersing and wetting agents include, for example, naturally-occurring phosphatides (for example, lecithin), condensation products of an alkylene oxide with fatty acids (for example, polyoxyethylene stearate), condensation products of ethylene oxide with long chain aliphatic alcohols (for example, hepta-decaethyleneoxycetanol), condensation products of ethylene oxide with partial esters derived from fatty acids and a hexitol (for example, polyoxyethylene sorbitol monooleate), or condensation products of ethylene oxide with partial esters derived from fatty acids and hexitol anhydrides (for example, polyethylene sorbitan monooleate). The aqueous suspensions may also contain one or more preservatives (for example ethyl, or n-propyl-p-hydroxybenzoate), one or more colouring agents, one or more flavouring agents and/or one or more sweetening agents (for example, sucrose or saccharin).

Oily suspensions may be formulated by suspending the active ingredient in a vegetable oil, for example peanut oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavouring agents may be added to provide palatable oral preparations. The suspensions may optionally be preserved by the addition of an anti-oxidant such as ascorbic acid.

Dispersible powders and granules suitable for preparation of an aqueous suspension by the addition of water typically provide the active ingredient in admixture with a dispersing or wetting agent, suspending agent and one or more preservatives. Suitable dispersing or wetting agents and suspending agents are exemplified by those already mentioned above. One or more additional excipients, for example sweetening, flavouring and/or colouring agents, may also be present.

Pharmaceutical compositions may also be in the form of oil-in-water emulsions. The oil phase may be a vegetable oil, for example olive oil or peanut oil, or a mineral oil, for example liquid paraffin, or mixtures of such oils. Suitable emulsifying agents for inclusion in oil-in-water emulsions include, for example, naturally-occurring gums (for example, gum acacia or gum tragacanth), naturally-occurring phosphatides (for example, soy bean, lecithin), or esters or partial esters derived from fatty acids and hexitol anhydrides (for example, sorbitan monooleate) or condensation products of such partial esters with ethylene oxide (for example polyoxyethylene sorbitan monooleate). The emulsions may also optionally contain sweetening and/or flavoring agents.

The pharmaceutical compositions may be in the form of a sterile injectable aqueous or oleaginous solution or suspension. Such suspensions may be formulated using suitable dispersing or wetting agents and suspending agents such as those described above. The sterile injectable solution or suspension may comprise the active ingredient in a non-toxic parentally acceptable carrier or diluent. Acceptable carriers and diluents that may be employed include, for example, 1,3-butanediol, water, Ringer's solution or isotonic sodium chloride solution. In addition, sterile, fixed oils may be employed as carriers. For this purpose, various bland fixed oils may be employed including synthetic mono- or diglycerides. In addition, fatty acids such as oleic acid find use in the preparation of injectables. Adjuvants such as local anaesthetics, preservatives and/or buffering agents may also be included in the injectable solution or suspension.

Pharmaceutical compositions may also be formulated as suppositories for rectal administration. These compositions can be prepared by mixing the active ingredient with a suitable non-irritating excipient which is solid at ordinary temperatures but liquid at physiological temperature and will therefore melt in the rectum to release the drug. Such materials include cocoa butter and polyethylene glycols.

Other pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philadelphia, PA (2000).

### Methods of Use

Certain embodiments of the present disclosure relate to the therapeutic use of camptothecin analogues of Formula (I) and conjugates comprising these compounds, such as conjugates of Formula (X). Some embodiments relate to the use of compounds of Formula (I) or conjugates of Formula (X) as therapeutic agents.

Camptothecin analogues of Formula (I) show cytotoxic activity against cancer cells, and compounds of Formula (I) and conjugates comprising these compounds, such as conjugates of Formula (X), are thus useful for inhibiting abnormal cancer cell or tumor cell growth; inhibiting cancer cell or tumor cell proliferation, or treating cancer in a patient. In certain embodiments, compounds of general Formula (I) and conjugates of Formula (X) may be used to treat cancer. Some embodiments of the present disclosure thus relate to the use of compounds of general Formula (I) and conjugates of general Formula (X) as anti-cancer agents.

Certain embodiments of the present disclosure relate to methods of inhibiting the proliferation of cancer or tumor cells comprising contacting the cells with a compound of Formula (I) or a conjugate of Formula (X). Some embodiments relate to a method of killing cancer or tumor cells comprising contacting the cells with a compound of Formula (I) or a conjugate of Formula (X).

Some embodiments relate to methods of treating a subject having a cancer by administering to the subject a compound of Formula (I) or a conjugate of Formula (X). In this context, treatment with a compound of Formula (I) or a conjugate of Formula (X) may result in one or more of a reduction in the size of a tumor, the slowing or prevention of an increase in the size of a tumor, an increase in the disease-free survival time between the disappearance or removal of a tumor and its reappearance, prevention of a subsequent occurrence of a tumor (for example, metastasis), an increase in the time to progression, reduction of one or more adverse symptom associated with a tumor, and/or an increase in the overall survival time of a subject having cancer.

Certain embodiments relate to the use of a compound of Formula (I) or a conjugate of Formula (X) in a method of inhibiting tumor growth in a subject. Some embodiments relate to the use of a compound of Formula (I) or a conjugate of Formula (X) in a method of inhibiting proliferation of and/or killing cancer cells in vitro. Some embodiments relate to the use of a compound of Formula (I) or a conjugate of Formula (X) in a method of inhibiting proliferation of and/or killing cancer cells in vivo in a subject having a cancer.

Examples of cancers which may be treated in certain embodiments include hematologic neoplasms, including leukemias, myelomas and lymphomas; carcinomas, including adenocarcinomas and squamous cell carcinomas; melanomas and sarcomas. Carcinomas and sarcomas are also frequently referred to as “solid tumors.” Examples of commonly occurring solid tumors that may be treated in certain embodiments include, but are not limited to, brain cancer, breast cancer, cervical cancer, colon cancer, head and neck cancer, kidney cancer, lung cancer, ovarian cancer, pancreatic cancer, prostate cancer, stomach cancer, uterine cancer, non-small cell lung cancer (NSCLC) and colorectal cancer. Various forms of lymphoma also may result in the formation of a solid tumor and, therefore, may also be considered to be solid tumors in certain situations.

Certain embodiments relate to the use of a compound of Formula (I) or a conjugate of Formula (X) in the treatment of an autoimmune disease, such as atopic dermatitis, rheumatoid arthritis, psoriasis or systemic lupus erythematosus.

Certain embodiments relate to the use of a compound of Formula (I) or a conjugate of Formula (X) in the treatment of a viral infection, such as an HIV infection or SARS coronavirus infection.

### Pharmaceutical Kits

In certain embodiments, a pharmaceutical composition comprising a compound of Formula (I) or a conjugate of Formula (X) may be provided as part of a pharmaceutical kit or pack. Individual components of the kit would typically be packaged in separate containers. Suitable containers include, for example, bottles, blister packs, intravenous solution bags, vials and the like, depending on the formulation of the pharmaceutical composition. In certain embodiments, the container may be in a form allowing for administration to a subject, for example, an inhaler, syringe, pipette, eye dropper, pre-soaked gauze or pad, or other such like apparatus, from which the contents may be administered to the subject.

The kit may further comprise a label or package insert on or associated with the container(s). The term “package insert” is used to refer to instructions customarily included in commercial packages of therapeutic products that contain information about the indications, usage, dosage, administration, contraindications and/or warnings concerning the use of such therapeutic products. The label or package insert may further include a notice in the form prescribed by a governmental agency regulating the manufacture, use or sale of pharmaceuticals or biological products, which notice reflects approval by the agency of manufacture, for use or sale for human or animal administration. The label or package insert typically indicates that the compound or conjugate is for use to treat the condition of choice, for example, cancer.

If appropriate, one or more components of the kit may be lyophilized or provided in a dry form, such as a powder or granules, and the kit can additionally contain a suitable solvent for reconstitution of the lyophilized or dried component(s).

The following Examples are provided for illustrative purposes and are not intended to limit the scope of the invention in any way.

### EXAMPLES

Examples 1-3 below illustrate various methods of preparing camptothecin analogues of Formula (I). It is understood that one skilled in the art may be able to make these compounds by similar methods or by combining other methods known in the art. It is also understood that one skilled in the art would be able to make, using the methods described below or similar methods, other compounds of Formula (I) not specifically illustrated below by using the appropriate starting components and modifying the parameters of the synthesis as needed. In general, starting components may be obtained from commercial sources such as Sigma Aldrich (Merck KGaA), Alfa Aesar and Maybridge (Thermo Fisher Scientific Inc.), Matrix Scientific, Tokyo Chemical Industry Ltd. (TCI) and Fluorochem Ltd., or synthesized according to sources known to those skilled in the art (see, for example, March's Advanced Organic Chemistry: Reactions, Mechanisms, and Structure, 7th edition, John Wiley & Sons, Inc., 2013) or prepared as described herein.

### ABBREVIATIONS

The following abbreviations are used throughout the Examples section:

BCA: bicinchonic acid; Boc: di-tert-butyl dicarbonate; CE-SDS: capillary electrophoresis sodium dodecyl sulfate; DCM: dichloromethane; DTPA: diethylenetriamine pentaacetic acid; DIPEA: N,N-diisopropylethylamine; DMF: dimethylformamide; DMM™: (4-(4,6-dimethoxy-1,3,5-triazin-2-yl)-4-methyl-morpholinium chloride; EDC: 1-ethyl-3-(3-dimethylaminopropyl)carbodiimide; Fmoc: fluorenylmethyloxycarbonyl; HATU: hexafluorophosphate azabenzotriazole tetramethyl uronium; HIC: hydrophobic interaction chromatography; HOAt: 1-hydroxy-7-azabenzotriazole; HPLC: high-performance liquid chromatography; LCMS: liquid chromatography mass spectrometry; MC: maleimidocaproyl; MT: maleimidotriethylene glycolate; NMM: N-methylmorpholine; PNP: p-nitrophenol; RP-UPLC-MS: reversed-phase ultra-high performance chromatography mass spectrometry; SEC: size exclusion chromatography; TCEP: tris(2-carboxyethyl) phosphine; Tfp: tetrafluorophenyl; TLC: thin layer chromatography; TFA: trifluoracetic acid.

### General Chemistry Procedures

### General Procedure 1: Conversion of Chloride to Amine (Synthetic Scheme I; FIG. 1A)

To a stirring solution of chloride compound in dimethylformamide (0.05-0.1 M) was added the appropriate secondary amine (3 eq.). Upon completion (determined by LCMS, typically 1-3 h), the reaction mixture was purified by reverse-phase HPLC to provide the desired product after lyophilization.

### General Procedure 2: Conversion of Amine to Amide (Synthetic Scheme II; FIG. 1B)

To a stirring solution of amine compound in dimethylformamide (0.05-0.1 M) was added triethylamine (1.2 eq.), the appropriate carboxylic acid (1.1 eq.) followed by a solution of DMM™ (2 eq.) in water (1 M). Upon completion (determined by LCMS, typically 16 h), the reaction mixture was purified by reverse-phase HPLC to provide the desired product after lyophilization.

### General Procedure 3: Conversion of Amine to Sulfonamide (Synthetic Scheme III; FIG. 1C)

To a stirring solution of amine compound in dimethylformamide (0.05-0.1 M) was added DIPEA (3 eq.) followed by the appropriate sulfonyl chloride. Upon completion (determined by LCMS, typically 16 h), the reaction mixture was purified by reverse-phase HPLC to provide the desired product after lyophilization.

### General Procedure 4: 2-Step Conversion of Amine to Urea (Synthetic Scheme IV; FIG. 1D)

Step 1: To a stirring solution of amine compound in dichloromethane or dimethylformamide (0.05-0.1 M) was added p-nitrophenyl carbonate (1 eq.) then triethylamine (2 eq.). Upon completion (determined by LCMS typically 1-4 h), the reaction mixture was concentrated to dryness then was purified by reverse-phase HPLC to provide the desired PNP-carbamate intermediate after lyophilization. This intermediate can either used to generate a single analogue or be divided into multiple batches in order to generate multiple analogues in the second step.

Step 2: To the PNP-carbamate intermediate in dimethylformamide (0.1-0.2 M) was added the appropriate primary amine (3 eq.). Upon completion (determined by LCMS, typically 1 h), the reaction mixture was purified by reverse-phase HPLC to provide the desired product after lyophilization.

### General Procedure 5: Conversion of Amine to Carbamate (Synthetic Scheme V; FIG. 1E)

To a stirring solution of amine compound in dichloromethane or dimethylformamide (0.05-0.1 M) was added p-nitrophenyl carbonate (1 eq.) then triethylamine (2 eq.). Upon completion (determined by LCMS, typically 1-4 h), the appropriate alcohol was added to the resultant PNP-carbamate intermediate. Upon completion (determined by LCMS, typically 1-16 h), the reaction mixture was purified by reverse-phase HPLC to provide the desired product after lyophilization.

### General Procedure 6: Removal of Boc Protecting Group

To a stirring solution of the Boc-protected amine compound in dichloromethane (0.1 M) was added TFA (20% by volume). Upon completion (determined by LCMS, typically 1 h), the reaction mixture was concentrated in vacuo to provide a crude solid or was purified as described in General Procedure 9.

### General Procedure 7. Copper-Mediated Amide Coupling (Synthetic Scheme VI; FIG. 1F)

To a rapidly stirring solution of Boc-GGFG-OH (3 eq.) and HOAt (3 eq.) in a 10% v/v mixture of dimethyl formamide in dichloromethane (0.02 M) was added EDC (HCl salt, 3 eq.). After 5 min, a solution of the amine containing payload (1 eq.) in a 10% v/v mixture of dimethyl formamide in dichloromethane (0.02 M) was added, followed immediately by the addition of CuCl₂ (4 eq.). Upon completion (determined by LCMS, typically 1-16 h), the reaction mixture was concentrated in vacuo to provide a crude solid or was purified by preparative HPLC to provide the desired product after lyophilization.

General Procedure 8: MT installation (Synthetic Scheme VII; FIG. 1G)

To a stirring solution of amine compound (1 eq.) in dimethylformamide (˜0.02 M) was added a solution of MT-OTfp (1.2-1.5 eq.) in acetonitrile (˜0.02 M) then DIPEA (10 uL, 4 eq.). Upon completion (determined by LCMS, typically 1-16 h), the reaction mixture was concentrated in vacuo to provide a crude solid which was purified by preparative HPLC to provide the desired product after lyophilization.

### General Procedure 9: Compound Purification

Flash Chromatography: Crude reaction products were purified with Biotage® Snap Ultra columns (10, 25, 50, or 100 g) (Biotage, Charlotte, NC), eluting with linear gradients of ethyl acetate/hexanes or methanol/dichloromethane on a Biotage® Isolera™ automated flash system (Biotage, Charlotte, NC). Alternatively, reverse-phase flash purification was conducting using Biotage® Snap Ultra C18 columns (12, 30, 60, or 120 g), eluting with linear gradients of 0.1% TFA in acetonitrile/0.1% TFA in water. Purified compounds were isolated by either removal of organic solvents by rotavap or lyophilization of acetonitrile/water mixtures.

Preparative HPLC: Reverse-phase HPLC of crude compounds was performed using a Luna® 5-μm C18 100 Å (150×30 mm) column (Phenomenex, Torrance, CA) on an Agilent 1260 Infinity II preparative LC/MSD system (Agilent Technologies, Inc., Santa Clara, CA), and eluting with linear gradients of 0.1% TFA in acetonitrile/0.1% TFA in water. Purified compounds were isolated by lyophilization of acetonitrile/water mixtures.

### General Procedure 10: Compound Analysis

LC/MS: Reactions were monitored for completion and purified compounds were analyzed using a Kinetex® 2.6-μm C18 100 Å (30×3 mm) column (Phenomenex, Torrance, CA) on an Agilent 1290 HPLC/6120 single quad LC/MS system (Agilent Technologies, Inc., Santa Clara, CA), eluting with a linear 10 to 100% gradient 0.1% formic acid in acetonitrile/0.1% formic acid in water.

NMR: ¹H NMR spectra were collected with a Bruker AVANCE III 300 Spectrometer (300 MHz) (Bruker Corporation, Billerica, MA). Chemical shifts are reported in parts per million (ppm).

### Example 1: Preparation of Camptothecin Analogues Having Methyl at the C10 Position

### 1.1: (S)-11-(chloromethyl)-4-ethyl-8-fluoro-4-hydroxy-9-methyl-1,12-dihydro-14H-pyrano[3′,4′:6,7]indolizino[1,2-b]quinoline-3,14(4H)-dione (Compound 1.1)

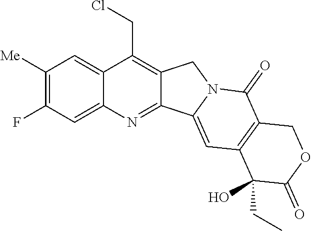 [SMILES: CC[C@@]1(O)C(=O)OCc2c1cc1n(c2=O)Cc2c-1nc1cc(F)c(C)cc1c2CCl]

The title compound was prepared according to the procedure provided in Li, et al., 2019, ACS Med. Chem. Lett., 10(10): 1386-1392.

### 1.2: (S)-11-(aminomethyl)-4-ethyl-8-fluoro-4-hydroxy-9-methyl-1,12-dihydro-14H-pyrano[3′,4′ 6,7]indolizino[1,2-b]quinoline-3,14(4H)-dione (Compound 1.2)

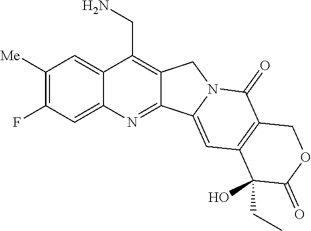 [SMILES: CC[C@@]1(O)C(=O)OCc2c1cc1n(c2=O)Cc2c-1nc1cc(F)c(C)cc1c2CN]

The title compound was prepared according to the procedure provided in Li, et al., 2019, ACS Med. Chem. Lett., 10(10): 1386-1392.

### 1.3: (S)-4-ethyl-8-fluoro-4-hydroxy-9-methyl-11-(morpholinomethyl)-1,12-dihydro-14H-pyrano[3′,4′: 6,7]indolizino[1,2-b]quinoline-3,14(4H)-dione (Compound 100)

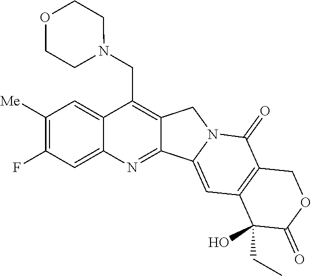 [SMILES: CC[C@@]1(O)C(=O)OCc2c1cc1n(c2=O)Cc2c-1nc1cc(F)c(C)cc1c2CN1CCOCC1]

The title compound was prepared according to General Procedure 1 starting from Compound 1.1 (10 mg) and morpholine. Preparative HPLC purification was accomplished as described in General Procedure 9, eluting with a 20 to 60% CH₃CN/H₂O+0.1% TFA gradient to give the title compound as an off-white solid (TFA salt, 3.6 mg, 26% yield).

LC/MS: Calc'd m/z=479.2 for C₂₆H₂₆FN₃O₅, found [M+H]⁺=480.4.

¹H NMR (300 MHz, CDCl₃) δ 8.20 (d, J=8.0 Hz, 1H), 7.82 (d, J=10.4 Hz, 1H), 7.67 (s, 1H), 5.77 (d, J=16.4 Hz, 1H), 5.42 (s, 2H), 5.33 (d, J=16.4 Hz, 1H), 4.26 (s, 2H), 3.81 (t, J=4.7 Hz, 4H), 2.82-2.76 (m, 4H), 2.57 (d, J=1.7 Hz, 3H), 1.99-1.82 (m, 2H), 1.06 (t, J=7.4 Hz, 3H).

### 1.4: (S)-4-ethyl-8-fluoro-4-hydroxy-9-methyl-11-((4-(phenylsulfonyl)piperazin-1-yl)methyl)-1,12-dihydro-14H-pyrano[3′,4′:6,7]indolizino[1,2-b]quinoline-3,14(4H)-dione (Compound 102)

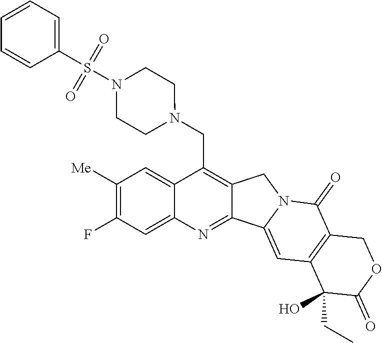 [SMILES: CC[C@@]1(O)C(=O)OCc2c1cc1n(c2=O)Cc2c-1nc1cc(F)c(C)cc1c2CN1CCN(S(=O)(=O)c2ccccc2)CC1]

The title compound was prepared according to General Procedure 1 starting from Compound 1.1 (10 mg) and 1-(phenylsulfonyl)piperazine. Preparative HPLC purification was accomplished as described in General Procedure 9, eluting with a 20 to 60% CH₃CN/H₂O+0.1% TFA gradient to give the title compound as an off-white solid (TFA salt, 3.6 mg, 21% yield).

LC/MS: Calc'd m/z=618.2 for C₃₂H₃₁FN₄O₆, found [M+H]⁺=619.4.

¹H NMR (300 MHz, CDCl₃) δ 8.07 (d, J=7.9 Hz, 1H), 7.88-7.44 (m, 7H), 5.73 (d, J=16.4 Hz, 1H), 5.33 (s, 2H), 5.33-5.26 (m, 1H), 4.19 (s, 2H), 3.12 (s, 4H), 2.80 (s, 4H), 2.54 (s, 3H), 1.90 (dt, J=11.6, 7.0 Hz, 2H), 1.04 (t, J=7.3 Hz, 3H).

### 1.5: (S)-11-((4-((4-aminophenyl)sulfonyl)piperazin-1-yl)methyl)-4-ethyl-8-fluoro-4-hydroxy-9-methyl-1,12-dihydro-14H-pyrano[3′,4′:6,7]indolizino[1,2-b]quinoline-3,14(4H)-dione (Compound 104)

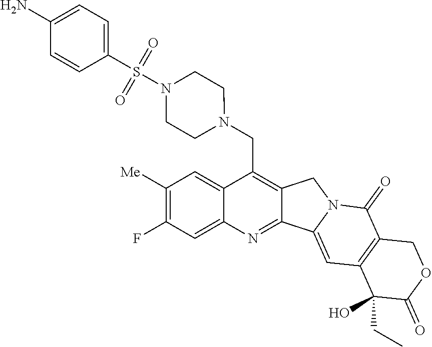 [SMILES: CC[C@@]1(O)C(=O)OCc2c1cc1n(c2=O)Cc2c-1nc1cc(F)c(C)cc1c2CN1CCN(S(=O)(=O)c2ccc(N)cc2)CC1]

The title compound was prepared according to General Procedure 1 starting from Compound 1.1 (10 mg) and 4-(piperazin-1-ylsulfonyl)aniline. Preparative HPLC purification was accomplished as described in General Procedure 9, eluting with a 20 to 60% CH₃CN/H₂O+0.1% TFA gradient to give the title compound as an off-white solid (TFA salt, 4.7 mg, 27% yield).

LC/MS: Calc'd m/z=633.2 for C₃₂H₃₂FN₅O₆, found [M+H]⁺=634.4.

¹H NMR (300 MHz, MeOD) δ 8.32 (d, J=8.0 Hz, 1H), 7.85 (d, J=10.5 Hz, 1H), 7.65 (s, 1H), 7.46 (d, J=8.7 Hz, 2H), 6.74 (d, J=8.7 Hz, 2H), 5.61 (d, J=16.5 Hz, 1H), 5.44 (s, 2H), 5.41 (d, J=16.5 Hz, 1H), 4.51 (s, 2H), 3.22-3.07 (m, 8H), 2.58 (s, 3H), 2.03-1.93 (m, 2H), 1.02 (t, J=7.3 Hz, 3H).

### 1.6: (S)-4-ethyl-8-fluoro-4-hydroxy-9-methyl-11-((4-methylpiperazin-1-yl)methyl)-1,12-dihydro-14H-pyrano[3′,4′:6,7]indolizino[1,2-b]quinoline-3,14(4H)-dione (Compound 106)

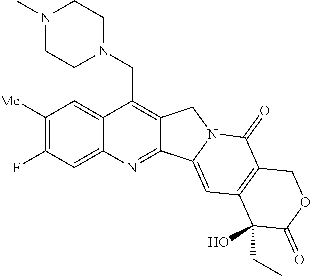 [SMILES: CC[C@@]1(O)C(=O)OCc2c1cc1n(c2=O)Cc2c-1nc1cc(F)c(C)cc1c2CN1CCN(C)CC1]

The title compound was prepared according to General Procedure 1 starting from Compound 1.1 (10 mg) and N-methylpiperazine. Preparative HPLC purification was accomplished as described in General Procedure 9, eluting with a 20 to 50% CH₃CN/H₂O+0.1% TFA gradient to give the title compound as an off-white solid (TFA salt, 3.6 mg, 25% yield).

LC/MS: Calc'd m/z=492.2 for C₂₇H₂₉FN₄O₄, found [M+H]⁺=493.4.

### 1.7. (S)-11-((4-(4-aminophenyl)piperazin-1-yl)methyl)-4-ethyl-8-fluoro-4-hydroxy-9-methyl-1,12-dihydro-14H-pyrano[3′,4′:6,7]indolizino[1,2-b]quinoline-3,14(4H)-dione (Compound 108)

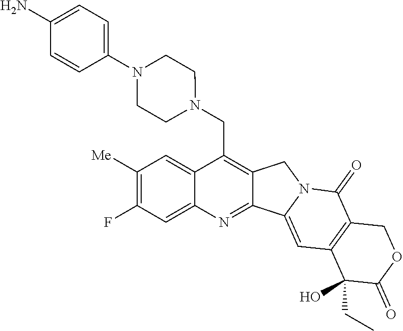 [SMILES: CC[C@@]1(O)C(=O)OCc2c1cc1n(c2=O)Cc2c-1nc1cc(F)c(C)cc1c2CN1CCN(c2ccc(N)cc2)CC1]

The title compound was prepared according to General Procedure 1 starting from Compound 1.1 (10 mg) and 4-(piperazin-1-yl)aniline. Preparative HPLC purification was accomplished as described in General Procedure 9, eluting with a 20 to 50% CH₃CN/H₂O+0.1% TFA gradient to give the title compound as an off-white solid (TFA salt, 3.7 mg, 23% yield).

LC/MS: Calc'd m/z=569.2 for C₃₂H₃₂FN₅O₄, found [M+H]⁺=570.4.

¹H NMR (300 MHz, MeOD) δ 8.39 (d, J=8.1 Hz, 1H), 7.79 (d, J=10.6 Hz, 1H), 7.21 (d, J=9.0 Hz, 2H), 7.14 (d, J=9.0 Hz, 2H), 5.62 (d, J=16.4 Hz, 1H), 5.49 (s, 2H), 5.41 (d, J=16.4 Hz, 1H), 4.45 (s, 2H), 3.44-3.38 (m, 4H), 3.06-3.00 (m, 4H), 2.58 (d, J=1.8 Hz, 3H), 2.00-1.89 (m, 2H), 1.03 (t, J=7.3 Hz, 3H).

### 1.8: (S)-4-ethyl-8-fluoro-4-hydroxy-9-methyl-11-(piperidin-1-ylmethyl)-1,12-dihydro-14H-pyrano[3′,4′:6,7]indolizino[1,2-b]quinoline-3,14(4H)-dione (Compound 110)

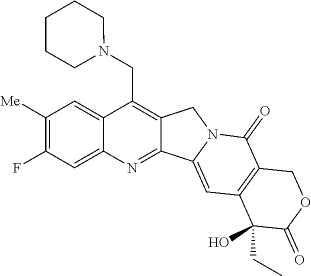 [SMILES: CC[C@@]1(O)C(=O)OCc2c1cc1n(c2=O)Cc2c-1nc1cc(F)c(C)cc1c2CN1CCCCC1]

The title compound was prepared according to General Procedure 1 starting from Compound 1.1 (10 mg) and piperidine. Preparative HPLC purification was accomplished as described in General Procedure 9, eluting with a 10 to 60% CH₃CN/H₂O+0.1% TFA gradient to give the title compound as an off-white solid (TFA salt, 1.5 mg, 11% yield).

LC/MS: Calc'd m/z=477.2 for C₂₇H₂₈FN₃O₄, found [M+H]⁺=478.2.

¹H NMR (300 MHz, MeOD) δ 8.34 (d, J=7.6 Hz, 1H), 7.94 (d, J=10.3 Hz, 1H), 7.70 (s, 1H), 5.63 (d, J=16.4 Hz, 1H), 5.52 (s, 2H), 5.44 (d, J=16.5 Hz, 1H), 4.99 (s, 2H), 3.73-3.46 (m, 4H), 2.64 (s, 3H), 2.03-1.90 (m, 2H), 1.90-1.84 (m, 6H), 1.03 (t, J=7.4 Hz, 3H).

### 1.9: tert-butyl (S)-4-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)piperazine-1-carboxylate (Compound 111)

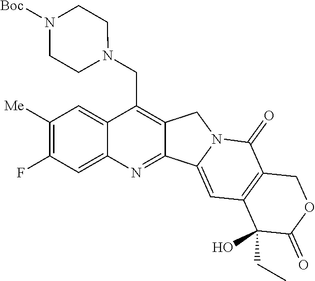 [SMILES: CC[C@@]1(O)C(=O)OCc2c1cc1n(c2=O)Cc2c-1nc1cc(F)c(C)cc1c2CN1CCN(C(=O)OC(C)(C)C)CC1]

The title compound was prepared according to General Procedure 1 starting from Compound 1.1 (10 mg) and tert-butyl piperazine-1-carboxylate. Preparative HPLC purification was accomplished as described in General Procedure 9, eluting with a 10 to 60% CH₃CN/H₂O+0.1% TFA gradient to give the title compound as an off-white solid (TFA salt, 6.6 mg, 40% yield).

LC/MS: Calc'd m/z=578.2 for C₃₁H₃₅FN₄O₆, found [M+H]⁺=579.4.

### 1.10: (S)-4-ethyl-8-fluoro-4-hydroxy-9-methyl-11-(piperazin-1-ylmethyl)-1,12-dihydro-14H-pyrano[3′,4′: 6,7]indolizino[1,2-b]quinoline-3,14(4H)-dione (Compound 112)

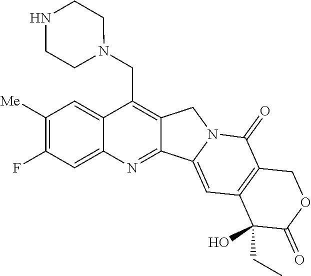 [SMILES: CC[C@@]1(O)C(=O)OCc2c1cc1n(c2=O)Cc2c-1nc1cc(F)c(C)cc1c2CN1CCNCC1]

The title compound was prepared according to General Procedure 6 starting from Compound 111 (5.0 mg) to give the title compound as an off-white solid (TFA salt, 4.4 mg).

LC/MS: Calc'd m/z=478.2 for C₂₆H₂₇FN₄O₄, found [M+H]⁺=479.2.

### 1.11: (S)-4-ethyl-8-fluoro-4-hydroxy-11-(((R)-2-(hydroxymethyl)morpholino)methyl)-9-methyl-1,12-dihydro-14H-pyrano[3′,4′:6,7]indolizino[1,2-b]quinoline-3,14(4H)-dione (Compound 113)

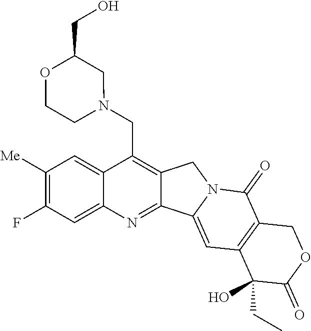 [SMILES: CC[C@@]1(O)C(=O)OCc2c1cc1n(c2=O)Cc2c-1nc1cc(F)c(C)cc1c2CN1CCO[C@@H](CO)C1]

The title compound was prepared according to General Procedure 1 starting from Compound 1.1 (10 mg) and (R)-morpholin-2-yl methanol. Preparative HPLC purification was accomplished as described in General Procedure 9, eluting with a 10 to 60% CH₃CN/H₂O+0.1% TFA gradient to give the title compound as an off-white solid (TFA salt, 4.6 mg, 32% yield).

LC/MS: Calc'd m/z=509.2 for C₂₇H₂₈FN₃O₆, found [M+H]⁺=510.4.

### 1.12: (4S)-4-ethyl-8-fluoro-4-hydroxy-11-((3-(hydroxymethyl)thiomorpholino)methyl)-9-methyl-1,12-dihydro-14H-pyrano[3′,4′:6,7]indolizino[1,2-b]quinoline-3,14(4H)-dione (Compound 114)

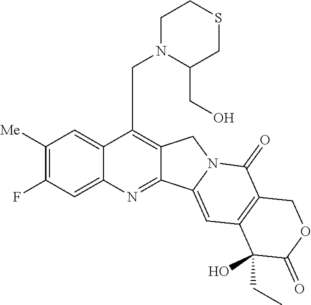 [SMILES: CC[C@@]1(O)C(=O)OCc2c1cc1n(c2=O)Cc2c-1nc1cc(F)c(C)cc1c2CN1CCSCC1CO]

The title compound was prepared according to General Procedure 1 starting from Compound 1.1 (10 mg) and thiomorpholin-3-ylmethanol. Preparative HPLC purification was accomplished as described in General Procedure 9, eluting with a 10 to 60% CH₃CN/H₂O+0.1% TFA gradient to give the title compound as an off-white solid (TFA salt, 1.5 mg, 12% yield).

LC/MS: Calc'd m/z=525.6 for C₂₇H₂₈FN₃O₅S, found [M+H]⁺=526.5.

¹H NMR (300 MHz, 10% D₂O/CD₃CN) 8.36 (d, J=8.1 Hz, 1H), 7.83 (d, J=10.7 Hz, 1H), 7.50 (s, 1H), 5.57 (d, J=16.4 Hz, 1H), 5.52-5.29 (m, 3H), 5.02 (d, J=14.6 Hz, 1H), 4.71-4.54 (m, 1H), 4.27 (dd, J=12.4, 5.0 Hz, 1H), 3.98 (dd, J=12.3, 3.4 Hz, 1H), 3.55 (s, 1H), 3.30-3.03 (m, 4H) 2.97-2.72 (m, 3H), 2.62 (s, 1H), 2.55 (s, 3H), 0.95 (t, J=7.4 Hz, 3H).

### 1.13: (4S)-4-ethyl-8-fluoro-4-hydroxy-11-((4-(hydroxymethyl)-2-oxa-5-azabicyclo[2.2.1]heptan-5-yl)methyl)-9-methyl-1,12-dihydro-14H-pyrano[3′,4′:6,7]indolizino[1,2-b]quinoline-3,14(4H)-dione (Compound 115)

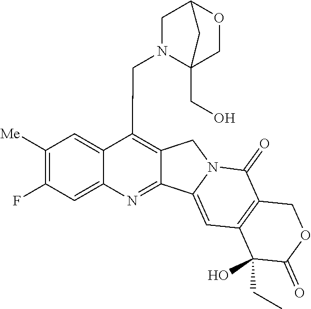 [SMILES: CC[C@@]1(O)C(=O)OCc2c1cc1n(c2=O)Cc2c-1nc1cc(F)c(C)cc1c2CN1CC2CC1(CO)CO2]

The title compound was prepared according to General Procedure 1 starting from Compound 1.1 (10 mg) and 2-oxa-5-azabicyclo[2.2.1]heptan-4-yl methanol. Preparative HPLC purification was accomplished as described in General Procedure 9, eluting with a 10 to 60% CH₃CN/H₂O+0.1% TFA gradient to give the title compound as an off-white solid (TFA salt, 3.5 mg, 29% yield).

LC/MS: Calc'd m/z=521.5 for C₂₈H₂₈FN₃O₆, found [M+H]⁺=522.5.

¹H NMR (300 MHz, 10% D₂O/CD₃CN) δ 8.36 (d, J=7.9 Hz, 1H), 7.86 (dd, J=10.6, 5.0 Hz, 1H), 7.50 (d, J=1.8 Hz, 1H), 5.63-5.49 (m, 2H), 5.37 (dd, J=17.8, 14.1 Hz, 2H), 5.05 (s, 2H), 4.63 (d, J=2.5 Hz, 1H), 4.55 (d, J=10.7 Hz, 1H), 4.33 (s, 2H), 3.92 (d, J=10.7 Hz, 1H), 3.36 (s, 2H), 2.57 (s, 3H), 2.41-2.13 (m, 2H), 1.97-1.85 (m, 2H), 0.95 (t, J=7.4 Hz, 3H).

### 1.14: (4S)-4-ethyl-8-fluoro-4-hydroxy-11-((3-(hydroxymethyl)-1,1-dioxidothiomorpholino) methyl)-9-methyl-1,12-dihydro-14H-pyrano[3′,4′:6,7]indolizino[1,2-b]quinoline-3,14(4H)-dione (Compound 116)

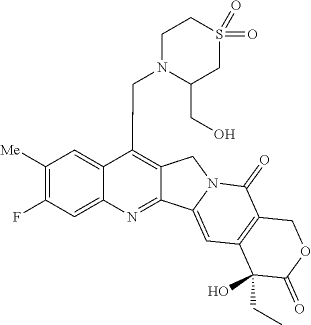 [SMILES: CC[C@@]1(O)C(=O)OCc2c1cc1n(c2=O)Cc2c-1nc1cc(F)c(C)cc1c2CN1CCS(=O)(=O)CC1CO]

The title compound was prepared according to General Procedure 1 starting from Compound 1.1 (10 mg) and 3-(hydroxymethyl)-1λ⁶-thiomorpholine-1,1-dione. Purification was accomplished as described in General Procedure 9, eluting with a 10 to 60% CH₃CN/H₂O+0.1% TFA gradient to give the title compound as an off-white solid (TFA salt, 0.2 mg, 2% yield).

LC/MS: Calc'd m/z=557.6 for C₂₇H₂₈FN₃O₇S, found [M+H]⁺=558.4.

¹H NMR (300 MHz, 10% D₂O/CD₃CN) δ 8.44 (d, J=8.2 Hz, 1H), 7.80 (d, J=11.0 Hz, 1H), 7.50 (s, 1H), 5.58 (d, J=16.5 Hz, 1H), 5.45-5.26 (m, 3H), 4.60 (d, J=14.9 Hz, 1H), 4.33 (d, J=14.7 Hz, 1H), 3.88 (d, J=4.8 Hz, 2H), 3.41-2.85 (m, 4H), 2.53 (s, 2H), 2.19 (p, J=2.5 Hz, 2H), 1.74 (p, J=2.5 Hz, 2H), 1.27 (s, 2H), 0.95 (t, J=7.4 Hz, 3H).

### 1.15: (4S)-4-ethyl-8-fluoro-4-hydroxy-11-((6-hydroxy-3-azabicyclo[3.1.1]heptan-3-yl)methyl)-9-methyl-1,12-dihydro-14H-pyrano[3′,4′:6,7]indolizino[1,2-b]quinoline-3,14(4H)-dione (Compound 117)

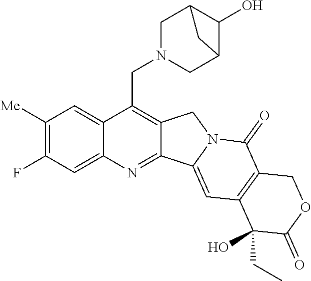 [SMILES: CC[C@@]1(O)C(=O)OCc2c1cc1n(c2=O)Cc2c-1nc1cc(F)c(C)cc1c2CN1CC2CC(C1)C2O]

The title compound was prepared according to General Procedure 1 starting from Compound 1.1 (10 mg) and 3-azabicyclo[3.1.1]heptan-6-ol. Preparative HPLC purification was accomplished as described in General Procedure 9, eluting with a 10 to 60% CH₃CN/H₂O+0.1% TFA gradient to give the title compound as an off-white solid (TFA salt, 1.3 mg, 11% yield).

LC/MS: Calc'd m/z=505.5 for C₂₈H₂₈FN₃O₅, found [M+H]⁺=506.6.

¹H NMR (300 MHz, 10% D₂O/CD₃CN) δ 8.25 (d, J=7.9 Hz, 1H), 7.87 (d, J=10.6 Hz, 1H), 7.50 (s, 1H), 5.65-5.27 (m, 4H), 4.98 (s, 2H), 4.24 (s, 1H), 3.83-3.57 (m, 4H), 2.54 (s, 5H), 2.01-1.86 (m, 2H), 1.70 (s, 2H), 0.95 (t, J=7.3 Hz, 3H).

### 1.16: (S)-4-ethyl-8-fluoro-11-((3-fluoro-3-(hydroxymethyl)azetidin-1-yl)methyl)-4-hydroxy-9-methyl-1,12-dihydro-14H-pyrano[3′,4′:6,7]indolizino[1,2-b]quinoline-3,14(4H)-dione (Compound 118)

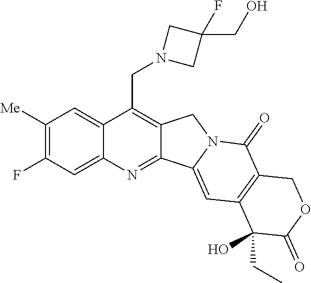 [SMILES: CC[C@@]1(O)C(=O)OCc2c1cc1n(c2=O)Cc2c-1nc1cc(F)c(C)cc1c2CN1CC(F)(CO)C1]

The title compound was prepared according to General Procedure 1 starting from Compound 1.1 (10 mg) and 3-fluoroazetidin-3-yl methanol. Preparative HPLC purification was accomplished as described in General Procedure 9, eluting with a 10 to 60% CH₃CN/H₂O+0.1% TFA gradient to give the title compound as an off-white solid (TFA salt, 1.4 mg, 12% yield).

LC/MS: Calc'd m/z=497.5 for C₂₆H₂₅F₂N₃O₅, found [M+H]⁺=498.4.

¹H NMR (300 MHz, 10% D₂O/CD₃CN) δ 8.24 (d, J=7.9 Hz, 1H), 7.85 (d, J=10.7 Hz, 1H), 7.50 (s, 1H), 5.57 (d, J=16.5 Hz, 1H), 5.48-5.28 (m, 3H), 4.98 (s, 2H), 4.44-4.14 (m, 4H), 3.78 (d, J=14.9 Hz, 2H), 2.01-1.86 (m, 2H), 0.95 (t, J=7.4 Hz, 3H).

### 1.17. (S)-4-ethyl-8-fluoro-4-hydroxy-11-((3-(hydroxymethyl)azetidin-1-yl)methyl)-9-methyl-1,12-dihydro-14H-pyrano[3′,4′: 6,7]indolizino[1,2-b]quinoline-3,14(4H)-dione (Compound 119)

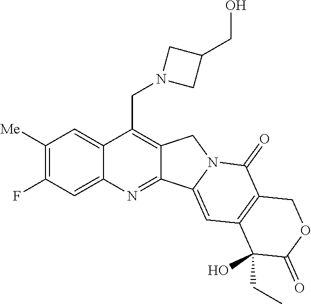 [SMILES: CC[C@@]1(O)C(=O)OCc2c1cc1n(c2=O)Cc2c-1nc1cc(F)c(C)cc1c2CN1CC(CO)C1]

The title compound was prepared according to General Procedure 1 starting from Compound 1.1 (10 mg) and azetidin-3-ylmethanol. Preparative HPLC purification was accomplished as described in General Procedure 9, eluting with a 10 to 60% CH₃CN/H₂O+0.1% TFA gradient to give the title compound as an off-white solid (TFA salt, 0.5 mg, 4.5% yield).

LC/MS: Calc'd m/z=479.5 for C₂₆H₂₆FN₃O₅, found [M+H]⁺=480.4.

¹H NMR (300 MHz, 10% D₂O/CD₃CN) δ 8.23 (d, J=7.8 Hz, 1H), 7.90 (d, J=10.6 Hz, 1H), 7.53 (s, 1H), 5.58 (d, J=16.5 Hz, 1H), 5.50-5.28 (m, 3H), 5.01 (s, 2H), 4.31-4.17 (m, 2H), 4.15-4.00 (m, 2H), 3.62 (d, J=3.9 Hz, 2H), 2.58 (s, 3H), 2.01-1.86 (m, 2H), 0.96 (t, J=7.4 Hz, 3H).

### 1.18: (4S)-11-((4,4-difluoro-3-(hydroxymethyl)piperidin-1-yl)methyl)-4-ethyl-8-fluoro-4-hydroxy-9-methyl-1,12-dihydro-14H-pyrano[3′,4′:6,7]indolizino[1,2-b]quinoline-3,14(4H)-dione (Compound 120)

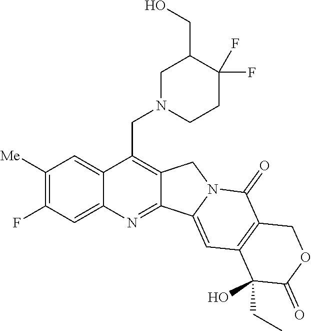 [SMILES: CC[C@@]1(O)C(=O)OCc2c1cc1n(c2=O)Cc2c-1nc1cc(F)c(C)cc1c2CN1CCC(F)(F)C(CO)C1]

The title compound was prepared according to General Procedure 1 starting from Compound 1.1 (10 mg) and 4,4-difluoropiperidin-3-yl methanol. Preparative HPLC purification was accomplished as described in General Procedure 9, eluting with a 10 to 60% CH₃CN/H₂O+0.1% TFA gradient to give the title compound as an off-white solid (TFA salt, 4 mg, 32% yield).

LC/MS: Calc'd m/z=543.5 for C₂₈H₂₈F₃N₃O₅, found [M+H]⁺=544.4.

¹H NMR (300 MHz, 10% D₂O/CD₃CN) δ 8.25 (d, J=8.0 Hz, 1H), 7.77 (dd, J=10.7, 1.4 Hz, 1H), 7.47 (s, 1H), 5.55 (d, J=16.5 Hz, 1H), 5.42-5.25 (m, 3H), 4.66 (d, J=3.2 Hz, 2H), 3.90-3.77 (m, 1H), 3.71-3.45 (m, 4H), 2.24 (q, J=11.8, 9.2 Hz, 2H), 2.01-1.86 (m, 2H), 0.94 (t, J=7.4 Hz, 3H).

### 1.19: (S)-4-ethyl-8-fluoro-4-hydroxy-11-((1-(hydroxymethyl)-7-azabicyclo[2.2.1]heptan-7-yl)methyl)-9-methyl-1,12-dihydro-14H-pyrano[3′,4′: 6,7]indolizino[1,2-b]quinoline-3,14(4H)-dione (Compound 121)

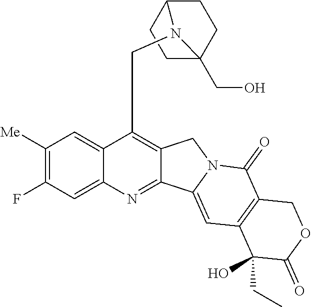 [SMILES: CC[C@@]1(O)C(=O)OCc2c1cc1n(c2=O)Cc2c-1nc1cc(F)c(C)cc1c2CN1C2CCC1(CO)CC2]

The title compound was prepared according to General Procedure 1 starting from Compound 1.1 (10 mg) and 7-azabicyclo[2.2.1]heptan-1-ylmethanol. Preparative HPLC purification was accomplished as described in General Procedure 9, eluting with a 10 to 60% CH₃CN/H₂O+0.1% TFA gradient to give the title compound as an off-white solid (TFA salt, 0.8 mg, 6.6% yield).

LC/MS: Calc'd m/z=519.6 for C₂₉H₃₀FN₃O₅, found [M+H]⁺=520.4.

¹H NMR (300 MHz, 10% D₂O/CD₃CN) δ 8.22 (s, 1H), 7.92 (d, J=10.7 Hz, 1H), 7.54 (s, 1H), 5.59 (dd, J=17.6, 7.6 Hz, 2H), 5.33 (t, J=17.4 Hz, 2H), 4.98-4.81 (m, 1H), 4.67-4.44 (m, 2H), 4.28-3.93 (m, 4H), 2.73 (s, 2H), 2.34-2.03 (m, 4H), 1.91 (d, J=14.0 Hz, 5H), 0.96 (t, J=7.4 Hz, 3H).

### 1.20: (S)-N-((4-ethyl-8-fluoro-4-hydroxy-9-methyl-3,14-dioxo-3,4,12,14-tetrahydro-1H-pyrano[3,4′:6,7]indolizino[1,2-b]quinolin-11-yl)methyl)methanesulfonamide (Compound 122)

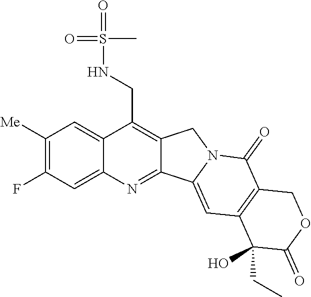 [SMILES: CC[C@@]1(O)C(=O)OCc2c1cc1n(c2=O)Cc2c-1nc1cc(F)c(C)cc1c2CNS(C)(=O)=O]

The title compound was prepared according to General Procedure 3 starting from Compound 1.2 (10 mg) and methane sulfonyl chloride. Preparative HPLC purification was accomplished as described in General Procedure 9, eluting with a 10 to 50% CH₃CN/H₂O+0.1% TFA gradient to give the title compound as an off-white solid (0.8 mg, 7% yield).

LC/MS: Calc'd m/z=487.1 for C₂₃H₂₂FN₃O₆S, found [M+H]⁺=488.2.

¹H NMR (300 MHz, MeOD) δ 8.33 (d, J=8.1 Hz, 1H), 7.83 (d, J=10.8 Hz, 1H), 7.68 (s, 1H), 5.62 (d, J=16.3 Hz, 1H), 5.52 (s, 2H), 5.42 (d, J=16.4 Hz, 1H), 4.87 (s, 2H), 3.06 (s, 3H), 2.59 (s, 3H), 2.06-1.93 (m, 2H), 1.03 (t, J=7.4 Hz, 3H).

### 1.21: (S)-N-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)-1-(4-nitrophenyl) methanesulfonamide (Compound 124)

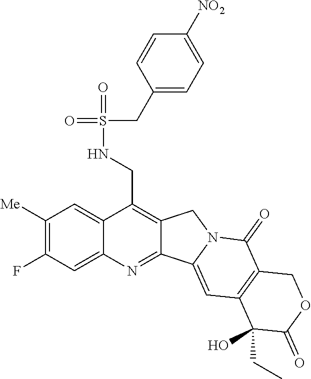 [SMILES: CC[C@@]1(O)C(=O)OCc2c1cc1n(c2=O)Cc2c-1nc1cc(F)c(C)cc1c2CNS(=O)(=O)Cc1ccc([N+](=O)[O-])cc1]

The title compound was prepared according to General Procedure 3 starting from Compound 1.2 (20 mg) and (4-nitrophenyl)methanesulfonyl chloride. Preparative HPLC purification was accomplished as described in General Procedure 9, eluting with a 10 to 50% CH₃CN/H₂O+0.1% TFA gradient to give the title compound as an off-white solid (5.0 mg, 17% yield).

LC/MS: Calc'd m/z=608.1 for C₂₉H₂₅FN₄O₈S, found [M+H]⁺=609.2.

¹H NMR (300 MHz, CDCl₃) δ 8.02-7.92 (m, 3H), 7.74 (d, J=10.5 Hz, 1H), 7.65 (s, 1H), 7.33 (d, J=8.6 Hz, 2H), 5.66 (d, J=16.8 Hz, 1H), 5.28 (d, J=16.5 Hz, 1H), 5.14 (d, J=5.4 Hz, 2H), 4.67 (s, 2H), 4.28 (d, J=6.3 Hz, 2H), 3.39 (s, 3H), 2.03-1.83 (m, 2H), 1.04 (t, J=7.4 Hz, 3H).

### 1.22: (S)-N-((4-ethyl-8-fluoro-4-hydroxy-9-methyl-3,14-dioxo-3,4,12,14-tetrahydro-1H-pyrano[3,4′:6,7]indolizino[1,2-b]quinolin-11-yl)methyl)benzenesulfonamide (Compound 125)

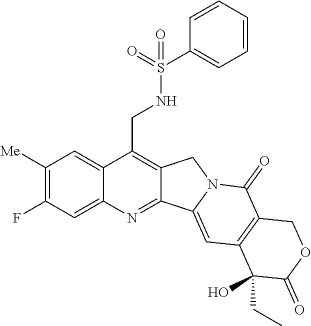 [SMILES: CC[C@@]1(O)C(=O)OCc2c1cc1n(c2=O)Cc2c-1nc1cc(F)c(C)cc1c2CNS(=O)(=O)c1ccccc1]

The title compound was prepared according to General Procedure 3 starting from Compound 1.2 (10 mg) and benzenesulfonyl chloride. Preparative HPLC purification was accomplished as described in General Procedure 9, eluting with a 10 to 50% CH₃CN/H₂O+0.1% TFA gradient to give the title compound as an off-white solid (9.8 mg, 73% yield).

LC/MS: Calc'd m/z=549.6 for C₂₈H₂₄FN₃O₆S, found [M+H]⁺=550.6.

¹H NMR (300 MHz, DMSO-d6) δ 8.60 (t, J=6.2 Hz, 1H), 8.17 (d, J=8.1 Hz, 1H), 7.83 (d, J=10.8 Hz, 1H), 7.71 (dd, J=7.1, 1.7 Hz, 2H), 7.66-7.48 (m, 2H), 7.46 (dd, J=8.3, 6.8 Hz, 2H), 7.40-7.27 (m, 2H), 7.18 (s, 1H), 7.01 (s, 1H), 5.45 (s, 2H), 5.33 (s, 2H), 4.63 (d, J=6.2 Hz, 2H), 2.48 (s, 3H), 1.98-1.76 (m, 2H), 0.89 (t, J=7.3 Hz, 3H).

### 1.23: (S)-N-((4-ethyl-8-fluoro-4-hydroxy-9-methyl-3,14-dioxo-3,4,12,14-tetrahydro-1H-pyrano[3,4′:6,7]indolizino[1,2-b]quinolin-11-yl)methyl)-4-nitrobenzenesulfonamide (Compound 1.23)

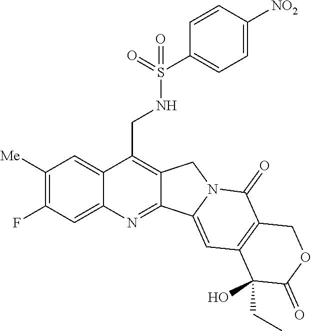 [SMILES: CC[C@@]1(O)C(=O)OCc2c1cc1n(c2=O)Cc2c-1nc1cc(F)c(C)cc1c2CNS(=O)(=O)c1ccc([N+](=O)[O-])cc1]

The title compound was prepared according to General Procedure 3 starting from Compound 1.2 (75 mg) and 4-nitrobenzenesulfonyl chloride. Purification of the title compound was accomplished as described in General Procedure 9, using a 12 g C₁₈ column and eluting with a 5 to 75% CH₃CN/H₂O+0.1% TFA gradient to give the title compound as an off-white solid (37.8 mg, 47% yield).

LC/MS: Calc'd m/z=594.6 for C₂₈H₂₃FN₄O₈S, found [M+H]⁺=595.2.

### 1.24: (S)-4-amino-N-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)benzenesulfonamide (Compound 127)

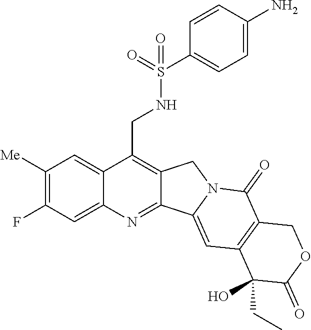 [SMILES: CC[C@@]1(O)C(=O)OCc2c1cc1n(c2=O)Cc2c-1nc1cc(F)c(C)cc1c2CNS(=O)(=O)c1ccc(N)cc1]

To a solution of Compound 1.23 (37.8 mg, 0.064 mmol) in methanol (6.4 mL) was added platinum 1% vanadium 2% on carbon (75 mg). The flask was purged with H₂ then stirred at room temperature under an H₂ atmosphere for 45 min. The mixture was filtered through a pad of celite, washed with DMF, and the filtrate was evaporated to give the title compound as a pale yellow solid (30 mg, 84% yield).

LC/MS: Calc'd m/z=564.6 for C₂₈H₂₄FN₄O₆S, found [M+H]⁺=565.2.

¹H NMR (300 MHz, DMSO-d6) δ 8.13 (d, J=8.2 Hz, 1H), 8.02 (t, J=6.2 Hz, 1H), 7.88 (d, J=10.8 Hz, 1H), 7.48-7.35 (m, 2H), 7.31 (d, J=8.4 Hz, 1H), 6.63-6.45 (m, 2H), 5.45 (s, 2H), 5.36 (s, 2H), 4.50 (d, J=6.3 Hz, 2H), 1.98-1.75 (m, 2H), 0.89 (t, J=7.3 Hz, 3H).

### 1.25: (S)-N-((4-ethyl-8-fluoro-4-hydroxy-9-methyl-3,14-dioxo-3,4,12,14-tetrahydro-1H-pyrano[3,4′:6,7]indolizino[1,2-b]quinolin-11-yl)methyl)-2-hydroxyethane-1-sulfonamide (Compound 129)

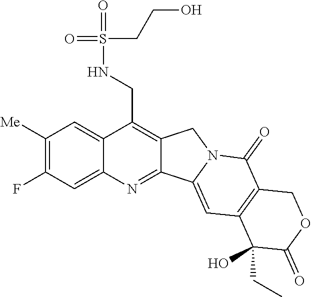 [SMILES: CC[C@@]1(O)C(=O)OCc2c1cc1n(c2=O)Cc2c-1nc1cc(F)c(C)cc1c2CNS(=O)(=O)CCO]

The title compound was prepared according to General Procedure 3 starting from Compound 1.2 (20 mg) and 2-hydroxyethanesulfonyl chloride. Preparative HPLC purification was accomplished as described in General Procedure 9, eluting with a 25 to 50% CH₃CN/H₂O+0.1% TFA gradient to give the title compound as an off-white solid (1.3 mg, 13% yield).

LC/MS: Calc'd m/z=517.1 for C₂₄H₂₄FN₃O₇S, found [M+H]⁺=518.2.

¹H NMR (300 MHz, DMSO-d6) δ 8.30 (d, J=8.4 Hz, 1H), 7.91 (d, J=10.9 Hz, 1H), 7.84 (t, J=6.3 Hz, 1H), 7.33 (s, 1H), 5.50-5.33 (m, 4H), 5.07 (t, J=5.4 Hz, 1H), 4.78 (d, J=6.0 Hz, 2H), 4.07 (s, 3H), 3.80 (dt, J=6.3 Hz, J=5.8 Hz, 2H), 1.86 (m, 2H), 0.87 (d, J=7.3 Hz, 3H).

### 1.26: (S)-N-((4-ethyl-8-fluoro-4-hydroxy-9-methyl-3,14-dioxo-3,4,12,14-tetrahydro-1H-pyrano[3,4′:6,7]indolizino[1,2-b]quinolin-11-yl)methyl)methanesulfamide (Compound 131)

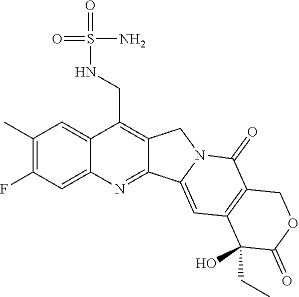 [SMILES: CC[C@@]1(O)C(=O)OCc2c1cc1n(c2=O)Cc2c-1nc1cc(F)c(C)cc1c2CNS(N)(=O)=O]

To a solution of chlorosulfonyl isocyanate (3 uL) in dichloromethane (1 mL) was added tert-butanol (3 uL). This solution was stirred for 1 h, then Compound 1.2 (13 mg) dissolved in dichloromethane (1 mL) was added followed by triethylamine (13 uL). The reaction was stirred for 1 hr then concentrated to dryness. Preparative HPLC purification of the intermediate Boc compound was accomplished as described in General Procedure 9, eluting with a 10 to 50% CH₃CN/H₂O+0.1% TFA gradient. To the purified solid in dichloromethane (1 mL) was added trifluoroacetic acid (200 uL). The reaction was stirred for 16 h then concentrated to dryness to provide the title compound as an off-white solid (7.5 mg, 48% yield).

LC/MS: Calc'd m/z=488.1 for C₂₂H₂₁FN₄O₆S, found [M+H]⁺=489.0.

¹H NMR (300 MHz, MeOD) δ 8.25 (d, J=8.1 Hz, 1H), 7.73 (d, J=10.7 Hz, 1H), 7.62 (s, 1H), 5.59 (d, J=16.4 Hz, 1H), 5.45 (s, 2H), 5.39 (d, J=16.4 Hz, 1H), 4.81 (s, 2H), 2.55 (d, J=1.7 Hz, 3H), 2.07-1.89 (m, 2H), 1.03 (t, J=7.4 Hz, 3H).

### 1.27: 4-nitrophenyl-(S)-((4-ethyl-8-fluoro-4-hydroxy-9-methyl-3,14-dioxo-3,4,12,14-tetrahydro-1H-pyrano[3,4′:6,7]indolizino[1,2-b]quinolin-11-yl)methyl)carbamate (Compound 1.27)

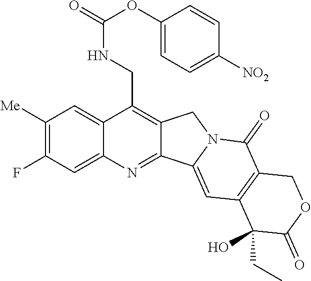 [SMILES: CC[C@@]1(O)C(=O)OCc2c1cc1n(c2=O)Cc2c-1nc1cc(F)c(C)cc1c2CNC(=O)Oc1ccc([N+](=O)[O-])cc1]

The title PNP-carbamate intermediate compound was prepared according to Step 1 of General Procedure 4 starting from Compound 1.2 (24 mg). Flash purification was accomplished as described in General Procedure 9, using a 12 g column C₁₈ column and eluting with a 10 to 50% CH₃CN/H₂O+0.1% TFA gradient to give the title compound as an off-white solid (14 mg, 53% yield).

LC/MS: Calc'd m/z=574.2 for C₂₉H₂₃FN₄O₈S, found [M+H]⁺=575.2

### 1.28: (S)-1-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)-3-methylurea (Compound 132)

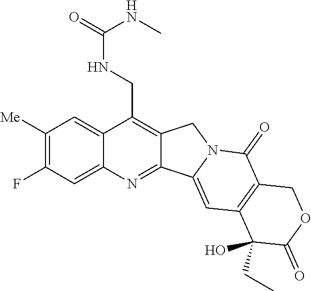 [SMILES: CC[C@@]1(O)C(=O)OCc2c1cc1n(c2=O)Cc2c-1nc1cc(F)c(C)cc1c2CNC(=O)NC]

The title compound was prepared according to General Procedure 4 using Compound 1.2 (25 mg) and aqueous methyl amine (500 uL, 40 wt. % in water) as the primary amine. In this instance, the intermediate PNP-carbamate was used crude. Preparative HPLC purification was accomplished as described in General Procedure 9, eluting with a 10 to 50% CH₃CN/H₂O+0.1% TFA gradient to give the title compound as an off-white solid (8.9 mg, 31% yield).

LC/MS: Calc'd m/z=466.2 for C₂₄H₂₃FN₄O₅, found [M+H]⁺=467.2.

¹H NMR (300 MHz, MeOD) δ 8.26 (d, J=8.2 Hz, 1H), 7.79 (d, J=10.7 Hz, 1H), 7.66 (s, 1H), 5.61 (d, J=16.3 Hz, 1H), 5.48 (s, 2H), 5.41 (d, J=16.4 Hz, 1H), 4.97 (s, 2H), 2.73 (s, 3H), 2.57 (s, 3H), 2.08-1.93 (m, 2H), 1.03 (t, J=7.4 Hz, 3H).

### 1.29: (S)-1-(4-aminobenzyl)-3-((4-ethyl-8-fluoro-4-hydroxy-9-methyl-3,14-dioxo-3,4,12,14-tetrahydro-1H-pyrano[3,4′:6,7]indolizino[1,2-b]quinolin-11-yl)methyl)urea (Compound 134)

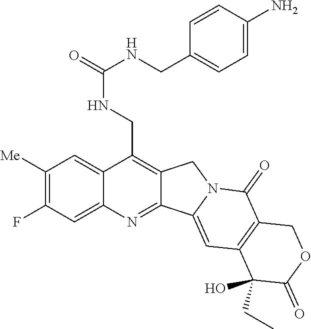 [SMILES: CC[C@@]1(O)C(=O)OCc2c1cc1n(c2=O)Cc2c-1nc1cc(F)c(C)cc1c2CNC(=O)NCc1ccc(N)cc1]

The title compound was prepared according to Step 2 of General Procedure 4 using Compound 1.27 (4 mg) as the PNP-carbamate and 4-(aminomethyl)aniline as the primary amine. Preparative HPLC purification was accomplished as described in General Procedure 9, eluting with a 20 to 50% CH₃CN/H₂O+0.1% TFA gradient to give the title compound as an off-white solid (0.6 mg, 12% yield).

LC/MS: Calc'd m/z=557.2 for C₃₀H₂₈FN₅O₅, found [M+H]⁺=558.4.

¹H NMR (300 MHz, MeOD) δ 8.25 (d, J=8.1 Hz, 1H), 7.80 (d, J=10.8 Hz, 1H), 7.67 (s, 1H), 7.43 (d, J=8.2 Hz, 2H), 7.24 (d, J=8.3 Hz, 2H), 5.63 (d, J=16.4 Hz, 1H), 5.48 (s, 2H), 5.43 (d, J=16.4 Hz, 1H), 5.01 (s, 2H), 4.37 (s, 2H), 2.56 (d, J=1.7 Hz, 3H), 2.05-1.94 (m, 2H), 1.03 (t, J=7.3 Hz, 3H).

### 1.30: (S)-1-((4-ethyl-8-fluoro-4-hydroxy-9-methyl-3,14-dioxo-3,4,12,14-tetrahydro-1H-pyrano[3′,4′ 6,7]indolizino[1,2-b]quinolin-11-yl)methyl)-3-(2-hydroxyethyl)urea (Compound 136)

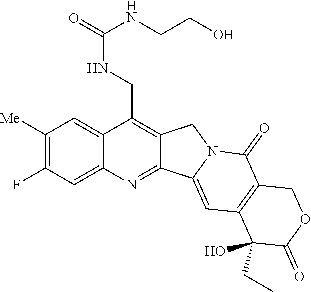 [SMILES: CC[C@@]1(O)C(=O)OCc2c1cc1n(c2=O)Cc2c-1nc1cc(F)c(C)cc1c2CNC(=O)NCCO]

The title compound was prepared according to Step 1 of General Procedure 4 using Compound 1.27 (4 mg) as the PNP-carbamate and hydroxyethylamine as the primary amine. Preparative HPLC purification was accomplished as described in General Procedure 9, eluting with a 10 to 50% CH₃CN/H₂O+0.1% TFA gradient to give the title compound as an off-white solid (2.4 mg, 66% yield).

LC/MS: Calc'd m/z=496.2 for C₂₅H₂₅FN₄O₆, found [M+H]⁺=497.2.

¹H NMR (300 MHz, MeOD) δ 8.08 (d, J=8.0 Hz, 1H), 7.74 (d, J=10.5 Hz, 1H), 7.68 (s, 1H), 5.64 (d, J=16.4 Hz, 1H), 5.41 (s, 2H), 5.31 (d, J=16.4 Hz, 1H), 4.96 (s, 2H), 3.63 (t, J=5.2 Hz, 2H), 3.29 (t, J=5.3 Hz, 2H), 2.54 (s, 3H), 1.98-1.87 (m, 2H), 1.01 (t, J=7.4 Hz, 3H).

### 1.31: Methyl-(S)-((4-ethyl-8-fluoro-4-hydroxy-9-methyl-3,14-dioxo-3,4,12,14-tetrahydro-1H-pyrano[3,4′:6,7]indolizino[1,2-b]quinolin-11-yl)methyl)carbamate (Compound 138)

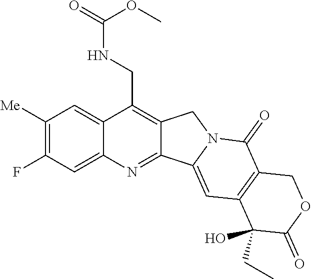 [SMILES: CC[C@@]1(O)C(=O)OCc2c1cc1n(c2=O)Cc2c-1nc1cc(F)c(C)cc1c2CNC(=O)OC]

The title compound was prepared according to General Procedure 5 starting from Compound 1.2 (50 mg) and reacting methanol with the intermediate PNP-carbamate. Preparative HPLC purification was accomplished as described in General Procedure 9, eluting with a 20 to 50% CH₃CN/H₂O+0.1% TFA gradient to give the title compound as an off-white solid (3.5 mg, 6% yield).

LC/MS: Calc'd m/z=467.2 for C₂₄H₂₂FN₃O₆, found [M+H]⁺=468.2.

¹H NMR (300 MHz, MeOD) δ 8.17 (d, J=8.2 Hz, 1H), 7.77 (d, J=10.5 Hz, 1H), 7.69 (s, 1H), 5.65 (d, J=16.5 Hz, 1H), 5.48 (s, 2H), 5.33 (d, J=16.4 Hz, 1H), 4.86 (d, J=5.6 Hz, 2H), 3.65 (s, 3H), 2.56 (s, 3H), 2.02-1.89 (m, 2H), 1.02 (t, J=7.4 Hz, 3H).

### 1.32: 2-hydroxyethyl (S)-((4-ethyl-8-fluoro-4-hydroxy-9-methyl-3,14-dioxo-3,4,12,14-tetrahydro-1H-pyrano[3,4′:6,7]indolizino[1,2-b]quinolin-11-yl)methyl)carbamate (Compound 139)

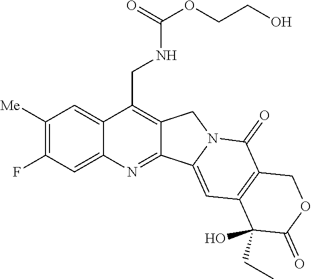 [SMILES: CC[C@@]1(O)C(=O)OCc2c1cc1n(c2=O)Cc2c-1nc1cc(F)c(C)cc1c2CNC(=O)OCCO]

The title compound was prepared according to General Procedure 5 starting from Compound 1.2 (18 mg) and reacting 1,2-ethanediol with the intermediate PNP-carbamate. Preparative HPLC purification was accomplished as described in General Procedure 9, eluting with a 10 to 60% CH₃CN/H₂O+0.1% TFA gradient to give the title compound as an off-white solid (4.2 mg, 19% yield).

LC/MS: Calc'd m/z=497.2 for C₂₅H₂₄FN₃O₇, found [M+H]⁺=498.2.

¹H NMR (300 MHz, DMSO) δ 8.23 (d, J=8.2 Hz, 1H), 7.78 (d, J=10.7 Hz, 1H), 7.40 (s, 1H), 5.47 (d, J=16.5 Hz, 1H), 5.42 (s, 2H), 5.34 (d, J=16.4 Hz, 1H), 4.77 (s, 2H), 3.99 (t, J=4.9 Hz, 2H), 3.64-3.38 (m, 2H), 2.48 (s, 3H), 2.02-1.67 (m, 2H), 0.89 (t, J=7.3 Hz, 3H).

### Example 2: Preparation of Camptothecin Analogues Having Methoxy at the C10 Position

### 2.1: 1-(2-amino-4-fluoro-5-methoxyphenyl)-2-chloroethan-1-one (Compound 2.1)

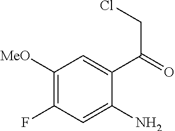 [SMILES: COc1cc(C(=O)CCl)c(N)cc1F]

A solution of 3-fluoro-4-methoxyaniline (10 g, 71 mmol) in DCM (100 mL) was cooled to 0° C. To this solution was first added 1 M BCl₃ in DCM (71 mL, 71 mmol), followed by a 1 M chloro(diethyl)alumane in DCM (71 mL, 71 mmol), then finally 2-chloroacetonitrile (6.4 g, 85 mmol). The solution was heated at reflux for 3 h, cooled to room temperature and quenched by the addition of an aqueous 2 M HCl solution. The resulting heterogenous mixture was heated to reflux for 1 h, cooled to room temperature, then the pH was adjusted to ˜12 with Na₂CO₃. The layers were separated, and the aqueous layer extracted with DCM (3×100 mL). The combined organic layers were dried over Na₂SO₄, concentrated and flash purified as described in General Procedure 9, eluting with 0 to 20% EtOAc/Hexanes to give the title compound (6 g, 28 mmol, 39% yield).

LC/MS: Calc'd m/z=217.1 for C₉H₉ClFNO₂, found [M+H]⁺=218.1.

¹H NMR (400 MHz, CDCl₃) δ 7.19 (d, J=9.2 Hz, 1H), 6.44 (d, J=12.8 Hz, 1H), 4.59 (s, 2H), 3.86 (s, 3H)

### 2.2: (S)-11-(chloromethyl)-4-ethyl-8-fluoro-4-hydroxy-9-methoxy-1,12-dihydro-14H-pyrano[3′,4′:6,7]indolizino[1,2-b]quinoline-3,14(4H)-dione (Compound 2.2)

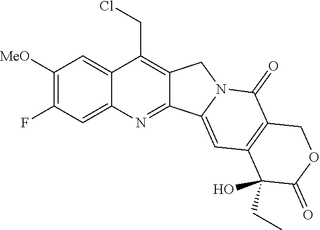 [SMILES: CC[C@@]1(O)C(=O)OCc2c1cc1n(c2=O)Cc2c-1nc1cc(F)c(OC)cc1c2CCl]

To a solution of Compound 2.1 (1.65 g, 7.6 mmol) and (S)-4-ethyl-4-hydroxy-7,8-dihydro-1H-pyrano[3,4-f]indolizine-3,6,10(4H)-trione (2 g, 7.6 mmol) in toluene (200 mL) was added toluene-4-sulfonic acid (157 mg, 0.9 mmol). This solution was heated at 140° C. for 3 h then cooled to room temperature. The product as yellow precipitate was collected by filtration to give the title compound (1.27 g, 2.85 mmol, 37.5% yield).

LC/MS: Calc'd m/z=445.2 for C₂₂H₁₈ClFN₂O₅, found [M+H]⁺=445.1.

¹H NMR (400 MHz, DMSO-d6) δ 7.99 (d, J=12.0 Hz, 1H) 7.80 (d, J=9.2 Hz, 1H) 7.27 (s, 1H), 6.50 (s, 1H), 5.45 (s, 2H), 5.41 (s, 2H), 5.33 (s, 2H) 4.08 (s, 3H), 1.87-1.83 (m, 2H), 0.87 (t, J=7.2 Hz, 3H)

### 2.3: (S)-4-ethyl-8-fluoro-4-hydroxy-9-methoxy-11-(morpholinomethyl)-1,12-dihydro-14H-pyrano[3′,4′:6,7]indolizino[1,2-b]quinoline-3,14(4H)-dione (Compound 101)

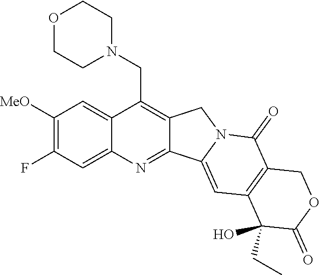 [SMILES: CC[C@@]1(O)C(=O)OCc2c1cc1n(c2=O)Cc2c-1nc1cc(F)c(OC)cc1c2CN1CCOCC1]

The title compound was prepared according to General Procedure 1 starting from Compound 2.2 (10 mg) and morpholine. Preparative HPLC purification was accomplished as described in General Procedure 9, eluting with a 20 to 60% CH₃CN/H₂O+0.1% TFA gradient to give the title compound as an off-white solid (5.6 mg, 41% yield).

LC/MS: Calc'd m/z=495.2 for C₂₆H₂₆FN₃O₆, found [M+H]⁺=496.4.

¹H NMR (300 MHz, MeOD) δ 7.84-7.70 (m, 2H), 7.59 (s, 1H), 5.62 (d, J=16.3 Hz, 1H), 5.45-5.36 (m, 3H), 4.29 (s, 2H), 4.12 (s, 3H), 3.58-3.48 (m, 2H), 3.28-3.09 (m, 2H), 2.75-2.61 (m, 2H), 2.05-1.91 (m, 2H), 1.02 (t, J=7.4 Hz, 3H).

### 2.4: (S)-4-ethyl-8-fluoro-4-hydroxy-9-methoxy-11-((4-(phenylsulfonyl)piperazin-1-yl)methyl)-1,12-dihydro-14H-pyrano[3′,4′: 6,7]indolizino[1,2-b]quinoline-3,14(4H)-dione (Compound 103)

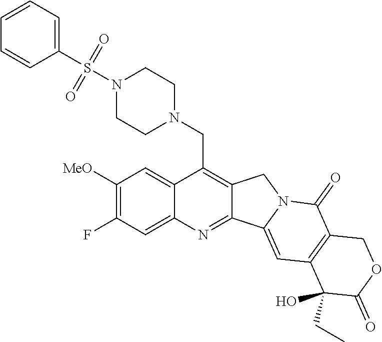 [SMILES: CC[C@@]1(O)C(=O)OCc2c1cc1n(c2=O)Cc2c-1nc1cc(F)c(OC)cc1c2CN1CCN(S(=O)(=O)c2ccccc2)CC1]

The title compound was prepared according to General Procedure 1 starting from Compound 2.2 (10 mg) and 1-(phenylsulfonyl)piperazine. Preparative HPLC purification was accomplished as described in General Procedure 9, eluting with a 20 to 60% CH₃CN/H₂O+0.1% TFA gradient to give the title compound as an off-white solid (2.5 mg, 14% yield).

LC/MS: Calc'd m/z=634.2 for C₃₂H₃₁FN₄O₇S, found [M+H]⁺=635.4.

### 2.5: (S)-11-((4-((4-aminophenyl)sulfonyl)piperazin-1-yl)methyl)-4-ethyl-8-fluoro-4-hydroxy-9-methoxy-1,12-dihydro-14H-pyrano[3′,4′:6,7]indolizino[1,2-b]quinoline-3,14(4H)-dione (Compound 105)

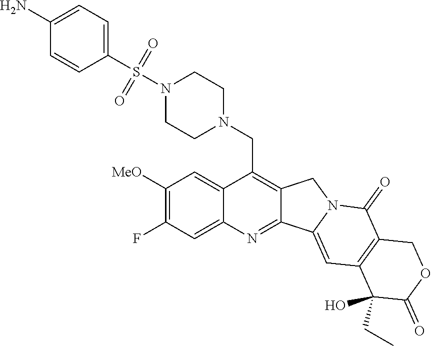 [SMILES: CC[C@@]1(O)C(=O)OCc2c1cc1n(c2=O)Cc2c-1nc1cc(F)c(OC)cc1c2CN1CCN(S(=O)(=O)c2ccc(N)cc2)CC1]

The title compound was prepared according to General Procedure 1 starting from Compound 2.2 (10 mg) and 4-(piperazin-1-ylsulfonyl)aniline. Preparative HPLC purification was accomplished as described in General Procedure 9, eluting with a 20 to 60% CH₃CN/H₂O+0.1% TFA gradient to give the title compound as an off-white solid (4.0 mg, 23% yield).

LC/MS: Calc'd m/z=649.2 for C₃₂H₃₂FN₅O₇S, found [M+H]⁺=650.4.

¹H NMR (300 MHz, DMSO) δ 8.08 (s, 2H), 7.90-7.67 (m, 2H), 7.35 (s, 1H), 7.32-7.26 (m, 2H), 6.67-6.57 (m, 2H), 5.46 (d, J=16.5 Hz, 1H), 5.33-5.22 (m, 3H), 3.92 (s, 3H), 3.02-2.72 (m, 4H), 2.75-2.58 (m, 4H), 1.97-1.70 (m, 2H), 0.90 (t, J=7.3 Hz, 3H).

### 2.6: (S)-4-ethyl-8-fluoro-4-hydroxy-9-methoxy-11-((4-methylpiperazin-1-yl)methyl)-1,12-dihydro-14H-pyrano[3′,4′:6,7]indolizino[1,2-b]quinoline-3,14(4H)-dione (Compound 107)

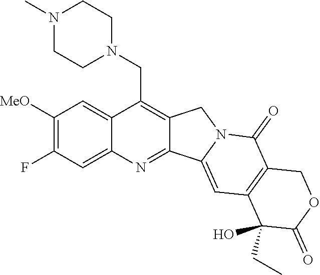 [SMILES: CC[C@@]1(O)C(=O)OCc2c1cc1n(c2=O)Cc2c-1nc1cc(F)c(OC)cc1c2CN1CCN(C)CC1]

The title compound was prepared according to General Procedure 1 starting from Compound 2.2 (10 mg) and N-methylpiperazine. Preparative HPLC purification was accomplished as described in General Procedure 9, eluting with a 20 to 60% CH₃CN/H₂O+0.1% TFA gradient to give the title compound as an off-white solid (2.1 mg, 19% yield).

LC/MS: Calc'd m/z=508.2 for C₂₇H₂₉FN₄O₅, found [M+H]⁺=509.4.

### 2.7: (S)-11-((4-(4-aminophenyl)piperazin-1-yl)methyl)-4-ethyl-8-fluoro-4-hydroxy-9-methoxy-1,12-dihydro-14H-pyrano[3′,4′: 6,7]indolizino[1,2-b]quinoline-3,14(4H)-dione (Compound 109)

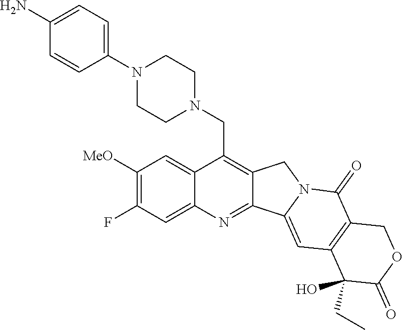 [SMILES: CC[C@@]1(O)C(=O)OCc2c1cc1n(c2=O)Cc2c-1nc1cc(F)c(OC)cc1c2CN1CCN(c2ccc(N)cc2)CC1]

The title compound was prepared according to General Procedure 1 starting from Compound 2.2 (10 mg) and 4-(piperazin-1-yl)aniline. Preparative HPLC purification was accomplished as described in General Procedure 9, eluting with a 20 to 60% CH₃CN/H₂O+0.1% TFA gradient to give the title compound as an off-white solid (3.2 mg, 20% yield).

LC/MS: Calc'd m/z=585.2 for C₃₂H₃₂FN₅O₅, found [M+H]⁺=586.4.

¹H NMR (300 MHz, MeOD) δ 7.83-7.74 (m, 2H), 7.62 (s, 1H), 7.06 (d, J=8.9 Hz, 2H), 6.98 (d, J=8.9 Hz, 2H), 5.65 (d, J=16.4 Hz, 1H), 5.36 (s, 2H), 5.27 (d, J=16.4 Hz, 1H), 4.13 (s, 2H), 4.06 (s, 3H), 3.26 (br s, 4H), 2.79 (br s, 4H), 1.97-1.83 (m, 2H), 1.00 (t, J=7.4 Hz, 3H).

### 2.8: (S)-11-(aminomethyl)-4-ethyl-8-fluoro-4-hydroxy-9-methoxy-1,12-dihydro-14H-pyrano[3′,4′:6,7]indolizino[1,2-b]quinoline-3,14(4H)-dione (Compound 2.8)

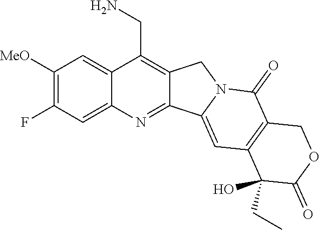 [SMILES: CC[C@@]1(O)C(=O)OCc2c1cc1n(c2=O)Cc2c-1nc1cc(F)c(OC)cc1c2CN]

To a solution of Compound 2.2 (250 mg, 0.56 mmol) in ethanol (7 mL) was added hexamethylenetetramine (236 mg, 1.7 mmol) followed by iPr₂NEt (100 uL, 0.56 mmol). This solution was heated at reflux for 5 h, cooled to room temperature and quenched with 12 M aqueous HCl (60 uL). This solution was concentrated to ˜½ volume and 1 M aqueous HCl (1.5 mL) was added, stirred for 5 min, then concentrated to give a brown residue. Purification was accomplished as described in General Procedure 9, using a 12 g C18 flash column and eluting with a 5 to 40% CH₃CN/H₂O+0.1% TFA gradient to give the title compound as pale-yellow solid (179 mg, 75% yield).

LC/MS: Calc'd m/z=425.4 for C₂₂H₂₀FN₃O₅, found [M+H]⁺=426.2

### 2.9: (S)-N-((4-ethyl-8-fluoro-4-hydroxy-9-methoxy-3,14-dioxo-3,4,12,14-tetrahydro-1H-pyrano[3,4′:6,7]indolizino[1,2-b]quinolin-11-yl)methyl)methanesulfonamide (Compound 123)

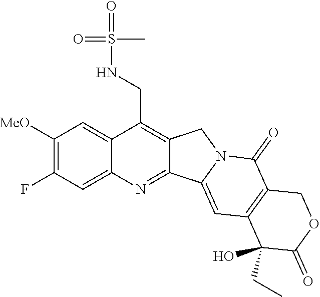 [SMILES: CC[C@@]1(O)C(=O)OCc2c1cc1n(c2=O)Cc2c-1nc1cc(F)c(OC)cc1c2CNS(C)(=O)=O]

The title compound was prepared according to General Procedure 3 starting from Compound 2.8 (10 mg) and methanesulfonyl chloride. Preparative HPLC purification was accomplished as described in General Procedure 9, eluting with a 5 to 65% CH₃CN/H₂O+0.1% TFA gradient to give the title compound as an off-white solid (8.5 mg, 91% yield).

LC/MS: Calc'd m/z=503.1 for C₂₃H₂₂FN₃O₇S, found [M+H]⁺=504.2.

¹H NMR (300 MHz, DMSO-d6) δ 7.98 (d, J=12.1 Hz, 1H), 7.89 (t, J=6.4 Hz, 1H), 7.80 (d, J=9.1 Hz, 1H), 7.28 (s, 1H), 5.42 (s, 2H), 5.39 (s, 2H), 4.77 (d, J=6.4 Hz, 2H), 4.06 (s, 3H), 3.06 (s, 3H), 1.95-1.73 (m, 2H), 0.88 (d, J=7.3 Hz, 3H).

### 2.10: (S)-N-((4-ethyl-8-fluoro-4-hydroxy-9-methoxy-3,14-dioxo-3,4,12,14-tetrahydro-1H-pyrano[3,4′:6,7]indolizino[1,2-b]quinolin-11-yl)methyl)benzenesulfonamide (Compound 126)

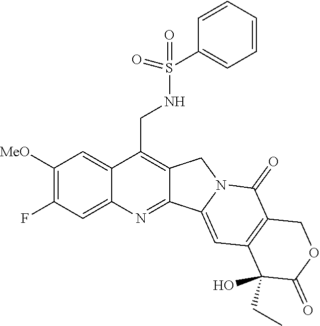 [SMILES: CC[C@@]1(O)C(=O)OCc2c1cc1n(c2=O)Cc2c-1nc1cc(F)c(OC)cc1c2CNS(=O)(=O)c1ccccc1]

The title compound was prepared according to General Procedure 3 starting from Compound 2.8 (7.5 mg) and benzenesulfonyl chloride. Preparative HPLC purification was accomplished as described in General Procedure 9, eluting with a 5 to 70% CH₃CN/H₂O+0.1% TFA gradient to give the title compound as an off-white solid (4.6 mg, 46% yield).

LC/MS: Calc'd m/z=565.6 for C₂₈H₂₄FN₃O₇S, found [M+H]⁺=566.2.

¹H NMR (300 MHz, DMSO-d6) δ 8.59 (t, J=6.3 Hz, 1H), 7.94 (d, J=12.2 Hz, 1H), 7.82-7.68 (m, 2H), 7.62-7.46 (m, 1H), 7.51-7.40 (m, 1H), 7.28 (d, J=8.3 Hz, 1H), 6.52 (s, 1H), 5.44 (s, 1H), 5.36 (s, 1H), 4.64 (d, J=6.3 Hz, 1H), 4.09 (s, 2H), 1.95-1.81 (m, 1H), 0.89 (t, J=7.3 Hz, 2H).

### 2.11: (S)-N-((4-ethyl-8-fluoro-4-hydroxy-9-methoxy-3,14-dioxo-3,4,12,14-tetrahydro-1H-pyrano[3,4′:6,7]indolizino[1,2-b]quinolin-11-yl)methyl)-4-nitrobenzenesulfonamide (Compound 2.11)

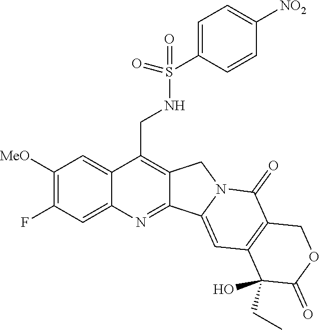 [SMILES: CC[C@@]1(O)C(=O)OCc2c1cc1n(c2=O)Cc2c-1nc1cc(F)c(OC)cc1c2CNS(=O)(=O)c1ccc([N+](=O)[O-])cc1]

The title compound was prepared according to General Procedure 3 starting from Compound 2.8 (12 mg) and 4-nitrobenzenesulfonyl chloride. Purification was accomplished as described in General Procedure 9 using a 12 g C18 flash column and eluting with a 5 to 75% CH₃CN/H₂O+0.1% TFA gradient to give the title compound as pale-yellow solid (9.7 mg, 71% yield).

LC/MS: Calc'd m/z=610.6 for C₂₈H₂₃FN₄09S, found [M+H]⁺=611.5.

### 2.12: (S)-4-amino-N-((4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)benzenesulfonamide (Compound 128)

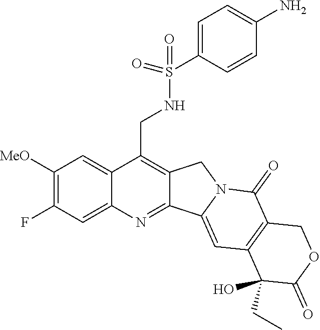 [SMILES: CC[C@@]1(O)C(=O)OCc2c1cc1n(c2=O)Cc2c-1nc1cc(F)c(OC)cc1c2CNS(=O)(=O)c1ccc(N)cc1]

To a solution of Compound 2.11 (9.7 mg, 0.016 mmol) in methanol (1.6 mL) was added platinum 1% vanadium 2% on carbon (15 mg). The flask was purged with H₂ then stirred at room temperature under an H₂ atmosphere for 45 min. The mixture was filtered through a pad of celite, washed with DMF, then the filtrate was evaporated to give the title compound as a pale-yellow solid (1.5 mg, 16% yield).

LC/MS: Calc'd m/z=580.6 for C₂₈H₂₅FN₄O₇S, found [M+H]⁺=581.4.

¹H NMR (300 MHz, MeOD) δ 7.77 (d, J=11.0 Hz, 1H), 7.58 (s, 1H), 7.48 (d, J=8.6 Hz, 1H), 6.61 (d, J=8.6 Hz, 1H), 5.59 (d, J=16.3 Hz, 1H), 5.39 (d, J=16.4 Hz, 1H), 5.30 (s, 1H), 4.56 (s, 1H), 4.10 (d, J=3.7 Hz, 3H), 2.04-1.91 (m, 2H), 1.31 (s, 1H), 1.02 (t, J=7.3 Hz, 3H), 0.90 (s, 1H).

### 2.13: (S)-N-((4-ethyl-8-fluoro-4-hydroxy-9-methoxy-3,14-dioxo-3,4,12,14-tetrahydro-1H-pyrano[3,4′:6,7]indolizino[1,2-b]quinolin-11-yl)methyl)-2-hydroxyethane-1-sulfonamide (Compound 130)

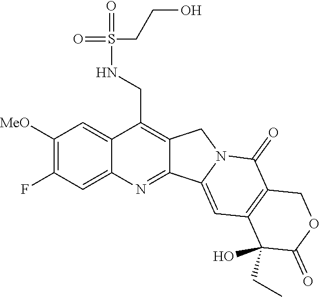 [SMILES: CC[C@@]1(O)C(=O)OCc2c1cc1n(c2=O)Cc2c-1nc1cc(F)c(OC)cc1c2CNS(=O)(=O)CCO]

The title compound was prepared according to General Procedure 3 starting from Compound 2.8 (8 mg) and 2-hydroxyethanesulfonyl chloride. Preparative HPLC purification was accomplished as described in General Procedure 9, eluting with a 15 to 50% CH₃CN/H₂O+0.1% TFA gradient to give the title compound as an off-white solid (2.2 mg, 22% yield).

LC/MS: Calc'd m/z=533.1 for C₂₄H₂₄FN₃O₈S found [M+H]⁺=534.2.

¹H NMR (300 MHz, DMSO-d6) δ 7.99 (d, J=12.2 Hz, 1H), 7.89-7.79 (m, 2H), 7.29 (s, 1H), 5.43 (s, 2H), 5.40 (s, 2H), 4.76 (d, J=6.4 Hz, 2H), 4.06 (s, 3H), 3.81 (t, J=6.3 Hz, 2H), 3.34 (t, J=6.3 Hz, 2H), 1.94-1.75 (m, 2H), 0.87 (d, J=7.4 Hz, 3H).

### 2.14: 4-nitrophenyl-(S)-((4-ethyl-8-fluoro-4-hydroxy-9-methyl-3,14-dioxo-3,4,12,14-tetrahydro-1H-pyrano[3,4′:6,7]indolizino[1,2-b]quinolin-11-yl)methyl)carbamate (Compound 2.14)

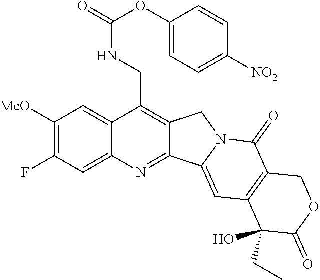 [SMILES: CC[C@@]1(O)C(=O)OCc2c1cc1n(c2=O)Cc2c-1nc1cc(F)c(OC)cc1c2CNC(=O)Oc1ccc([N+](=O)[O-])cc1]

The title PNP-carbamate intermediate compound was prepared according to Step 1 of General Procedure 4 starting from Compound 2.8 (65 mg) and using a 1:1 mixture of dimethylformamide and dichloromethane as the solvent. Flash purification was accomplished as described in General Procedure 9, using a 12 g C12 column and eluting with a 10 to 50% CH₃CN/H₂O+0.1% TFA gradient to give the title compound as an off-white solid (61 mg, 86% yield). This intermediate was divided and used to generate the following compounds.

LC/MS: Calc'd m/z=590.1 for C₂₉H₂₃FN₄O₉, found [M+H]⁺=591.2.

### 2.15: (S)-1-((4-ethyl-8-fluoro-4-hydroxy-9-methoxy-3,14-dioxo-3,4,12,14-tetrahydro-1H-pyrano[3′,4′ 6,7]indolizino[1,2-b]quinolin-11-yl)methyl)-3-methylurea (Compound 133)

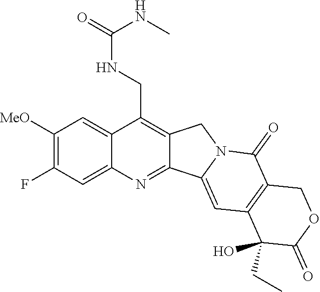 [SMILES: CC[C@@]1(O)C(=O)OCc2c1cc1n(c2=O)Cc2c-1nc1cc(F)c(OC)cc1c2CNC(=O)NC]

The title compound was prepared according to Step 2 of General Procedure 4 using Compound 2.14 (15 mg) as the PNP-carbamate and aqueous methyl amine (500 uL, 40 wt. % in water) as the primary amine. Preparative HPLC purification was accomplished as described in General Procedure 9, eluting with a 20 to 60% CH₃CN/H₂O+0.1% TFA gradient to give the title compound as an off-white solid (5.8 mg, 47% yield).

LC/MS: Calc'd m/z=482.2 for C₂₄H₂₃FN₄O₆, found [M+H]⁺=483.2.

¹H NMR (300 MHz, DMSO-d6) δ 8.00-7.87 (m, 2H), 7.31 (s, 1H), 5.48-5.39 (m, 3H), 4.81 (s, 3H), 2.56 (s, 3H), 1.93-1.81 (m, 2H), 0.89 (t, J=7.3 Hz, 3H).

### 2.16: (S)-1-(4-aminobenzyl)-3-((4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)urea (Compound 135)

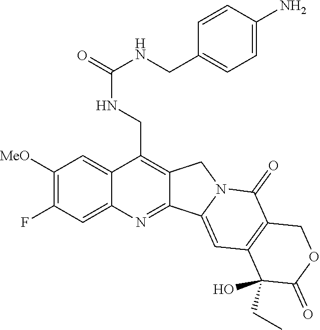 [SMILES: CC[C@@]1(O)C(=O)OCc2c1cc1n(c2=O)Cc2c-1nc1cc(F)c(OC)cc1c2CNC(=O)NCc1ccc(N)cc1]

The title compound was prepared according to Step 2 of General Procedure 4 using Compound 2.14 (15 mg) as the PNP-carbamate and 4-(aminomethyl)aniline as the primary amine. Preparative HPLC purification was accomplished as described in General Procedure 9, eluting with a 20 to 60% CH₃CN/H₂O+0.1% TFA gradient to give the title compound as an off-white solid (TFA salt, 2.1 mg, 12% yield).

LC/MS: Calc'd m/z=573.2 for C₃₀H₂₈FN₅O₆, found [M+H]⁺=574.2.

¹H NMR (300 MHz, MeOD) δ 7.79 (d, J=11.9 Hz, 1H), 7.74 (d, J=9.0 Hz, 1H), 7.59 (s, 1H), 7.43 (d, J=8.2 Hz, 2H), 7.25 (d, J=8.2 Hz, 2H), 5.61 (d, J=16.3 Hz, 1H), 5.52-5.35 (m, 3H), 4.98 (s, 2H), 4.39 (s, 2H), 4.01 (s, 3H), 2.03-1.93 (m, 2H), 1.03 (t, J=7.4 Hz, 3H).

### 2.17: (S)-1-((4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-3-(2-hydroxyethyl)urea (Compound 137)

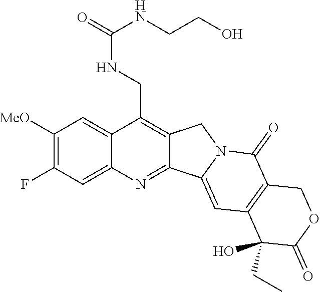 [SMILES: CC[C@@]1(O)C(=O)OCc2c1cc1n(c2=O)Cc2c-1nc1cc(F)c(OC)cc1c2CNC(=O)NCCO]

The title compound was prepared according to Step 2 of General Procedure 4 using Compound 2.14 (15 mg) as the PNP-carbamate and hydroxyethylamine as the primary amine. Preparative HPLC purification was accomplished as described in General Procedure 9, eluting with a 20 to 60% CH₃CN/H₂O+0.1% TFA gradient to give the title compound as an off-white solid (1.5 mg, 12% yield).

LC/MS: Calc'd m/z=512.2 for C₂₅H₂₅FN₄O₇, found [M+H]⁺=513.2.

¹H NMR (300 MHz, MeOD) δ 7.93 (d, J=12.1 Hz, 1H), 7.88 (d, J=9.2 Hz, 1H), 7.56 (s, 1H), 5.62 (d, J=16.2 Hz, 1H), 5.52 (s, 2H), 5.45 (d, J=16.3 Hz, 1H), 4.98 (s, 2H), 4.17 (s, 3H), 3.59 (t, J=5.6 Hz, 2H), 3.28 (t, J=5.6 Hz, 2H), 2.10-1.91 (m, 2H), 1.05 (t, J=7.3 Hz, 3H).

### Example 3: Preparation of Camptothecin Analogues Having Amino at the C10 Position

### 3.1: 5-bromo-4-fluoro-2-nitrobenzaldehyde (Compound 3.1)

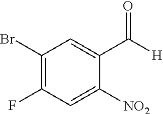 [SMILES: O=Cc1cc(Br)c(F)cc1[N+](=O)[O-]]

To a stirring solution of HNO₃ (121.2 mL, 67% purity, 2.0 eq.) in H₂SO₄ (500 mL) at 0° C. was added 3-bromo-4-fluorobenzaldehyde (180 g, 1.0 eq.). After the addition was complete, the ice bath was removed, and the reaction was allowed to stir for 5 h at 25° C. The mixture was poured into ice (5 L), filtered and then dried under vacuum. The title compound was obtained as a yellow solid (219 g).

¹H NMR (400 MHz, CDCl₃) δ 10.39 (s, 1H), 8.23 (d, J=6.8 Hz, 1H), 7.91 (d, J=7.6 Hz, 1H).

### 3.2: tert-butyl (2-fluoro-5-formyl-4-nitrophenyl)carbamate (Compound 3.2)

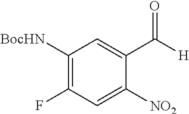 [SMILES: CC(C)(C)OC(=O)Nc1cc(C=O)c([N+](=O)[O-])cc1F]

A mixture of Compound 3.1 (219 g, 1.0 eq.), tert-butyl carbamate (124 g, 1.20 eq.), Cs₂CO₃ (575 g, 2.0 eq.), Pd₂(dba)₃ (40 g, 0.05 eq.) and XPhos (84 g, 0.2 eq.) in toluene (2000 mL) was degassed and purged with N₂ for three cycles. The mixture was then stirred at 90° C. for 15 h under N₂ atmosphere. The reaction mixture was diluted with H₂O (800 mL) and extracted with EtOAc (300 mL×2). The combined organic layers were washed with brine (200 mL×2), then dried over sodium sulfate, filtered and concentrated under reduced pressure. The residue was purified by column chromatography (SiO₂, petroleum ether: ethyl acetate=100: 1 to 20:1) to afford the title compound as a yellow solid (140 g, 56% yield).

¹H NMR (400 MHz, DMSO-d6) δ 10.24 (s, 1H), 9.94 (s, 1H), 8.42 (d, J=7.6 Hz, 1H), 8.16 (d, J=10.8 Hz, 1H), 1.50 (s, 9H)

### 3.3: tert-butyl (4-amino-2-fluoro-5-formylphenyl)carbamate (Compound 3.3)

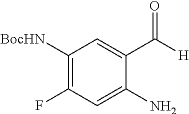 [SMILES: CC(C)(C)OC(=O)Nc1cc(C=O)c(N)cc1F]

To a solution of Compound 3.2 (100 g, 1.0 eq.) in H₂O (300 mL) and EtOH (1200 mL) was added NH₄Cl (30.5 g, 1.62 eq.). Iron (78.6 g, 4.0 eq.) was added in portions at 80° C. The mixture was stirred at 80° C. for 6 h. The mixture was filtered, water was added to the filtrate, and the resulting mixture was extracted with ethyl acetate. The organic layer was washed with brine, dried over sodium sulfate, and concentrated under vacuum. The residue was purified by column chromatography (SiO₂, Petroleum ether: ethyl acetate=1: 0 to 0: 1), TLC (petroleum ether) to afford the title compound as a yellow solid (19.0 g, 21% yield).

LC/MS: Calc'd m/z=254.1 for Cl₂H₁₅FN₂O₃, found [M+H]⁺=255.0.

¹H NMR (400 MHz, DMSO-d6) δ 9.73 (s, 1H), 8.57 (s, 1H), 7.58 (d, J=4.8 Hz, 1H), 7.21 (s, 2H), 6.53 (d, J=12.8 Hz, 1H), 1.43 (s, 9H).

### 3.4: tert-butyl (S)-(4-ethyl-8-fluoro-4-hydroxy-3,14-dioxo-3,4,12,14-tetrahydro-1H-pyrano[3,4′:6,7]indolizino[1,2-b]quinolin-9-yl)carbamate (Compound 3.4)

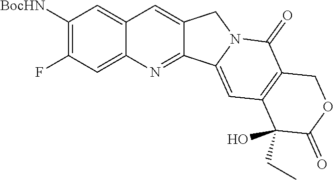 [SMILES: CC[C@@]1(O)C(=O)OCc2c1cc1n(c2=O)Cc2cc3cc(NC(=O)OC(C)(C)C)c(F)cc3nc2-1]

A mixture of Compound 3.3 (4.20 g, 1.2 eq.), (S)-4-ethyl-4-hydroxy-7,8-dihydro-1H-pyrano[3,4-f]indolizine-3,6,10(4H)-trione (3.5 g, 1 eq.) and TsOH (monohydrate, 253 mg, 0.1 eq.) in toluene (350 mL) was stirred at 110° C. for 2 hrs. The reaction solution was cooled to 25° C. and filtered. The solid was washed with methyl-t-butyl ether (30 mL) and then dried under vacuum. The title compound was obtained as a yellow solid (4.5 g, 62% yield).

LC/MS: Calc'd m/z=481.2 for C₂₅H₂₄FN₃O₆, found [M+H]⁺=482.1.

¹H NMR (400 MHz, DMSO-d6) δ 9.49 (s, 1H), 8.65 (s, 1H), 8.43 (d, J=8.4 Hz, 1H), 7.95 (d, J=12.0 Hz, 1H), 7.30 (s, 1H), 6.51 (s, 1H), 5.42 (s, 2H), 5.25 (s, 2H), 1.80-1.92 (m, 2H), 1.52 (s, 9H), 0.88 (t, J=7.2 Hz, 3H)

### 3.5: tert-butyl (S)-(4-ethyl-8-fluoro-4-hydroxy-11-(hydroxymethyl)-3,14-dioxo-3,4,12,14-tetrahydro-1H-pyrano[3,4′:6,7]indolizino[1,2-b]quinolin-9-yl)carbamate (Compound 3.5)

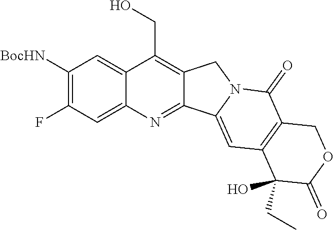 [SMILES: CC[C@@]1(O)C(=O)OCc2c1cc1n(c2=O)Cc2c-1nc1cc(F)c(NC(=O)OC(C)(C)C)cc1c2CO]

To a mixture of Compound 3.4 (4.00 g) in MeOH (360 mL) was added a solution of FeSO₄ (heptahydrate, 1.2 g), H₂SO₄ (280 μL) in H₂O (4 mL). The reaction mixture was heated at 65° C. while H₂O₂ (24 mL, 30% purity) was added dropwise over 30 min and then stirred 0.5 h.

The reaction solution was cooled to 25° C., then filtered to provide the title compound as a yellow solid (1.53 g, 33.2% yield). To the filtrate was added H₂O (400 mL), then quenched with saturated aqueous Na₂S₂O₃. The pH was adjusted to 7-8 with saturated aqueous Na₂CO₃ then the solution was concentrated and filtered. The solid was triturated with MeOH (30 mL) at 55° C. for 1 h, then filtered, to provide a second batch of the title compound as a brown solid (1.09 g, 26% yield).

LC/MS: Calc'd m/z=511.2 for C₂₆H₂₆FN₃O₇, found [M+H]⁺=512.2.

¹H NMR (300 MHz, d6-DMSO) δ 9.47 (s, 1H), 8.47 (d, J=7.6 Hz, 1H), 7.94 (d, J=12.0 Hz, 1H), 7.29 (d, J=1.6 Hz, 1H), 6.49 (s, 1H), 5.86-5.76 (m, 1H), 5.42 (s, 2H), 5.38 (s, 2H), 5.16 (d, J=4.4 Hz, 2H), 1.90-1.83 (m, 2H), 1.52 (s, 9H), 0.88 (t, J=6.4 Hz, 3H).

### 3.6: tert-butyl(S)-(4-ethyl-8-fluoro-11-formyl-4-hydroxy-3,14-dioxo-3,4,12,14-tetrahydro-1H-pyrano[3,4′:6,7]indolizino[1,2-b]quinolin-9-yl)carbamate (Compound 3.6)

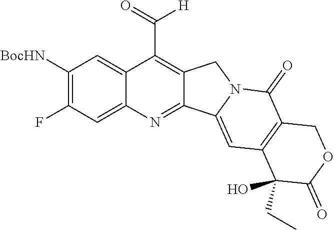 [SMILES: CC[C@@]1(O)C(=O)OCc2c1cc1n(c2=O)Cc2c-1nc1cc(F)c(NC(=O)OC(C)(C)C)cc1c2C=O]

In a 50 mL round-bottom flask containing Compound 3.5 (150 mg, 0.293 mmol) was added DCM (2.9 mL) followed by Dess-Martin periodinane (0.56 g, 1.32 mmol) and water (15.8 μL, 0.88 mmol). This solution was stirred at room temperature for 18 h then diluted with DCM, washed with saturated aqueous NaHCO₃ and brine. The layers were separated, and the combined organic layers were evaporated onto celite. Flash purification was accomplished as described in General Procedure 9, using a 10 g silica column and eluting with 0 to 10% DCM/MeOH to give the title product as an orange powder (42.5 mg, 28%).

LC/MS: Calc'd m/z=509.2 for C₂₆H₂₄FN₃O₇, found [M+H]⁺=510.4.

¹H NMR (300 MHz, Acetone-d6) δ 11.10 (s, 1H), 9.68 (d, J=8.6 Hz, 1H), 8.81 (s, 1H), 8.04 (d, J=11.9 Hz, 1H), 7.63 (s, 1H), 5.73 (s, 2H), 5.69 (d, J=16.2 Hz, 1H), 5.42 (d, J=16.2 Hz, 1H), 2.02-1.95 (m, 2H), 8.47 (d, J=7.6 Hz, 1H), 7.94 (d, J=12.0 Hz, 1H), 7.29 (d, J=1.6 Hz, 1H), 6.49 (s, 1H), 5.86-5.76 (m, 1H), 5.42 (s, 2H), 5.38 (s, 2H), 5.16 (d, J=4.4 Hz, 2H), 1.90-1.83 (m, 2H), 1.52 (s, 9H), 0.88 (t, J=6.4 Hz, 3H).

### 3.7: (S)-9-amino-4-ethyl-8-fluoro-4-hydroxy-1,12-dihydro-14H-pyrano[3,4′:6,7]indolizino [1,2-b]quinoline-3,14(4H)-dione (Compound 140)

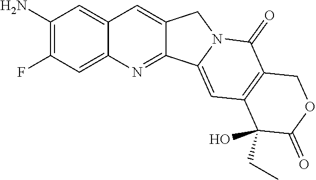 [SMILES: CC[C@@]1(O)C(=O)OCc2c1cc1n(c2=O)Cc2cc3cc(N)c(F)cc3nc2-1]

The title compound was prepared according to General Procedure 6 starting from Compound 3.4 (40 mg) to give the title compound as a red solid (TFA salt, 36 mg, 87% yield).

LC/MS: Calc'd m/z=381.1 for C₂₀H₁₆FN₃O₄, found [M+H]⁺=382.2.

¹H NMR (300 MHz, DMSO) δ 8.28 (s, 1H), 7.72 (d, J=12.5 Hz, 1H), 7.21 (d, J=7.3 Hz, 1H), 5.43 (d, J=16.2 Hz, 1H), 5.34 (d, J=16.2 Hz, 1H), 5.17 (s, 2H), 1.92-1.74 (m, 2H), 0.88 (t, J=7.3 Hz, 3H).

### 3.8: (S)-9-amino-4-ethyl-8-fluoro-4-hydroxy-11-(hydroxymethyl)-1,12-dihydro-14H-pyrano[3′,4′ 6,7]indolizino[1,2-b]quinoline-3,14(4H)-dione (Compound 141)

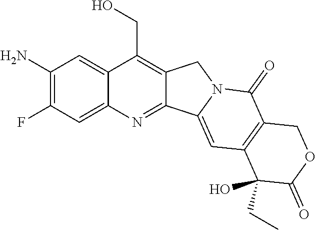 [SMILES: CC[C@@]1(O)C(=O)OCc2c1cc1n(c2=O)Cc2c-1nc1cc(F)c(N)cc1c2CO]

The title compound was prepared according to General Procedure 6 starting from Compound 3.5 (5 mg) to give the title compound as a red solid (TFA salt, 4.1 mg, 78% yield).

LC/MS: Calc'd m/z=411.2 for C₂₁H₁₈FN₃O₅, found [M+H]⁺=412.2.

¹H NMR (300 MHz, MeOD) δ 7.71 (d, J=12.2 Hz, 1H), 7.60 (s, 1H), 7.29 (d, J=9.5 Hz, 1H), 5.61 (d, J=16.3 Hz, 1H), 5.47 (s, 2H), 5.40 (d, J=16.3 Hz, 1H), 5.25 (s, 2H), 2.03-1.94 (m, 2H), 1.03 (t, J=7.4 Hz, 3H).

### 3.9: tert-butyl (S)-(11-(chloromethyl)-4-ethyl-8-fluoro-4-hydroxy-3,14-dioxo-3,4,12,14-tetrahydro-1H-pyrano[3,4′:6,7]indolizino[1,2-b]quinolin-9-yl)carbamate (Compound 3.9)

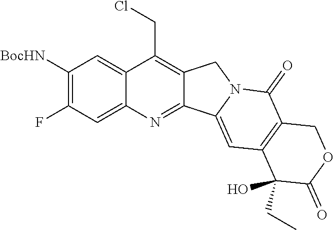 [SMILES: CC[C@@]1(O)C(=O)OCc2c1cc1n(c2=O)Cc2c-1nc1cc(F)c(NC(=O)OC(C)(C)C)cc1c2CCl]

To a stirring solution of Compound 3.5 (100 mg) in dichloromethane (5 mL) was added a solution of thionyl chloride (14 uL) in dichloromethane (0.1 mL). After 1 h, additional thionyl chloride (14 uL) in dichloromethane (0.1 mL) was added. After another 1 h the reaction was diluted with dichloromethane (10 mL) and toluene (1 mL) then concentrated in vacuo to provide the title compound as a red solid that was used in subsequent reactions without additional purification.

LC/MS: Calc'd m/z=529.1 for C₂₆H₂₅ClFN₃O₆, found [M+H]⁺=530.2.

### 3.10: tert-butyl (S)-(11-(aminomethyl)-4-ethyl-8-fluoro-4-hydroxy-3,14-dioxo-3,4,12,14-tetrahydro-1H-pyrano[3,4′:6,7]indolizino[1,2-b]quinolin-9-yl)carbamate (Compound 3.10)

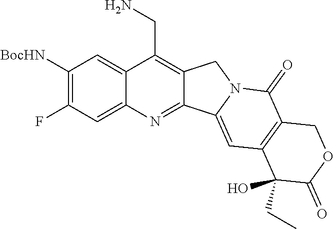 [SMILES: CC[C@@]1(O)C(=O)OCc2c1cc1n(c2=O)Cc2c-1nc1cc(F)c(NC(=O)OC(C)(C)C)cc1c2CN]

To Compound 3.9 (100 mg) in ethanol (500 uL) was added hexamethylenetetramine (79 mg) then DIPEA (99 uL). This solution was heated at 60° C. for 16 h then concentrated to dryness in vacuo. Flash purification was accomplished as described in General Procedure 9, using a 12 g C₁₈ column and eluting with a 10 to 50% CH₃CN/H₂O+0.1% TFA gradient to give the title compound as an off-white solid (TFA salt, 29 mg, 24% yield).

LC/MS: Calc'd m/z=510.2 for C₂₆H₂₇FN₄O₆, found [M+H]⁺=511.4.

¹H NMR (300 MHz, MeOD) δ 8.88 (d, J=8.2 Hz, 1H), 7.96 (d, J=11.9 Hz, 1H), 7.62 (s, 1H), 5.60 (d, J=16.4 Hz, 1H), 5.48 (s, 2H), 5.41 (d, J=16.4 Hz, 1H), 4.80 (s, 2H), 2.07-1.89 (m, 2H), 1.64 (s, 9H), 1.02 (t, J=7.3 Hz, 3H).

### 3.11: (S)-9-amino-11-(aminomethyl)-4-ethyl-8-fluoro-4-hydroxy-1,12-dihydro-14H-pyrano[3′,4′ 6,7]indolizino[1,2-b]quinoline-3,14(4H)-dione (Compound 145)

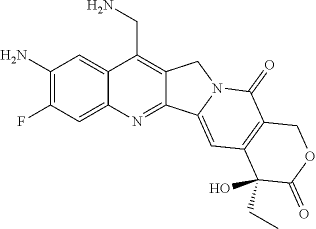 [SMILES: CC[C@@]1(O)C(=O)OCc2c1cc1n(c2=O)Cc2c-1nc1cc(F)c(N)cc1c2CN]

The title compound was prepared according to General Procedure 6 starting from Compound 3.10 (2.1 mg) to give the title compound as a red solid (TFA salt, 1.8 mg, 100% yield).

LC/MS: Calc'd m/z=410.1 for C₂₁H₁₉FN₄O₄, found [M+H]⁺=411.2.

¹H NMR (300 MHz, MeOD) δ 7.82 (d, J=12.1 Hz, 1H), 7.60 (s, 1H), 7.37 (d, J=9.1 Hz, 1H), 5.61 (d, J=16.3 Hz, 1H), 5.42 (s, 2H), 5.41 (d, J=16.3 Hz, 1H), 4.69 (s, 2H), 2.08-1.94 (m, 2H), 1.03 (t, J=7.4 Hz, 3H).

### Example 3.12: (S)-9-amino-4-ethyl-8-fluoro-4-hydroxy-11-(morpholinomethyl)-1,12-dihydro-14H-pyrano[3′,4′:6,7]indolizino[1,2-b]quinoline-3,14(4H)-dione (Compound 3.12)

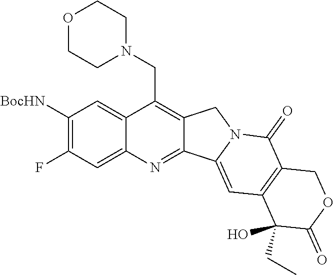 [SMILES: CC[C@@]1(O)C(=O)OCc2c1cc1n(c2=O)Cc2c-1nc1cc(F)c(NC(=O)OC(C)(C)C)cc1c2CN1CCOCC1]

The title compound was prepared according to General Procedure P I starting from Compound 3.9 (150 mg) and morpholine. Preparative HPLC purification was accomplished as described in General Procedure 9, eluting with a 10 to 60% CH₃CN/H₂O+0.1% TFA gradient to give the title compound as a red solid (TFA salt, 103 mg, 52% yield).

LC/MS: Calc'd m/z=580.2 for C₃₀H₃₃FN₄O₇, found [M+H]⁺=581.4.

¹H NMR (300 MHz, MeOD) δ 9.06 (d, J=8.3 Hz, 1H), 7.93 (d, J=12.0 Hz, 1H), 7.66 (s, 1H), 5.63 (d, J=16.3 Hz, 1H), 5.51 (s, 2H), 5.43 (d, J=16.4 Hz, 1H), 4.92 (s, 2H), 3.84 (s, 4H), 3.10 (s, 4H), 1.99 (d, J=5.5 Hz, 2H), 1.63 (s, 9H), 1.03 (t, J=7.4 Hz, 3H).

### 3.13: (S)-9-amino-4-ethyl-8-fluoro-4-hydroxy-11-(morpholinomethyl)-1,12-dihydro-14H-pyrano[3′,4′:6,7]indolizino[1,2-b]quinoline-3,14(4H)-dione (Compound 142)

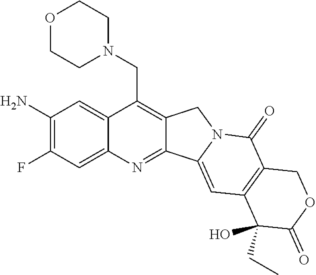 [SMILES: CC[C@@]1(O)C(=O)OCc2c1cc1n(c2=O)Cc2c-1nc1cc(F)c(N)cc1c2CN1CCOCC1]

The title compound was prepared according to General Procedure 6 starting from Compound 3.12 (45 mg) to give the title compound as a red solid (TFA salt, 37 mg, 99% yield).

LC/MS: Calc'd m/z=480.2 for C₂₅H₂₅FN₄O₅, found [M+H]⁺=481.4.

¹H NMR (300 MHz, MeOD) δ 7.73 (d, J=12.0 Hz, 1H), 7.54 (s, 1H), 7.48 (d, J=9.2 Hz, 1H), 5.60 (d, J=16.3 Hz, 1H), 5.47-5.34 (m, 3H), 4.65 (s, 2H), 3.91-3.85 (m, 4H), 3.30-3.24 (m, 4H), 2.08-1.91 (m, 2H), 1.02 (t, J=7.3 Hz, 3H).

### 3.14: (S)-9-amino-4-ethyl-8-fluoro-4-hydroxy-11-(piperidin-1-ylmethyl)-1,12-dihydro-14H-pyrano[3′,4′ 6,7]indolizino[1,2-b]quinoline-3,14(4H)-dione (Compound 148)

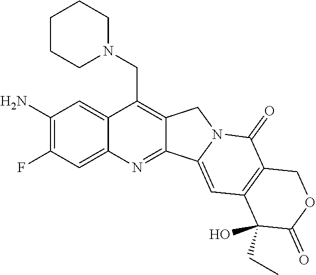 [SMILES: CC[C@@]1(O)C(=O)OCc2c1cc1n(c2=O)Cc2c-1nc1cc(F)c(N)cc1c2CN1CCCCC1]

To a 5 mL flask containing Compound 3.6 (37 mg, 0.067 mmol) was added dichloromethane (1.45 mL) followed by acetic acid (18.69 μL, 0.327 mmol), piperidine (21.52 μL, 0.218 mmol), and sodium triacetoxyborohydride (23.0 mg, 0.109 mmol). This solution was then stirred at room temperature for 2 h, quenched by the addition of water+0.1% TFA and DMF (1:1, 1.0 mL), and partially evaporated. Purification was accomplished as described in General Procedure 9, using a 12 g C18 flash column and eluting with a 5 to 40% CH₃CN/H₂O+0.1% TFA gradient to give the Boc-protected intermediate as a yellow powder. This intermediate was then deprotected according to General Procedure 6 to give the title compound as a yellow solid (TFA salt, 32.5 mg, 98% yield).

LC/MS: Calc'd m/z=478.2 for C₂₆H₂₇FN₄O₄, found [M+H]⁺=479.4.

¹H NMR (300 MHz, MeOD) δ 7.78 (d, J=12.1 Hz, 1H), 7.56 (s, 1H), 7.41 (d, J=9.1 Hz, 1H), 5.60 (d, J=16.4 Hz, 1H), 5.47-5.35 (m, 3H), 4.86 (s, 2H), 3.80-3.68 (m, 2H), 3.28-3.19 (m, 2H), 2.02-1.68 (m, 8H), 1.01 (t, J=7.4 Hz, 3H).

### 3.15: (S)-9-amino-4-ethyl-8-fluoro-4-hydroxy-11-((4-methylpiperazin-1-yl)methyl)-1,12-dihydro-14H-pyrano[3′,4′: 6,7]indolizino[1,2-b]quinoline-3,14(4H)-dione (Compound 149)

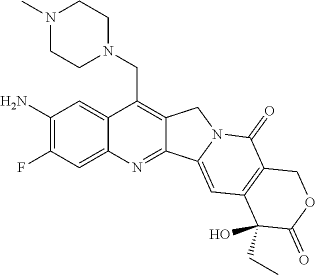 [SMILES: CC[C@@]1(O)C(=O)OCc2c1cc1n(c2=O)Cc2c-1nc1cc(F)c(N)cc1c2CN1CCN(C)CC1]

To a 2 mL vial containing Compound 3.6 (15 mg, 0.029 mmol) was added dichloromethane (0.59 mL), acetic acid (7.58 μL, 0.132 mmol), and N-methylpiperazine (4.90 μL, 0.044 mmol). This solution was stirred at room temperature for 4 h then sodium triacetoxyborohydride (7.8 mg, 0.037 mmol) was added and stirred for an additional 45 min. Excess hydride was quenched by the addition of a 0.1% aqueous TFA solution (0.5 mL). Purification was accomplished as described in General Procedure 9 using a 12 g C18 flash column and eluting with a 5 to 40% CH₃CN/H₂O+0.1% TFA gradient to give the Boc-protected intermediate as a yellow powder. This intermediate was deprotected according to General Procedure 6 to give the title product as a yellow solid (TFA salt, 1.5 mg, 7.1% yield).

LC/MS: Calc'd m/z=493.2 for C₂₆H₂₈FN₅O₄, found [M+H]⁺=494.4.

¹H NMR (300 MHz, MeOD) δ 7.68 (d, J=12.2 Hz, 1H), 7.56 (s, 1H), 7.53 (d, J=9.5 Hz, 1H), 5.60 (d, J=16.3 Hz, 1H), 5.45-5.30 (m, 3H), 4.15 (s, 2H), 3.55-3.44 (m, 2H), 3.18-3.07 (m, 2H), 2.93 (s, 3H), 2.70-2.51 (m, 2H), 2.03-1.89 (m, 2H), 1.02 (t, J=7.4 Hz, 3H).

### 3.16: (S)-9-amino-4-ethyl-8-fluoro-4-hydroxy-11-((4-(phenylsulfonyl)piperazin-1-yl)methyl)-1,12-dihydro-14H-pyrano[3′,4′:6,7]indolizino[1,2-b]quinoline-3,14(4H)-dione (Compound 153)

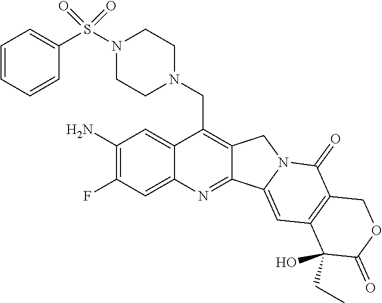 [SMILES: CC[C@@]1(O)C(=O)OCc2c1cc1n(c2=O)Cc2c-1nc1cc(F)c(N)cc1c2CN1CCN(S(=O)(=O)c2ccccc2)CC1]

The Boc-protected precursor of the title compound was prepared according to General Procedure 1 starting from Compound 3.9 (10 mg) and 1-(phenylsulfonyl)piperazine. Preparative HPLC was accomplished as described in General Procedure 9, eluting with a 35 to 44% CH₃CN/H₂O+0.1% TFA gradient to give the Boc-protected intermediate as a yellow powder. This intermediate was then deprotected according to General Procedure 6 to give the title compound (TFA salt, 2.4 mg, 17% yield over 2 steps).

LC/MS: Calc'd m/z=619.2 for C₃₁H₃₀FN₅O₆S, found [M+H]⁺=520.4.

¹H NMR (300 MHz, MeOD) δ 7.81-7.60 (m, 7H), 7.34 (s, 1H), 5.51 (d, J=16.4 Hz, 1H), 5.35 (d, J=16.4 Hz, 1H), 5.22 (s, 2H), 4.10 (s, 2H), 3.15-3.02 (m, 4H), 2.79-2.71 (m, 4H), 2.00-1.93 (m, 2H), 1.00 (t, J=7.4 Hz, 3H).

### 3.17: (S)-N-((9-amino-4-ethyl-8-fluoro-4-hydroxy-3,14-dioxo-3,4,12,14-tetrahydro-1H-pyrano[3,4′:6,7]indolizino[1,2-b]quinolin-11-yl)methyl)acetamide (Compound 147)

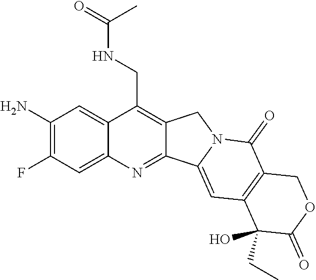 [SMILES: CC[C@@]1(O)C(=O)OCc2c1cc1n(c2=O)Cc2c-1nc1cc(F)c(N)cc1c2CNC(C)=O]

The title compound was prepared according to General Procedure 2 followed by General Procedure 6 starting from Compound 3.10 (8 mg) and acetic acid. Preparative HPLC purification of the intermediate Boc-protected compound was accomplished as described in General Procedure 9, eluting with a 10 to 60% CH₃CN/H₂O+0.1% TFA gradient. The title compound was obtained as a red solid (4.0 mg, 56% yield).

LC/MS: Calc'd m/z=452.2 for C₂₃H₂₁FN₄O₅, found [M+H]⁺=453.2.

¹H NMR (300 MHz, MeOD) δ 7.69 (d, J=12.1 Hz, 1H), 7.56 (s, 1H), 7.38 (d, J=9.3 Hz, 1H), 5.59 (d, J=16.3 Hz, 1H), 5.44-5.33 (m, 3H), 4.85 (s, 3H), 2.03 (s, 3H), 2.00-1.84 (m, 2H), 1.03 (t, J=7.4 Hz, 3H).

### 3.18: (S)-N-((9-amino-4-ethyl-8-fluoro-4-hydroxy-3,14-dioxo-3,4,12,14-tetrahydro-1H-pyrano[3,4′:6,7]indolizino[1,2-b]quinolin-11-yl)methyl)methanesulfonamide (Compound

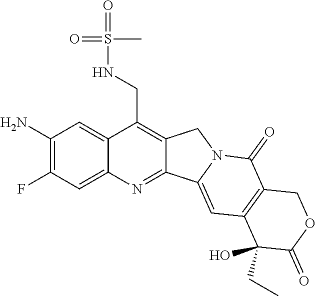 [SMILES: CC[C@@]1(O)C(=O)OCc2c1cc1n(c2=O)Cc2c-1nc1cc(F)c(N)cc1c2CNS(C)(=O)=O]

The title compound was prepared according to General Procedure 3 followed by General Procedure 6 starting from Compound 3.10 (8 mg) and methane sulfonyl chloride. Preparative HPLC purification of the intermediate Boc-protected compound was accomplished as described in General Procedure 9, eluting with a 10 to 60% CH₃CN/H₂O+0.1% TFA gradient. The title compound was obtained as a red solid (4.4 mg, 57% yield).

LC/MS: Calc'd m/z=488.1 for C₂₂H₂₁FN₄O₆S, found [M+H]⁺=489.2.

¹H NMR (300 MHz, MeOD) δ 7.74 (d, J=12.2 Hz, 1H), 7.60 (s, 1H), 7.49 (d, J=9.3 Hz, 1H), 5.61 (d, J=16.2 Hz, 1H), 5.45 (s, 2H), 5.40 (d, J=16.2 Hz, 1H), 4.78 (s, 2H), 3.05 (s, 3H), 2.08-1.94 (m, 2H), 1.03 (t, J=7.4 Hz, 3H).

### 3.19: (S)-N-((9-amino-4-ethyl-8-fluoro-4-hydroxy-3,14-dioxo-3,4,12,14-tetrahydro-1H-pyrano[3,4′:6,7]indolizino[1,2-b]quinolin-11-yl)methyl)-2-hydroxyethane-1-sulfonamide (Compound 150)

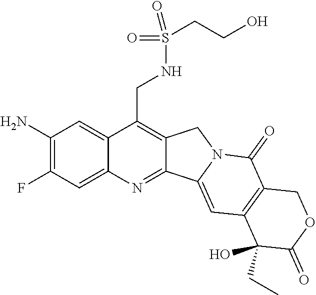 [SMILES: CC[C@@]1(O)C(=O)OCc2c1cc1n(c2=O)Cc2c-1nc1cc(F)c(N)cc1c2CNS(=O)(=O)CCO]

The title compound was prepared according to General Procedure 3 followed by General Procedure 6 starting from Compound 3.10 (6 mg) and 2-hydroxyethanesulfonyl chloride. Preparative HPLC purification of the intermediate Boc-protected compound was accomplished as described in General Procedure 9, eluting with a 10 to 60% CH₃CN/H₂O+0.1% TFA gradient. The title compound was obtained as a red solid (1 mg, 16% yield).

LC/MS: Calc'd m/z=518.5 for C₂₃H₂₃FN₄O₇S, found [M+H]⁺=519.5.

¹H NMR (300 MHz, 10% D₂O/CD₃CN) δ 7.77-7.61 (m, 1H), 7.48-7.30 (m, 2H), 5.53 (d, J=16.3 Hz, 1H), 5.31 (d, J=15.4 Hz, 3H), 4.69 (s, 2H), 3.97 (dd, J=6.6, 4.9 Hz, 2H), 3.39 (t, J=5.8 Hz, 2H), 2.93 (s, 1H), 1.99-1.83 (m, 2H), 0.94 (t, J=7.3 Hz, 3H).

### 3.20: 4-nitrophenyl (S)-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)carbamate (Compound 3.20)

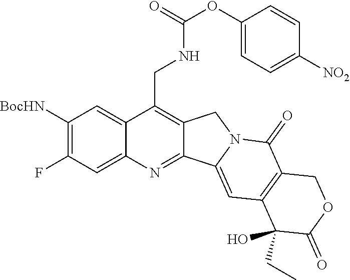 [SMILES: CC[C@@]1(O)C(=O)OCc2c1cc1n(c2=O)Cc2c-1nc1cc(F)c(NC(=O)OC(C)(C)C)cc1c2CNC(=O)Oc1ccc([N+](=O)[O-])cc1]

To a solution of Compound 3.10 (10 mg, 0.02 mmol) in DMF (400 uL, 0.05 M) was added 4-nitrophenyl carbonate (12 mg, 0.04 mmol) and diisopropylethylamine (6.8 uL, 0.04 mmol). This solution was stirred at room temperature for ˜30 min, then used directly in subsequent reactions.

### 3.21: Methyl (S)-((9-amino-4-ethyl-8-fluoro-4-hydroxy-3,14-dioxo-3,4,12,14-tetrahydro-1H-pyrano[3,4′:6,7]indolizino[1,2-b]quinolin-11-yl)methyl)carbamate (Compound 143)

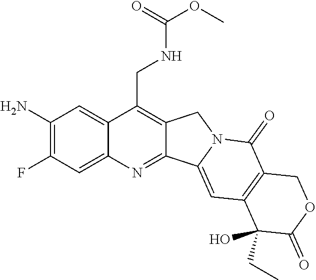 [SMILES: CC[C@@]1(O)C(=O)OCc2c1cc1n(c2=O)Cc2c-1nc1cc(F)c(N)cc1c2CNC(=O)OC]

The title compound was prepared by addition of MeOH (100 uL) to 200 ul of the solution of Compound 3.20. This solution was stirred at room temperature for 30 min. Preparative HPLC purification of the intermediate Boc-protected compound was accomplished as described in General Procedure 9, eluting with a 10 to 60% CH₃CN/H₂O+0.1% TFA gradient. The title compound was obtained according to General Procedure 6 as a red solid (2.1 mg, 47% yield).

LC/MS: Calc'd m/z=468.4 for C₂₃H₂₁FN₄O₆, found [M+H]⁺=468.3.

¹H NMR (300 MHz, 10% D₂O/CD₃CN) δ 7.72 (d, J=12.2 Hz, 1H), 7.41 (d, J=18.1 Hz, 1H), 6.96 (s, 1H), 5.52 (d, J=3.6 Hz, 1H), 5.39-5.23 (m, 3H), 4.82 (s, 1H), 4.73 (s, 1H), 3.63 (d, J=1.2 Hz, 3H), 1.56 (s, 3H), 1.27 (s, 2H), 0.94 (t, J=7.4 Hz, 3H).

### 3.22: (S)-1-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)-3-methylurea (Compound 144)

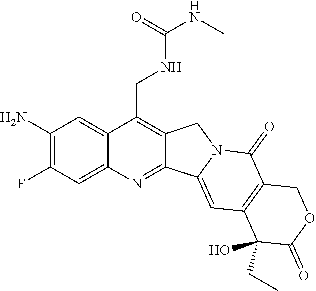 [SMILES: CC[C@@]1(O)C(=O)OCc2c1cc1n(c2=O)Cc2c-1nc1cc(F)c(N)cc1c2CNC(=O)NC]

The title compound was prepared by addition of methylamine hydrochloride (10 mg) to 200 ul of the solution of Compound 3.20, followed by iPr₂NEt (5 uL). This solution was stirred at room temperature for 30 min. Preparative HPLC purification of the intermediate Boc-protected compound was accomplished as described in General Procedure 9, eluting with a 10 to 60% CH₃CN/H₂O+0.1% TFA gradient. The title compound was obtained according to General Procedure 6 as a red solid (2.9 mg, 64.5% yield).

LC/MS: Calc'd m/z=467.5 for C₂₃H₂₁FN₅O₅, found [M+H]⁺=468.5.

¹H NMR (300 MHz, 10% D₂O/CD₃CN) δ 8.13 (d, J=9.2 Hz, 1H), 7.92 (s, 1H), 7.73 (d, J=12.3 Hz, 1H), 7.52-7.35 (m, 2H), 6.94 (d, J=9.2 Hz, 2H), 5.55 (d, J=16.5 Hz, 2H), 5.44-5.27 (m, 4H), 4.85 (s, 2H), 4.78 (s, 1H), 1.56 (d, J=2.5 Hz, 3H), 1.27 (s, 2H), 0.93 (q, J=11.7, 9.5 Hz, 3H).

### 3.23: (S)-1-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)-3-(2-hydroxyethyl)urea (Compound 151)

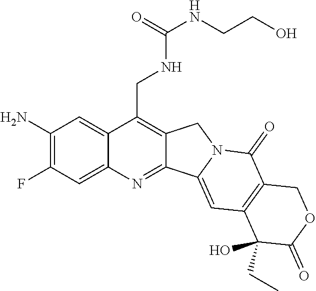 [SMILES: CC[C@@]1(O)C(=O)OCc2c1cc1n(c2=O)Cc2c-1nc1cc(F)c(N)cc1c2CNC(=O)NCCO]

The title compound was prepared by addition of ethanolamine (100 uL) to 200 ul of the solution of Compound 3.20. This solution was stirred at room temperature for 30 min. Preparative HPLC purification of the intermediate Boc-protected compound was accomplished as described in General Procedure 9, eluting with a 10 to 60% CH₃CN/H₂O+0.1% TFA gradient. The title compound was obtained according to General Procedure 6 as a red solid (0.5 mg, 8.5% yield).

LC/MS: Calc'd m/z=497.5 for C₂₄H₂₄FN₅O₆, found [M+H]⁺=498.5.

¹H NMR (300 MHz, 10% D₂O/CD₃CN) δ 7.77-7.61 (m, 1H), 7.48-7.30 (m, 2H), 5.53 (d, J=16.3 Hz, 1H), 5.31 (d, J=15.4 Hz, 1H), 5.19 (s, 2H), 4.69 (s, 2H), 3.97 (dd, J=6.6, 4.9 Hz, 2H), 3.39 (t, J=5.8 Hz, 2H), 2.93 (s, 1H), 2.01-1.83 (m, 2H), 0.94 (t, J=7.3 Hz, 3H).

### 3.24: (S)-9-amino-11-(azidomethyl)-4-ethyl-8-fluoro-4-hydroxy-1,12-dihydro-14H-pyrano[3′,4′ 6,7]indolizino[1,2-b]quinoline-3,14(4H)-dione (Compound 152)

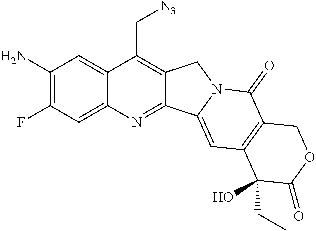 [SMILES: CC[C@@]1(O)C(=O)OCc2c1cc1n(c2=O)Cc2c-1nc1cc(F)c(N)cc1c2CN=[N+]=[N-]]

To a stirring solution of Compound 3.5 (100 mg) in 2 mL dichloromethane was added thionyl chloride (35 μL, 2.5 eq.). The solution was stirred at room temperature for 20 min, then additional thionyl chloride (35 μL, 2.5 eq.) was added. After 20 minutes, toluene (1 mL) was added, and the reaction mixture was concentrated in vacuo. The crude solid was suspended in DMSO (1 mL) and sodium azide (19 mg, 1.5 eq.) was added. This solution was stirred at room temperature for 16 h. Purification was accomplished as described in General Procedure 9, eluting with a 5 to 50% CH₃CN/H₂O+0.1% TFA gradient to give the title compound as an off-white solid (20 mg, 23% yield).

LC/MS: Calc'd m/z=436.1 for C₂₁H₁₇FN₆O₄, found [M+H]⁺=437.2.

¹H NMR (300 MHz, MeOD) δ 7.75 (d, J=12.2 Hz, 1H), 7.60 (s, 1H), 7.38 (d, J=9.3 Hz, 1H), 5.61 (d, J=16.3 Hz, 1H), 5.46-5.35 (m, 3H), 5.07 (s, 2H), 2.03-1.97 (m, 2H), 1.03 (t, J=7.3 Hz, 3H).

### 3.25: (S)-N-((9-amino-4-ethyl-8-fluoro-4-hydroxy-3,14-dioxo-3,4,12,14-tetrahydro-1H-pyrano[3,4′:6,7]indolizino[1,2-b]quinolin-11-yl)methyl)acetamide (Compound 164)

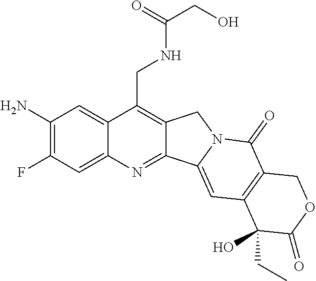 [SMILES: CC[C@@]1(O)C(=O)OCc2c1cc1n(c2=O)Cc2c-1nc1cc(F)c(N)cc1c2CNC(=O)CO]

The title compound was prepared according to General Procedure 2 starting from Compound 145 (10 mg) and glycolic acid. Preparative HPLC purification was accomplished as described in General Procedure 9, eluting with a 10 to 45% CH₃CN/H₂O+0.1% TFA gradient. The title compound was obtained as a yellow solid (6.9 mg, 60% yield).

LC/MS: Calc'd m/z=468.1 for C₂₃H₂₁FN₄O₆, found [M+H]⁺=469.2.

¹H NMR (300 MHz, MeOD) 7.70 (d, J=12.2 Hz, 1H), 7.60 (s, 1H), 7.42 (d, J=9.4 Hz, 1H), 5.62 (d, J=16.3 Hz, 1H), 5.43 (s, 2H), 5.36 (d, J=16.2 Hz, 1H), 4.95 (d, J=5.9 Hz, 2H), 4.08 (s, 2H), 2.04-1.90 (m, 1H), 1.03 (t, J=7.4 Hz, 3H).

### 3.26: (S)-1-((9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)-3-methylthiourea (Compound 161)

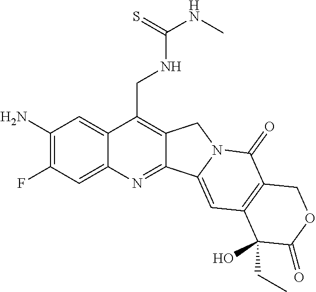 [SMILES: CC[C@@]1(O)C(=O)OCc2c1cc1n(c2=O)Cc2c-1nc1cc(F)c(N)cc1c2CNC(=S)NC]

To a solution of Compound 145 (9 mg, 1.0 eq.) in DMF (1 mL) was added thiocarbonyldiimidazole (6 mg, 1.5 eq.) then DIPEA (8 μL, 2.0 eq.). The resulting solution was stirred at 25° C. for 2 h, after which complete conversion to the isothiocyanate intermediate was observed. Methylammonium chloride (3 mg, 2.0 eq.) was then added and the reaction mixture was heated at 60° C. for 30 min. Preparative HPLC purification was accomplished as described in General Procedure 9, eluting with a 10 to 45% CH₃CN/H₂O+0.1% TFA gradient. The title compound was obtained as a yellow solid (2.3 mg, 22% yield).

LC/MS: Calc'd m/z=483.1 for C₂₃H₂₂FN₅O₄S found [M+H]⁺=484.2.

¹H NMR (300 MHz, MeOD) δ 7.70 (d, J=12.0 Hz, 1H), 7.60 (s, 1H), 7.38 (d, J=9.3 Hz, 1H), 5.62 (d, J=16.2 Hz, 1H), 5.36 (s, 2H), 5.31 (d, J=16.2 Hz, 1H), 5.30 (s, 2H), 3.04 (s, 3H), 1.99-1.90 (m, 2H), 1.02 (t, J=7.4 Hz, 3H).

### 3.27: S-(2-hydroxyethyl)-(S)-((9-amino-4-ethyl-8-fluoro-4-hydroxy-3,14-dioxo-3,4,12,14-tetrahydro-1H-pyrano[3,4′:6,7]indolizino[1,2-b]quinolin-11-yl)methyl)carbamothioate (Compound 160)

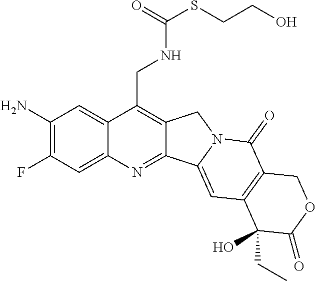 [SMILES: CC[C@@]1(O)C(=O)OCc2c1cc1n(c2=O)Cc2c-1nc1cc(F)c(N)cc1c2CNC(=O)SCCO]

The title compound was prepare according to General Procedure 5 starting from Compound 145 (10 mg) and 2-mercaptoethanol. Preparative HPLC purification was accomplished as described in General Procedure 9, eluting with a 10 to 45% CH₃CN/H₂O+0.1% TFA gradient.

The title compound was obtained as a yellow solid (4.2 mg, 43% yield).

LC/MS: Calc'd m/z=514.1 for C₂₄H₂₃FN₄O₆S found [M+H]⁺=515.2.

¹H NMR (300 MHz, MeOD) δ 7.71 (d, J=12.1 Hz, 1H), 7.60 (s, 1H), 7.36 (d, J=9.4 Hz, 1H), 5.62 (d, J=16.3 Hz, 1H), 5.42 (s, 2H), 5.35 (d, J=16.2 Hz, 1H), 4.88 (d, J=4.6 Hz, 2H), 3.68 (t, J=6.4 Hz, 2H), 3.03 (t, J=6.5 Hz, 2H), 2.04-1.92 (m, 2H), 1.03 (t, J=7.4 Hz, 3H).

### 3.28: (S)-9-amino-4,11-diethyl-8-fluoro-4-hydroxy-1,12-dihydro-14H-pyrano[3,4′:6,7]indolizino[1,2-b]quinoline-3,14(4H)-dione (Compound 154)

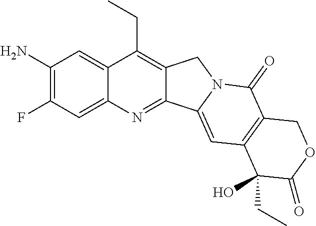 [SMILES: CCc1c2c(nc3cc(F)c(N)cc13)-c1cc3c(c(=O)n1C2)COC(=O)[C@]3(O)CC]

To a 5 mL flask containing Compound 140 (50 mg) was added water (0.72 mL), FeSO₄ (heptahydrate, 11.0 mg) and propionaldehyde (74 μL). The obtained suspension was cooled to −15° C. using an ice brine bath, then sulfuric acid (0.40 mL) was added dropwise. Hydrogen peroxide (95 μL) was then added dropwise. This mixture was stirred at −15° C. for 10 min then allowed to warm up to room temperature and stirred for 2 h. The reaction mixture was diluted with water (30 mL) and the obtained suspension was extracted with DCM (3×30 mL). The organic phase was then evaporated to dryness. Preparative HPLC purification was accomplished as described in General Procedure 9, eluting with a 25 to 70% CH₃CN/H₂O+0.1% TFA gradient to give the title compound as a dark orange solid (2.4 mg, 4.4% yield).

LC/MS: Calc'd m/z=410.1 for C₂₂H₂₀FN₃O₄ found [M+H]⁺=410.2.

¹H NMR (300 MHz, MeOD) δ 7.63 (d, J=12.3 Hz, 1H), 7.55 (s, 1H), 7.36 (d, J=9.4 Hz, 1H), 5.57 (d, J=16.4 Hz, 1H), 5.37 (d, J=16.4 Hz, 1H), 5.21 (s, 2H), 3.13 (q, J=7.7 Hz, 2H), 2.02-1.90 (m, 2H), 1.38 (t, J=7.7 Hz, 3H), 1.01 (t, J=7.3 Hz, 3H).

### 3.29: tert-butyl-(S)-(11-((carbamoyloxy)methyl)-4-ethyl-8-fluoro-4-hydroxy-3,14-dioxo-3,4,12,14-tetrahydro-1H-pyrano[3,4′:6,7]indolizino[1,2-b]quinolin-9-yl)carbamate (Compound 3.29)

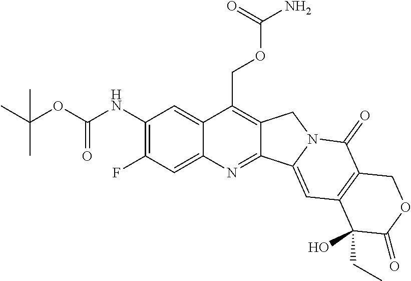 [SMILES: CC[C@@]1(O)C(=O)OCc2c1cc1n(c2=O)Cc2c-1nc1cc(F)c(NC(=O)OC(C)(C)C)cc1c2COC(N)=O]

In a 5 mL conical flask containing a solution of chlorosulfonyl isocyanate (7.7 μL) in dimethylformamide (0.29 mL), at −20° C., was added Compound 3.5 (15 mg). The obtained suspension was stirred at −20° C. for 5 min. Water (59 μL) was added, and the reaction mixture was allowed to warm up to room temperature and stirred for 2 h, then heated at 70° C. for 1 h. The reaction mixture was allowed to cool down to room temperature and partially evaporated. Preparative HPLC purification was accomplished as described in General Procedure 9, eluting with a 40 to 55% CH₃CN/H₂O+0.1% TFA gradient to give the title compound as a dark orange solid (5.1 mg, 31% yield).

LC/MS: Calc'd m/z=555.2 for C₂₇H₂₇FN₄O₈ found [M+H]⁺=555.2.

¹H NMR (300 MHz, DMSO-d6) δ 9.53 (s, 1H), 8.56 (d, J=8.5 Hz, 1H), 8.00 (d, J=12.0 Hz, 1H), 7.31 (s, 1H), 7.11-6.62 (m, 2H), 6.52 (s, 1H), 5.58 (s, 2H), 5.49-5.27 (m, 4H), 1.94-1.77 (m, 2H), 1.52 (s, 9H), 1.38 (t, J=7.7 Hz, 3H), 0.87 (t, J=7.2 Hz, 3H).

### 3.30: (S)-(9-amino-4-ethyl-8-fluoro-4-hydroxy-3,14-dioxo-3,4,12,14-tetrahydro-1H-pyrano[3,4′:6,7]indolizino[1,2-b]quinolin-11-yl)methyl carbamate (Compound 169)

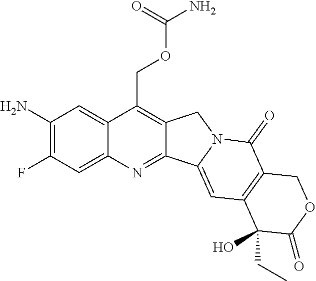 [SMILES: CC[C@@]1(O)C(=O)OCc2c1cc1n(c2=O)Cc2c-1nc1cc(F)c(N)cc1c2COC(N)=O]

The title compound was prepared according to General Procedure 6 starting from Compound 3.29 (5.1 mg) to give the title compound as yellow powder (TFA salt, 3.8 mg, 73% yield).

LC/MS: Calc'd m/z=455.1 for C₂₂H₁₉FN₄O₆ found [M+H]⁺=455.2.

¹H NMR (300 MHz, DMSO-d6) δ 7.79 (d, J=12.4 Hz, 1H), 7.29 (d, J=9.7 Hz, 1H), 7.21 (s, 1H), 7.0-6.50 (m, 2H), 5.45 (s, 2H), 5.40 (s, 2H), 5.33 (s, 2H), 1.95-1.77 (m, 2H), 0.87 (t, J=7.3 Hz, 3H).

### 3.31: ((S)-9-amino-4-ethyl-8-fluoro-4-hydroxy-11-(methoxymethyl)-1,12-dihydro-14H-pyrano[3′,4′:6,7]indolizino[1,2-b]quinoline-3,14(4H)-dione (Compound 155)

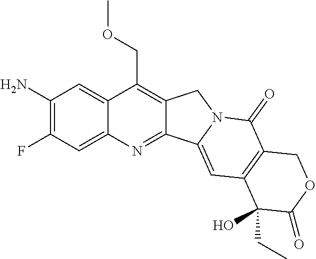 [SMILES: CC[C@@]1(O)C(=O)OCc2c1cc1n(c2=O)Cc2c-1nc1cc(F)c(N)cc1c2COC]

In a 50 mL flask containing Compound 3.5 (30 mg) was added MeOH/Dioxane (1:1) (9.8 mL) and sulfuric acid (0.73 mL). The reaction mixture was then stirred at reflux for 24 h. The reaction mixture was concentrated, poured into water (30 mL), and extracted with DCM (3×50 mL). The organic phases were combined and dried over MgSO₄. Preparative HPLC purification was accomplished as described in General Procedure 9, eluting with a 25 to 40% CH₃CN/H₂O+0.1% TFA gradient to give the title compound as a dark orange solid (5.1 mg, 16% yield).

LC/MS: Calc'd m/z=426.1 for C₂₂H₂₀FN₃O₅ found [M+H]⁺=426.2.

¹H NMR (300 MHz, DMSO-d6) δ 7.75 (d, J=12.3 Hz, 1H), 7.24 (d, J=9.9 Hz, 1H), 7.20 (s, 1H), 6.47 (s, 1H), 6.30-5.92 (brs, 2H), 5.40 (s, 2H), 5.24 (s, 2H), 4.93 (s, 2H), 3.43 (s, 3H), 1.95-1.75 (m, 2H), 0.87 (t, J=7.3 Hz, 3H).

### 3.32: (4S)-9-amino-4-ethyl-8-fluoro-4-hydroxy-11-(((1R,5S)-6-hydroxy-3-azabicyclo[3.1.1]heptan-3-yl)methyl)-1,12-dihydro-14H-pyrano[3′,4′:6,7]indolizino[1,2-b]quinoline-3,14(4H)-dione (Compound 158)

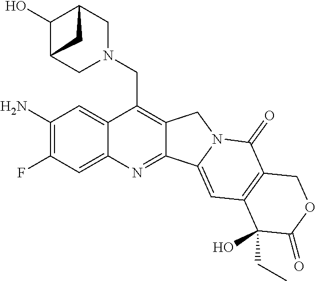 [SMILES: CC[C@@]1(O)C(=O)OCc2c1cc1n(c2=O)Cc2c-1nc1cc(F)c(N)cc1c2CN1C[C@H]2C[C@@H](C1)C2O]

In a 5 mL conical flask containing Compound 3.6 (15 mg) was added dichloromethane (0.6 mL) followed by 3-azabicyclo[3.1.1]heptan-6-ol (10 mg) and acetic acid (7.6 μL). The reaction was stirred at room temperature and sodium triacetoxyborohydride (9.4 mg) was added. After 1 hour at room temperature, the reaction was quenched by addition of water+0.1% TFA and diluted with DMF. The reaction mixture was then partially evaporated. Preparative HPLC purification was accomplished as described in General Procedure 9, eluting with a 20 to 50% CH₃CN/H₂O+0.1% TFA gradient to give the Boc-protected title compound as a yellow powder. Deprotection was performed according to General Procedure 6, and the obtained residue was purified by preparative HPLC purification as described in General Procedure 9, eluting with a 20 to 50% CH₃CN/H₂O+0.1% TFA gradient to give the title compound as yellow powder (TFA salt, 7.1 mg, 39% yield).

LC/MS: Calc'd m/z=507.2 for C₂₇H₂₇FN₄O₅ found [M+H]⁺=507.4.

¹H NMR (300 MHz, DMSO-d6) δ 7.85 (d, J=12.1 Hz, 1H), 7.46 (d, J=9.4 Hz, 1H), 7.23 (s, 1H), 6.64-5.85 (m, 3H), 5.60-5.25 (m, 4H), 4.85 (s, 1H), 4.10-3.95 (m, 1H), 3.68 (s, 2H), 2.45-2.33 (m, 2H), 1.96-1.72 (m, 2H), 0.87 (t, J=7.3 Hz, 3H).

### 3.33: (S)-9-amino-4-ethyl-8-fluoro-11-((3-fluoro-3-(hydroxymethyl)azetidin-1-yl)methyl)-4-hydroxy-1,12-dihydro-14H-pyrano[3′,4′:6,7]indolizino[1,2-b]quinoline-3,14(4H)-dione (Compound 159)

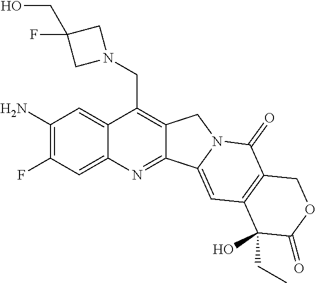 [SMILES: CC[C@@]1(O)C(=O)OCc2c1cc1n(c2=O)Cc2c-1nc1cc(F)c(N)cc1c2CN1CC(F)(CO)C1]

In a 5 mL conical flask containing Compound 3.6 (15 mg) was added dichloromethane (0.6 mL) followed by (3-fluoroazetidin-3-yl)methanol (9.3 mg) and acetic acid (7.6 μL). The reaction was stirred at room temperature and sodium triacetoxyborohydride (9.4 mg) was added. After 1 hour at room temperature, the reaction was quenched by addition of water+0.1% TFA, diluted with DMF, then partially evaporated. Preparative HPLC purification was accomplished as described in General Procedure 9, eluting with a 20 to 50% CH₃CN/H₂O+0.1% TFA gradient to give the Boc-protected title compound as a yellow powder. Deprotection was then performed according to General Procedure 6. The obtained residue was purified by preparative HPLC purification as described in General Procedure 9, eluting with a 20 to 50% CH₃CN/H₂O+0.1% TFA gradient to give the title compound as yellow powder (TFA salt, 1.8 mg, 10% yield).

LC/MS: Calc'd m/z=499.2 for C₂₅H₂₄F₂N₄O₅ found [M+H]⁺=499.4.

¹H NMR (300 MHz, DMSO-d6) δ 7.82 (d, J=12.4 Hz, 1H), 7.45 (d, J=9.5 Hz, 1H), 7.21 (s, 1H), 5.45-5.33 (m, 4H), 3.75-3.61 (m, 2H), 1.93-1.78 (m, 2H), 0.87 (t, J=7.3 Hz, 3H).

### 3.34: tert-butyl-(S)-(4-ethyl-8-fluoro-4-hydroxy-11-((methylamino)methyl)-3,14-dioxo-3,4,12,14-tetrahydro-1H-pyrano[3,4′:6,7]indolizino[1,2-b]quinolin-9-yl)carbamate (Compound 3.34)

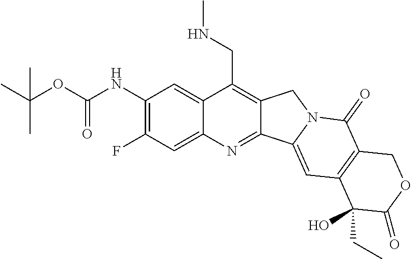 [SMILES: CC[C@@]1(O)C(=O)OCc2c1cc1n(c2=O)Cc2c-1nc1cc(F)c(NC(=O)OC(C)(C)C)cc1c2CNC]

To a stirring solution of Compound 3.9 (210 mg) in DMF (5 mL) was added sodium iodide (5.9 mg) followed by methylammonium chloride (107 mg). The reaction mixture was then stirred at room temperature overnight. Reverse phase purification was accomplished as described in General Procedure 9 using a 30 g C₁₈ column and eluting with a 10 to 65% CH₃CN/H₂O+0.1% TFA gradient to give the title compound as a yellow solid (15.0 mg, 7.2% yield).

LC/MS: Calc'd m/z=524.2 for C₂₇H₂₉FN₄O₆, found [M+H]⁺=525.4.

### 3.35: (S)-N-((9-amino-4-ethyl-8-fluoro-4-hydroxy-3,14-dioxo-3,4,12,14-tetrahydro-1H-pyrano[3′,4′: 6,7]indolizino[1,2-b]quinolin-11-yl)methyl)-2-hydroxy-N-methylacetamide (Compound 165)

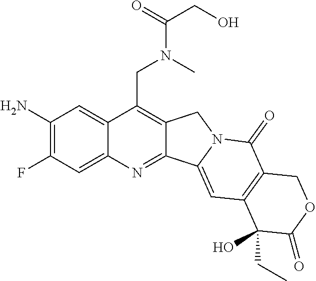 [SMILES: CC[C@@]1(O)C(=O)OCc2c1cc1n(c2=O)Cc2c-1nc1cc(F)c(N)cc1c2CN(C)C(=O)CO]

The Boc-protected version of the title compound was prepared according to General Procedure 2 starting from Compound 3.34 (6.4 mg) and glycolic acid. Preparative HPLC purification was accomplished as described in General Procedure 9, eluting with a 20 to 50% CH₃CN/H₂O+0.1% TFA gradient. Deprotection was then performed according to General Procedure 6 to give the title compound as yellow powder (TFA salt, 2.0 mg, 28% yield).

LC/MS: Calc'd m/z=482.2 for C₂₄H₂₃FN₄O₆, found [M+H]⁺=483.2.

¹H NMR (300 MHz, DMSO-d6) δ 7.79 (d, J=12.3 Hz, 1H), 7.27 (d, J=9.5 Hz, 1H), 7.22 (s, 1H), 6.48 (s, 1H), 6.28-6.02 (m, 2H), 5.40 (s, 2H), 5.21 (s, 2H), 5.06-4.93 (m, 2H), 4.18 (s, 2H), 2.80 (s, 3H), 1.92-1.78 (m, 2H), 0.87 (t, J=7.3 Hz, 3H).

### 3.36: (S)-N-((9-amino-4-ethyl-8-fluoro-4-hydroxy-3,14-dioxo-3,4,12,14-tetrahydro-1H-pyrano[3,4′:6,7]indolizino[1,2-b]quinolin-11-yl)methyl)-N-methylmethanesulfonamide (Compound 166)

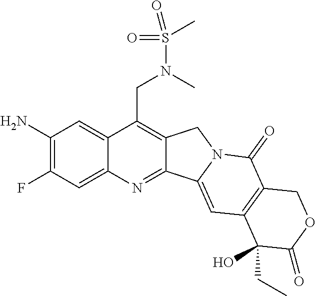 [SMILES: CC[C@@]1(O)C(=O)OCc2c1cc1n(c2=O)Cc2c-1nc1cc(F)c(N)cc1c2CN(C)S(C)(=O)=O]

The Boc-protected version of the title compound was prepared according to General Procedure 3 starting from Compound 3.34 (8.0 mg) and methanesulfonyl chloride. Preparative HPLC purification was accomplished as described in General Procedure 9, eluting with a 10 to 50% CH₃CN/H₂O+0.1% TFA gradient. Deprotection was then performed according to General Procedure 6 to give the title compound as yellow powder (TFA salt, 2.6 mg, 34% yield).

LC/MS: Calc'd m/z=502.1 for C₂₃H₂₃FN₄O₆S, found [M+H]⁺=503.2.

¹H NMR (300 MHz, DMSO-d6) δ 7.81 (d, J=12.3 Hz, 1H), 7.41 (d, J=9.4 Hz, 1H), 7.23 (s, 1H), 6.63-5.84 (m, 2H), 5.42 (s, 2H), 5.29 (s, 2H), 4.81-4.64 (m, 2H), 3.14 (s, 3H), 2.67 (s, 3H), 1.96-1.76 (m, 2H), 0.88 (t, J=7.3 Hz, 3H).

### 3.37: (S)-9-amino-4-ethyl-8-fluoro-4-hydroxy-11-(2-methoxyethyl)-1,12-dihydro-14H-pyrano[3′,4′:6,7]indolizino[1,2-b]quinoline-3,14(4H)-dione (Compound 170)

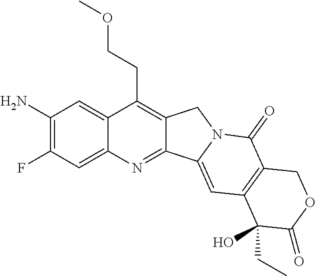 [SMILES: CC[C@@]1(O)C(=O)OCc2c1cc1n(c2=O)Cc2c-1nc1cc(F)c(N)cc1c2CCOC]

To a 10 mL round bottom flask containing Compound 3.4 (62.0 mg) was added water (0.89 mL), FeSO₄ (heptahydrate, 18.0 mg), and 3-methoxypropanal (113.0 mg). To the obtained suspension was added sulfuric acid (0.495 mL) dropwise while stirring at −15° C. in an ice salt bath. Hydrogen peroxide (0.118 mL) was then added dropwise. The mixture was stirred at −15° C. for 10 min and was then allowed to warm up to room temperature and stirred for 1 h. The reaction mixture was then diluted with water (30 mL) and the obtained suspension was extracted with DCM (3×30 mL). The organic phase was evaporated to dryness. Preparative HPLC purification was accomplished as described in General Procedure 9, eluting with a 25 to 45% CH₃CN/H₂O+0.1% TFA gradient to give the title compound as a dark orange solid (TFA salt, 3.1 mg, 4.4% yield).

LC/MS: Calc'd m/z=440.2 for C₂₃H₂₂FN₃O₅, found [M+H]⁺=440.2.

¹H NMR (300 MHz, DMSO-d6) δ 7.75 (d, J=12.4 Hz, 1H), 7.33 (d, J=9.4 Hz, 1H), 7.20 (s, 1H), 6.60-6.42 (m, 2H), 5.40 (s, 2H), 5.25 (s, 2H), 3.69 (t, J=6.5 Hz, 2H), 3.24 (s, 3H), 3.23 (t, J=6.5 Hz, 2H), 1.96-1.76 (m, 2H), 0.88 (t, J=7.3 Hz, 3H).

### 3.38: (S)-N-(4-ethyl-8-fluoro-4-hydroxy-3,14-dioxo-3,4,12,14-tetrahydro-1H-pyrano[3,4′:6,7]indolizino[1,2-b]quinolin-9-yl)acetamide (Compound 171)

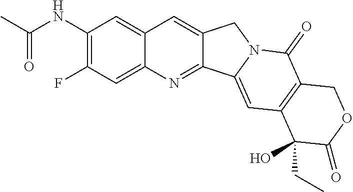 [SMILES: CC[C@@]1(O)C(=O)OCc2c1cc1n(c2=O)Cc2cc3cc(NC(C)=O)c(F)cc3nc2-1]

To a 25 mL round bottom flask containing acetic acid (0.071 mL) in dimethylformamide (0.69 mL) was added N-methylmorpholine (0.343 mL), HOAt (0.142 g), and HATU (0.435 g). After stirring at room temperature for 5 min, this solution was added to a 10 mL cone-shaped flask containing Compound 140 (0.127 g). This solution was stirred at room temperature for 24 h then directly purified by preparative HPLC as described in General Procedure 9, eluting with a 25 to 45% CH₃CN/H₂O+0.1% TFA gradient to give the title compound as a bright yellow powder (43.0 mg, 38% yield).

LC/MS: Calc'd m/z=424.1 for C₂₂H₁₈FN₃O₅, found [M+H]⁺=424.2.

¹H NMR (300 MHz, DMSO-d6) δ 10.13 (s, 1H), 8.73 (d, J=8.5 Hz, 1H), 8.61 (s, 1H), 7.96 (d, J=912.1 Hz, 1H), 7.29 (s, 1H), 6.60-6.42 (m, 2H), 5.41 (s, 2H), 5.21 (s, 2H), 2.20 (s, 3H), 1.96-1.76 (m, 2H), 0.88 (t, J=7.3 Hz, 3H).

### 3.39: tert-butyl (5-formyl-2-methoxy-4-nitrophenyl)carbamate (Compound 3.39)

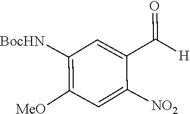 [SMILES: COc1cc([N+](=O)[O-])c(C=O)cc1NC(=O)OC(C)(C)C]

To a solution of Compound 3.2 (1.3 g, 1.0 eq.) in MeOH (12 mL) at 0° C. was added sodium methoxide (0.74 g, 3.0 eq.). After the addition was complete, the ice bath was removed and the resulting solution was stirred at room temperature for 72 h. The reaction was then quenched with ice water (50 mL) and extracted with DCM (3×100 mL). The combined organic layers were washed with brine (50 mL), dried over sodium sulfate, filtered, and concentrated in vacuo to yield the title compound as an orange solid (1.2 g, 89% yield).

LC/MS: Calc'd m/z=296.10 for C₁₃H₁₆N₂O₆, found [M+H]⁺=297.1.

¹H NMR (300 MHz, MeOD) δ 10.29 (s, 1H), 8.61 (s, 1H), 7.73 (s, 1H), 4.08 (s, 3H), 1.57 (s, 9H)

### 3.40: tert-butyl (4-amino-5-formyl-2-methoxyphenyl)carbamate (Compound 3.40)

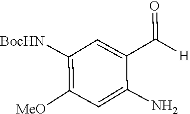 [SMILES: COc1cc(N)c(C=O)cc1NC(=O)OC(C)(C)C]

To a solution of Compound 3.39 (500 mg, 1 eq.) in MeOH (10 mL) and H₂O (1 mL) was added B₂(OH)₄ (454 mg, 3 eq.). The resulting mixture was cooled to 0° C. and an aqueous 5M NaOH solution (2.75 mL) was added with stirring over the course of 10 min. The reaction mixture was stirred for an additional 5 min then quenched by pouring the solution into ice (40 mL). The resulting mixture was extracted with DCM (3×50 mL), dried over sodium sulfate, filtered, and concentrated in vacuo. Flash purification was accomplished as described in General Procedure 9, using a 25 g silica column and eluting with 10 to 50% hexanes/EtOAc to give the title compound as an orange solid (386 mg, 86%).

LC/MS: Calc'd m/z=266.1 for C₁₃H₁₈N₂O₄, found [M+H]⁺=297.2.

### 3.41: (S)-9-amino-4-ethyl-8-fluoro-4-hydroxy-1,12-dihydro-14H-pyrano[3′,4′:6,7]indolizine o[1,2-b]quinoline-3,14(4H)-dione (Compound 168)

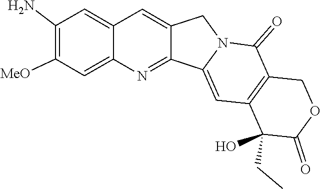 [SMILES: CC[C@@]1(O)C(=O)OCc2c1cc1n(c2=O)Cc2cc3cc(N)c(OC)cc3nc2-1]

A mixture of Compound 3.40 (385 mg, 1.0 eq.) and (S)-4-ethyl-4-hydroxy-7,8-dihydro-1H-pyrano[3,4-f]indolizine-3,6,10(4H)-trione (362 mg, 0.95 eq.), TsOH (monohydrate, 25 mg, 0.1 eq.) and toluene (30 mL) in a 250 mL round bottom flask equipped with a Dean-Stark apparatus was stirred at 110° C. for 2 h. The reaction mixture was then cooled to 25° C. and concentrated in vacuo. Purification was accomplished as described in General Procedure 9, using a 25 g silica column and eluting with a 0 to 50% DCM/MeOH gradient to provide the Boc-protected intermediate as a red solid. This material was then deprotected according to General Procedure 6 followed by preparative HPLC purification as described in General Procedure 9, eluting with a 20 to 65% CH₃CN/H₂O+0.1% TFA gradient to give the title compound as a red solid (TFA salt, 300 mg, 53% yield).

LC/MS: Calc'd m/z=393.2 for C₂₁H₁₉N₃O₅, found [M+H]⁺=393.2.

¹H NMR (300 MHz, MeOD) δ 8.27 (s, 1H), 7.62 (s, 1H), 7.42 (s, 1H), 7.11 (s, 1H), 5.61 (d, J=16.2 Hz, 1H), 5.38 (d, J=16.2 Hz, 1H), 5.24 (s, 2H), 4.11 (s, 3H), 2.06-1.91 (m, 2H), 1.04 (t, J=7.4 Hz, 3H).

### 3.42: 5-bromo-2-nitro-4-(trifluoromethyl)benzaldehyde (Compound 3.42)

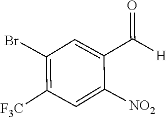 [SMILES: O=Cc1cc(Br)c(C(F)(F)F)cc1[N+](=O)[O-]]

To a stirring solution of HNO₃ (2.0 g, 1.4 mL, 67% purity, 2 eq.) in H₂SO₄ (8 mL) at 0° C. was added 3-bromo-4-(trifluoromethyl)benzaldehyde (4 g, 1 eq.). After the addition was complete, the ice bath was removed, and the reaction was allowed to stir for 5 h at room temperature. The mixture was poured into ice (100 mL) and the precipitate extracted with DCM (3×100 mL). The combined organic fractions were then washed with brine (50 mL), dried over Na₂SO₄, and concentrated in vacuo to yield the title compound as a yellow solid (4.4 g, 93% yield).

LC/MS: Calc'd m/z=296.90 for C₈H₃BrF₃NO₃, found [M+H]⁺=298.0.

¹H NMR (300 MHz, MeOD) δ 10.35 (s, 1H), 8.29 (s, 1H), 8.23 (s, 1H).

### 3.43: tert-butyl (5-formyl-4-nitro-2-(trifluoromethyl)phenyl)carbamate (Compound 3.43)

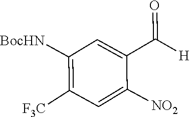 [SMILES: CC(C)(C)OC(=O)Nc1cc(C=O)c([N+](=O)[O-])cc1C(F)(F)F]

A mixture of Compound 3.42 (800 mg, 1 eq.), tert-butyl carbamate (378 mg, 1.2 eq.), Cs₂CO₃ (1.7 g, 2 eq.), Pd₂(dba)₃ (122 mg, 0.05 eq.), and dicyclohexyl[2′,4′,6′-tris(propan-2-yl)[1,1′-biphenyl]-2-yl]phosphane (XPhos) (256 mg, 0.2 eq.) in toluene (5 mL) was degassed and purged with N₂ for three cycles. The mixture was then stirred at 90° C. for 15 h under N₂ atmosphere. The reaction mixture was diluted with H₂O (25 mL) and extracted with EtOAc (3×50 mL). The combined organic layers were washed with brine (2×25 mL), dried over sodium sulfate, filtered, and concentrated under reduced pressure. Flash purification was achieved according to General Procedure 9, using a 25 g silica column and eluting with 0 to 25% DCM/MeOH to give the title compound as an orange solid (750 mg, 84% yield).

LC/MS: Calc'd m/z=334.1 for C₁₃H₁₃FN₂O₅, found [M−H]⁻=333.1.

### 3.44: tert-butyl (4-amino-5-formyl-2-(trifluoromethyl)phenyl)carbamate (Compound 3.44)

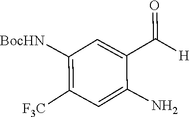 [SMILES: CC(C)(C)OC(=O)Nc1cc(C=O)c(N)cc1C(F)(F)F]

To a solution of Compound 3.43 (750 mg, 1 eq.) in MeOH (16 mL) and H₂O (1.6 mL) was added B₂(OH)₄ (603 mg, 3 eq.). The resulting mixture was cooled to 0° C. and an aqueous 5M NaOH solution (2.75 mL) was added with stirring over the course of 10 min. The reaction mixture was stirred for an additional 5 min then quenched by pouring the solution into ice (50 mL). The resulting mixture was extracted with DCM (3×75 mL), dried over sodium sulfate, filtered, and concentrated in vacuo. Flash purification was accomplished as described in General Procedure 9, using a 25 g silica column and eluting with 10 to 50% hexanes/EtOAc to give the title compound as an orange solid (460 mg, 67%).

LC/MS: Calc'd m/z=304.1 for C₁₃H₁₅F₃N₂O₃, found [M+H]⁺=305.2

### 3.45: (S)-9-amino-4-ethyl-4-hydroxy-8-(trifluoromethyl)-1,12-dihydro-14H-pyrano[3′,4′:6,7]indolizino [1,2-b]quinoline-3,14(4H)-dione (Compound 167)

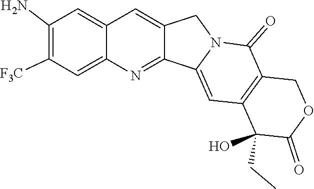 [SMILES: CC[C@@]1(O)C(=O)OCc2c1cc1n(c2=O)Cc2cc3cc(N)c(C(F)(F)F)cc3nc2-1]

A mixture of Compound 3.44 (460 mg, 1 eq.) and (S)-4-ethyl-4-hydroxy-7,8-dihydro-1H-pyrano[3,4-f]indolizine-3,6,10(4H)-trione (378 mg, 0.95 eq.), TsOH (monohydrate, 26 mg, 0.1 eq.) and toluene (35 mL) in a 250 mL round bottom flask equipped with a Dean-Stark apparatus was stirred at 110° C. for 2 h. The reaction mixture was then cooled to 25° C. and concentrated in vacuo. Purification was accomplished as described in General Procedure 9, using a 25 g silica column and eluting with a 0 to 50% DCM/MeOH gradient to provide the Boc-protected intermediate as a red solid. This material was then deprotecting according to General Procedure 6 followed by preparative HPLC purification as described in General Procedure 9, eluting with a 20 to 65% CH₃CN/H₂O+0.1% TFA gradient to give the title compound as a yellow solid (6.2 mg, 48%).

LC/MS: Calc'd m/z=431.1 for C₂₁H₁₆F₃N₃O₄, found [M+H]⁺=432.2.

¹H NMR (300 MHz, MeOD) δ 8.29 (s, 1H), 8.27 (s, 1H), 7.59 (s, 1H), 7.24 (s, 1H), 5.59 (d, J=16.3 Hz, 1H), 5.39 (d, J=16.3 Hz, 1H), 5.28 (s, 2H), 2.00-1.89 (m, 2H), 1.03 (t, J=7.4 Hz, 3H).

### Example 4: Preparation of Drug-Linkers

### 4.1: 2,5-dioxopyrrolidin-1-yl (((9H-fluoren-9-yl)methoxy)carbonyl)glycylglycinate (Compound 4.1)

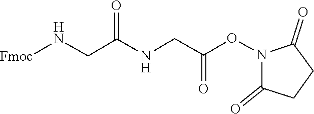

The title compound was prepared according to the procedure described in Chinese Patent Publication No. CN105218644.

### 4.2: (((9H-fluoren-9-yl)methoxy)carbonyl)glycylglycyl-L-phenylalanine (Fmoc-GGF-OH; Compound 4.2)

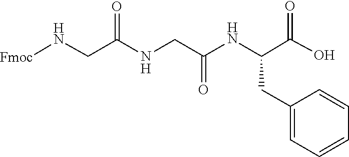

To L-phenylalanine (965 mg) in acetonitrile (10 mL) and dimethyl formamide (0.5 mL) was added DIPEA (1.51 mL) then Compound 4.1 (1.3 g). After 1 h the reaction was concentrated to dryness. Flash purification was accomplished as described in General Procedure 9, eluting with a 10 to 50% CH₃CN/H₂O+0.1% TFA gradient to provide the title compound as a white solid (430 mg, 30% yield).

LC/MS: Calc'd m/z=501.2 for C₂₈H₇₁N₃O₆S, found [M+H]⁺=502.4.

¹H NMR (300 MHz, DMSO) δ 8.16 (d, J=8.1 Hz, 1H), 8.04 (t, J=5.8 Hz, 1H), 7.90 (d, J=7.5 Hz, 2H), 7.72 (d, J=7.4 Hz, 2H), 7.59 (t, J=6.0 Hz, 1H), 7.54-7.39 (m, 2H), 7.33 (t, J=7.6 Hz, 2H), 7.28-7.13 (m, 5H), 4.44 (td, J=8.5, 5.1 Hz, 1H), 4.33-4.13 (m, 3H), 3.83-3.59 (m, 4H), 3.06 (dd, J=13.7, 5.1 Hz, 1H), 2.88 (dd, J=13.8, 9.0 Hz, 1H).

### 4.3: 2,3,5,6-tetrafluorophenyl 3-(2-(2-(2-(2,5-dioxo-2,5-dihydro-1H-pyrrol-1-yl)ethoxy) ethoxy)ethoxy)propanoate (MT-OTfp; Compound 4.3)

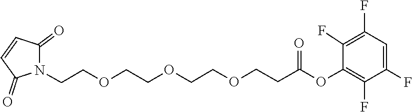 [SMILES: O=C(CCOCCOCCOCCN1C(=O)C=CC1=O)Oc1c(F)c(F)cc(F)c1F]

The title compound was prepared according to the procedure described in International (PCT) Publication No. WO 2017/054080.

### 4.4: (3-(2-(2-(2-(2,5-dioxo-2,5-dihydro-1H-pyrrol-1-yl)ethoxy)ethoxy)ethoxy)propanoyl) glycylglycyl-L-phenylalanine (Compound 4.4)

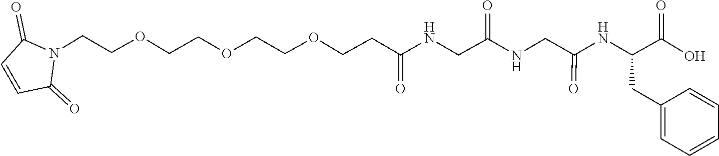 [SMILES: O=C(CCOCCOCCOCCN1C(=O)C=CC1=O)NCC(=O)NCC(=O)N[C@@H](Cc1ccccc1)C(=O)O]

To a solution of Compound 4.3 (1.61 g, 3.58 mmol) in DMF (35 mL) was added Gly-Gly-Phe (1 g, 3.58 mmol) as a single portion followed by iPr₂NEt (1.25 mL, 7.2 mmol). This solution was stirred at room temperature for 1 h, then evaporated to dryness. Purification was accomplished as described in General Procedure 9 using a 30 g C18 flash column and eluting with a 10 to 90% CH₃CN/H₂O+0.1% TFA gradient to provide the title compound as a white solid (400 mg, 20% yield).

LC/MS: Calc'd m/z=562.6 for C₂₆H₃₄N₄₀₁₀, found [M−H]⁻=561.5.

¹H NMR (300 MHz, CDCl₃) δ 7.60 (t, J=5.6 Hz, 2H), 7.41 (d, J=7.7 Hz, 1H), 7.32-7.07 (m, 5H), 6.70 (s, 2H), 6.33-6.07 (m, 3H), 4.72 (td, J=7.6, 5.3 Hz, 1H), 4.12-3.78 (m, 4H), 3.72 (ddd, J=15.2, 6.9, 4.8 Hz, 5H), 3.60 (dd, J=11.6, 6.1 Hz, 10H), 3.12 (ddd, J=48.2, 14.0, 6.5 Hz, 2H), 2.52 (d, J=11.7 Hz, 2H).

### 4.5: (S)-11-benzyl-1-(9H-fluoren-9-yl)-3,6,9,12,15-pentaoxo-2-oxa-4,7,10,13,16-pentaazaheptadecan-17-yl acetate (Compound 4.5)

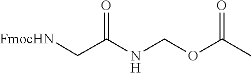

The title compound was prepared according to the procedure described in US Patent Publication No. US 2017/021031.

### 4.6: (S)-11-benzyl-1-(9H-fluoren-9-yl)-3,6,9,12,15-pentaoxo-2-oxa-4,7,10,13,16-pentaazaheptadecan-17-yl acetate (Compound 4.6)

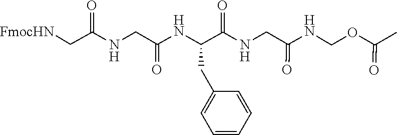

The title compound was prepared according to the procedure described in US Patent Publication No. US 2017/021031 using Fmoc-GGFGG-OH as the starting peptide.

### 4.7. tert-butyl (2-((2-(((S)-1-((2-((4-((4-(((S)-4-ethyl-8-fluoro-4-hydroxy-9-methyl-3,14-dioxo-3,4,12,14-tetrahydro-1H-pyrano[3,4′:6,7]indolizino[1,2-b]quinolin-11-yl)methyl)piperazin-1-yl)sulfonyl)phenyl)amino)-2-oxoethyl)amino)-1-oxo-3-phenylpropan-2-yl)amino)-2-oxoethyl)amino)-2-oxoethyl)carbamate (Compound 4.7)

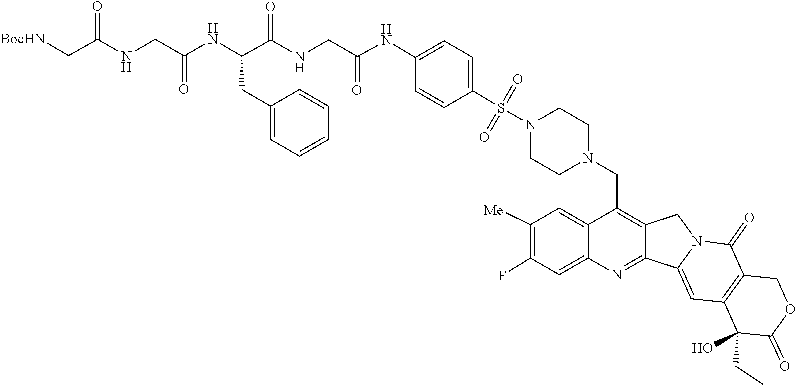 [SMILES: CC[C@@]1(O)C(=O)OCc2c1cc1n(c2=O)Cc2c-1nc1cc(F)c(C)cc1c2CN1CCN(S(=O)(=O)c2ccc(NC(=O)CNC(=O)[C@H](Cc3ccccc3)NC(=O)CNC(=O)CNC(=O)OC(C)(C)C)cc2)CC1]

The title compound was prepared according to General Procedure 7 starting from Compound 104 (20 mg). Preparative HPLC purification was accomplished as described in General Procedure 9, eluting with a 10 to 60% CH₃CN/H₂O+0.1% TFA gradient to provide the title compound as a white solid (14 mg, 42% yield).

LC/MS: Calc'd m/z=1051.4 for C₅₂H₅₈N₉O₁₂S, found [M+H]⁺=1052.6.

### 4.8: (S)-2-(1-(2,5-dioxo-2,5-dihydro-1H-pyrrol-1-yl)-12,15-dioxo-3,6,9-trioxa-13,16-diazaoctadecan-18-amido)-N-(2-((4-((4-(((S)-4-ethyl-8-fluoro-4-hydroxy-9-methyl-3,14-dioxo-3,4,12,14-tetrahydro-1H-pyrano[3,4′:6,7]indolizino[1,2-b]quinolin-11-yl)methyl)piperazin-1-yl)sulfonyl)phenyl)amino)-2-oxoethyl)-3-phenylpropanamide (MT-GGFG-Compound 104)

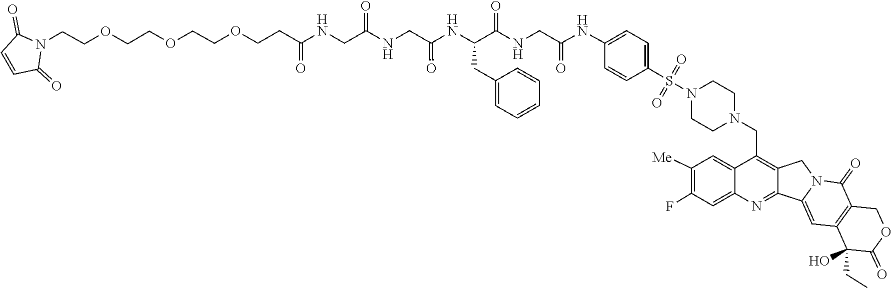 [SMILES: CC[C@@]1(O)C(=O)OCc2c1cc1n(c2=O)Cc2c-1nc1cc(F)c(C)cc1c2CN1CCN(S(=O)(=O)c2ccc(NC(=O)CNC(=O)[C@H](Cc3ccccc3)NC(=O)CNC(=O)CNC(=O)CCOCCOCCOCCN3C(=O)C=CC3=O)cc2)CC1]

The title compound was prepared according to Procedure 6 followed by Procedure 8 starting from Compound 4.7 (14 mg). Preparative HPLC purification was accomplished as described in General Procedure 9, eluting with a 10 to 60% CH₃CN/H₂O+0.1% TFA gradient to provide the title compound as a white solid (9.1 mg, 56% yield).

LC/MS: Calc'd m/z=1234.4 for C₆₀H₆₇FN₁₀O₁₆S, found [M+H]⁺=1235.8.

### 4.9: tert-butyl (2-((2-(((S)-1-((2-((4-(4-(((S)-4-ethyl-8-fluoro-4-hydroxy-9-methyl-3,14-dioxo-3,4,12,14-tetrahydro-1H-pyrano[3,4′:6,7]indolizino[1,2-b]quinolin-11-yl)methyl)piperazin-1-yl)phenyl)amino)-2-oxoethyl)amino)-1-oxo-3-phenylpropan-2-yl)amino)-2-oxoethyl)amino)-2-oxoethyl)carbamate (Compound 4.9)

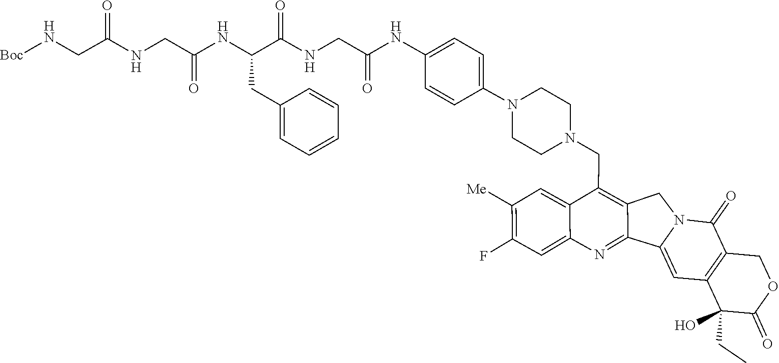 [SMILES: CC[C@@]1(O)C(=O)OCc2c1cc1n(c2=O)Cc2c-1nc1cc(F)c(C)cc1c2CN1CCN(c2ccc(NC(=O)CNC(=O)[C@H](Cc3ccccc3)NC(=O)CNC(=O)CNC(=O)OC(C)(C)C)cc2)CC1]

The title compound was prepared according to General Procedure 7 starting from Compound 108 (12 mg). Preparative HPLC purification was accomplished as described in General Procedure 9, eluting with a 10 to 60% CH₃CN/H₂O+0.1% TFA gradient to provide the title compound as a white solid (13 mg, 62% yield).

LC/MS: Calc'd m/z=987.4 for C₅₂H₅₈N₉O₁₀, found [M+H]⁺=988.6.

### 4.10: (S)-2-(1-(2,5-dioxo-2,5-dihydro-1H-pyrrol-1-yl)-12,15-dioxo-3,6,9-trioxa-13,16-diazaoctadecan-18-amido)-N-(2-((4-(4-(((S)-4-ethyl-8-fluoro-4-hydroxy-9-methyl-3,14-dioxo-3,4,12,14-tetrahydro-1H-pyrano[3,4′:6,7]indolizino[1,2-b]quinolin-11-yl)methyl)piperazin-1-yl)phenyl)amino)-2-oxoethyl)-3-phenylpropanamide (MT-GGFG-Compound 108)

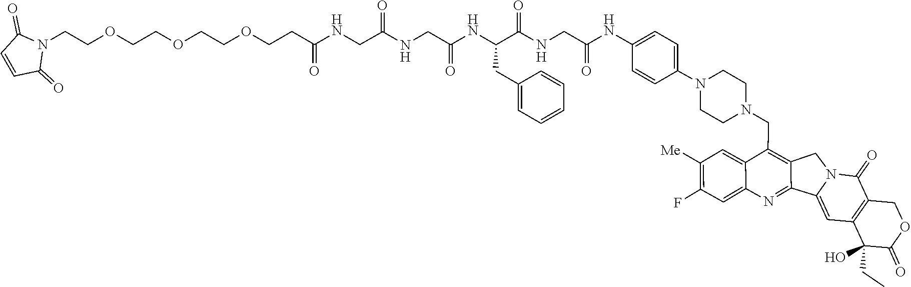 [SMILES: CC[C@@]1(O)C(=O)OCc2c1cc1n(c2=O)Cc2c-1nc1cc(F)c(C)cc1c2CN1CCN(c2ccc(NC(=O)CNC(=O)[C@H](Cc3ccccc3)NC(=O)CNC(=O)CNC(=O)CCOCCOCCOCCN3C(=O)C=CC3=O)cc2)CC1]

The title compound was prepared according to Procedure 6 followed by Procedure 8 starting from Compound 4.9 (13 mg). Preparative HPLC purification was accomplished as described in General Procedure 9, eluting with a 10 to 50% CH₃CN/H₂O+0.1% TFA gradient to provide the title compound as a white solid (3.1 mg, 20% yield).

LC/MS: Calc'd m/z=1170.5 for C₆₀H₆₇FN₁₀O₁₄, found [M+H]⁺=1171.6.

### 4.11: (9H-fluoren-9-yl)methyl (S)-(1-(4-ethyl-8-fluoro-4-hydroxy-9-methyl-3,14-dioxo-3,4,12,14-tetrahydro-1H-pyrano[3,4′:6,7]indolizino[1,2-b]quinolin-11-yl)-3,10-dioxo-7-oxa-2,4,9-triazaundecan-11-yl)carbamate (Compound 4.11)

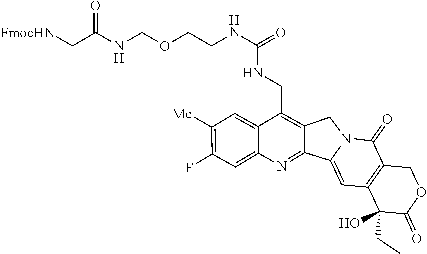

To a solution of Compound 1.2 (31 mg, 0.076 mmol) in DMF (750 uL) was added (9H-fluoren-9-yl)methyl (2-(((2-(((4-nitrophenoxy)carbonyl)amino)ethoxy)methyl)amino)-2-oxoethyl)carbamate (41 mg, 0.076 mmol) followed by iPr₂NEt (26 uL, 0.15 mmol). This solution was stirred at room temperature for 2 h and then applied directly to 12 g C₁₈ column. Purification was accomplished as described in General Procedure 9, eluting with a 10 to 100% CH₃CN/H₂O+0.1% TFA gradient to provide the title compound as a white solid (21 mg, 35% yield).

LC/MS: Calc'd m/z=804.87 for C₄₃H₄₁FN₆O₉, found [M+H]⁺=805.6.

### 4.12: (S)-2-(1-(2,5-dioxo-2,5-dihydro-1H-pyrrol-1-yl)-12,15-dioxo-3,6,9-trioxa-13,16-diazaoctadecan-18-amido)-N-(1-((S)-4-ethyl-8-fluoro-4-hydroxy-9-methyl-3,14-dioxo-3,4,12,14-tetrahydro-1H-pyrano[3,4′:6,7]indolizino[1,2-b]quinolin-11-yl)-3,10-dioxo-7-oxa-2,4,9-triazaundecan-11-yl)-3-phenylpropanamide (MT-GGFG-AM-Compound 136)

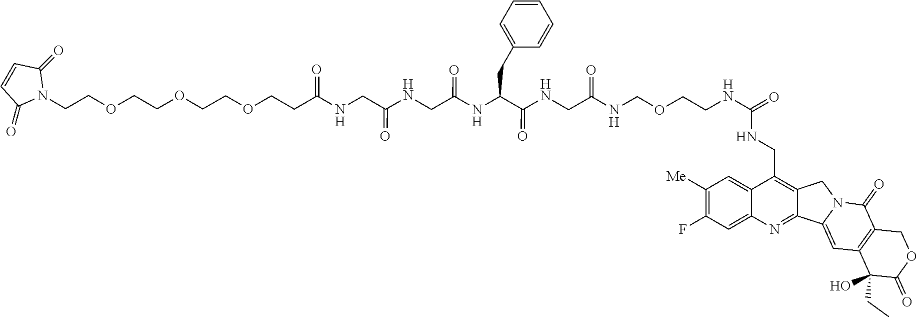 [SMILES: CC[C@@]1(O)C(=O)OCc2c1cc1n(c2=O)Cc2c-1nc1cc(F)c(C)cc1c2CNC(=O)NCCOCNC(=O)CNC(=O)[C@H](Cc1ccccc1)NC(=O)CNC(=O)CNC(=O)CCOCCOCCOCCN1C(=O)C=CC1=O]

Compound 4.11 (21 mg, 0.026 mmol) was taken up in a 10% solution of piperidine in DMF (1 mL) and stirred for 10 min. The piperidine solution was evaporated, the resulting residue was redissolved in DMF (5 mL), and then evaporated to dryness once more. To this residue was added DMF (50 uL) and DCM (450 uL) followed by Compound 4.4 (15 mg, 0.026 mmol), NMM (10 uL) and HATU (10 mg, 0.026 mmol). Preparative HPLC purification was accomplished as described in General Procedure 9, eluting with a 30 to 60% CH₃CN/H₂O+0.1% TFA gradient to provide the title compound as a yellow solid (7.6 mg, 26% yield).

LC/MS: Calc'd m/z=1127.1 for C₅₄H₆₃FN₁₀O₁₆, found [M+H]⁺=1128.2.

### 4.13: (9H-fluoren-9-yl)methyl (2-(((2-(chlorosulfonyl)ethoxy)methyl)amino)-2-oxoethyl)carbamate (Compound 4.13)

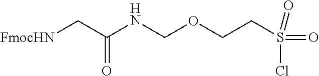

To a solution of Compound 4.5 (50 mg, 0.14 mmol), in DCM (800 uL) was added 2-hydroxyethane-1-sulfonyl chloride (100 mg, 0.7 mmol) followed by TFA (200 uL). This solution was stirred at room temperature for 30 min then evaporated to dryness. Purification was accomplished as described in General Procedure 9, using a 10 g silica column and eluting with a 10 to 100% EtOAc/hexanes gradient to provide the title compound as a clear film (31 mg, 50% yield).

LC/MS: Calc'd m/z=452.1 for C₂₀H₂₁ClN₂O₆S, found [M+Na]⁺=472.9

### 4.14: (9H-fluoren-9-yl)methyl (S)-(2-(((2-(N-((4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)sulfamoyl)ethoxy)methyl)amino)-2-oxoethyl)carbamate (Compound 4.14)

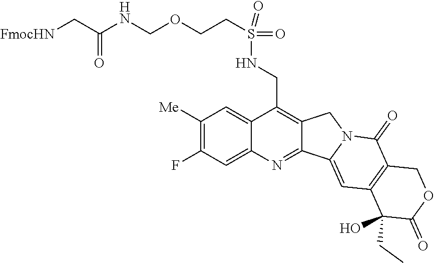

The title compound was prepared as described in General Procedure 3, using Compound 1.2 (28 mg, 0.07 mmol) and Compound 4.13 (31 mg, 0.07 mmol). Purification was accomplished as described in General Procedure 9, using a 12 g C₁₈ column and eluting with a 10 to 100% CH₃CN/H₂O+0.1% TFA gradient to provide the title compound as a yellow solid (22 mg, 39% yield).

LC/MS: Calc'd m/z=825.9 for C₄₂H₄₀FN₅O₁₀S, found [M+H]⁺=826.7.

### 4.15: (S)-2-(1-(2,5-dioxo-2,5-dihydro-1H-pyrrol-1-yl)-12,15-dioxo-3,6,9-trioxa-13,16-diazaoctadecan-18-amido)-N-(2-(((2-(N-(((S)-4-ethyl-8-fluoro-4-hydroxy-9-methyl-3,14-dioxo-3,4,12,14-tetrahydro-1H-pyrano[3,4′:6,7]indolizino[1,2-b]quinolin-11-yl)methyl)sulfamoyl) ethoxy)methyl)amino)-2-oxoethyl)-3-phenylpropanamide (MT-GGFG-AM-Compound 129)

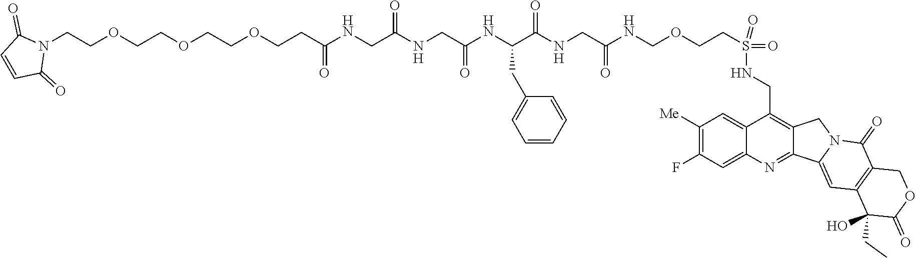 [SMILES: CC[C@@]1(O)C(=O)OCc2c1cc1n(c2=O)Cc2c-1nc1cc(F)c(C)cc1c2CNS(=O)(=O)CCOCNC(=O)CNC(=O)[C@H](Cc1ccccc1)NC(=O)CNC(=O)CNC(=O)CCOCCOCCOCCN1C(=O)C=CC1=O]

Compound 4.14 (22 mg, 0.027 mmol) was taken up in a 10% solution of piperidine in DMF (1 mL) and stirred for 10 min. The piperidine solution was evaporated, the resulting residue was redissolved in DMF (5 mL), and then evaporated to dryness once more. To this residue was added DMF (50 uL) and DCM (450 uL) followed by Compound 4.4 (30 mg, 0.053 mmol), NMM (10 uL) and HATU (18 mg, 0.048 mmol). Preparative HPLC purification was accomplished as described in General Procedure 9, eluting with a 30 to 60% CH₃CN/H₂O+0.1% TFA gradient to provide the title compound as a yellow solid (6.4 mg, 21% yield).

LC/MS: Calc'd m/z=1148.2 for C₅₃H₆₂FN₉O₁₇S, found [M+H]⁺=1148.6.

¹H NMR (300 MHz, MeOD) δ 8.60 (t, J=6.5 Hz, 1H), 8.36 (t, J=8.6 Hz, 2H), 8.13 (d, J=6.6 Hz, 1H), 7.77 (d, J=10.6 Hz, 1H), 7.65 (d, J=4.8 Hz, 1H), 7.28-7.00 (m, 6H), 6.80 (s, 2H), 5.69-5.50 (m, 3H), 5.45-5.33 (m, 2H), 4.44 (dd, J=8.7, 5.7 Hz, 1H), 3.96 (t, J=5.3 Hz, 2H), 3.90-3.76 (m, 5H), 3.76-3.57 (m, 7H), 3.09-2.81 (m, 3H), 2.61-2.45 (m, 5H), 2.04-1.90 (m, 2H), 1.03 (t, J=7.3 Hz, 3H).

### 4.16: (9H-fluoren-9-yl)methyl (S)-(2-(((morpholin-2-ylmethoxy)methyl)amino)-2-oxoethyl)carbamate (Compound 4.16)

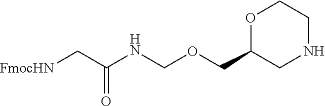

To a solution of Compound 4.5 (100 mg, 0.27 mmol) in DCM (800 uL) was added (S)-morpholin-2-ylmethanol (160 mg, 1.36 mmol) followed by TFA (200 uL). This solution was stirred at room temperature for 1 h then evaporated to dryness. Purification was accomplished as described in General Procedure 9, using a 12 g C18 flash column and eluting with a 10 to 90% CH₃CN/H₂O+0.1% TFA gradient to provide the title compound as a yellow solid (TFA salt, 105 mg, 72% yield).

LC/MS: Calc'd m/z=425.2 for C₂₃H₂₇N₃O₅, found [M+Na]⁺=448.0.

### 4.17. (9H-fluoren-9-yl)methyl (2-(((((S)-4-(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)morpholin-2-yl)methoxy)methyl)amino)-2-oxoethyl)carbamate (Compound 4.17)

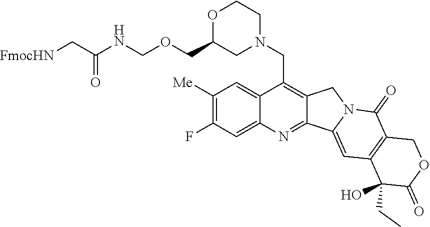

The title compound was prepared according to General Procedure 1 starting from Compound 1.1 (50 mg, 0.117 mmol) and Compound 4.16 (63 mg, 0.117 mmol). Preparative HPLC purification was accomplished as described in General Procedure 9, eluting with a 10 to 100% CH₃CN/H₂O+0.1% TFA gradient to give the title compound as an off-white solid (TFA salt, 33 mg, 35% yield).

LC/MS: Calc'd m/z=817.9 for C₄₅H₄₄FN₅09, found [M+H]⁺=818.7.

### 4.18: (S)-2-(1-(2,5-dioxo-2,5-dihydro-1H-pyrrol-1-yl)-12,15-dioxo-3,6,9-trioxa-13,16-diazaoctadecan-18-amido)-N-(2-(((((S)-4-(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)morpholin-2-yl)methoxy)methyl)amino)-2-oxoethyl)-3-phenylpropanamide (MT-GGFG-AM-Compound-113)

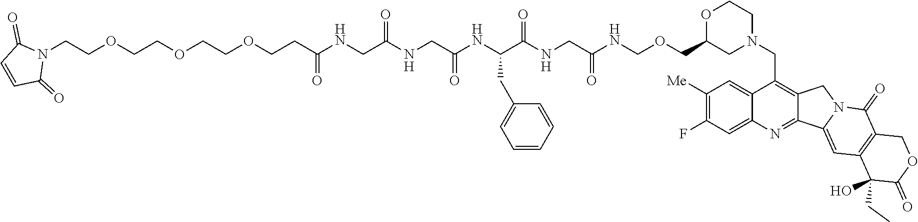 [SMILES: CC[C@@]1(O)C(=O)OCc2c1cc1n(c2=O)Cc2c-1nc1cc(F)c(C)cc1c2CN1CCO[C@H](COCNC(=O)CNC(=O)[C@H](Cc2ccccc2)NC(=O)CNC(=O)CNC(=O)CCOCCOCCOCCN2C(=O)C=CC2=O)C1]

Compound 4.17 (33 mg, 0.04 mmol) was taken up in a 10% solution of piperidine in DMF (1 mL) and stirred for 10 min. The piperidine solution was evaporated, the resulting residue was redissolved in DMF (5 mL), and then evaporated to dryness once more. To this residue was added DMF (100 uL) and DCM (900 uL) followed by Compound 4.4 (45 mg, 0.08 mmol), NMM (20 uL) and HATU (28 mg, 0.073 mmol). Preparative HPLC purification was accomplished as described in General Procedure 9, eluting with a 30 to 60% CH₃CN/H₂O+0.1% TFA gradient to provide the title compound as a yellow solid (22 mg, 48% yield).

LC/MS: Calc'd m/z=1140.2 for C₅₆H₆₆FN₉O₁₆, found [M+H]⁺=1141.1.

¹H NMR (300 MHz, MeOD) δ 8.35 (d, J=7.5 Hz, 2H), 7.74-7.61 (m, 1H), 7.53 (s, 1H), 7.34-7.10 (m, 6H), 6.81 (s, 2H), 5.65-5.30 (m, 4H), 4.64 (t, J=3.4 Hz, 2H), 4.42 (tt, J=6.3, 2.5 Hz, 1H), 4.09 (d, J=12.3 Hz, 1H), 3.98-3.76 (m, 8H), 3.72 (t, J=6.0 Hz, 2H), 3.69-3.44 (m, 17H), 3.21-2.85 (m, 3H), 2.64-2.42 (m, 5H), 2.03-1.84 (m, 2H), 0.98 (t, J=7.3 Hz, 3H).

### 4.19: (9H-fluoren-9-yl)methyl (S)-(2-((((9-amino-4-ethyl-8-fluoro-4-hydroxy-3,14-dioxo-3,4,12,14-tetrahydro-1H-pyrano[3,4′:6,7]indolizino[1,2-b]quinolin-11-yl)methoxy)methyl) amino)-2-oxoethyl)carbamate (Compound 4.19)

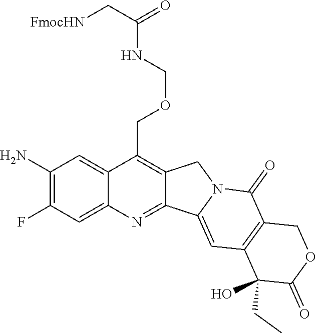

Compound 3.5 (55 mg, 0.11 mmol) was dissolved in TFA (500 uL) and stirred at room temperature for 20 min, then hexafluoroisopropanol (2 mL) was added followed by Compound 4.5 (40 mg, 0.11 mmol). This solution was stirred at room temperature for ˜16 h then concentrated to dryness. Purification was accomplished as described in General Procedure 9, using a 12 g C₁₈ flash column and eluting with a 10 to 50% CH₃CN/H₂O+0.1% TFA gradient to provide the title compound as a yellow solid (11 mg, 14% yield).

LC/MS: Calc'd m/z=719.7 for C₃₉H₃₄FN₅O₈, found [M+H]⁺=720.6.

### 4.20: (S)-N-(2-(((((S)-9-amino-4-ethyl-8-fluoro-4-hydroxy-3,14-dioxo-3,4,12,14-tetrahydro-1H-pyrano[3′,4′:6,7]indolizino[1,2-b]quinolin-11-yl)methoxy)methyl)amino)-2-oxoethyl)-2-(1-(2,5-dioxo-2,5-dihydro-1H-pyrrol-1-yl)-12,15-dioxo-3,6,9-trioxa-13,16-diazaoctadecan-18-amido)-3-phenylpropanamide (MT-GGFG-AM-Compound 141)

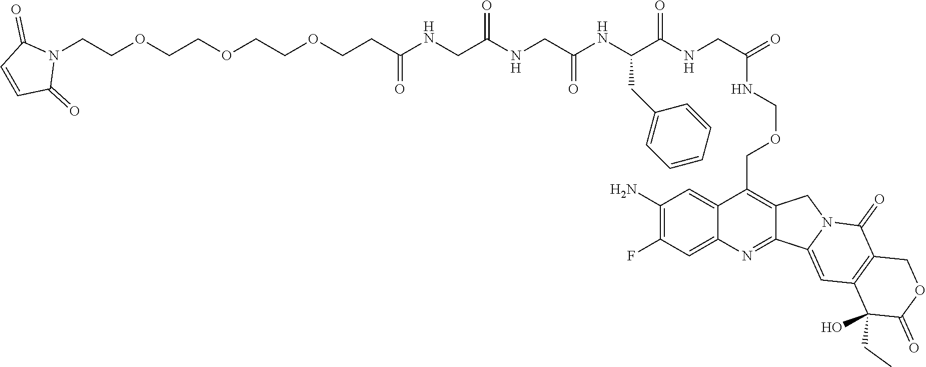 [SMILES: CC[C@@]1(O)C(=O)OCc2c1cc1n(c2=O)Cc2c-1nc1cc(F)c(N)cc1c2COCNC(=O)CNC(=O)[C@H](Cc1ccccc1)NC(=O)CNC(=O)CNC(=O)CCOCCOCCOCCN1C(=O)C=CC1=O]

Compound 4.19 (11 mg, 0.015 mmol) was taken up in a 10% solution of piperidine in DMF (1 mL) and stirred for 10 min. The piperidine solution was evaporated, the resulting residue was redissolved in DMF (5 mL), and then evaporated to dryness once more. To this residue was added DMF (50 uL) and DCM (450 uL) followed by Compound 4.4 (26 mg, 0.045 mmol), NMM (5 uL) and HATU (18 mg, 0.045 mmol). Preparative HPLC purification was accomplished as described in General Procedure 9, eluting with a 32 to 45% CH₃CN/H₂O+0.1% TFA gradient to provide the title compound as a yellow solid (4.6 mg, 29% yield).

LC/MS: Calc'd m/z=1142.0 for C₅₀H₅₆FN₉O₁₅, found [M+H]⁺=1143.1.

¹H NMR (300 MHz, MeOD) δ 8.36 (s, 1H), 8.28 (d, J=6.1 Hz, 1H), 8.16 (dd, J=20.1, 6.8 Hz, 3H), 7.59-7.44 (m, 2H), 7.31-7.08 (m, 6H), 6.79 (s, 2H), 5.58 (d, J=16.1 Hz, 1H), 5.37 (d, J=16.1 Hz, 1H), 5.30-5.16 (m, 3H), 4.56-4.39 (m, 1H), 4.07-3.90 (m, 2H), 3.85 (dt, J=11.5, 5.4 Hz, 4H), 3.79-3.67 (m, 4H), 3.67-3.55 (m, 7H), 3.54 (d, J=6.5 Hz, 8H), 3.10 (dd, J=14.0, 6.1 Hz, 1H), 2.92 (dd, J=13.9, 9.1 Hz, 1H), 2.53 (t, J=6.0 Hz, 2H), 1.98 (q, J=7.2 Hz, 2H), 1.31 (s, 1H), 1.04 (t, J=7.3 Hz, 3H).

### 4.21: N-((S)-1-((S)-9-amino-4-ethyl-8-fluoro-4-hydroxy-3,14-dioxo-3,4,12,14-tetrahydro-1H-pyrano[3,4′:6,7]indolizino[1,2-b]quinolin-11-yl)-9-benzyl-5,8,11,14-tetraoxo-2-oxa-4,7,10,13-tetraazapentadecan-15-yl)-6-(2,5-dioxo-2,5-dihydro-1H-pyrrol-1-yl)hexanamide (MC-GGFG-AM-Compound 141)

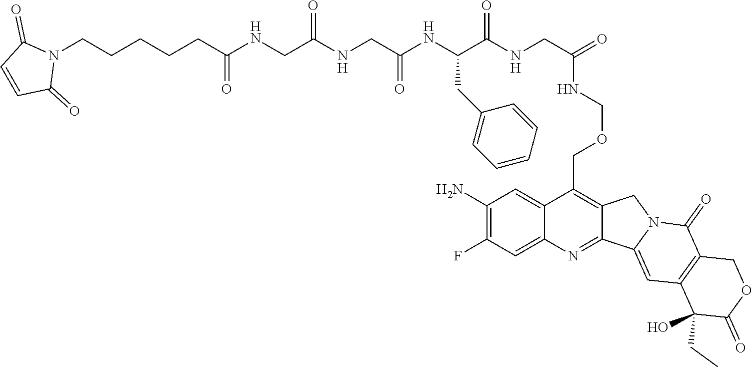 [SMILES: CC[C@@]1(O)C(=O)OCc2c1cc1n(c2=O)Cc2c-1nc1cc(F)c(N)cc1c2COCNC(=O)CNC(=O)[C@H](Cc1ccccc1)NC(=O)CNC(=O)CNC(=O)CCCCCN1C(=O)C=CC1=O]

Compound 4.19 (25 mg, 0.035 mmol) was taken up in a 10% solution of piperidine in DMF (1 mL) and stirred for 10 min. The piperidine solution was evaporated, the resulting residue was redissolved in DMF (5 mL), and then evaporated to dryness once more. To this residue was added DMF (50 uL) and DCM (450 uL), followed by MC-GGF-OH (33 mg, 0.07 mmol), NMM (20 uL) and HATU (25 mg, 0.066 mmol). Preparative HPLC purification was accomplished as described in General Procedure 9, eluting with a 30 to 50% CH₃CN/H₂O+0.1% TFA gradient to provide the title compound as a yellow solid (4.3 mg, 13% yield).

LC/MS: Calc'd m/z=952.0 for C₄₇H₅₀FN₉O₁₂, found [M+H]⁺=952.9.

¹H NMR (300 MHz, CD₃CN) δ 7.96-7.72 (m, 1H), 7.39-7.07 (m, 8H), 6.94 (d, J=9.1 Hz, 1H), 6.73 (s, 2H), 5.44 (d, J=16.2 Hz, 1H), 5.25 (d, J=16.2 Hz, 1H), 5.06 (d, J=4.4 Hz, 2H), 4.81 (d, J=26.1 Hz, 4H), 4.61 (s, 1H), 3.96 (s, 1H), 3.77 (d, J=8.1 Hz, 7H), 3.02 (d, J=5.6 Hz, 5H), 2.19 (t, J=7.7 Hz, 3H), 1.50 (dp, J=14.8, 7.4 Hz, 6H), 1.32-1.12 (m, 3H), 0.96 (t, J=7.2 Hz, 3H).

### 4.22: tert-butyl (2-((2-(((S)-1-((2-(((S)-4-ethyl-8-fluoro-4-hydroxy-3,14-dioxo-3,4,12,14-tetrahydro-1H-pyrano[3,4′:6,7]indolizino[1,2-b]quinolin-9-yl)amino)-2-oxoethyl)amino)-1-oxo-3-phenylpropan-2-yl)amino)-2-oxoethyl)amino)-2-oxoethyl)carbamate (Compound 4.22)

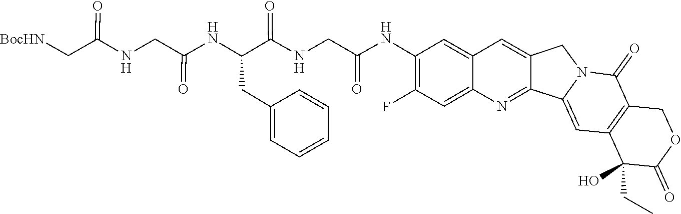 [SMILES: CC[C@@]1(O)C(=O)OCc2c1cc1n(c2=O)Cc2cc3cc(NC(=O)CNC(=O)[C@H](Cc4ccccc4)NC(=O)CNC(=O)CNC(=O)OC(C)(C)C)c(F)cc3nc2-1]

The title compound was prepared according to Procedure 7 starting from Compound 140 (28 mg). Preparative HPLC purification was accomplished as described in General Procedure 9, eluting with a 10 to 50% CH₃CN/H₂O+0.1% TFA gradient to provide the title compound as a white solid (10 mg, 17% yield).

LC/MS: Calc'd m/z=799.3 for C₄₀H₄₂N₇O₁₀, found [M+H]⁺=800.6.

### 4.23: (S)-2-(1-(2,5-dioxo-2,5-dihydro-1H-pyrrol-1-yl)-12,15-dioxo-3,6,9-trioxa-13,16-diazaoctadecan-18-amido)-N-(2-(((S)-4-ethyl-8-fluoro-4-hydroxy-3,14-dioxo-3,4,12,14-tetrahydro-1H-pyrano[3,4′:6,7]indolizino[1,2-b]quinolin-9-yl)amino)-2-oxoethyl)-3-phenylpropanamide (MT-GGFG-Compound 140)

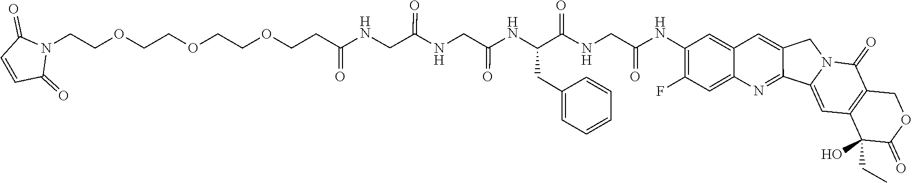 [SMILES: CC[C@@]1(O)C(=O)OCc2c1cc1n(c2=O)Cc2cc3cc(NC(=O)CNC(=O)[C@H](Cc4ccccc4)NC(=O)CNC(=O)CNC(=O)CCOCCOCCOCCN4C(=O)C=CC4=O)c(F)cc3nc2-1]

The title compound was prepared according to General Procedure 6 followed by General Procedure 8 starting from Compound 4.22 (10 mg). Preparative HPLC purification was accomplished as described in General Procedure 9, eluting with a 10 to 50% CH₃CN/H₂O+0.1% TFA gradient to provide the title compound as a white solid (6.8 mg, 55% yield).

LC/MS: Calc'd m/z=982.4 for C₄₈H₅₁FN₈O₁₄, found [M+H]⁺=983.6.

### 4.24: tert-butyl (2-((2-(((S)-1-((2-(((S)-4-ethyl-8-fluoro-4-hydroxy-11-(morpholinomethyl)-3,14-dioxo-3,4,12,14-tetrahydro-1H-pyrano[3,4′:6,7]indolizino[1,2-b]quinolin-9-yl)amino)-2-oxoethyl)amino)-1-oxo-3-phenylpropan-2-yl)amino)-2-oxoethyl)amino)-2-oxoethyl)carbamate (Compound 4.24)

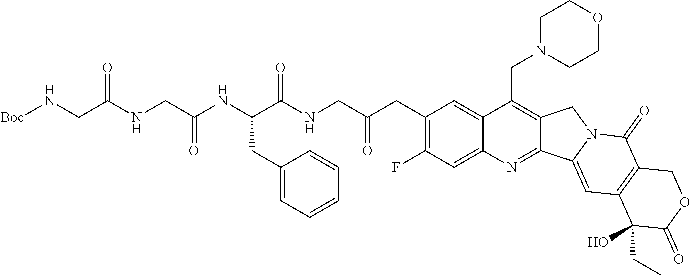 [SMILES: CC[C@@]1(O)C(=O)OCc2c1cc1n(c2=O)Cc2c-1nc1cc(F)c(CC(=O)CNC(=O)[C@H](Cc3ccccc3)NC(=O)CNC(=O)CNC(=O)OC(C)(C)C)cc1c2CN1CCOCC1]

The title compound was prepared according to General Procedure 7 starting from Compound 142 (TFA salt, 45 mg). Purification was accomplished as described in General Procedure 9, using a 12 g C18 flash column and eluting with a 10 to 50% CH₃CN/H₂O+0.1% TFA gradient to provide the title compound as a white solid (13 mg, 22% yield).

LC/MS: Calc'd m/z=898.4 for C₄₅H₅₁N₈O₁₁, found [M+H]⁺=899.6.

### 4.25: (S)-2-(1-(2,5-dioxo-2,5-dihydro-1H-pyrrol-1-yl)-12,15-dioxo-3,6,9-trioxa-13,16-diazaoctadecan-18-amido)-N-(2-(((S)-4-ethyl-8-fluoro-4-hydroxy-11-(morpholinomethyl)-3,14-dioxo-3,4,12,14-tetrahydro-1H-pyrano[3,4′:6,7]indolizino[1,2-b]quinolin-9-yl)amino)-2-oxoethyl)-3-phenylpropanamide (MT-GGFG-Compound 142)

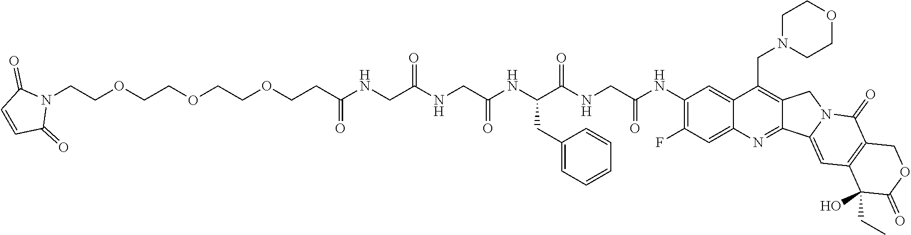 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCOCCOCCOCCN3C(=O)C=CC3=O)cc1c2CN1CCOCC1]

The title compound was prepared according to General Procedure 6 followed by General Procedure 8 starting from Compound 4.24 (13 mg). Preparative HPLC purification was accomplished as described in General Procedure 9, eluting with a 10 to 50% CH₃CN/H₂O+0.1% TFA gradient to provide the title compound as a white solid (2.6 mg, 17% yield).

LC/MS: Calc'd m/z=1081.4 for C₅₃H₆₀FN₉O₁₅, found [M+H]⁺=1082.6.

¹H NMR (300 MHz, MeOD) δ 9.34 (d, J=8.5 Hz, 1H), 7.87 (d, J=11.8 Hz, 1H), 7.62 (s, 1H), 7.33-7.19 (m, 5H), 6.80 (s, 2H), 5.62 (d, J=16.3 Hz, 1H), 5.51 (s, 2H), 5.47-5.35 (m, 3H), 4.73 (dd, J=9.6, 5.1 Hz, 1H), 4.61 (s, 3H), 4.30-4.15 (m, 2H), 4.11 (s, 2H), 4.00-3.82 (m, 4H), 3.82-3.70 (m, 7H), 3.70-3.50 (m, 13H), 3.18-3.04 (m, 1H), 2.88 (s, 1H), 2.64 (d, J=5.8 Hz, 4H), 2.54 (t, J=6.0 Hz, 2H), 2.09-1.92 (m, 2H), 1.03 (t, J=7.3 Hz, 3H).

### 4.26: (9H-fluoren-9-yl)methyl (S)-(12-benzyl-1-(4-nitrophenoxy)-1,8,11,14,17-pentaoxo-2,5-dioxa-7,10,13,16-tetraazaoctadecan-18-yl)carbamate (Compound 4.26)

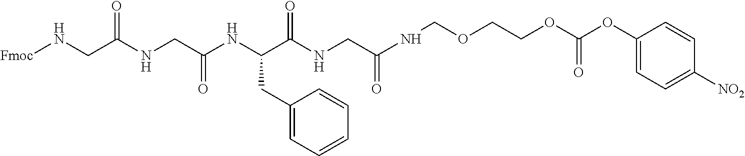

To a stirring solution of Compound 4.6 (60 mg) in dichloromethane (2 mL) was added ethylene glycol (100 uL) followed by trifluoracetic acid (0.4 mL). After 30 min the reaction was concentrated in vacuo. Purification of the intermediate compound was accomplished as described in General Procedure 9, using a 10 g flash column and eluting with a 0 to 20% dichloromethane/methanol gradient. To the purified intermediate in tetrahydrofuran (0.5 mL) was added bis-nitrophenol carbonate (58 mg) followed by DIPEA (50 uL). The solution was stirred for 16 h, quenched with acetic acid (˜100 uL) then concentrated to dryness. Purification was accomplished as described in General Procedure 9, using a 10 g flash column and eluting with a 0 to 20% dichloromethane/methanol gradient to provide the title compound as a white solid (40 mg, 53% yield from Compound 4.6).

LC/MS: Calc'd m/z=796.3 for C₄₀H₄₀N₆O₁₂, found [M+Na]⁺=819.4.

### 4.27: (S)-16-amino-10-benzyl-6,9,12,15-tetraoxo-3-oxa-5,8,11,14-tetraazahexadecyl (((S)-4-ethyl-8-fluoro-4-hydroxy-9-methyl-3,14-dioxo-3,4,12,14-tetrahydro-1H-pyrano[3,4′:6,7]indolizino[1,2-b]quinolin-11-yl)methyl)carbamate (Compound 4.27)

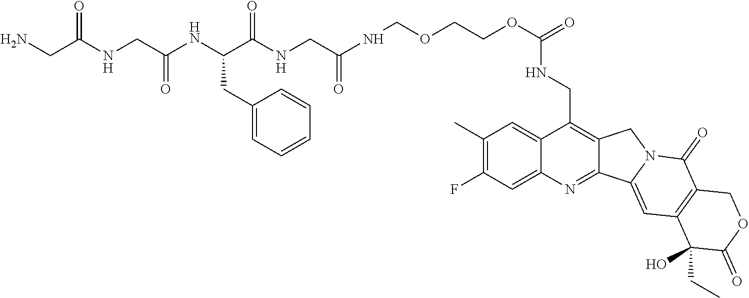 [SMILES: CC[C@@]1(O)C(=O)OCc2c1cc1n(c2=O)Cc2c-1nc1cc(F)c(C)cc1c2CNC(=O)OCCOCNC(=O)CNC(=O)[C@H](Cc1ccccc1)NC(=O)CNC(=O)CN]

To a solution of Compound 4.26 (40 mg) in dimethylformamide (1 mL) was added DIPEA (26 uL) then a solution of Compound 1.2 (24 mg) in dimethylformamide (0.5 mL). This solution was stirred for 4 h at room temperature then quenched with a 20% piperidine in dimethylformamide solution (0.5 mL) and stirred for an additional 20 min. Purification was accomplished as described in General Procedure 9, using a 12 g C18 flash column and eluting with a 10 to 50% CH₃CN/H₂O+0.1% TFA gradient to provide the title compound as a white solid (TFA salt, 19 mg, 39% yield).

LC/MS: Calc'd m/z=844.3 for C₄₁H₄₅FN₈O₁₁, found [M+H]⁺=845.6.

### 4.28: (S)-10-benzyl-29-(2,5-dioxo-2,5-dihydro-1H-pyrrol-1-yl)-6,9,12,15,18-pentaoxo-3,21,24,27-tetraoxa-5,8,11,14,17-pentaazanonacosyl (((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)carbamate (MT-GGFG-AM-Compound 139)

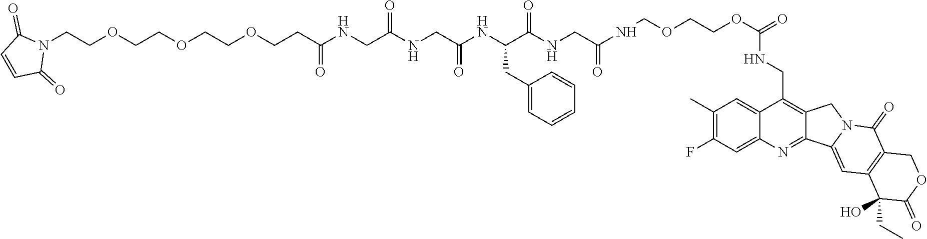 [SMILES: CC[C@@]1(O)C(=O)OCc2c1cc1n(c2=O)Cc2c-1nc1cc(F)c(C)cc1c2CNC(=O)OCCOCNC(=O)CNC(=O)[C@H](Cc1ccccc1)NC(=O)CNC(=O)CNC(=O)CCOCCOCCOCCN1C(=O)C=CC1=O]

The title compound was prepared according to General Procedure 8 starting from Compound 4.27 (10 mg). Preparative HPLC purification was accomplished as described in General Procedure 9, eluting with a 10 to 50% CH₃CN/H₂O+0.1% TFA gradient to provide the title compound as a white solid (8.8 mg, 75% yield).

LC/MS: Calc'd m/z=1127.4 for C₅₄H₆₂FN₉O₁₇, found [M+H]⁺=1128.6.

¹H NMR (300 MHz, MeOD) δ 8.27 (d, J=8.1 Hz, 1H), 7.81 (d, J=10.7 Hz, 1H), 7.65 (s, 1H), 7.32-7.16 (m, 5H), 6.81 (s, 2H), 5.62 (d, J=16.4 Hz, 1H), 5.53 (s, 2H), 5.42 (d, J=16.4 Hz, 1H), 4.93 (s, 2H), 4.67 (s, 1H), 4.51 (dd, J=9.3, 5.6 Hz, 1H), 4.18 (t, J=4.7 Hz, 2H), 4.01-3.44 (m, 19H), 3.17 (dd, J=13.9, 5.8 Hz, 1H), 2.97 (dd, J=13.9, 9.0 Hz, 1H), 2.57 (s, 3H), 2.52 (t, J=6.0 Hz, 2H), 2.03-1.91 (m, 2H), 1.03 (t, J=7.4 Hz, 3H). 4.29: (S)-2-amino-N-(4-ethyl-8-fluoro-4-hydroxy-11-(hydroxymethyl)-3,14-dioxo-3,4,12,14-tetrahydro-1H-pyrano[3,4′:6,7]indolizino[1,2-b]quinolin-9-yl)acetamide (Compound 4.29)

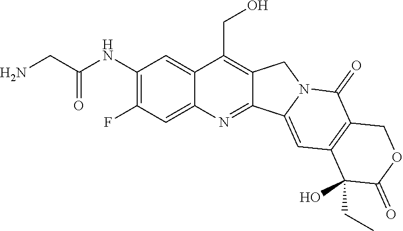 [SMILES: CC[C@@]1(O)C(=O)OCc2c1cc1n(c2=O)Cc2c-1nc1cc(F)c(NC(=O)CN)cc1c2CO]

To a stirring solution of Fmoc-glycine (217 mg) in dimethylformamide (2.5 mL) was added HATU (254 mg), HOAt (83 mg) then NMM (188 uL). This solution was stirred for 10 min then Compound 141 (50 mg) was added and the reaction was stirred at room temperature for 16 h. Lithium hydroxide (2.5 mL, 1 M in water) was added, and the reaction mixture was stirred for 2 h. This solution was partially concentrated, then a solution of 20% piperidine in dimethylformamide (0.5 mL) was added and was stirred for another 20 min. The reaction was then evaporated onto celite and purification was accomplished as described in General Procedure 9, using a 12 g C18 flash column and eluting with a 0 to 40% CH₃CN/H₂O+0.1% TFA gradient to provide the title compound as a white solid (TFA salt, 44 mg, 62% yield).

LC/MS: Calc'd m/z=468.1 for C₂₃H₂₁FN₄O₆, found [M+H]⁺=469.4.

¹H NMR (300 MHz, MeOD) δ 8.99 (d, J=8.3 Hz, 1H), 7.99 (s, 1H), 7.87 (d, J=12.0 Hz, 1H), 7.55 (s, 1H), 5.60 (d, J=16.3 Hz, 1H), 5.46-5.35 (m, 3H), 5.30 (s, 2H), 3.53-3.45 (m, 1H), 3.43-3.38 (m, 1H), 2.03-1.87 (m, 2H), 1.02 (t, J=7.3 Hz, 3H).

### 4.30: (S)-2-(1-(2,5-dioxo-2,5-dihydro-1H-pyrrol-1-yl)-12,15-dioxo-3,6,9-trioxa-13,16-diazaoctadecan-18-amido)-N-(2-(((S)-4-ethyl-8-fluoro-4-hydroxy-11-(hydroxymethyl)-3,14-dioxo-3,4,12,14-tetrahydro-1H-pyrano[3′,4′:6,7]indolizino[1,2-b]quinolin-9-yl)amino)-2-oxoethyl)-3-phenylpropanamide (MT-GGFG-Compound 141)

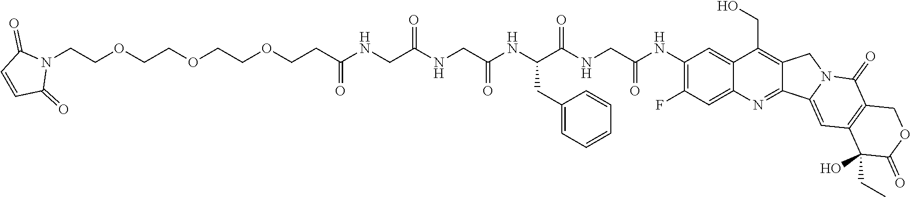 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCOCCOCCOCCN3C(=O)C=CC3=O)cc1c2CO]

To a stirring solution of Compound 4.4 (23 mg) in a mixture of dimethylformamide (0.1 mL) and dichloromethane (0.9 mL) was added HATU (14 mg), a solution of Compound 4.29 (20 mg) in dimethyl formamide (0.1 mL) and dichloromethane (0.9 mL), and DIPEA (24 uL). The mixture was stirred for 15 min, then the reaction was partially concentrated. Preparative HPLC purification was accomplished as described in General Procedure 9, eluting with a 0 to 40% CH₃CN/H₂O+0.1% TFA gradient to provide the title compound as a white solid (7.1 mg, 20% yield).

LC/MS: Calc'd m/z=1012.4 for C₄₉H₅₃FN₈O₁₅, found [M+H]⁺=1013.6.

¹H NMR (300 MHz, MeOD) δ 9.89 (s, 1H), 8.75 (d, J=8.3 Hz, 1H), 8.44-8.32 (m, 1H), 8.27-8.14 (m, 2H), 7.78 (d, J=11.9 Hz, 1H), 7.53 (s, 1H), 7.39-7.20 (m, 5H), 6.82 (s, 2H), 5.57 (d, J=16.3 Hz, 1H), 5.39 (d, J=16.3 Hz, 1H), 5.34-5.25 (m, 2H), 5.22 (s, 2H), 4.32-4.09 (m, 2H), 3.96-3.83 (m, 3H), 3.76 (t, J=6.0 Hz, 2H), 3.69-3.62 (m, 2H), 3.62-3.47 (m, 9H), 3.40-3.33 (m, 1H), 3.08 (dd, J=14.0, 9.6 Hz, 1H), 2.56 (t, J=6.1 Hz, 2H), 2.03-1.91 (m, 2H), 1.04 (t, J=7.3 Hz, 3H).

### 4.31: tert-butyl (S)-((9-amino-4-ethyl-8-fluoro-4-hydroxy-3,14-dioxo-3,4,12,14-tetrahydro-1H-pyrano[3,4′:6,7]indolizino[1,2-b]quinolin-11-yl)methyl)carbamate (Compound 4.31)

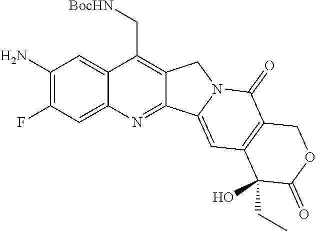 [SMILES: CC[C@@]1(O)C(=O)OCc2c1cc1n(c2=O)Cc2c-1nc1cc(F)c(N)cc1c2CNC(=O)OC(C)(C)C]

To a stirring solution of Compound 145 (32 mg) in dichloromethane (2 mL) and acetonitrile (0.5 mL) was added di-tert-butyl dicarbonate (20 uL) followed by DIPEA (42 uL). The reaction mixture was stirred at room temperature for 3 h then concentrated to dryness to provide the title compound as a red solid (34 mg, 87%).

LC/MS: Calc'd m/z=510.2 for C₂₆H₂₇FN₄O₆, found [M+H]⁺=511.2.

### 4.32: tert-butyl (S)-((9-(2-aminoacetamido)-4-ethyl-8-fluoro-4-hydroxy-3,14-dioxo-3,4,12,14-tetrahydro-1H-pyrano[3,4′:6,7]indolizino[1,2-b]quinolin-11-yl)methyl)carbamate (Compound

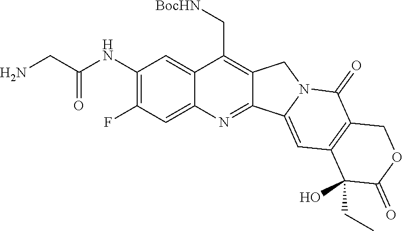 [SMILES: CC[C@@]1(O)C(=O)OCc2c1cc1n(c2=O)Cc2c-1nc1cc(F)c(NC(=O)CN)cc1c2CNC(=O)OC(C)(C)C]

To a stirring solution of Fmoc-glycine (98 mg) in dimethylformamide (1 mL) was added HATU (115 mg), HOAt (37 mg) then NMM (85 μL). This solution was stirred for 10 min, then Compound 4.31 (28 mg) was added. The reaction was stirred at room temperature for 16 h then quenched with a solution of 20% piperidine in dimethylformamide (1 mL) and stirred for an additional 20 min. Purification was accomplished as described in General Procedure 9, using a 12 g C18 flash column and eluting with a 5 to 40% CH₃CN/H₂O+0.1% TFA gradient to provide the title compound as a white solid (TFA salt, 25 mg, 67% yield).

LC/MS: Calc'd m/z=567.2 for C₂₈H₃₀FN₆O₇, found [M+H]⁺=568.4.

¹H NMR (300 MHz, MeOD) δ 9.01 (d, J=8.3 Hz, 1H), 7.83 (d, J=11.9 Hz, 1H), 7.52 (s, 1H), 5.57 (d, J=16.4 Hz, 1H), 5.38 (d, J=16.3 Hz, 1H), 5.27 (d, J=3.1 Hz, 2H), 4.80 (s, 2H), 4.10 (s, 2H), 1.97 (q, J=7.4 Hz, 2H), 1.50 (s, 9H), 1.02 (t, J=7.3 Hz, 3H).

### 4.33: tert-butyl (((S)-9-(2-((S)-2-(2-(2-aminoacetamido)acetamido)-3-phenylpropanamido)acetamido)-4-ethyl-8-fluoro-4-hydroxy-3,14-dioxo-3,4,12,14-tetrahydro-1H-pyrano[3′,4′:6,7]indolizino[1,2-b]quinolin-11-yl)methyl)carbamate (Compound 4.33)

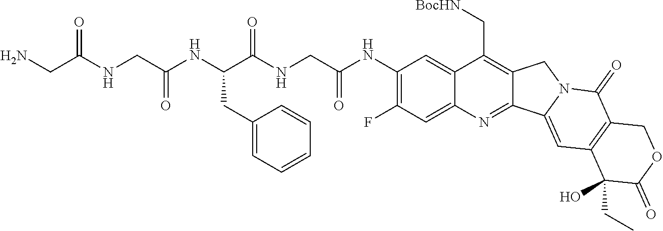 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CN)cc1c2CNC(=O)OC(C)(C)C]

To a stirring solution of Fmoc-GGF-OH (28 mg) and HATU (20 mg) in a mixture of DMF (0.2 mL) and dichloromethane (1.8 mL) was added Compound 4.32 (25 mg) followed by DIPEA (32 uL). This solution was stirred for 15 min at room temperature, quenched with a solution of 20% piperidine in dimethylformamide (0.250 mL), stirred for an additional 20 min, then partially concentrated in vacuo. Purification was accomplished as described in General Procedure 9, using a 12 g C18 flash column and eluting with a 10 to 45% CH₃CN/H₂O+0.1% TFA gradient to provide the title compound as a white solid (TFA salt, 22 mg, 64% yield).

LC/MS: Calc'd m/z=828.3 for C₄₁H₄₅FN₈O₁₀, found [M+H]⁺=829.6.

### 4.34: (S)-N-(2-(((S)-11-(aminomethyl)-4-ethyl-8-fluoro-4-hydroxy-3,14-dioxo-3,4,12,14-tetrahydro-1H-pyrano[3,4′:6,7]indolizino[1,2-b]quinolin-9-yl)amino)-2-oxoethyl)-2-(1-(2,5-dioxo-2,5-dihydro-1H-pyrrol-1-yl)-12,15-dioxo-3,6,9-trioxa-13,16-diazaoctadecan-18-amido)-3-phenylpropanamide (MT-GGFG-Compound 145)

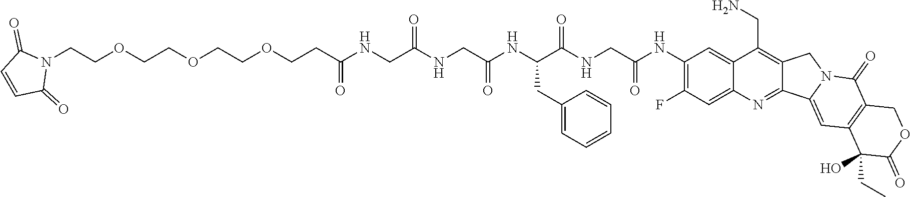 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCOCCOCCOCCN3C(=O)C=CC3=O)cc1c2CN]

The title compound was prepared according to Procedure 6 followed by Procedure 8 starting from Compound 4.33 (15 mg). Preparative HPLC purification of the intermediate Boc-protected compound was accomplished as described in General Procedure 9, eluting with a 10 to 50% CH₃CN/H₂O+0.1% TFA gradient. The title compound was obtained post Boc-deprotection as a white-solid (TFA salt, 8.5 mg, 52% yield).

LC/MS: Calc'd m/z=1011.4 for C₄₉H₅₃FN₈O₁₅, found [M+H]⁺=1012.6.

¹H NMR (300 MHz, MeOD) δ 9.04 (d, J=8.0 Hz, 1H), 8.40 (d, J=5.7 Hz, 1H), 8.21 (d, J=7.7 Hz, 1H), 8.05 (d, J=11.5 Hz, 1H), 7.67 (s, 1H), 7.42-7.03 (m, 5H), 6.81 (s, 2H), 5.63 (d, J=16.4 Hz, 1H), 5.51 (s, 1H), 5.43 (d, J=16.5 Hz, 1H), 4.81 (s, 2H), 4.75-4.58 (m, 1H), 4.29-4.10 (m, 2H), 3.98-3.81 (m, 4H), 3.78-3.71 (m, 2H), 3.71-3.63 (m, 2H), 3.62-3.53 (m, 9H), 3.14-2.98 (m, 1H), 2.54 (t, J=6.0 Hz, 2H), 2.08-1.93 (m, 2H), 1.03 (t, J=7.3 Hz, 3H).

### 4.355: (9H-fluoren-9-yl)methyl(S)-(2-((4-ethyl-8-fluoro-4-hydroxy-3,14-dioxo-11-(piperidin-1-ylmethyl)-3,4,12,14-tetrahydro-1H-pyrano[3′,4′:6,7]indolizino[1,2-b]quinolin-9-yl)amino)-2-oxoethyl)carbamate (Compound 4.35)

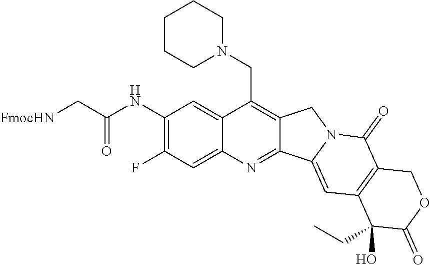

To a solution of Fmoc-Gly-OH (100.9 mg, 0.34 mmol) in dimethylformamide (550 uL) was added NMM (0.112 mL, 1.02 mmol) and HATU (0.103 g, 0.272 mmol). This solution was stirred at room temperature for 20 min, then a solution of Compound 148 (32.5 mg, 0.068 mmol) in DMF (250 uL) was added, and the reaction mixture was stirred for 16 h. Purification was accomplished as described in General Procedure 9, using a 12 g C18 flash column and eluting with a 5 to 40% CH₃CN/H₂O+0.1% TFA gradient. The obtained residue was re-purified according to General Procedure 9, using a 10 g flash column and eluting with a 0 to 10% MeOH/DCM gradient to provide the title compound as a yellow powder (15.3 mg, 30% yield).

LC/MS: Calc'd m/z=757.3 for C₄₃H₄₀FN₅O₇, found [M+H]⁺=758.6.

### 4.36: (S)-2-(1-(2,5-dioxo-2,5-dihydro-1H-pyrrol-1-yl)-12,15-dioxo-3,6,9-trioxa-13,16-diazaoctadecan-18-amido)-N-(2-(((S)-4-ethyl-8-fluoro-4-hydroxy-3,14-dioxo-11-(piperidin-1-ylmethyl)-3,4,12,14-tetrahydro-1H-pyrano[3′,4′:6,7]indolizino[1,2-b]quinolin-9-yl)amino)-2-oxoethyl)-3-phenylpropanamide (MT-GGFG-Compound 148)

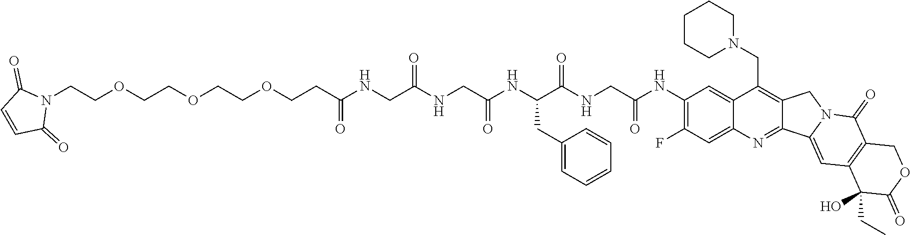 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCOCCOCCOCCN3C(=O)C=CC3=O)cc1c2CN1CCCCC1]

To a 50 mL flask containing Compound 4.35 (15.3 mg, 0.02 mmol) was added a solution of 20% piperidine in DMF (2.0 mL). This solution was stirred at room temperature for 5 min then evaporated to dryness. The obtained residue was then dissolved in 10% DMF/DCM (1.0 mL), then NMM (5.50 μL, 0.05 mmol), Compound 4.4 (11.2 mg, 0.02 mmol) and HATU (8.7 mg, 0.02 mmol) were added. This solution was stirred for 45 min, then partially evaporated. Preparative HPLC purification was accomplished as described in General Procedure 9, eluting with a 15 to 45% CH₃CN/H₂O+0.1% TFA gradient to give the title product as a yellow powder (7.8 mg, 33% yield).

LC/MS: Calc'd m/z=1079.4 for C₅₄H₆₂FN₉O₁₄, found [M+H]⁺=1080.8.

¹H NMR (300 MHz, MeOD) δ 8.99 (d, J=8.2 Hz, 1H), 7.52 (d, J=12.3 Hz, 1H), 7.39-7.25 (m, 5H), 7.25-7.17 (m, 1H), 6.79 (s, 2H), 5.53 (d, J=16.4 Hz, 1H), 5.33 (d, J=16.5 Hz, 1H), 4.80-4.72 (m, 1H), 4.32-4.11 (m, 2H), 3.98-3.79 (m, 6H), 3.76 (t, J=6.0 Hz, 2H), 3.66-3.60 (m, 2H), 3.62-3.49 (m, 10H), 3.15-3.03 (m, 1H), 2.65-2.47 (m, 6H), 1.96 (q, J=7.4 Hz, 2H), 1.72-1.57 (m, 4H), 1.57-1.42 (m, 2H), 1.03 (t, J=7.4 Hz, 3H).

### 4.37. tert-butyl (2-((2-(((S)-1-((2-((4-(N-(((S)-4-ethyl-8-fluoro-4-hydroxy-9-methyl-3,14-dioxo-3,4,12,14-tetrahydro-1H-pyrano[3,4′:6,7]indolizino[1,2-b]quinolin-11-yl)methyl)sulfamoyl)phenyl)amino)-2-oxoethyl)amino)-1-oxo-3-phenylpropan-2-yl)amino)-2-oxoethyl)amino)-2-oxoethyl)carbamate (Compound 4.37)

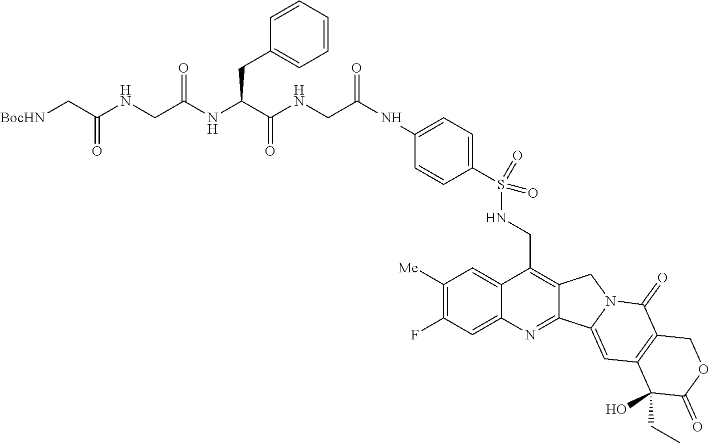 [SMILES: CC[C@@]1(O)C(=O)OCc2c1cc1n(c2=O)Cc2c-1nc1cc(F)c(C)cc1c2CNS(=O)(=O)c1ccc(NC(=O)CNC(=O)[C@H](Cc2ccccc2)NC(=O)CNC(=O)CNC(=O)OC(C)(C)C)cc1]

The title compound was prepared according to General Procedure 7 starting from Compound 127 (46 mg). Preparative HPLC purification was accomplished as described in General Procedure 9, eluting with a 10 to 60% CH₃CN/H₂O+0.1% TFA gradient to provide the title compound as a white solid (7.2 mg, 21% yield).

LC/MS: Calc'd m/z=983.0 for C₄₈H₅₁N₈O₁₂S, found [M+H]⁺=983.9.

### 4.38: (S)-2-(1-(2,5-dioxo-2,5-dihydro-1H-pyrrol-1-yl)-12,15-dioxo-3,6,9-trioxa-13,16-diazaoctadecan-18-amido)-N-(2-((4-(N-(((S)-4-ethyl-8-fluoro-4-hydroxy-9-methyl-3,14-dioxo-3,4,12,14-tetrahydro-1H-pyrano[3,4′:6,7]indolizino[1,2-b]quinolin-11-yl)methyl)sulfamoyl) phenyl)amino)-2-oxoethyl)-3-phenylpropanamide (MT-GGFG-Compound 127)

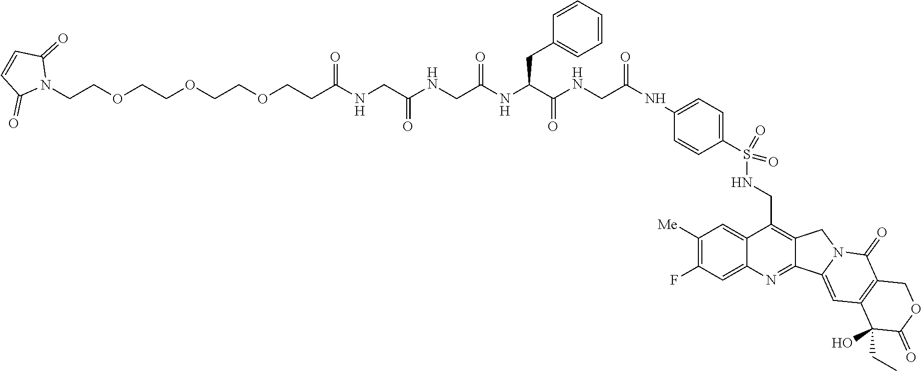 [SMILES: CC[C@@]1(O)C(=O)OCc2c1cc1n(c2=O)Cc2c-1nc1cc(F)c(C)cc1c2CNS(=O)(=O)c1ccc(NC(=O)CNC(=O)[C@H](Cc2ccccc2)NC(=O)CNC(=O)CNC(=O)CCOCCOCCOCCN2C(=O)C=CC2=O)cc1]

The title compound was prepared according to Procedure 6 followed by Procedure 8 starting from Compound 4.37 (7.2 mg). Preparative HPLC purification was accomplished as described in General Procedure 9, eluting with a 10 to 50% CH₃CN/H₂O+0.1% TFA gradient to provide the title compound as a white solid (1 mg, 12% yield).

LC/MS: Calc'd m/z=1166.2 for C₅₆H₆₀FN₉O₁₆, found [M+H]⁺=1167.1

### 4.39: (9H-fluoren-9-yl)methyl ((7S)-1-((3-azabicyclo[3.1.1]heptan-6-yl)oxy)-7-benzyl-3,6,9,12-tetraoxo-2,5,8,11-tetraazatridecan-13-yl)carbamate (Compound 4.39)

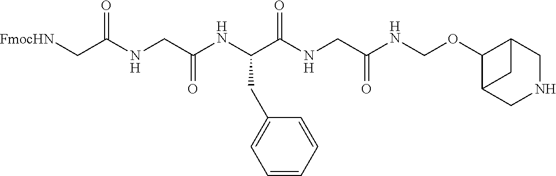

To a stirring solution of Compound 4.6 (44 mg) in dichloromethane (2 mL) was added 3-azabicyclo[3.1.1]heptan-6-ol (5.3 mg) followed by trifluoracetic acid (0.4 mL). After 30 min the reaction was concentrated in vacuo. Purification was accomplished as described in General Procedure 9, using a 10 g flash column and eluting with a 0 to 20% dichloromethane/methanol gradient to provide the title compound as a white solid (14.7 mg, 46% yield).

LC/MS: Calc'd m/z=682.8 for C₃₇H₄₂N₆O₇, found [M+H]⁺=683.6.

### 4.40: (2S)-2-(2-(2-aminoacetamido)acetamido)-N-(2-((((3-(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)-3-azabicyclo[3.1.1]heptan-6-yl)oxy)methyl)amino)-2-oxoethyl)-3-phenylpropanamide (Compound 4.40)

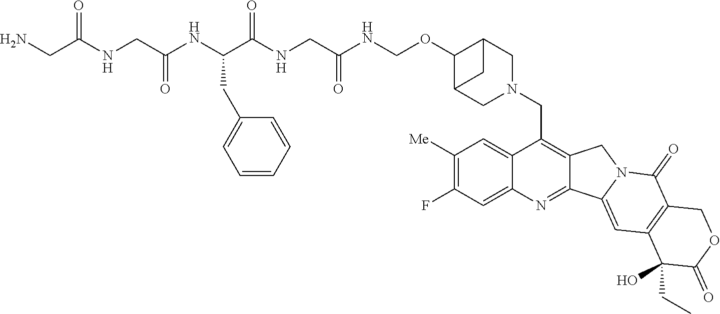 [SMILES: CC[C@@]1(O)C(=O)OCc2c1cc1n(c2=O)Cc2c-1nc1cc(F)c(C)cc1c2CN1CC2CC(C1)C2OCNC(=O)CNC(=O)[C@H](Cc1ccccc1)NC(=O)CNC(=O)CN]

The title compound was prepared according to General Procedure 1 starting from Compound 1.1 (3 mg, 0.007 mmol) and Compound 4.39 (14.7 mg, 0.022 mmol) and utilizing 200 uL DMF. Following complete consumption of Compound 1.1, a solution of 20% piperidine in DMF (200 uL) was added and this solution was stirred at room temperature for 10 min. Preparative HPLC purification was accomplished as described in General Procedure 9, eluting with a 20 to 37% CH₃CN/H₂O+0.1% TFA gradient to give the title compound as an off-white solid (TFA salt, 1.8 mg, 29% yield).

LC/MS: Calc'd m/z=852.9 for C₄₄H₄₉FN₈O₉, found [M+H]⁺=853.7.

### 4.41: (2S)-2-(1-(2,5-dioxo-2,5-dihydro-1H-pyrrol-1-yl)-12,15-dioxo-3,6,9-trioxa-13,16-diazaoctadecan-18-amido)-N-(2-((((3-(((S)-4-ethyl-8-fluoro-4-hydroxy-9-methyl-3,14-dioxo-3,4,12,14-tetrahydro-1H-pyrano[3,4′:6,7]indolizino[1,2-b]quinolin-11-yl)methyl)-3-azabicyclo[3.1.1]heptan-6-yl)oxy)methyl)amino)-2-oxoethyl)-3-phenylpropanamide (MT-GGFG-AM-Compound 117)

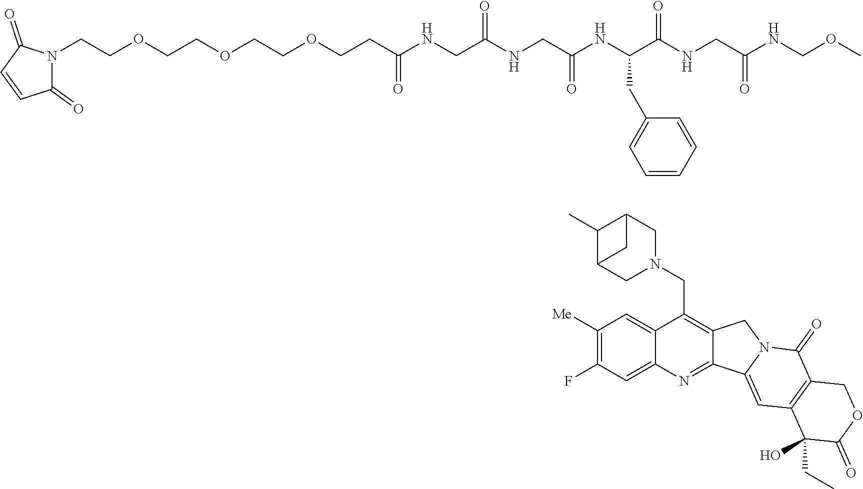

The title compound was prepared according to Procedure 8 starting from Compound 4.40 (1.8 mg). Preparative HPLC purification was accomplished as described in General Procedure 9, eluting with a 25 to 45% CH₃CN/H₂O+0.1% TFA gradient to provide the title compound as a white-solid (TFA salt, 0.5 mg, 22% yield).

LC/MS: Calc'd m/z=1135.5 for C₅₇H₆₆FN₉O₁₅, found [M+H]⁺=1136.3.

### 4.42: (9H-fluoren-9-yl)methyl (S)-(9-benzyl-1-(3-fluoroazetidin-3-yl)-5,8,11,14-tetraoxo-2-oxa-4,7,10,13-tetraazapentadecan-15-yl)carbamate (Compound 4.42)

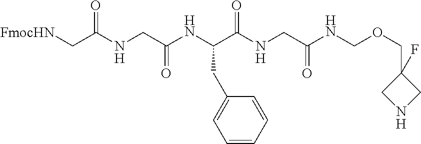

To a stirring solution of Compound 4.6 (144 mg) in dichloromethane (2 mL) was added (3-fluoroazetidin-3-yl)methanol (16 mg) followed by trifluoracetic acid (0.4 mL). After 30 min the reaction was concentrated in vacuo. Purification was accomplished as described in General Procedure 9, using a 10 g flash column and eluting with a 0 to 20% dichloromethane/methanol gradient to provide the title compound as a white solid (55 mg, 54% yield).

LC/MS: Calc'd m/z=674.7 for C₃₅H₃₉N₆FO₇, found [M+H]⁺=675.6.

### 4.43: (S)-2-(2-(2-aminoacetamido)acetamido)-N-(2-((((1-(((S)-4-ethyl-8-fluoro-4-hydroxy-9-methyl-3,14-dioxo-3,4,12,14-tetrahydro-1H-pyrano[3′,4′:6,7]indolizino[1,2-b]quinolin-11-yl)methyl)-3-fluoroazetidin-3-yl)methoxy)methyl)amino)-2-oxoethyl)-3-phenylpropanamide (Compound 4.43)

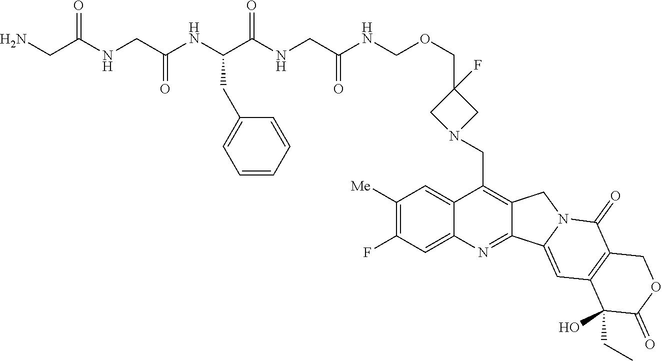 [SMILES: CC[C@@]1(O)C(=O)OCc2c1cc1n(c2=O)Cc2c-1nc1cc(F)c(C)cc1c2CN1CC(F)(COCNC(=O)CNC(=O)[C@H](Cc2ccccc2)NC(=O)CNC(=O)CN)C1]

The title compound was prepared according to General Procedure 1 starting from Compound 1.1 (11.6 mg, 0.027 mmol) and Compound 4.42 (55 mg, 0.082 mmol) and utilizing 500 uL DMF. Following complete consumption of Compound 1.1, a solution of 20% piperidine in DMF (500 uL) was added and this solution was stirred at room temperature for 10 min. Preparative HPLC purification was accomplished as described in General Procedure 9, eluting with a 25 to 32% CH₃CN/H₂O+0.1% TFA gradient to give the title compound as an off-white solid (TFA salt, 8.1 mg, 28% yield).

LC/MS: Calc'd m/z=844.3 for C₄₂H₄₆F₂N₈O₉, found [M+H]⁺=845.3

### 4.44: (S)-2-(1-(2,5-dioxo-2,5-dihydro-1H-pyrrol-1-yl)-12,15-dioxo-3,6,9-trioxa-13,16-diazaoctadecan-18-amido)-N-(2-((((1-(((S)-4-ethyl-8-fluoro-4-hydroxy-9-methyl-3,14-dioxo-3,4,12,14-tetrahydro-1H-pyrano[3,4′:6,7]indolizino[1,2-b]quinolin-11-yl)methyl)-3-fluoroazetidin-3-yl)methoxy)methyl)amino)-2-oxoethyl)-3-phenylpropanamide (MT-GGFG-AM-Compound 118)

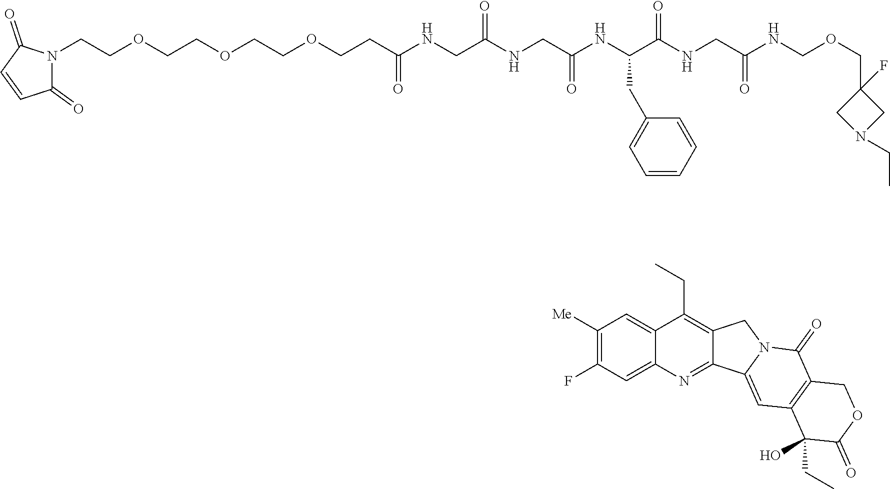 [SMILES: CCN1CC(F)(COCNC(=O)CNC(=O)[C@H](Cc2ccccc2)NC(=O)CNC(=O)CNC(=O)CCOCCOCCOCCN2C(=O)C=CC2=O)C1 ; CCc1c2c(nc3cc(F)c(C)cc13)-c1cc3c(c(=O)n1C2)COC(=O)[C@]3(O)CC]

The title compound was prepared according to Procedure 8 starting from Compound 4.43 (8.1 mg). Preparative HPLC purification was accomplished as described in General Procedure 9, eluting with a 25 to 45% CH₃CN/H₂O+0.1% TFA gradient to provide the title compound as a white-solid (TFA salt, 2.9 mg, 28% yield).

LC/MS: Calc'd m/z=1127.4 for C₅₅H₆₃F₂N₉O₁₅, found [M+H]⁺=1128.8.

### 4.45: (S)-10-benzyl-23-(2,5-dioxo-2,5-dihydro-1H-pyrrol-1-yl)-6,9,12,15,18-pentaoxo-3-oxa-5,8,11,14,17-pentaazatricosyl (((S)-4-ethyl-8-fluoro-4-hydroxy-9-methyl-3,14-dioxo-3,4,12,14-tetrahydro-1H-pyrano[3,4′:6,7]indolizino[1,2-b]quinolin-11-yl)methyl)carbamate (MC-GGFG-AM-Compound 139)

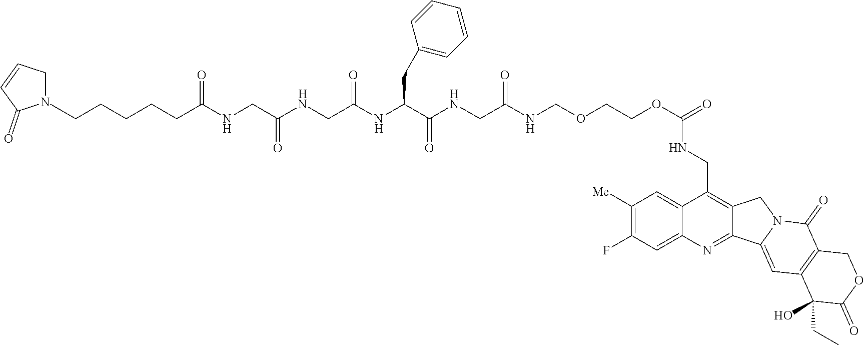

To Compound 4.27 (450 mg) was added a solution of 2,5-dioxopyrrolidin-1-yl 6-(2,5-dioxopyrrol-1-yl)hexanoate (130 mg) and N-ethyldiisopropylamine (250 uL) in DMF (10 mL). This solution was stirred at room temperature for 30 min then concentrated to −1 mL volume. Purification was accomplished as described in General Procedure 9 first using a 60 g C₁₈ flash column and eluting with a 10 to 60% CH₃CN/H₂O+0.1% TFA gradient followed by preparative HPLC of impure fractions using a 20 to 50% CH₃CN/H₂O+0.1% TFA gradient to provide the title compound as a white solid (320 mg, 66% yield).

LC/MS: Calc'd m/z=1037.4 for C₅₁H₅₆FN₉O₁₄, found [M+H]⁺=1038.6.

¹H NMR (300 MHz, MeOD) δ 8.10 (d, J=8.1 Hz, 2H), 8.01 (s, 1H), 7.95 (d, J=7.0 Hz, 1H), 7.74 (d, J=10.4 Hz, 1H), 7.66 (s, 1H), 7.56 (s, 1H), 7.32-7.10 (m, 5H), 6.69 (s, 2H), 5.63 (d, J=16.4 Hz, 1H), 5.46 (s, 2H), 5.32 (s, 1H), 5.28 (d, J=16.5 Hz, 1H), 4.88 (s, 2H), 4.67 (d, J=6.4 Hz, 2H), 4.48 (d, J=7.1 Hz, 2H), 4.15 (t, J=4.2 Hz, 2H), 3.92 (dd, J=17.1, 6.2 Hz, 2H), 3.83-3.57 (m, 6H), 3.46 (t, J=7.1 Hz, 2H), 3.16 (dd, J=14.0, 5.9 Hz, 1H), 2.95 (dd, J=13.9, 8.9 Hz, 1H), 2.53 (s, 3H), 2.21 (t, J=7.6 Hz, 2H), 1.97-1.79 (m, 2H), 1.58 (dp, J=15.0, 7.6 Hz, 4H), 1.29 (dd, J=16.6, 9.3 Hz, 3H), 1.01 (t, J=7.3 Hz, 3H).

### 4.46: tert-butyl (S)-(2-((4-ethyl-8-fluoro-4-hydroxy-3,14-dioxo-3,4,12,14-tetrahydro-1H-pyrano[3,4′:6,7]indolizino[1,2-b]quinolin-9-yl)amino)-2-oxoethyl)carbamate (Compound 4.46)

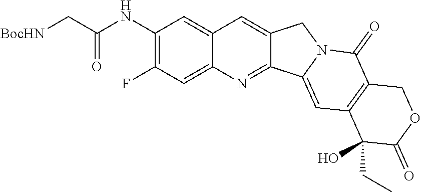 [SMILES: CC[C@@]1(O)C(=O)OCc2c1cc1n(c2=O)Cc2cc3cc(NC(=O)CNC(=O)OC(C)(C)C)c(F)cc3nc2-1]

A solution of Compound 140 (860 mg, 1.7 mmol, TFA salt), Boc-Gly-OH (760 mg, 4.3 mmol), HATU (1.6 g, 4.1 mmol), and N-ethyldiisopropylamine (0.6 mL) in DMF (4 mL) was stirred at room temperature for 24 h then poured into water (50 mL). The resulting solid was collected by filtration, redissolved in 10% MeOH/DCM and purification was accomplished as described in General Procedure 9, using a 30 g silica column and eluting with a 0 to 10% MeOH/DCM to provide the title compound as a yellow solid (750 mg, 80% yield).

LC/MS: Calc'd m/z=538.5 for C₂₇H₂₇FN₄O₇, found [M+H]⁺=539.4.

¹H NMR (300 MHz, MeOD) δ 8.84 (d, J=8.4 Hz, 1H), 8.52 (s, 1H), 8.00 (s, 1H), 7.87 (d, J=12.1 Hz, 1H), 7.62 (s, 1H), 5.60 (d, J=16.3 Hz, 1H), 5.40 (d, J=16.4 Hz, 1H), 5.27 (s, 2H), 4.02 (s, 2H), 1.99 (dt, J=8.7, 6.7 Hz, 2H), 1.52 (s, 9H), 1.03 (t, J=7.4 Hz, 3H).

### 4.47: (S)-1-((2-(((S)-4-ethyl-8-fluoro-4-hydroxy-3,14-dioxo-3,4,12,14-tetrahydro-1H-pyrano[3,4′:6,7]indolizino[1,2-b]quinolin-9-yl)amino)-2-oxoethyl)amino)-1-oxo-3-phenylpropan-2-aminium (Compound 4.47)

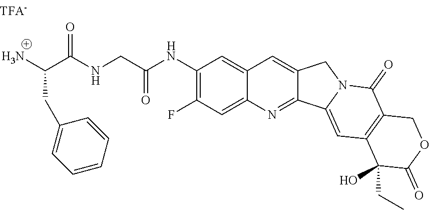 [SMILES: CC[C@@]1(O)C(=O)OCc2c1cc1n(c2=O)Cc2cc3cc(NC(=O)CNC(=O)[C@@H]([NH3+])Cc4ccccc4)c(F)cc3nc2-1]

The title compound was prepared in three steps from Compound 4.46 (750 mg). The Boc protecting group was cleaved in neat TFA (2 mL) followed by precipitation in Et₂O (50 mL). The solid was collected by filtration and added to a solution of 2,5-dioxopyrrolidin-1-yl (2S)-2-[(tert-butoxycarbonyl)amino]-3-phenylpropanoate (340 mg, 1.1 equiv) and N-ethyldiisopropylamine (300 uL) in DMF (1.7 mL). This solution was stirred at room temperature for 30 min then pipetted into Et₂O (50 mL). The precipitate was collected by filtration, dried under vacuum then dissolved in neat TFA (2 mL). After 20 min, Et₂O (50 mL) was added and the precipitate collected by filtration to provide the title compound as a yellow solid (531 mg, 54% yield).

LC/MS: Calc'd m/z=585.2 for C₃₁H₂₈FN₅O₆, found [M+H]⁺=586.1.

### 4.48: 2-((2-(((S)-1-((2-(((S)-4-ethyl-8-fluoro-4-hydroxy-3,14-dioxo-3,4,12,14-tetrahydro-1H-pyrano[3,4′:6,7]indolizino[1,2-b]quinolin-9-yl)amino)-2-oxoethyl)amino)-1-oxo-3-phenylpropan-2-yl)amino)-2-oxoethyl)amino)-2-oxoethan-1-aminium (Compound 4.48)

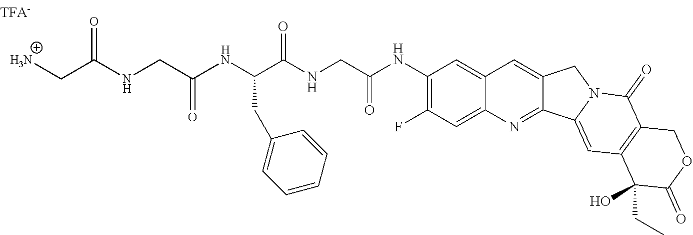 [SMILES: CC[C@@]1(O)C(=O)OCc2c1cc1n(c2=O)Cc2cc3cc(NC(=O)CNC(=O)[C@H](Cc4ccccc4)NC(=O)CNC(=O)C[NH3+])c(F)cc3nc2-1]

To Compound 4.47 (490 mg) was added a solution of Boc-gly-gly-NHS (250 mg, 1.1 equiv) and N-ethyldiisopropylamine (250 uL) in DMF (3 mL). This solution was stirred at room temperature for 30 min then pipetted into Et₂O (50 mL). The precipitate was collected by filtration then dissolved in neat TFA (2 mL). After 20 min, Et₂O (50 mL) was added and the precipitate collected by filtration to provide the title compound as a yellow solid (500 mg, 88% yield).

LC/MS: Calc'd m/z=699.2 for C₃₅H₃₄FN₇O₈, found [M+H]⁺=700.4.

### 4.49: 6-(2,5-dioxo-2,5-dihydro-1H-pyrrol-1-yl)-N-(2-((2-(((S)-1-((2-(((S)-4-ethyl-8-fluoro-4-hydroxy-3,14-dioxo-3,4,12,14-tetrahydro-1H-pyrano[3′,4′:6,7]indolizino[1,2-b]quinolin-9-yl)amino)-2-oxoethyl)amino)-1-oxo-3-phenylpropan-2-yl)amino)-2-oxoethyl)amino)-2-oxoethyl)hexanamide (MC-GGFG-Compound 140)

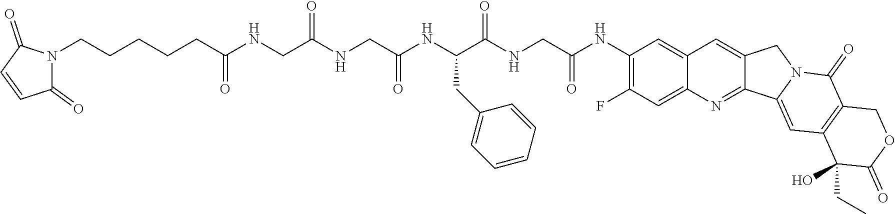 [SMILES: CC[C@@]1(O)C(=O)OCc2c1cc1n(c2=O)Cc2cc3cc(NC(=O)CNC(=O)[C@H](Cc4ccccc4)NC(=O)CNC(=O)CNC(=O)CCCCCN4C(=O)C=CC4=O)c(F)cc3nc2-1]

To Compound 4.48 (500 mg) was added a solution of 2,5-dioxocyclopentyl 6-(2,5-dioxopyrrol-1-yl)hexanoate (210 mg, 1.1 equiv) and N-ethyldiisopropylamine (215 uL) in DMF (4 mL). This solution was stirred at room temperature for 30 min then pipetted into Et₂O (50 mL). The precipitate was collected by filtration then dissolved in DMF (2 mL). Preparative HPLC purification was accomplished as described in General Procedure 9, eluting with a 24 to 38% CH₃CN/H₂O+0.1% TFA gradient to provide the title compound as a yellow solid (190 mg, 40% yield).

LC/MS: Calc'd m/z=892.9 for C₄₅H₄₅FN₈O₁₁, found [M+H]⁺=893.6.

¹H NMR (300 MHz, CD₃CN) δ 8.67 (d, J=8.4 Hz, 1H), 8.44 (s, 1H), 7.78 (d, J=12.1 Hz, 1H), 7.41 (s, 1H), 7.30 (d, J=4.3 Hz, 4H), 7.26-7.16 (m, 1H), 6.72 (s, 2H), 5.52 (d, J=16.4 Hz, 1H), 5.31 (d, J=16.4 Hz, 1H), 5.12 (s, 2H), 4.64 (dd, J=9.7, 5.0 Hz, 1H), 4.11 (d, J=3.2 Hz, 2H), 3.87-3.68 (m, 4H), 3.37 (t, J=7.1 Hz, 2H), 3.00 (dd, J=14.0, 9.7 Hz, 1H), 2.20 (t, J=7.6 Hz, 2H), 1.49 (dq, J=19.5, 7.4 Hz, 4H), 1.22 (p, J=7.6, 7.1 Hz, 2H), 0.94 (t, J=7.3 Hz, 3H).

### 4.50: tert-butyl (S)-(2-((4-ethyl-8-fluoro-4-hydroxy-11-(hydroxymethyl)-3,14-dioxo-3,4,12,14-tetrahydro-1H-pyrano[3,4′:6,7]indolizino[1,2-b]quinolin-9-yl)amino)-2-oxoethyl)carbamate (Compound 4.50)

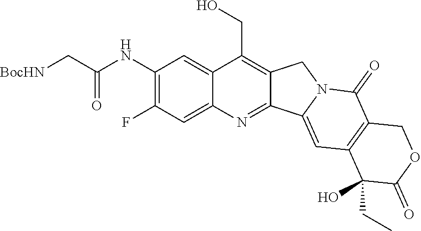 [SMILES: CC[C@@]1(O)C(=O)OCc2c1cc1n(c2=O)Cc2c-1nc1cc(F)c(NC(=O)CNC(=O)OC(C)(C)C)cc1c2CO]

A solution of Compound 4.46 (1.8 g), iron (II) sulfate heptahydrate (1.4 g, 1.5 equiv), and sulfuric acid (450 uL, 2.5 equiv) in MeOH (33 mL) was heated to 60° C. and hydrogen peroxide (1.25 mL, 12 equiv) was added dropwise over 10 min. This solution was heated for another 20 min then cooled to room temperature and poured into ice water (˜200 mL). The brown precipitate was collected by filtration and the filtrate was quenched with saturated aqueous Na₂S₂O₃. MeOH was evaporated and the solution allowed to stand for 2 h while a second brown precipitate formed. This precipitate was collected by filtration and the combined precipitates were purified as described in General Procedure 9 using a 50 g silica column and eluting with a 0 to 15% MeOH/DCM gradient to provide the title compound as a yellow solid (860 mg, 45% yield).

LC/MS: Calc'd m/z=568.5 for C₂₈H₂₉FN₄O₈, found [M+H]⁺=569.7.

### 4.51: (S)-1-((2-(((S)-4-ethyl-8-fluoro-4-hydroxy-11-(hydroxymethyl)-3,14-dioxo-3,4,12,14-tetrahydro-1H-pyrano[3,4′:6,7]indolizino[1,2-b]quinolin-9-yl)amino)-2-oxoethyl)amino)-1-oxo-3-phenylpropan-2-aminium (Compound 4.51)

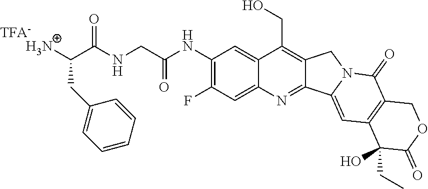

The title compound was prepared in three steps from Compound 4.50 (750 mg). The Boc protecting group was cleaved in neat TFA (2 mL) followed by precipitation in Et₂O (100 mL). The solid was collected by filtration and added to a solution of 2,5-dioxopyrrolidin-1-yl (2S)-2-[(tert-butoxycarbonyl)amino]-3-phenylpropanoate (600 mg, 1.1 equiv) and N-ethyldiisopropylamine (300 uL) in DMF (7 mL). This solution was stirred at room temperature for 30 min then pipetted into Et₂O (100 mL). The precipitate was collected by filtration, dried under vacuum then dissolved in neat TFA (2 mL). After 20 min, Et₂O (100 mL) was added and the precipitate collected by filtration to provide the title compound as a yellow solid (756 mg, 78% yield).

LC/MS: Calc'd m/z=615.2 for C₃₂H₃₀FN₅O₇, found [M+H]⁺=616.3.

### 4.52: 2-((2-(((S)-1-((2-(((S)-4-ethyl-8-fluoro-4-hydroxy-11-(hydroxymethyl)-3,14-dioxo-3,4,12,14-tetrahydro-1H-pyrano[3,4′:6,7]indolizino[1,2-b]quinolin-9-yl)amino)-2-oxoethyl)amino)-1-oxo-3-phenylpropan-2-yl)amino)-2-oxoethyl)amino)-2-oxoethan-1-aminium (Compound 4.52)

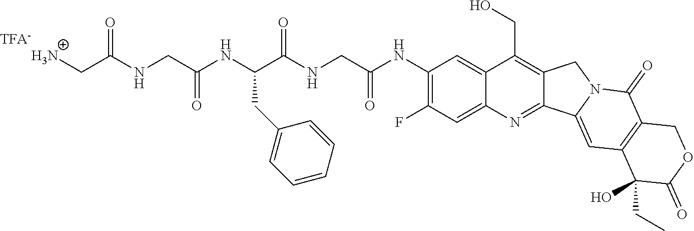 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)C[NH3+])cc1c2CO ; "TFA-"]

To Compound 4.51 (756 mg) was added a solution of Boc-gly-gly-NHS (375 mg, 1.1 equiv) and N-ethyldiisopropylamine (400 uL) in DMF (5 mL). This solution was stirred at room temperature for 30 min then pipetted into Et₂O (75 mL). The precipitate was collected by filtration then dissolved in neat TFA (4 mL). After 20 min, Et₂O (100 mL) was added and the precipitate collected by filtration to provide the title compound as a yellow solid (826 mg, 95% yield).

LC/MS: Calc'd m/z=729.2 for C₃₆H₃₆FN₇O₉, found [M+H]⁺=730.2.

### 4.53: 6-(2,5-dioxo-2,5-dihydro-1H-pyrrol-1-yl)-N-(2-((2-(((S)-1-((2-(((S)-4-ethyl-8-fluoro-4-hydroxy-11-(hydroxymethyl)-3,14-dioxo-3,4,12,14-tetrahydro-1H-pyrano[3,4′:6,7]indolizino[1,2-b]quinolin-9-yl)amino)-2-oxoethyl)amino)-1-oxo-3-phenylpropan-2-yl)amino)-2-oxoethyl)amino)-2-oxoethyl)hexanamide (MC-GGFG-Compound 141)

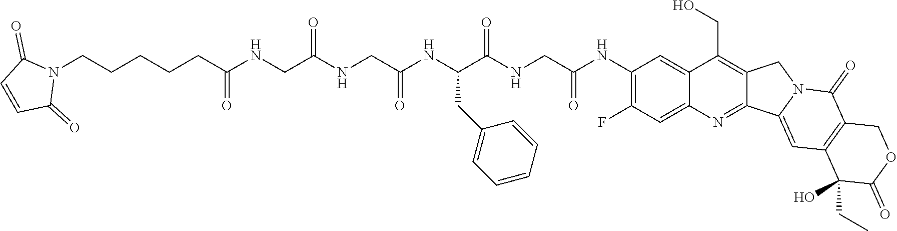 [SMILES: CC[C@@]1(O)C(=O)OCc2c1cc1n(c2=O)Cc2c-1nc1cc(F)c(NC(=O)CNC(=O)[C@H](Cc3ccccc3)NC(=O)CNC(=O)CNC(=O)CCCCCN3C(=O)C=CC3=O)cc1c2CO]

To Compound 4.52 (826 mg) was added a solution of 2,5-dioxocyclopentyl 6-(2,5-dioxopyrrol-1-yl)hexanoate (382 mg, 1.1 equiv) and N-ethyldiisopropylamine (300 uL) in DMF (5.5 mL). This solution was stirred at room temperature for 30 min then pipetted into Et₂O (100 mL). The precipitate was collected by filtration then dissolved in DMF (2 mL). Preparative HPLC purification was accomplished as described in General Procedure 9, eluting with a 25 to 40% CH₃CN/H₂O+0.1% TFA gradient to provide the title compound as a yellow solid (370 mg, 35% yield).

LC/MS: Calc'd m/z=922.9 for C₄₆H₄₇FN₈O₁₂, found [M+H]⁺=923.8.

¹H NMR (300 MHz, CD₃CN) δ 8.63 (d, J=8.4 Hz, 1H), 7.67 (d, J=11.9 Hz, 1H), 7.38-7.27 (m, 5H), 7.24 (d, J=4.3 Hz, 1H), 6.72 (s, 2H), 5.48 (d, J=16.4 Hz, 1H), 5.28 (d, J=16.3 Hz, 1H), 5.24-5.01 (m, 4H), 4.65 (dd, J=9.7, 4.9 Hz, 1H), 4.13 (s, 2H), 3.85-3.75 (m, 3H), 3.37 (t, J=7.1 Hz, 2H), 3.00 (dd, J=14.0, 9.8 Hz, 1H), 2.21 (t, J=7.6 Hz, 2H), 1.51 (dp, J=22.0, 7.4 Hz, 4H), 1.22 (p, J=7.4, 7.0 Hz, 2H), 0.94 (t, J=7.3 Hz, 3H).

### 4.54: tert-butyl ((S)-1-(((S)-4-ethyl-8-fluoro-4-hydroxy-3,14-dioxo-3,4,12,14-tetrahydro-1H-pyrano[3,4′:6,7]indolizino[1,2-b]quinolin-9-yl)amino)-1-oxopropan-2-yl)carbamate (Compound 4.54)

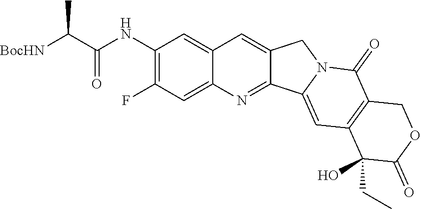 [SMILES: CC[C@@]1(O)C(=O)OCc2c1cc1n(c2=O)Cc2cc3cc(NC(=O)[C@H](C)NC(=O)OC(C)(C)C)c(F)cc3nc2-1]

To Compound 3.4 (500 mg, 1.0 mmol) was added TFA (4 mL) and this solution was allowed to stand at rt for 1 h, then Et₂O (100 mL) was added, and the precipitate was collected by filtration. This solid was taken up in DMF (3.4 mL) and Boc-Ala-OH (590 mg, 3.1 mmol, 3 equiv) and HATU (1.2 g, 3.1 mmol, 3equiv) were added followed by N-ethyldiisopropylamine (0.9 mL, 5.2 mmol, 5 equiv). This solution was stirred at rt for 3 days then poured into ice water (50 mL) and the precipitate was collected by filtration to give the title compound as a brown solid (125 mg, 22% yield).

LC/MS: Calc'd m/z=552.6 for C₂₈H₂₉FN₄O₇, found [M+H]⁺=553.7.

### 4.55: (S)-2-amino-N-((S)-1-(((S)-4-ethyl-8-fluoro-4-hydroxy-3,14-dioxo-3,4,12,14-tetrahydro-1H-pyrano[3,4′:6,7]indolizino[1,2-b]quinolin-9-yl)amino)-1-oxopropan-2-yl)-3-methylbutanamide (Compound 4.55)

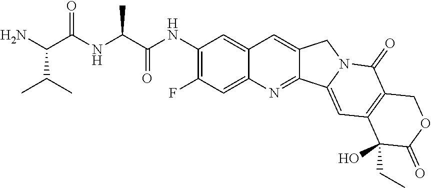 [SMILES: CC[C@@]1(O)C(=O)OCc2c1cc1n(c2=O)Cc2cc3cc(NC(=O)[C@H](C)NC(=O)[C@@H](N)C(C)C)c(F)cc3nc2-1]

To Compound 4.54 (125 mg, 0.225 mmol) in a 100 mL round bottom flask was added TFA (2 mL). This solution was allowed to stand for 10 min, then Et₂O (50 mL) was added, and the precipitate collected by filtration. The resulting orange solid was added to a solution of Boc-Val-NHS (78 mg, 0.25 mmol, 1.1 equiv) and N-ethyldiisopropylamine (80 uL, 0.45 mmol, 2 equiv) in DMF (2 mL). This solution was stirred at rt for 30 min, then pipetted into Et₂O (40 mL) in a 50 mL falcon tube and the precipitate was collected by centrifugation and decanting of the Et₂O. The pellet was dissolved in TFA (2 mL) and allowed to stand for 10 min prior to the addition of Et₂O (40 mL). The precipitate was collected by centrifugation and decanting the Et₂O. The pellet was dried under high vacuum to give the title compound as an orange solid (135 mg, 90% yield over 3 steps).

LC/MS: Calc'd m/z=551.2 for C₂₈H₃₀FN₅O₆, found [M+H]⁺=552.2.

### 4.56: 6-(2,5-dioxo-2,5-dihydro-1H-pyrrol-1-yl)-N-((S)-1-(((S)-1-(((S)-4-ethyl-8-fluoro-4-hydroxy-3,14-dioxo-3,4,12,14-tetrahydro-1H-pyrano[3′,4′:6,7]indolizino[1,2-b]quinolin-9-yl)amino)-1-oxopropan-2-yl)amino)-3-methyl-1-oxobutan-2-yl)hexanamide (MC-VA-Compound 140)

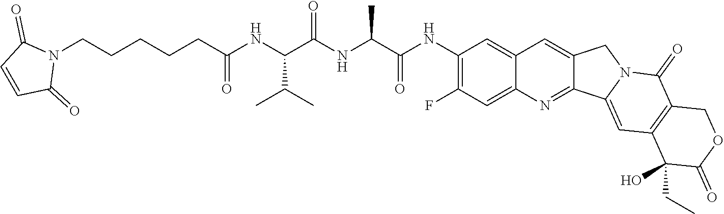 [SMILES: CC[C@@]1(O)C(=O)OCc2c1cc1n(c2=O)Cc2cc3cc(NC(=O)[C@H](C)NC(=O)[C@@H](NC(=O)CCCCCN4C(=O)C=CC4=O)C(C)C)c(F)cc3nc2-1]

To Compound 4.55 (20 mg, 0.03 mmol) was added a solution of 2,5-dioxopyrrolidin-1-yl 6-(2,5-dioxopyrrol-1-yl)hexanoate (11 mg, 0.036 mmol) and N-ethyldiisopropylamine (10 uL) in DMF (1 mL). This solution was stirred at rt for 30 min then purified directly. Preparative HPLC purification was accomplished as described in General Procedure 9, eluting with a 20 to 60% CH₃CN/H₂O+0.1% TFA gradient to provide the title compound as a yellow solid (8.8 mg, 40% yield).

LC/MS: Calc'd m/z=744.8 for C₃₈H₄₁FN₆O₉, found [M+H]⁺=745.6.

¹H NMR (300 MHz, 10% D₂O/CD₃CN) δ 8.60 (d, J=8.5 Hz, 1H), 8.31 (s, 1H), 7.96 (d, J=6.5 Hz, 1H), 7.65 (d, J=12.0 Hz, 1H), 7.37-7.26 (m, 2H), 6.75 (s, 2H), 5.45 (d, J=16.6 Hz, 1H), 5.25 (d, J=16.3 Hz, 1H), 5.04 (d, J=4.0 Hz, 2H), 4.78-4.58 (m, 1H), 4.30-4.13 (m, 1H), 2.32-2.16 (m, 2H), 2.10 (dt, J=13.6, 6.8 Hz, 1H), 1.88 (q, J=7.4 Hz, 2H), 1.57 (dq, J=15.5, 7.6 Hz, 4H), 1.45 (d, J=7.1 Hz, 3H), 1.26 (tt, J=10.1, 6.1 Hz, 2H), 1.05-0.83 (m, 9H).

### 4.57: 2,5-dioxopyrrolidin-1-yl 6-(((S)-1-(((S)-1-(((S)-4-ethyl-8-fluoro-4-hydroxy-3,14-dioxo-3,4,12,14-tetrahydro-1H-pyrano[3,4′:6,7]indolizino[1,2-b]quinolin-9-yl)amino)-1-oxopropan-2-yl)amino)-3-methyl-1-oxobutan-2-yl)amino)-6-oxohexanoate (NHC-C-VA-Compound 140)

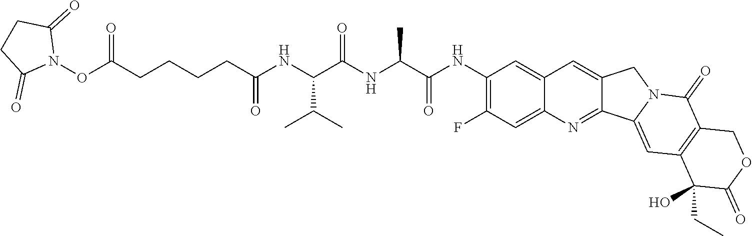 [SMILES: CC[C@@]1(O)C(=O)OCc2c1cc1n(c2=O)Cc2cc3cc(NC(=O)[C@H](C)NC(=O)[C@@H](NC(=O)CCCCC(=O)ON4C(=O)CCC4=O)C(C)C)c(F)cc3nc2-1]

To Compound 4.55 (20 mg, 0.03 mmol) was added a solution of bis(2,5-dioxopyrrolidin-1-yl) adipate (30 mg, 0.09 mmol, 3 equiv) and N-ethyldiisopropylamine (10 uL) in DMF (1 mL). This solution was stirred at rt for 30 min then purified directly. Preparative HPLC purification was accomplished as described in General Procedure 9, eluting with a 25 to 35% CH₃CN/H₂O+0.1% TFA gradient to provide the title compound as a yellow solid (4.1 mg, 18% yield).

LC/MS: Calc'd m/z=776.8 for C₃₈H₄₁FN₆01r, found [M+H]⁺=777.6.

¹H NMR (300 MHz, 10% D₂O/CD₃CN) δ 8.65 (dd, J=8.4, 2.3 Hz, 1H), 8.38 (s, 1H), 7.95 (d, J=6.5 Hz, 1H), 7.72 (d, J=12.0 Hz, 1H), 7.37 (d, J=11.8 Hz, 2H), 5.58-5.19 (m, 2H), 5.10 (s, 2H), 4.78-4.56 (m, 1H), 4.23 (dd, J=8.4, 7.0 Hz, 1H), 2.80 (s, 4H), 2.65 (t, J=6.9 Hz, 2H), 2.39-2.22 (m, 2H), 2.11 (q, J=6.8 Hz, 1H), 1.94-1.81 (m, 2H), 1.79-1.57 (m, 4H), 1.45 (d, J=7.1 Hz, 3H), 1.10-0.78 (m, 9H).

### 4.58: (S)-2-(32-azido-5-oxo-3,9,12,15,18,21,24,27,30-nonaoxa-6-azadotriacontanamido)-N-((S)-1-(((S)-4-ethyl-8-fluoro-4-hydroxy-3,14-dioxo-3,4,12,14-tetrahydro-1H-pyrano[3,4′:6,7]indolizino[1,2-b]quinolin-9-yl)amino)-1-oxopropan-2-yl)-3-methylbutanamide (2-((Azido-PEG8-carbamoyl)methoxy)acetamido-VA-Compound 140)

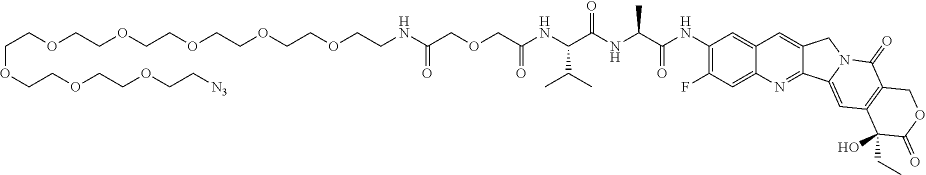 [SMILES: CC[C@@]1(O)C(=O)OCc2c1cc1n(c2=O)Cc2cc3cc(NC(=O)[C@H](C)NC(=O)[C@@H](NC(=O)COCC(=O)NCCOCCOCCOCCOCCOCCOCCOCCOCCN=[N+]=[N-])C(C)C)c(F)cc3nc2-1]

A solution of Compound 4.55 (20 mg, 0.03 mmol), 32-azido-5-oxo-3,9,12,15,18,21,24,27,30-nonaoxa-6-azadotriacontanoic acid (17 mg, 0.03 mmol) and HATU (13 mg, 0.03 mmol) in DMF (300 uL) was cooled to 0° C. and N-ethyldiisopropylamine (16 uL, 0.09 mmol) was added. This solution was stirred for 30 min then purified directly. Preparative HPLC purification was accomplished as described in General Procedure 9, eluting with a 20 to 50% CH₃CN/H₂O+0.1% TFA gradient to provide the title compound as a yellow solid (13.7 mg, 42% yield).

LC/MS: Calc'd m/z=1088.2 for C₅₀H₇₀FN₉O₁₇, found [M+H]⁺=1088.8.

¹H NMR (300 MHz, 10% D₂O/CD₃CN) δ 8.62 (d, J=8.5 Hz, 1H), 8.33 (s, 1H), 7.66 (d, J=12.2 Hz, 1H), 7.35 (s, 1H), 5.52-5.18 (m, 2H), 5.04 (s, 2H), 4.72 (q, J=7.1 Hz, 1H), 4.32 (d, J=7.3 Hz, 1H), 4.15-3.98 (m, 4H), 3.68-3.48 (m, 35H), 3.41-3.34 (m, 6H), 2.18 (h, J=6.8 Hz, 1H), 1.88 (q, J=7.4 Hz, 2H), 1.47 (d, J=7.1 Hz, 3H), 1.13-0.84 (m, 9H).

### 4.58: tert-butyl (2-(pyridin-2-yldisulfanyl)ethyl)carbamate (Compound 4.58)

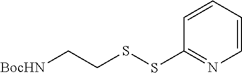 [SMILES: CC(C)(C)OC(=O)NCCSSc1ccccn1]

The title compound was prepared as described in Wang, et al., Nano Lett., 2014, 14(10):5577-5583.

### 4.59: tert-butyl (2-((2-hydroxyethyl)disulfaneyl)ethyl)carbamate (Compound 4.59)

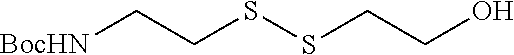 [SMILES: CC(C)(C)OC(=O)NCCSSCCO]

To a solution of Compound 4.58 (200 mg, 0.7 mmol) in DCM (1.4 mL) was added β-mercaptoethanol (50 μL, 0.7 mmol) and this solution was stirred at rt for 5 h. The solution was diluted with DCM (10 mL), washed with a water (3×10 mL), dried over Na₂SO₄ and concentrated to an oil. Purification was accomplished as described in General Procedure 9, using a 10 g silica column and eluting with a 0 to 10% MeOH/DCM to give the title compound as a colorless solid (212 mg, 82% yield).

LC/MS: Calc'd m/z=253.1 for C₁₁H₂₃NO₃S₂, found [M+H,-Boc]⁺=154.0.

¹H NMR (300 MHz, Chloroform-d) δ 4.94 (s, 1H), 3.91 (t, J=5.7 Hz, 2H), 3.49 (q, J=6.4 Hz, 2H), 2.86 (dt, J=23.7, 6.1 Hz, 4H), 2.15 (s, 2H), 1.47 (s, 9H).

### 4.60: 2-((2-(3-(2,5-dioxo-2,5-dihydro-1H-pyrrol-1-yl)propanamido)ethyl)disulfaneyl)ethyl (4-nitrophenyl) carbonate (Compound 4.60)

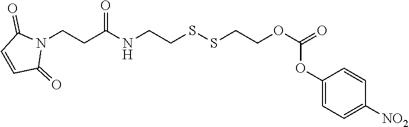 [SMILES: O=C(CCN1C(=O)C=CC1=O)NCCSSCCOC(=O)Oc1ccc([N+](=O)[O-])cc1]

To Compound 4.59 (212 mg, 0.837 mmol) in a 25 mL round bottom flask was added a 4M HCl/dioxane solution (5 mL) and the solution was stirred at rt for 30 min, then evaporated to dryness. The residue was suspended in EtOAc (10 mL) and evaporated to dryness to give the amine as the HCl salt and as a white powder. To this solid was added a solution of 2,5-dioxopyrrolidin-1-yl 3-(2,5-dioxopyrrol-1-yl)propanoate (245 mg, 0.92 mmol, 1.1 equiv.) and N-ethyldiisopropylamine (0.438 mL, 2.51 mmol) in DMF (1.7 mL). This solution was stirred at rt for 20 min then 4-nitrophenyl carbonate (280 mg, 0.92 mmol) was added and the reaction was then left to stir overnight. Purification of the crude reaction mixture was accomplished as described in General Procedure 9, using a 12 g C18 flash column, and eluting with a 10 to 100% CH₃CN/H₂O+0.1% TFA gradient to provide the title compound as a white solid (141 mg, 36% yield).

LC/MS: Calc'd m/z=469.5 for C₁₈H₁₉N₃O₈S₂, found [M+H]⁺=470.2.

¹H NMR (300 MHz, Chloroform-d) δ 8.37-8.25 (m, 2H), 7.46-7.35 (m, 2H), 6.71 (d, J=2.1 Hz, 2H), 6.32 (s, 1H), 4.55 (t, J=6.6 Hz, 2H), 3.83 (t, J=7.0 Hz, 2H), 3.65-3.50 (m, 2H), 3.09-2.99 (m, 2H), 2.84 (q, J=6.1 Hz, 2H), 2.52 (td, J=7.1, 3.1 Hz, 2H).

### 4.61: 2-((2-(3-(2,5-dioxo-2,5-dihydro-1H-pyrrol-1-yl)propanamido)ethyl)disulfaneyl)ethyl (S)-((9-amino-4-ethyl-8-fluoro-4-hydroxy-3,14-dioxo-3,4,12,14-tetrahydro-1H-pyrano[3,4′:6,7]indolizino[1,2-b]quinolin-11-yl)methyl)carbamate (DiS-Compound 145)

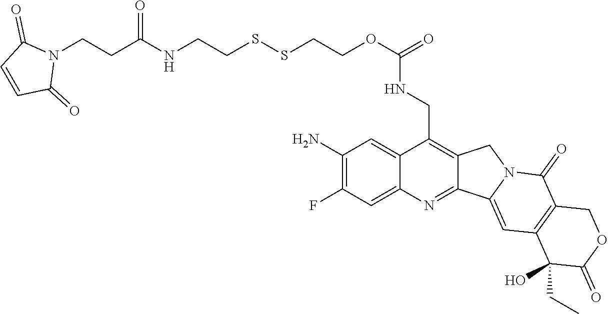 [SMILES: CC[C@@]1(O)C(=O)OCc2c1cc1n(c2=O)Cc2c-1nc1cc(F)c(N)cc1c2CNC(=O)OCCSSCCNC(=O)CCN1C(=O)C=CC1=O]

A solution of Compound 4.60 (18 mg, 0.038 mmol) and N-ethyldiisopropylamine (15 uL, 0.087 mmol) in DMF (300 uL) was added to Compound 145 (13 mg, 0.029 mmol) and this solution was stirred at rt for 20 min. The solution was acidified with an aqueous 1M HCl solution (100 uL) and purified directly. Preparative HPLC purification was accomplished as described in General Procedure 9, eluting with a 20 to 45% CH₃CN/H₂O+0.1% TFA gradient to provide the title compound as a yellow solid (6.8 mg, 32% yield).

LC/MS: Calc'd m/z=740.8 for C₃₃H₃₃FN₆O₉S₂, found [M+H]⁺=741.5.

¹H NMR (300 MHz, 10% D₂O/CD₃CN) δ 7.63 (d, J=12.1 Hz, 1H), 7.39-7.22 (m, 2H), 6.74 (d, J=6.7 Hz, 2H), 5.50 (d, J=16.2 Hz, 1H), 5.26 (d, J=16.2 Hz, 1H), 5.20 (s, 2H) 4.69 (s, 2H), 4.28 (t, J=6.3 Hz, 2H), 3.62 (t, J=7.0 Hz, 2H), 3.31 (t, J=6.6 Hz, 2H), 2.74-2.64 (m, 2H), 2.35 (t, J=7.0 Hz, 2H), 1.90 (dd, J=15.5, 8.1 Hz, 2H), 1.23-1.04 (m, 6H), 0.93 (t, J=7.4 Hz, 3H).

### Example 5: In Vitro Cytotoxicity of Camptothecin Analogues

Cytotoxicity of the camptothecin analogues was assessed in vitro on the following cancer cell lines: SK-BR-3 (breast cancer), SKOV-3 (ovarian cancer), Calu-3 (lung cancer), ZR-75-1 (breast cancer) and MDA-MB-468 (breast cancer).

Briefly, serial dilutions of camptothecin analogues shown in Table 5.1 were prepared in RPMI 1640+10% FBS, and 20 uL of each dilution was added to 384-well plates. Cells cultured in log-phase growth were detached by brief incubation in 0.05% Trypsin and resuspended in respective culturing media at 20,000 cells/mL (with the exception of ZR-75 cells, which were resuspended at 10,000 cells/mL). 50 uL of cell suspension was then added to the plates containing test articles. Cells were incubated with test articles for 4 d at 37° C. (with the exception of ZR-75 cells, which were incubated for 5 d). Growth inhibition was assessed by CellTiter-Glo® (Promega Corporation, Madison, WI) and luminescence was measured on a Synergy™ H1 plate reader (BioTek Instruments, Winooski, VT). IC50 values were determined by GraphPad Prism (GraphPad Software, San Diego, CA). The calculated pIC50 values are shown in Table 5.1.

Table 5.1: In vitro Cytotoxicity of Camptothecin Analogues (pIC50)

TABLE 5.1
In vitro Cytotoxicity of Camptothecin Analogues (pIC50)

| | pIC50 | | | | |
|---|---|---|---|---|---|
| Camptothecin | | | | | MDA-MB- |
| Analogue | Calu-3 | SKBR-3 | SKOV-3 | ZR-75 | 468 |
| Compound 100 | 8.95 | 8.96 | 8.64 | 8.91 | 8.60 |
| Compound 102 | ND* | 9.03 | 8.67 | 9.10 | 8.63 |
| Compound 104 | 8.28 | 8.57 | 8.11 | 8.59 | 8.58 |
| Compound 122 | 8.43 | 8.95 | 8.04 | 8.89 | 8.96 |
| Compound 132 | 7.29 | 7.64 | 6.98 | 7.81 | 7.57 |
| Compound 106 | 8.34 | 8.47 | 8.13 | 8.42 | 8.03 |
| Compound 108 | 7.39 | 7.53 | 7.26 | 7.73 | 7.32 |
| Compound 101 | ND | 8.97 | 8.80 | 8.97 | 8.59 |
| Compound 103 | 8.63 | 8.77 | 8.36 | 8.62 | 8.33 |
| Compound 105 | 8.35 | 8.44 | 7.97 | 8.51 | 8.56 |
| Compound 107 | 8.39 | 8.51 | ND | 8.56 | 8.41 |
| Compound 109 | ND | 8.24 | 8.15 | 8.30 | 8.09 |
| Compound 134 | 7.61 | 8.02 | 7.11 | 7.92 | 7.94 |
| Compound 138 | 9.41 | 9.32 | 9.21 | 9.71 | 9.06 |
| Compound 124 | 8.77 | 8.92 | ND | 8.64 | 8.46 |
| Compound 136 | 7.58 | 8.40 | 7.29 | 8.30 | 8.20 |
| Compound 133 | 7.92 | 8.17 | 7.13 | 8.01 | 7.97 |
| Compound 135 | 7.60 | 7.91 | ND | 7.88 | 7.97 |
| Compound 137 | 7.20 | 7.77 | ND | 7.73 | 7.55 |
| Compound 123 | 8.43 | 8.72 | 7.71 | 8.61 | 8.70 |
| Compound 125 | 8.55 | 8.88 | 8.15 | 8.88 | 8.42 |
| Compound 128 | ND | 8.28 | 7.74 | 8.37 | 8.08 |
| Compound 126 | 8.53 | 8.96 | 8.20 | 9.14 | 8.71 |
| Compound 130 | 8.78 | 7.83 | 7.62 | ND | 8.19 |
| Compound 129 | 8.28 | 8.51 | 7.69 | 8.58 | 8.40 |
| Compound 127 | 8.03 | 8.48 | 7.84 | 8.28 | 8.42 |
| Compound 110 | 9.09 | 8.88 | 9.03 | 9.07 | 8.81 |
| Compound 111 | 7.71 | 8.07 | 7.78 | 8.04 | 7.54 |
| Compound 112 | 7.76 | 8.18 | 7.70 | 7.92 | 7.87 |
| Compound 113 | 8.52 | 8.71 | 8.37 | 8.66 | 8.47 |
| Compound 139 | 8.15 | 8.92 | 8.30 | 8.97 | 8.82 |
| Compound 140 | 9.51 | 9.50 | 9.34 | 9.48 | 9.15 |
| Compound 141 | 8.99 | 9.46 | 8.55 | 9.51 | 8.84 |
| Compound 142 | 8.89 | 9.20 | 8.89 | 9.12 | 8.89 |
| Compound 143 | 9.15 | 9.41 | 8.55 | 9.07 | 9.15 |
| Compound 144 | 7.65 | 9.10 | 7.16 | 7.88 | 7.65 |
| Compound 145 | 9.57 | 9.45 | 9.03 | 9.42 | 9.57 |
| Compound 146 | 8.36 | 8.76 | 7.95 | 8.22 | 8.36 |
| Compound 147 | 7.67 | 8.29 | 7.36 | ND | 7.67 |
| Compound 148 | 9.69 | 9.49 | ND | 9.66 | 9.69 |
| Compound 131 | 8.04 | 8.98 | ND | 9.02 | 8.04 |
| Compound 149 | 8.20 | 8.50 | 8.00 | 8.74 | 8.20 |
| Compound 150 | ND | 8.10 | 7.20 | 8.19 | ND |
| Compound 151 | 7.94 | 8.57 | 7.34 | 8.23 | 7.94 |
| Compound 152 | 8.83 | 8.49 | 8.54 | ND | 8.30 |
| Compound 153 | 9.94 | 9.29 | 9.03 | ND | ND |
| Compound 114 | 10.03 | 9.97 | 9.75 | ND | 9.38 |
| Compound 115 | 8.89 | 8.59 | 8.42 | ND | 8.38 |
| Compound 117 | 9.79 | 10.02 | 9.77 | ND | 9.33 |
| Compound 118 | 9.03 | 8.82 | 8.84 | ND | 8.73 |
| Compound 119 | 8.93 | 8.38 | 8.43 | ND | 8.56 |
| Compound 120 | 10.00 | 8.96 | 9.60 | ND | 9.72 |
| Compound 121 | 9.84 | 9.83 | 9.71 | ND | 9.57 |
| Compound 116 | 9.10 | 8.15 | 7.90 | ND | 8.03 |

*ND = not determined

### Example 6: Preparation of Anti-HER2 Antibody-Drug Conjugates Comprising Camptothecin Analogues

Exemplary antibody-drug conjugates (ADCs) comprising drug-linkers prepared as described in Example 4 were conjugated to trastuzumab as follows.

1 mg of a 21 mg/mL solution of trastuzumab (HERCEPTIN, manufactured by Roche, South San Francisco, CA) was diluted to 4 mg/mL with a 5 mM solution of DTPA in PBS (pH 7.4), and to this solution was added TCEP (12 eq). Following incubation for 3 hrs in a 37° C. water bath, the excess TCEP was removed using appropriate size 40 kD Zeba™ Spin Desalting Columns (Thermo Fisher Scientific, Waltham, MA) equilibrated with 10 mM sodium acetate buffer, pH 4.5. Alternatively, in some instances, the reduced antibody solution was buffer exchanged into PBS, pH 7.4 or into A5Su (10 mM acetate pH 5, 5% sucrose). Maleimide functionalized drug-linkers (15 eq) as 10 mM DMSO stocks were added together with as much as 10% DMSO (v/v) in two intervals (7.5 eq each) to the column-purified reduced trastuzumab solution. The conjugation reaction was mixed thoroughly by pipetting, the vial was protected from light and was rotated at room temperature for up to 2-2.5 h.

Purification of the ADCs was accomplished using an appropriate size 40 kD Zeba™ Spin Desalting Column (ThermoFisher Scientific, Waltham, MA) pre-equilibrated with 10 mM sodium acetate, pH 4.5. Alternatively, in some instances, ADCs were buffer exchanged to PBS, pH 7.4 or A5Su (10 mM sodium acetate, pH 5.0, 9% sucrose). The purified conjugates were stored at 4° C. and analyzed for total protein content with a BCA assay (either Pierce BCA Protein Assay (catalogue #23225) or Pierce microBCA Protein Assay (catalog #23235; ThermoFisher Scientific, Waltham, MA).

### Example 7: Characterization of Anti-HER2 ADCS

The ADCs from Example 6 were characterized by HPLC-HIC, SEC, CE-SDS and RP-UPLC-MS as described below. The average drug-to-antibody ratio (DAR) and DAR distribution were derived from interpretation of the HIC and LC-MS data. Unless otherwise indicated, the conjugation procedure resulted in modification of each interchain and hinge thiol yielding ADCs with DAR 8.

Endotoxin levels were assessed either using the ToxinSensor™ Single Test Kit (Genscript Biotech, Piscataway, NJ; Catalogue #L00450) or Endosafe® LAL Reagent Cartridges (sensitivity: 0.005 EU/mL, product code: PTS20005F) (Charles River Laboratories, Wilmington, MA) with a final threshold set at <0.5 EU/mg for antibody drug conjugates.

Residual free drug (FD) and drug-linker levels were assessed by RP-UPLC-MS and calculated based on the following equation, with a threshold set at 1%:

$$\% FD = \frac{[ Free drug ] + [ Free drug - linker ] + [ Drug - linker TCEP adduct ]}{[ ADC ] \times DAR}$$

### DAR Determination by HIC

The average DAR of the ADCs was assessed by HIC as described in Antibody Drug Conjugates, Methods in Molecular Biology, 2013, vol. 1045, pp. 275-284. L. Ducry, Ed. The experiments were performed on an Agilent Infinity II 1290 HPLC using a TSKgel Butyl-NPR column (2.5 μm, 4.6×35 mm, TOSOH Bioscience GmbH, Griesheim, Germany) pre-equilibrated with 5 column volumes of Buffer A (1.5 M (NH₄)₂SO₄, 25 mM PO₄³⁻, pH=6.95) at room temperature. In general, 20-30 μg of sample at 2-3 mg/mL concentration was loaded on the column with 95% Buffer A and 5% Buffer B (75% 25 mM PO₄³⁻ plus 25% isopropanol, pH 6.95) and run for 15 mins at 0.5 mL/min using the gradient shown in Table 7.1. HIC chromatograms were integrated using appropriate parameters that provided complete, baseline-to-baseline integration of each peak, followed by integration of each peak showing reasonable separation. As a reference, unconjugated trastuzumab was run on the same gradient to obtain the HIC retention time of DAR 0 species.

TABLE 7.1
Gradient used for HIC

| Time (min) | % Buffer A | % Buffer B |
|---|---|---|
| 0 | 95 | 5 |
| 0.1 | 95 | 5 |
| 5 | 80 | 20 |
| 9.5 | 65 | 35 |
| 11.5 | 50 | 50 |
| 12.5 | 5 | 95 |
| 13.5 | 5 | 95 |
| 12.6 | 95 | 5 |
| 15 | 95 | 5 |

### DAR Determination by LC/MS

ADCs were deglycosylated with Endo S for 1 h at room temperature. The deglycosylated ADCs were reduced with 50 mM TCEP for 1 h at room temperature and injected onto an Agilent 1290 Infinity II LC coupled to an Agilent 6545 Quadrupole Time of Flight (Q-TOF) mass spectrometer (Agilent Technologies, Inc., Santa Clara, CA). Heavy and light chains were separated using a PLRP-S column (1000 Å, 8 uM, 50×2.1 mm) at a flow rate of 0.3 ml/min and a linear gradient of 20 to 40% Mobile Phase A/Mobile Phase B. Mobile Phase A: 0.1% FA, 0.025% TFA and 10% IPA in water. Mobile Phase B: 0.1% FA and 10% IPA in acetonitrile. MassHunter (Agilent Technologies, Inc., Santa Clara, CA) qualitative analysis was used for deconvolution and data analysis.

### SEC-HPLC Analysis

Analytical SEC was performed using an Agilent Infinity II 1260 HPLC (Agilent Technologies, Inc., Santa Clara, CA) with Advance Bio SEC column (300 Å, 2.7 μm, 7.8×150 mm) equilibrated with 5 column volumes of buffer (150 mM Na₂PO₄, pH 6.95) at room temperature. In general, 20-30 mg of sample at 2-3 mg/mL concentration was eluted isostatically for 7 mins at 1 mL/min with absorbance monitoring at A280. Chromatograms were integrated to provide complete, baseline-to-baseline integration of each peak, with reasonably placed separation between partially resolved peaks. The peak corresponding to the major component for IgG (approximate retention time 3.3 min) was reported as the monomer based on the SEC profile of unmodified trastuzumab. Any peak occurring prior to 3.3 min was designated as HMWS (high molecular weight species), and any peak occurring after 3.3 min was designated as LMWS (low molecular weight species), excluding solvent peaks (over 5.2 min).

### CE-SDS Analysis

Initially, all ADC samples were diluted to 1 mg/mL before preparing the samples in a 96-well PCR plate following manufacturer's protocol (Protein Express Assay LabChip™; PerkinElmer, Inc., Waltham, MA). Briefly, 2 μg of ADC was mixed with 7 uL Protein Express buffer in the presence (reducing) or absence (non-reducing) of 400 mM dithiothreitol (DTT), followed by heat denaturation at 95° C. for 5 minutes. Samples were then diluted in dH₂O at a 1:2 ratio before data acquisition. After each CE-SDS run, the gel and corresponding electropherogram were analyzed using LabChip™ Reviewer (PerkinElmer, Inc., Waltham, MA).

The biophysical properties of the ADCs as determined by HPLC-HIC, LC-MS and HPLC-SEC are shown in Table 7.2. Also included in Table 7.2 are the properties for two control ADCs, T-MC-GGFG-AM-DXd and T-MT-GGFG-AM-DXd, which comprise trastuzumab (T) conjugated to either the drug-linker MC-GGFG-AM-DXd or the drug-linker MT-GGFG-AM-DXd. These drug-linkers have the following structures:

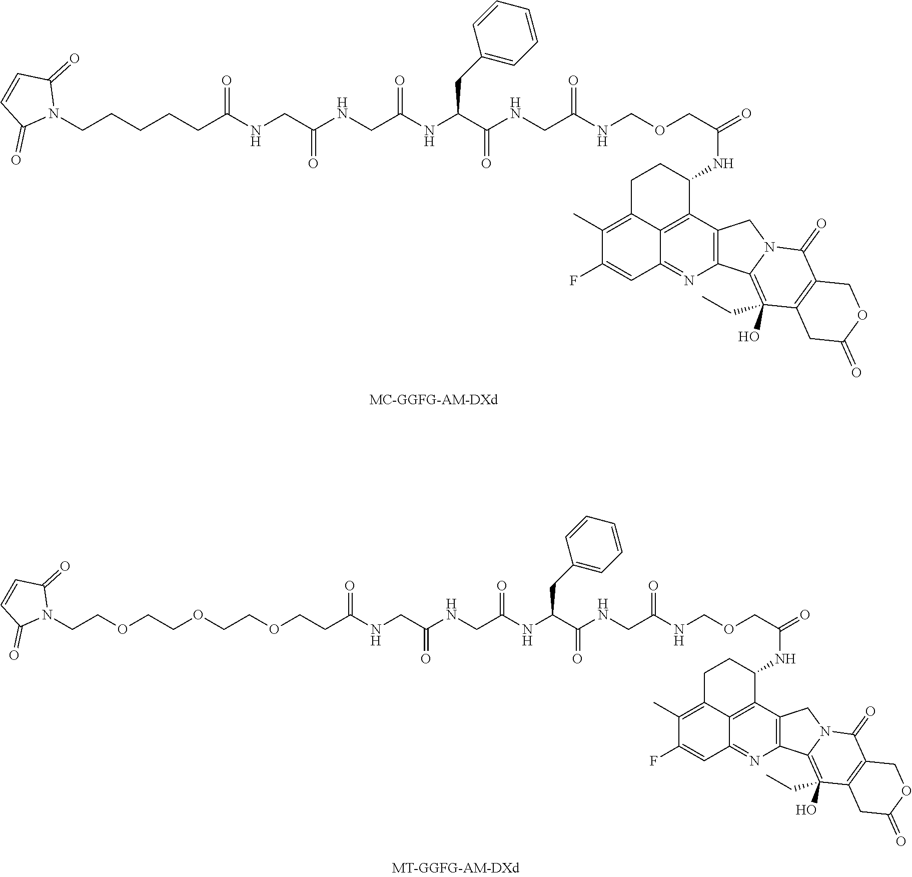

TABLE 7.2
Biophysical Properties of ADCs

| | | Reten- | | Monomer |
|---|---|---|---|---|
| | DAR | tion | DAR | % |
| | (HPLC- | Time | (LC- | (HPLC- |
| ADC | HIC) | (min)¹ | MS) | SEC) |
| Control (T-MC-GGFG-AM-DXd) | 8 | 5.4 | 8 | 100 |
| Control (T-MT-GGFG-AM-DXd) | 8 | 5.8 | 8 | 100 |
| T-MT-GGFG-Compound 104 | ND² | ND² | ND² | 77 |
| T-MT-GGFG-Compound 108 | ND² | ND² | ND² | 47 |
| T-MT-GGFG-Compound 127 | ND² | ND² | ND² | 9 |
| T-MT-GGFG-AM-Compound 136 | 8 | 5.7 | 8 | 100 |
| T-MT-GGFG-AM-Compound 139 | 8 | 5.9 | 8 | 100 |
| T-MT-GGFG-Compound 140 | 8 | 5.0 | 8 | 100 |
| T-MT-GGFG-AM-Compound 113 | 8 | 5.3 | 8 | 100 |
| T-MT-GGFG-AM-Compound 129 | 8 | 6.0 | 8 | 100 |
| T-MT-GGFG-AM-Compound 141 | 8 | 5.0 | 8 | 100 |
| T-MC-GGFG-AM-Compound 141 | 8 | 5.0 | 8 | 100 |
| T-MT-GGFG-Compound 141 | 8 | 4.9 | 8 | 100 |
| T-MT-GGFG-Compound 142 | 8 | 5.0 | 8 | 100 |
| T-MT-GGFG-Compound 148 | 8 | 7.0 | 8 | 100 |
| T-MT-GGFG-Compound 145 | 8 | 4.7 | 8 | 100 |

¹From HPLC-HIC
²Not determined due to high amounts of ADC aggregation

### Example 8: In vitro Cytotoxicity of Anti-HER2 ADCs

In vitro cytotoxicity of select ADCs from Example 6 was tested in SKBR-3 (breast cancer), Calu3 (lung cancer) and MIDA-MB-468 (breast cancer) cells using the procedure described in Example 5. The results are shown in Table 8.1.

TABLE 8.1
In vitro Cytotoxicity of ADCs (pIC50)

| | pIC50 | | |
|---|---|---|---|
| ADC | SKBR-3 | Calu3 | MDA-MB-468 |
| Control (T-MC-GGFG-AM-DXd) | 11.5 | 10.73 | 7.69 |
| T-MT-GGFG-Compound 140 | 9.3 | 10.24 | 7.63 |
| T-MT-GGFG-Compound 141 | 11.46 | 10.64 | 7.49 |
| T-MT-GGFG-AM-Compound 141 | 11.49 | 10.62 | 7.52 |
| T-MT-GGFG-Compound 142 | 9.55 | 9.94 | 7.5 |
| T-MT-GGFG-AM-Compound 136 | 11.25 | 10.25 | 7.57 |
| T-MT-GGFG-AM-Compound 129 | 10.97 | 10.27 | 8.37 |
| T-MT-GGFG-AM-Compound 113 | 8.76 | 9.22 | 9.88 |
| T-MT-GGFG-AM-Compound 139 | 10.97 | 10.3 | 7.3 |
| T-MT-GGFG-Compound 145 | 11.54 | 10.66 | 5.27 |
| T-MT-GGFG-Compound 148 | 8.54 | 9.74 | 7.55 |

### Example 9: Bystander Activity of Anti-HER2 ADCs

The ability of select ADCs from Example 6 to exert a bystander killing effect on cancer cells was assessed as described below. Bystander killing most commonly occurs after specific uptake of an ADC into an antigen-positive cell. Trafficking and degradation of the ADC results in release of free drug, which then crosses the cell membrane to kill nearby (bystander) cells.

The ADCs tested were: T-MT-GGFG-AM-Compound 136, T-MT-GGFG-AM-Compound 129, T-MT-GGFG-AM-Compound 139, T-MT-GGFG-Compound 141, T-MT-GGFG-AM-Compound 141, T-MT-GGFG-Compound 145, T-MT-GGFG-Compound 148, T-MT-GGFG-Compound 140, and controls T-MC-GGFG-AM-DXd and T-MT-GGFG-AM-DXd.

Also included was the ADC T-ME-PEG2-GGFG-DXd2. This ADC has been shown to lack bystander activity (see Ogitani, et al., 2016, Cancer Sci., 107:1039-1046) and was included as a negative control. The ADC comprises trastuzumab (T) conjugated to the drug-linker shown below:

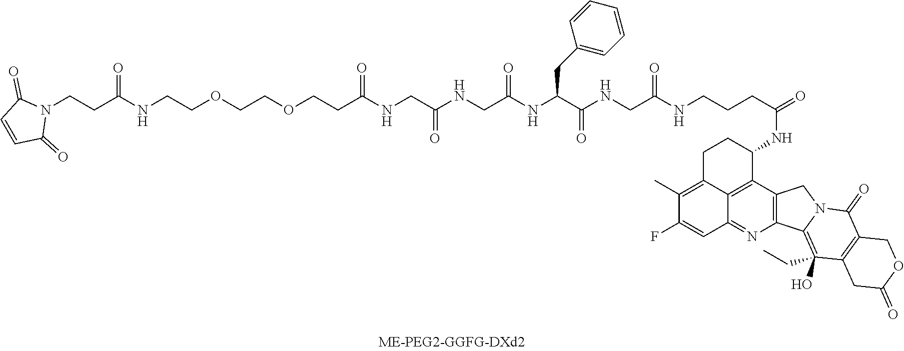

SK-BR3 (HER2+) and MDA-MB-468 (HER2−) cells were seeded either as mono-cultures or co-cultures in a 24-well plate at 30,000 cells and 10,000 cells, respectively, in 250 uL assay media (McCoy's+10% FBS). ADCs were diluted to 2 nM and 0.2 nM in assay media and 250 uL was added to the cell-containing plates (1 and 0.1 nM final ADC concentration). Cells were incubated with test ADCs for 4 d at 37° C. and detached by TrypLE™ Express Enzyme (ThermoFisher Scientific, Waltham, MA). Cells were stained using a viability dye, YO-PRO®-1 (ThermoFisher Scientific, Waltham, MA), and an anti-HER2 antibody conjugated to Alexa Fluor® 647 (Biolegend, Inc., San Diego, CA; Catalogue #324412). After 20 min incubation at room temperature, cells were washed in FACS buffer and resuspended in 100 uL FACS buffer per well. 50 uL were analyzed on the BD Fortessa™ flow cytometer (BD Biosciences, San Jose, CA). Dead cells were gated out by YO-PRO®-1 staining. The number of SK-BR3 and MDA-MB-468 cells was then determined by the number of events in the HER2+ and HER2− gates, respectively. % viability was calculated as the number of cells treated divided by the number of cells untreated.

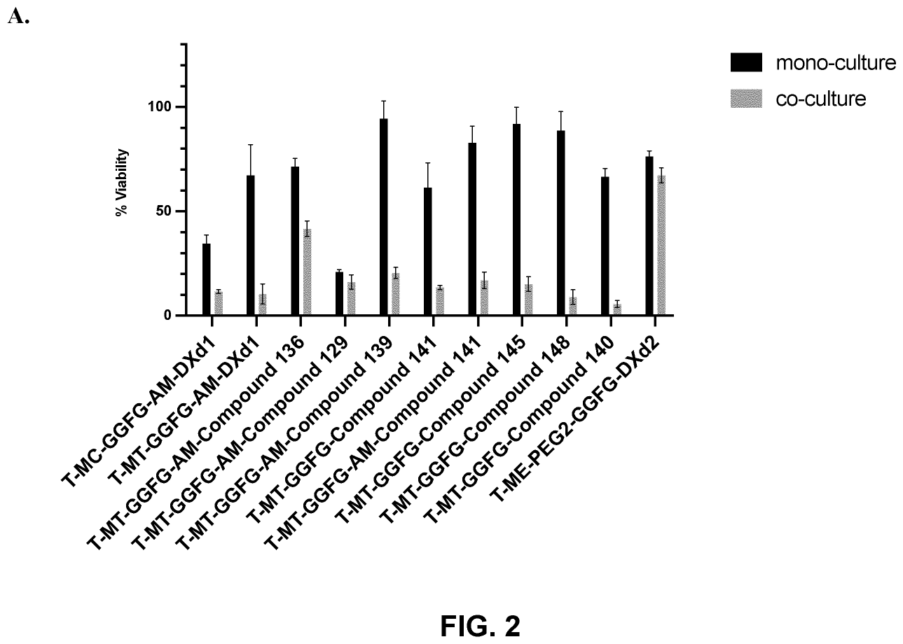

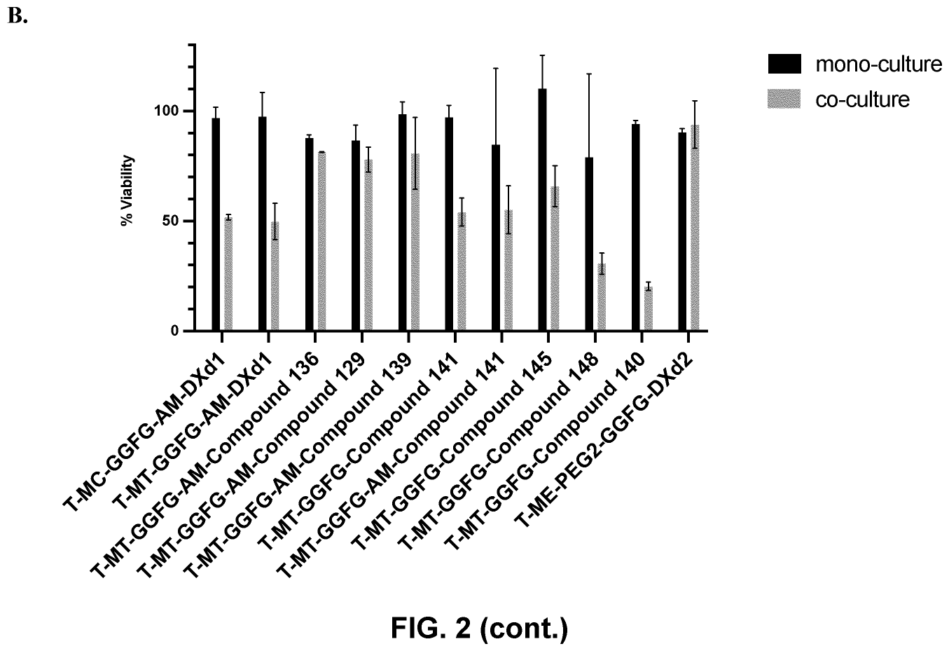

The results for are shown in FIG. 2. Bystander effect was evaluated by comparing the viability of HER2− MDA-MB-468 cells treated as a monoculture (black bars) with that of the cells treated as a co-culture with HER2+SK-BR-3 cells (grey bars). A greater decrease in viability in co-culture compared with monoculture indicates a higher bystander effect.

### Example 10: Plasma Stability of Anti-HER2 ADCs

The stability of select ADCs from Example 6 was tested in mouse plasma as follows. ADCs were diluted in mouse plasma (BioIVT, Westbury, NY) to 0.5 mg/mL and incubated in a 37° C. water bath. Aliquots were taken out at 10 min, 1.5 h, 8 h, 24 h, 72 h and 7 d and frozen at −80° C. Once all aliquots were collected, they were thawed and prepared for the affinity capture coupled to LC-MS analysis.

Biotinylated goat anti-human IgG F(ab′)2 (Jackson ImmunoResearch Laboratories, Inc., West Grove, PA) was coupled to Streptavidin Mag Sepharose® beads (GE Healthcare Bio Sciences, Uppsala, Sweden) for 30 min at room temperature prior to use. Mouse plasma samples containing approximately 2 ug ADC were diluted in PBS and deglycosylated with EndoS for 1 h at room temperature. Capture antibody-streptavidin bead mixture was added to the deglycosylated sample and incubated for 1.5 h at room temperature. Three PBS washes were performed, then the sample was reduced with 25 mM DTT for 1 h at room temperature, followed by an additional three PBS washes. To elute the captured ADC, samples were first washed once with water and then once with water+10% acetonitrile, then incubated in elution buffer (water+20% acetonitrile+1% formic acid) for 1 h at room temperature. Supernatant containing the eluted ADC was collected and injected onto an Agilent 1290 Infinity II LC coupled to an Agilent 6545 Quadrupole Time-of-Flight (Q-TOF) spectrometer (Agilent Technologies, Inc., Santa Clara, CA). A PLRP-S (1000 Å, 8 uM, 50×2.1 mm) column was used and the flow rate was set at 0.3 mL/min. Mobile Phase A: 0.1% FA, 0.025% TFA and 10% IPA in water, and Mobile Phase B: 0.1% FA and 10% IPA in acetonitrile. The elution gradient increased from 2% to 40% B over 20 min. MassHunter (Agilent Technologies, Inc., Santa Clara, CA) qualitative analysis was used for deconvolution and data analysis.

The results after a 7-day incubation are shown in Table 10.1.

TABLE 10.1
Stability of ADCs in Mouse Plasma after 7 Days

| | | % Male- | |
|---|---|---|---|
| | Remain- | imide | Other |
| | ing | Ring | Degradation |
| ADC | DAR % | Opening | Products |
| Control (T-MT-GGFG-AM-DXd) | 77 | 98 | — |
| Control (T-MC-GGFG-AM-DXd) | 68 | 39 | — |
| T-MT-GGFG-AM-Compound 136 | 76 | 98 | — |
| T-MT-GGFG-AM-Compound 139 | 76 | 98 | — |
| T-MT-GGFG-Compound 140 | 82 | 94 | — |
| T-MT-GGFG-AM-Compound 129 | 72 | 99 | −393 Da |
| | | | (11%) |
| T-MT-GGFG-AM-Compound 141 | 82 | 99 | — |
| T-MC-GGFG-AM-Compound 141 | 75 | 37 | — |
| T-MT-GGFG-Compound 141 | 81 | 100 | — |
| T-MT-GGFG-Compound 148 | 85 | 95 | — |
| T-MT-GGFG-Compound 145 | 76 | 98 | +16 Da |
| | | | (35%) |

### Example 10: In Vivo Evaluation of Ant-HER2 ADCs

The anti-tumor activity of select ADCs from Example 6 was investigated in a JIMT-1 xenograft model of breast cancer expressing HER2 (mid) as described below. The ADCs evaluated were: T-MT-GGFG-AM-Compound 136, T-MT-GGFG-AM-Compound 129, T-MT-GGFG-AM-Compound 139, T-MT-GGFG-Compound 140, T-MC-GGFG-AM-Compound 141, T-MT-GGFG-AM-Compound 141, T-MT-GGFG-Compound 141, T-MT-GGFG-Compound 145 and T-MT-GGFG-Compound 148, and control T-MC-GGFG-AM-DXd.

Tumor cell suspensions (5×10⁶ cells in 0.1 mL PBS) were implanted subcutaneously into female CB17/scid mice. When the mean tumor volume reached ˜150 mm³, the animals were randomized to dose groups (n=8 per group). ADCs were administered at approximately 3 mg/kg (iv). Due to formulation differences, actual dosages varied within a range of about ±30%. Tumor volume and body weight were measured twice weekly with a study duration of 28 days.

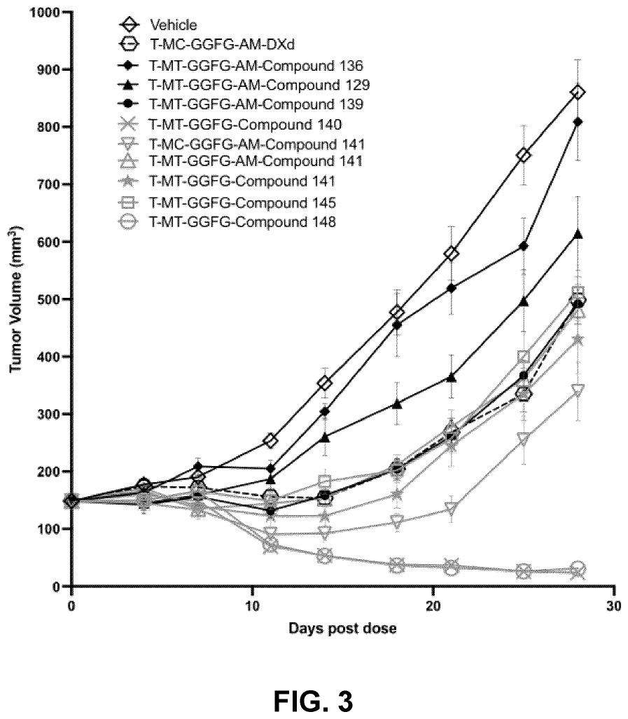

The results are shown in FIG. 3.

### Example 11: Preparation of Further Camptothecin Analogues Having Amino at the C10 Position

Additional examples of camptothecin analogues having an amino group at the C10 position are described below. These compounds may be prepared using starting materials and methods as described in Examples 1-3 above or similar methods as would be known to one skilled in the art.

| | | |
|---|---|---|
| 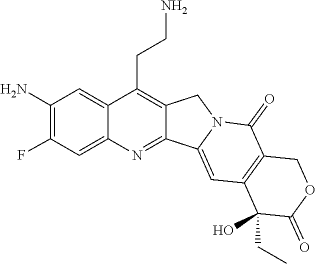 [SMILES: CC[C@@]1(O)C(=O)OCc2c1cc1n(c2=O)Cc2c-1nc1cc(F)c(N)cc1c2CCN] | (S)-9-amino-11-(2-aminoethyl)-4- ethyl-8-fluoro-4-hydroxy-1,12- dihydro-14H-pyrano[3′,4′:6,7]indol- izino[1,2-b]quinoline-3,14(4H)- dione | Compound 156 |
| 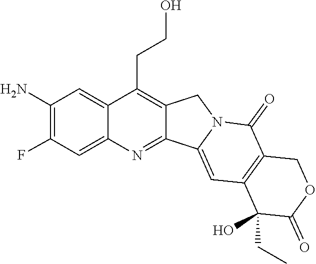 [SMILES: CC[C@@]1(O)C(=O)OCc2c1cc1n(c2=O)Cc2c-1nc1cc(F)c(N)cc1c2CCO] | (S)-9-amino-4-ethyl-8-fluoro-4- hydroxy-11-(2-hydroxyethyl)-1,12- dihydro-14H-pyrano[3′,4′:6,7]indol- izino-[1,2-b]quinoline-3,14(4H)- dione | Compound 157 |
| 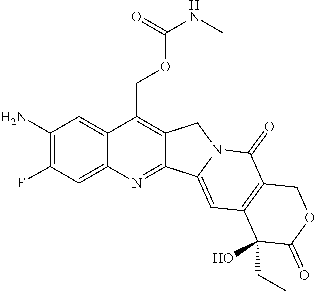 [SMILES: CC[C@@]1(O)C(=O)OCc2c1cc1n(c2=O)Cc2c-1nc1cc(F)c(N)cc1c2COC(=O)NC] | (S)-(9-amino-4-ethyl-8-fluoro-4- hydroxy-3,14-dioxo-3,4,12,14-tetra- hydro-1H-pyrano[3′,4′:6,7]indol- izino[1,2-b]quinolin-11-yl)methyl methylcarbamate | Compound 162 |
| 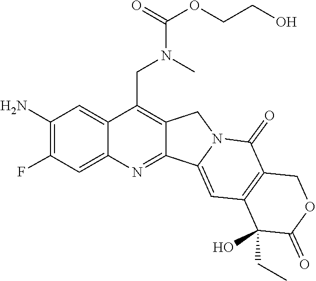 [SMILES: CC[C@@]1(O)C(=O)OCc2c1cc1n(c2=O)Cc2c-1nc1cc(F)c(N)cc1c2CN(C)C(=O)OCCO] | 2-hydroxyethyl (S)-((9-amino-4- ethyl-8-fluoro-4-hydroxy-3,14- dioxo-3,4,12,14-tetrahydro-1H- pyrano[3′,4′:6,7]indolizino[1,2-b]- quinolin-11-yl)methyl)(methyl)- carbamate | Compound 163 |

### Example 12: Preparation of Anti-Frα Antibody-Drug Conjugates

ADCs comprising select drug-linkers prepared as described in Example 4 were conjugated to two anti-folate receptor alpha (FRα) antibodies (v36675 and v30384; see Table 12.1). Exemplary conjugation protocols are provided below.

v36675-MC-GGFG-AM-DXd1: A solution (83.5 mL) of the anti-FRα antibody v36675 (1.5 g) in PBS, pH 7.4 was reduced by addition of 5 mM DTPA (24 mL in PBS, pH adjusted to 7.4) and 10 mM of an aqueous TCEP solution (12.5 mL, 12 eq.). After 3 hours at 37° C., the reduced antibody was diluted to 125 mL with PBS and purified using a Pellicon® XL Ultrafiltration Module (Ultracel 30 kDa 0.005 m²; MilliporeSigma, Burlington, MA; PXC030C50) with approximately 5 diavolumes of 10 mM NaOAc, pH 5.5. The purified antibody (1133 mg) was diluted to a final volume of 211 mL using 10 mM NaOAc, pH 5.5. To the antibody solution was added 6.4 mL of DMSO and an excess of drug-linker (9.43 mL; 12 eq.) from a 10 mM DMSO stock solution. The conjugation reaction proceeded at room temperature with mixing for 75 minutes. An excess of a 10 mM N-acetyl-L-cysteine solution (4.72 mL, 6 eq.) was added to quench the conjugation reaction.

v36675-MC-GGFG-AM-Compound 141, v36675-MC-GGFG-AM-Compound 139 & v36675-MC-GGFG-Compound 141: A solution (2.95 mL) of the anti-FRα antibody v36675 (60 mg) in PBS, pH 7.4 was reduced by addition of 5 mM DTPA (0.96 mL in PBS, pH adjusted to 7.4) and 1 mM of an aqueous TCEP solution (0.9 mL, 2.15 eq.). After 100 minutes at 37° C., 1.6 mL of the reduced antibody was diluted with 0.92 mL of PBS, pH 7.4 and 1.08 mL of 100 mM NaOAc, pH 5.5. To the antibody solution was added 289 uL of DMSO and an excess of drug-linker (111 uL; 8 eq.) from a 10 mM DMSO stock solution. The conjugation reaction proceeded at room temperature with mixing for 60 minutes. An excess of 10 mM cysteamine-HCl solution (444 uL, 32 eq.) was added to quench each conjugation reaction.

TABLE 12.1
Antibody-Drug Conjugates

| Antibody | Drug-Linker¹ | DAR |
|---|---|---|
| v36675 | MC-GGFG-AM-DXd1 | 8 |
| | MC-GGFG-AM-Compound 141 | 8 |
| | MC-GGFG-AM-Compound 139 | 8 |
| | MC-GGFG-Compound 141 | 8 |
| | MC-GGFG-Compound 140 | 8 |
| | MC-GGFG-AM-Compound 141 | 4 |
| | MC-GGFG-AM-Compound 139 | 4 |
| | MC-GGFG-Compound 141 | 4 |
| v30384 | MC-GGFG-AM-DXd1 | 8 |
| | MC-GGFG-AM-Compound 141 | 8 |
| | MT-GGFG-AM-Compound 139 | 8 |
| | MT-GGFG-Compound 141 | 8 |
| | MT-GGFG-Compound 140 | 8 |
| | MT-GGFG-Compound 148 | 8 |
| | MC-GGFG-Compound 140 | 8 |
| | MT-GGFG-AM-Compound 141 | 8 |
| v21995² | MT-GGFG-AM-DXd1 | 8 |
| | MC-GGFG-AM-DXd1 | 8 |
| | MT-GGFG-Compound 140 | 8 |
| | MT-GGFG-AM-Compound 141 | 8 |
| | MT-GGFG-Compound 141 | 8 |
| | MC-GGFG-Compound 140 | 8 |
| | MT-GGFG-AM-Compound 139 | 8 |

¹See Tables 4 & 5 (FIGS. 4 & 5)
²Palivizumab (anti-RSV; negative control)

### Example 13: Purification and Characterization of Anti-FRα ADCs

Large scale preparations of ADCs were purified using a Pellicon® XL Ultrafiltration Module (MilliporeSigma, Burlington, MA) and sterile filtered (0.22 m). An exemplary protocol is provided below.

The quenched ADC solution from Example 12 was diluted to approximately 5 mg/mL with 10 mM NaOAc, pH 5.5 and purified using a Pellicon® XL Ultrafiltration Module (Ultracel 30 kDa 0.005 m²; MilliporeSigma, Burlington, MA; PXC030C₅₀) with 11 diavolumes of 10 mM NaOAc, pH 4.5, followed by 4 diavolumes of 10 mM NaOAc, pH 4.5 with 9% (v/v) sucrose. The purified ADC was then sterile filtered (0.2 m).

Small scale preparations of ADCs were purified on an AKTA™ pure chromatography system (Cytiva Life Sciences, Marlborough, MA) using a 53 mL HiPrep 26/10 Desalting column (Cytiva Life Sciences, Marlborough, MA) and a mobile phase consisting of 10 mM NaOAc, pH 4.5 with 150 mM NaCl and a flow rate of 10 mL/min.

Following purification, the concentration of the ADCs was determined by a BCA assay with reference to a standard curve generated using the antibody v36675, estimated by measurement of absorption at 280 nm using extinction coefficients taken from the literature (European Patent No. 3 342 785, for MC-GGFG-AM-DXd1), or determined experimentally (for the remaining drug-linkers). ADCs were also characterized by hydrophobic interaction chromatography (HIC) and size exclusion chromatography (SEC) as described below.

### Hydrophobic Interaction Chromatography

Antibody and ADCs were analyzed by HIC to estimate the drug-to-antibody ratio (DAR). Chromatography was performed on an Agilent Infinity II 1290 HPLC (Agilent Technologies, Santa Clara, CA) using a TSKgel® Butyl-NPR column (2.5 μm, 4.6×35 mm; TOSOH Bioscience GmbH, Griesheim, Germany) and employing a gradient of 95/5% MPA/MPB to 5/95% MPA/MPB over a period of 12 minutes at a flow rate of 0.5 mL/min (MPA=1.5 M (NH₄)₂SO₄, 25 mM NaₓPO₄, pH 7 and MPB=75% 25 mM NaₓPO₄, pH 7, 25% isopropanol). Detection was by absorbance at 280 nm.

### Size Exclusion Chromatography

The extent of aggregation of the antibody and ADCs (˜15-150 μg, 5 μL injection volume) was assessed by SEC on an Agilent Infinity II 1260 HPLC (Agilent Technologies, Santa Clara, CA) using an AdvanceBio SEC column (300 angstroms, 2.7 μm, 7.8×150 mm) (Agilent, Santa Clara, California) and a mobile phase consisting of 150 mM phosphate, pH 6.95 and a flow rate of 1 mL/min. Detection was by absorbance at 280 nm.

### Results

The individual contributions of the DAR0, DAR2, DAR4, DAR6 and DAR8 species to the average DAR of the purified ADCs were assessed by integration of the HPLC-HIC chromatogram. The average DAR of each ADC was determined by the weighted average of each DAR species. The average DAR for each ADC, when rounded to the nearest integer, was the same as the target DAR shown in Table 12.1.

The extent of aggregation and monomer content was assessed by integration of the HPLC-SEC chromatogram. The monomer peak of each ADC was identified as the peak with the same retention time as the unconjugated antibody from which each ADC was derived. All peaks with an earlier retention time relative to the monomer species were determined to be aggregated species. Percent monomer species determined for each ADC is shown in Table 13.1. All ADC preparations showed >9500 monomer species.

TABLE 13.1
Target DAR and % Monomer Species for ADCs

| | | Target | % |
|---|---|---|---|
| Antibody | Drug-Linker | DAR | Monomer |
| v36675 | MC-GGFG-AM-DXd1 | 8 | 99.1% |
| | MC-GGFG-AM-Compound 141 | 8 | 98.3% |
| | MC-GGFG-AM-Compound 139 | 8 | 98.1% |
| | MC-GGFG-Compound 141 | 8 | 98.1% |
| | MC-GGFG-Compound 140 | 8 | 97.7% |
| | MC-GGFG-AM-Compound 141 | 4 | 98.6% |
| | MC-GGFG-AM-Compound 139 | 4 | 98.4% |
| | MC-GGFG-Compound 141 | 4 | 98.4% |
| v30384 | MC-GGFG-AM-DXd1 | 8 | 96.2% |
| | MC-GGFG-AM-Compound 141 | 8 | 96.7% |
| | MT-GGFG-AM-Compound 139 | 8 | 99.5% |
| | MT-GGFG-Compound 141 | 8 | 99.7% |
| | MT-GGFG-Compound 140 | 8 | 97.3% |
| | MT-GGFG-Compound 148 | 8 | 95.8% |
| | MC-GGFG-Compound 140 | 8 | 97.8% |
| | MT-GGFG-AM-Compound 141 | 8 | 99.6% |
| v21995 | MT-GGFG-AM-DXd1 | 8 | 100% |
| | MC-GGFG-AM-DXd1 | 8 | 100% |
| | MT-GGFG-Compound 140 | 8 | 98.8% |
| | MT-GGFG-AM-Compound 141 | 8 | 98.6% |
| | MT-GGFG-Compound 141 | 8 | 100% |
| | MC-GGFG-Compound 140 | 8 | 100% |
| | MT-GGFG-AM-Compound 139 | 8 | 100% |

### Example 14: In Vitro Cytotoxicity of Anti-FRα ADCs—2D Monolayer

The cell growth inhibition (cytotoxicity) capabilities of select ADCs from Example 12 were assessed in a panel of FRα-expressing cell lines following the protocol described in Example 5. Cell lines used were: KB-HeLa (endocervical carcinoma), JEG-3 (choriocarcinoma), T-47D (breast carcinoma) and MDA-MB-468 (breast adenocarcinoma; FRα-negative). ADCs comprising the antibody palivizumab (v21995) were used as non-targeted controls.

Based on blank wells (no test article added), % cytotoxicity values were calculated and plotted against test article concentration using GraphPad Prism 9 software (GraphPad Software, San Diego, CA). The results are shown in Table 14.1. All v30384 ADCs displayed significant cytotoxicity in the FRα-expressing cell lines KB-HeLa, JEG-3 and T-47D, yielding single-digit nM or lower EC50 values after the 4-day treatment. In the FRα-negative cell line, MDA-MB-468, the ADCs did not show target-dependent cytotoxicity. Both v30384 and control (palivizumab) ADCs showed comparable potency in this cell line.

TABLE 14.1
In vitro Cytotoxicity - 2D Monolayer

| | EC50 (nM) | | | |
|---|---|---|---|---|
| | | | | MDA-MB- |
| ADC | KB-HeLa | JEG-3 | T-47D | 468 |
| v30384-MC-GGFG-AM-DXd1 | 0.52 | 0.54 | 2.08 | 22.13 |
| v30384-MT-GGFG-AM-Compound 139 | 0.72 | 1.14 | 1.12 | 36.70 |
| v30384-MT-GGFG-Compound 140 | 3.75 | 1.29 | 12.43 | 23.33 |
| v30384-MC-GGFG-AM-Compound 141 | 0.77 | 3.45 | 0.43 | 83.19 |
| v30384-MT-GGFG-Compound 141 | 0.81 | 1.68 | 0.44 | 25.68 |
| v30384-MT-GGFG-Compound 148 | 0.73 | 0.54 | 3.45 | 21.20 |

Controls

| | | | | |
|---|---|---|---|---|
| v21995-MT-GGFG-DXd1 | 19.45 | 15.02 | 11.26 | 17.95 |
| v21995-MT-GGFG-Compound 140 | >100 | IC* | IC* | 20.05 |
| v21995-MT-GGFG-AM-Compound 141 | >100 | >100 | 17.71 | 34.28 |
| v21995-MT-GGFG-Compound 141 | >100 | >100 | 21.80 | 38.06 |

*Incomplete curve

### Example 15: In Vitro Cytotoxicity of Anti-FRα ADCs—3D Spheroids

The cytotoxicity capabilities of select ADCs from Example 12 were assessed in a panel of FRα-expressing cell line spheroids as described below. Cell lines used were IGROV-1 (ovarian adenocarcinoma), T-47D (breast carcinoma), OVCAR-3 (ovarian adenocarcinoma), HEC-1-A (uterine adenocarcinoma) and EBC-1 (lung carcinoma; FRα-negative). ADCs comprising the antibody palivizumab (v21995) were used as non-targeted controls.

Briefly, cells were seeded in Ultra-Low Attachment 384-well plates, centrifuged and incubated under standard culturing conditions to allow for spheroid formation and growth. Monoculture cell line spheroids were then treated with a titration of test article, generated in cell growth medium. Spheroids were incubated for 6 days under standard culturing conditions. After incubation, CellTiter-Glo® 3D reagent (Promega Corporation, Madison, WI) was spiked in all wells. Plates were incubated in the dark at room temperature for 1 hour and luminescence was quantified using a BioTek Cytation 5 Cell Imaging Multi-Mode Reader (Agilent Technologies, Inc., Santa Clara, CA). Based on blank wells (no test article added), percent cytotoxicity values were calculated and plotted against test article concentration using GraphPad Prism 9 software (GraphPad Software, San Diego, CA).

The results are shown in Table 15.1. All v30384 ADCs displayed significant cytotoxicity in the FRα-expressing monoculture spheroids (IGROV-1, T-47D, OVCAR-3 and HEC-1-A) yielding single-digit nM EC50 values in spheroids after 6-day treatment. In the FRα-negative cell line spheroids, EBC-1, v30384 ADCs did not show target-dependent cytotoxicity. Both v30384 and control (palivizumab) ADCs showed comparable potency in this cell line spheroid.

TABLE 15.1
In vitro Cytotoxicity - 3D Spheroids

| | 3D EC50 (nM) | | | | |
|---|---|---|---|---|---|
| | IGROV- | T- | OVCAR- | HEC- | EBC- |
| ADC | 1 | 47D | 3 | 1-A | 1 |
| v30384-MC-GGFG-AM- | 0.5 | 0.9 | 8.2 | 1.1 | 10.3 |
| DXd | | | | | |
| v30384-MC-GGFG- | 0.2 | 1.6 | 20.5 | 6.7 | 7.6 |
| Compound 140 | | | | | |
| v30384-MT-GGFG-AM- | 1.0 | 1.1 | 1.5 | 3.9 | 23.0 |
| Compound 139 | | | | | |
| v30384-MT-GGFG- | 1.0 | 0.4 | 1.6 | 5.0 | 22.5 |
| Compound 141 | | | | | |
| v30384-MC-GGFG-AM- | 0.6 | 0.4 | 1.8 | 8.5 | 12.8 |
| Compound 141 | | | | | |

Controls

| | | | | | |
|---|---|---|---|---|---|
| v21995-MC-GGFG-DXd | 16.7 | 17.8 | 46.0 | 27.6 | 24.4 |
| v21995-MC-GGFG- | 7.0 | 9.0 | 28.6 | 45.7 | 8.3 |
| Compound 140 | | | | | |
| v21995-MT-GGFG-AM- | 33.1 | 32.8 | 150.0 | 145.3 | 36.2 |
| Compound 139 | | | | | |
| v21995-MT-GGFG- | 79.2 | 15.3 | 36.6 | 150.0 | 35.6 |
| Compound 141 | | | | | |

### Example 16: In Vivo Evaluation of Anti-FRα ADCs

The in vivo anti-tumor activities of select ADCs from Example 12 were assessed in a number of xenograft models as described below. ADCs comprising the antibody palivizumab (v21995) were used as non-targeted controls in some models. The ADCs, xenograft models, dosages and study durations employed in each xenograft study are summarized in Table 16.1. For each xenograft study, tumor volume and body weight of the animals were measured twice weekly.

TABLE 16.1
Study Parameters

| | | | | | Study |
|---|---|---|---|---|---|
| Xenograft | Cancer | FRα | | Dose | Duration |
| Model | Type | Expression | ADC | (mg/kg) | (days) |
| OV90 #1 | Ovarian | Mid | v30384-MC-GGFG-AM-DXd1 | 3 | 60 |
| | | | v30384-MT-GGFG-AM- | 3 | |
| | | | Compound 139 | | |
| | | | v30384-MT-GGFG-AM- | 3 | |
| | | | Compound 141 | | |
| | | | v30384-MT-GGFG-Compound 141 | 3 | |
| OV90 #2 | Ovarian | Mid | v30384-MC-GGFG-AM-DXd1 | 3 | 60 |
| | | | v30384-MT-GGFG-Compound 140 | 3 | |
| | | | v30384-MT-GGFG-AM- | 3 | |
| | | | Compound 141 | | |
| | | | v30384-MC-GGFG-AM- | 3 | |
| | | | Compound 141 | | |
| | | | v21995-MC-GGFG-AM-DXd1 | 3 | |
| | | | v21995-MT-GGFG-AM- | 3 | |
| | | | Compound 141 | | |
| | | | v21995-MT-GGFG-Compound 140 | 3 | |
| NCI- | Lung | Mid/Low | v30384-MC-GGFG-AM-DXd1 | 6 | 28 |
| H2110 | | | v30384-MC-GGFG-AM- | 6 | |
| | | | Compound 141 | | |
| | | | v30384-MT-GGFG-AM- | 6 | |
| | | | Compound 141 | | |
| | | | v30384-MC-GGFG-Compound 140 | 6 | |
| | | | v30384-MT-GGFG-Compound 140 | 6 | |
| | | | v30384-MT-GGFG-Compound 148 | 6 | |
| | | | v30384-MT-GGFG-AM- | 6 | |
| | | | Compound 139 | | |
| | | | v21995-MC-GGFG-AM-DXd1 | 6 | |
| | | | v21995-MT-GGFG-AM- | 6 | |
| | | | Compound 141 | | |
| | | | v21995-MT-GGFG-Compound 140 | 6 | |

For both OV90 model studies, tumor cell suspensions (1×10⁷ cells in 0.1 ml 50% o Matrigel®) were implanted subcutaneously into female CB.17 SCID mice. When mean tumor volume reached 100-150 mm³, the animals were randomly assigned to groups (n=6 per group for OV90 #1, and n=8 per group for OV90 #2) and treated on study day 1 with a single IV dose of test article as shown in Table 16.1. Serum was collected at a number of timepoints for PK analysis.

For the NCI-H2110 CDX model study, tumor cell suspensions (1×10⁷ cells in 0.1 ml 50% Matrigel®) were implanted subcutaneously into CB.17 SCID mice. When mean tumor volume reached ˜140 mm³ the animals were randomly assigned to groups (n=6 per group) and treated with a single IV dose of test article on study day 0 as shown in Table 16.1.

### Results

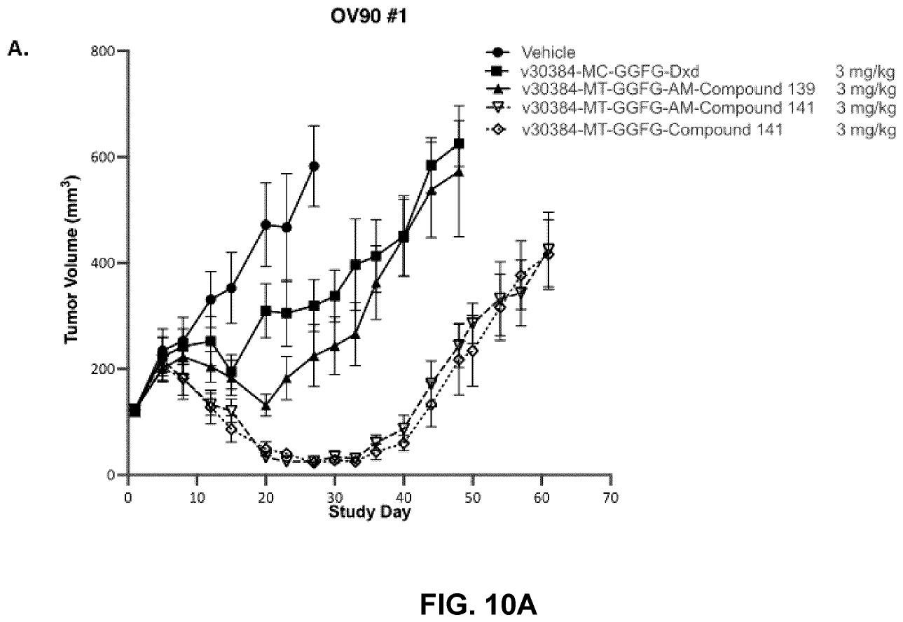

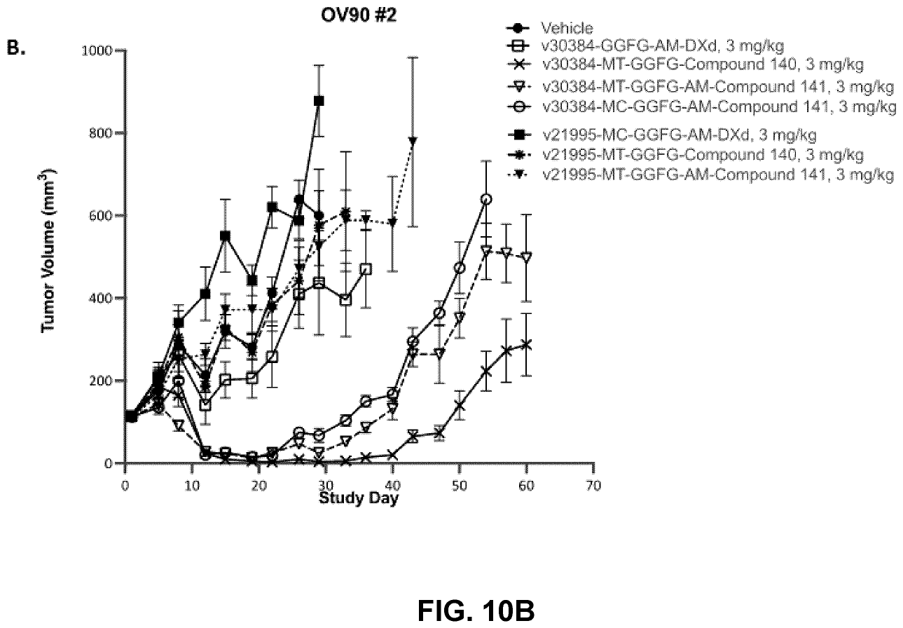

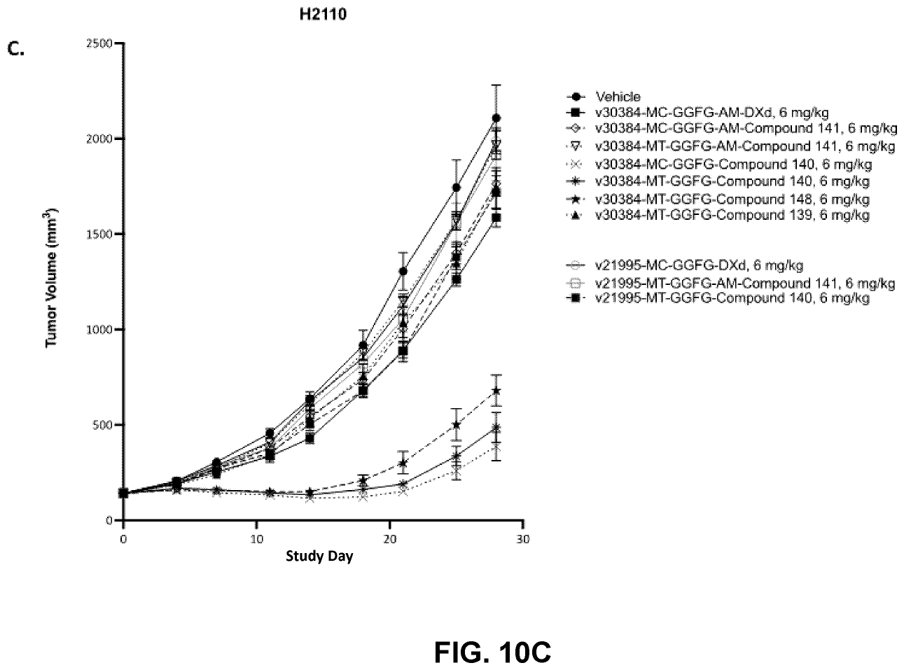

The results are shown in FIG. 10. In the OV90 model study #1 (see FIG. 10A), when dosed at 3 mg/kg, all ADCs resulted in a statistically significant reduction in the tumor growth rate compared to control (p<0.02). The ADCs v30384-MT-GGFG-AM-Compound 139, v30384-MT-GGFG-AM-Compound 141 and v30384-MT-GGFG-Compound 141 all resulted in superior inhibition of tumor growth rate compared to v30384-MC-GGFG-AM-DXd (p<0.01). Similarly, in the OV90 model study #2 (see FIG. 10B), when dosed at 3 mg/kg, v30384-MT-GGFG-Compound 140, v30384-MT-GGFG-AM-Compound 141 and v30384-MC-GGFG-AM-Compound 141 all resulted in tumor regressions, while v30384-MC-GGFG-AM-DXd had a marginal effect on tumor growth compared to control. Non-targeted v21995 ADCs did not substantially affect tumor growth.

In the NCI-H2110 CDX model study (see FIG. 10C), when dosed at 6 mg/kg, v30384-MT-GGFG-Compound 140, v30384-MC-GGFG-Compound 140 and v30384-MT-GGFG-Compound 148 all resulted in stasis of tumor growth for approximately 2 weeks post-dose, which represented a statistically significant inhibition of tumor growth rate compared to each of control, v30384-MC-GGFG-AM-DXd and non-targeted v21995 ADCs (p<0.01). v30384-GGFG-AM-DXd, v30384-MC-GGFG-AM-Compound 141, v30384-MT-GGFG-AM-Compound 141 and v30384-MT-GGFG-AM-Compound 139 did not result in significant tumor growth rate inhibition in this model.

### Example 17: Pharmacokinetics of Anti-FRα ADCs in In Vivo Efficacy Models

Serum was collected from the xenograft studies described in Example 16 as noted and analyzed for the pharmacokinetics (PK) of the ADCs as follows. Test article concentrations were measured in mouse serum by sandwich ELISA utilizing an anti-human IgG1 Fc capture antibody (Jackson Immuno Research Labs, West Grove, PA; Cat. 709-005-098) and a HRP-conjugated anti-IgG1 Fab detection antibody (Jackson Immuno Research Labs; Cat. 109-035-097) for total IgG levels. Absorbance at 450 nM was measured using a Synergy™ HI Hybrid Multi-Mode Plate Reader (BioTek Instruments, Winooski, VT). Pharmacokinetics parameters were calculated from non-compartmental analysis using Phoenix WinNonlin™ software (Certara, Princeton, NJ).

The calculated elimination half-lives of the ADCs are shown in Table 17.1. Overall, the OV90 studies in immunocompromised tumor-bearing mice demonstrate that v30384 ADCs utilizing camptothecin analogues Compound 141, Compound 139 and Compound 140 have favorable PK properties shown by longer or comparable elimination half-life compared to v30384-MC-GGFG-AM-DXd1 (control). Shorter elimination half-lives were observed for all v30384 ADCs, including DXd1 control, in the NCI-H2110 model compared to OV90 models. Elimination half-lives for non-targeting control v21995 ADCs were comparable in OV90 and NCI-H2110 models.

TABLE 17.1
Elimination Half-life of ADCs

| | | | Half- |
|---|---|---|---|
| Xenograft | | Dose | life |
| Model | ADC | (mg/kg) | (day) |
| OV90 #1 | v30384-MC-GGFG-AM-DXd1 | 3 | 4.42 |
| | v30384-MT-GGFG-AM-Compound 139 | 3 | 8.27 |
| | v30384-MT-GGFG-AM-Compound 141 | 3 | 6.57 |
| | v30384-MT-GGFG-Compound 141 | 3 | 5.63 |
| OV90 #2 | v30384-MC-GGFG-AM-DXd1 | 3 | 4.26 |
| | v30384-MT-GGFG-Compound 140 | 3 | 5.88 |
| | v30384-MT-GGFG-AM-Compound 141 | 3 | 4.62 |
| | v30384-MC-GGFG-AM-Compound 141 | 3 | 5.17 |
| | v21995-MC-GGFG-AM-DXd1 | 3 | 4.68 |
| | v21995-MT-GGFG-AM-Compound 141 | 3 | 7.90 |
| | v21995-MT-GGFG-Compound 140 | 3 | 4.01 |
| NCI- | v30384-MC-GGFG-AM-DXd1 | 6 | 2.35 |
| H2110 | v30384-MC-GGFG-AM-Compound 141 | 6 | 1.87 |
| | v30384-MT-GGFG-AM-Compound 141 | 6 | 2.13 |
| | v30384-MC-GGFG-Compound 140 | 6 | 2.70 |
| | v30384-MT-GGFG-Compound 140 | 6 | 4.80 |
| | v30384-MT-GGFG-Compound 148 | 6 | 3.64 |
| | v30384-MT-GGFG-AM-Compound 139 | 6 | 2.39 |
| | v21995-MC-GGFG-AM-DXd1 | 6 | 4.00 |
| | v21995-MT-GGFG-AM-Compound 141 | 6 | 5.60 |
| | v21995-MT-GGFG-Compound 140 | 6 | 4.94 |

### Example 18: Murine Tolerability of Anti-FRα ADCs

Select ADCs from Example 12 were assessed for tolerability in mice at single doses of 60 and 200 mg/kg as follows. Test articles were administered to mice (Balb/c, female, 6-8 weeks old, ˜20 g) via 20 ml/kg intraperitoneal injections at 60 and 200 mg/kg. From each dose group, 3 mice were subject to planned observation for 3 weeks post-dose. An additional 3 mice were subject to planned observation for 1 week post-dose, followed by termination and examination of formalin-fixed, paraffin-embedded organs. Mice were euthanized if body weight fell by ≥20% from pre-dose levels. Serum collection was planned for all mice at 24 hr and 7 days post-dose for pharmacokinetic analysis. Doses and unscheduled deaths are summarized in Table 18.1.

TABLE 18.1
ADCs, Doses and Unscheduled Deaths in Murine Tolerability Study

| | | | Number of females | | Unscheduled |
|---|---|---|---|---|---|
| Group | | Dose level | Dosing | Recovery | deaths - days |
| No. | ADC | (mg/kg) | phase | Phase | post dose |
| 1 | Vehicle Control | 0 | 6 | 6 | — |
| 2 | v30384-MC-GGFG-AM-DXd1 | 60 | 3 | 3 | — |
| | | 200 | 3 | 3 | — |
| 3 | v30384-MT-GGFG-AM- | 60 | 3 | 3 | — |
| | Compound 139 | 200 | 3 | 3 | — |
| 4 | v30384-MT-GGFG-AM- | 60 | 3 | 3 | — |
| | Compound 141 | 200 | 3 | 3 | — |
| 5 | v30384-MT-GGFG-Compound | 60 | 3 | 3 | — |
| | 141 | 200 | 3 | 3 | — |
| 6 | v30384-MT-GGFG-Compound | 60 | 3 | 3 | 4, 5, 5, 5, 6, 6 |
| | 140 | 200 | 3 | 3 | 4, 4, 4, 5, 5, 5 |
| 7 | v30384-MT-GGFG-Compound | 60 | 3 | 3 | 4, 5, 5, 5, 6, 6 |
| | 148 | 200 | 3 | 3 | 4, 4, 4, 5, 5, 5 |
| 8 | v30384-MC-GGFG-Compound | 60 | 3 | 3 | 3, 4, 5, 5, 6, 6 |
| | 140 | 200 | 3 | 3 | 3, 3, 3, 4, 4, 4 |
| 9 | v30384-MC-GGFG-Compound | 60 | 3 | 3 | — |
| | 141 | | | | |

### Results

The ADCs v30384-MT-GGFG-AM-Compound 139, v30384-MT-GGFG-AM-Compound 141, v30384-MT-GGFG-Compound 141 and v30384-MC-GGFG-Compound 141 were well tolerated at both 60 and 200 mg/kg, with no substantial body weight loss observed over 21 days, similar to mice administered vehicle control or the ADC 30384-MC-GGFG-AM-DXd1. ADCs v30384-MT-GGFG-Compound 140, v30384-MT-GGFG-Compound 148 and v30384-MC-GGFG-Compound 140 resulted in rapid body weight loss, mortality or sacrifice due to moribund condition between 3-6 days post dose (see Table 18.1).

No treatment-related macroscopic changes were observed in mice treated with the ADCs v30384-MT-GGFG-AM-Compound 139, v30384-MT-GGFG-AM-Compound 141, v30384-MT-GGFG-Compound 141 or v30384-MC-GGFG-Compound 141 at 60 or 200 mg/kg, as was the case for control ADC 30384-MC-GGFG-AM-DXd1. Macroscopic changes considered related to the ADCs were present in preterminal animals treated with 60 and/or 200 mg/kg of v30384-MT-GGFG-Compound 140, v30384-MT-GGFG-Compound 148 and v30384-MC-GGFG-Compound 140.

No treatment related microscopic findings were present in mice administered the ADCs v30384-MT-GGFG-AM-Compound 139 or v30384-MC-GGFG-Compound 141, or the control ADC v30384-MC-GGFG-AM-DXd1. Microscopic changes considered related to administration of ADCs v30384-MT-GGFG-Compound 140, v30384-MT-GGFG-Compound 148 and v30384-MC-GGFG-Compound 140 at ≥60 mg/kg dose were present in intestine, bone marrow, thymus, spleen and mesenteric lymph node.

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Modifications of the specific embodiments described herein that would be apparent to those skilled in the art are intended to be included within the scope of the following claims.

We claim:

1. A compound having Formula (I): 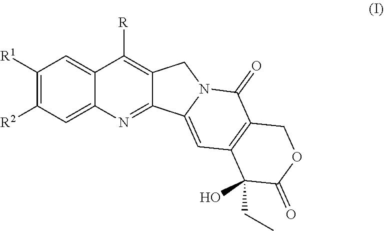
or a pharmaceutically acceptable salt or protected version thereof,
wherein:
R¹ is selected from: —H, —CH₃, —CHF₂, —CF₃, —F, —Br, —Cl, —OH, —OCH₃, —OCF₃ and —NH₂, and
R² is selected from: —H, —CH₃, —CF₃, —F, —Br, —Cl, —OH, —OCH₃ and —OCF₃, and wherein:
when R¹ is —NH₂, then R is R³ or R⁴, and when R¹ is other than —NH₂, then R is R⁴;
R³ is selected from: —H, —C₁-C₆ alkyl, —C₃-C₈ cycloalkyl, —(C₁-C₆ alkyl)-O—R⁵, 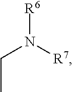
—CO₂R⁸, -aryl, -heteroaryl and —(C₁-C₆ alkyl)-aryl;
R⁴ is selected from: 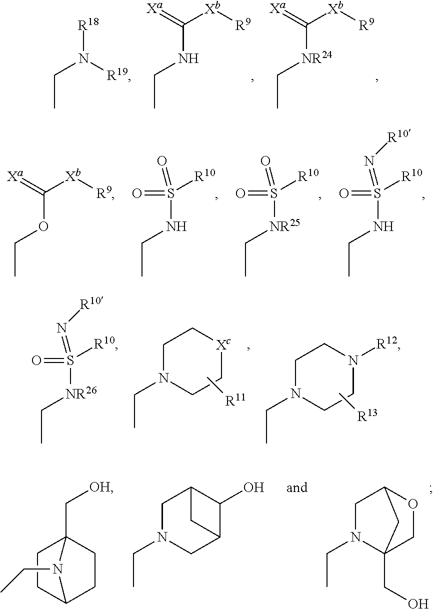
R⁵ is selected from: —H, —C₁-C₆ alkyl, —C₃-C₈ cycloalkyl, -aryl, -heteroaryl, -aryl and —(C₁-C₆ alkyl)-aryl;
R⁶ and R⁷ are each independently selected from: —H, —C₁-C₆ alkyl, —C₃-C₈ cycloalkyl, —(C₁-C₆ alkyl)-O—R⁵, —C₃-C₈ heterocycloalkyl and —C(O)R¹⁷;
R⁸ is selected from: —H, —C₁-C₆ alkyl, —C₃-C₈ cycloalkyl and —C₃-C₈ heterocycloalkyl;
each R⁹ is independently selected from: —H, —C₁-C₆ alkyl, —C₃-C₈ cycloalkyl, -aryl, -heteroaryl and —(C₁-C₆ alkyl)-aryl;
each R¹⁰ is independently selected from: —C₁-C₆ alkyl, —C₃-C₈ cycloalkyl, —NR¹⁴R^{14′}, -aryl, -heteroaryl and —(C₁-C₆ alkyl)-aryl;
each R^{10′} is independently selected from: —H, —C₁-C₆ alkyl, —C₃-C₈ cycloalkyl, -aryl, -heteroaryl, and —(C₁-C₆ alkyl)-aryl;
R¹¹ is selected from: —H and —C₁-C₆ alkyl;
R¹² is selected from: —H, —C₁-C₆ alkyl, —CO₂R⁸, -aryl, -heteroaryl, —(C₁-C₆ alkyl)-aryl, —S(O)₂R¹⁶ and 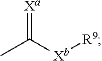
R¹³ is selected from: —H and —C₁-C₆ alkyl;
R¹⁴ and R^{14′} are each independently selected from: —H, C₁-C₆ alkyl, —C₃-C₈ cycloalkyl and —C₃-C₈ heterocycloalkyl;
R¹⁶ is selected from: —C₁-C₆ alkyl, —C₃-C₈ cycloalkyl, -aryl, -heteroaryl and —(C₁-C₆ alkyl)-aryl;
R¹⁷ is selected from: —C₁-C₆ alkyl, —C₃-C₈ cycloalkyl, —C₃-C₈ heterocycloalkyl, —(C₁-C₆ alkyl)-C₃-C₈ heterocycloalkyl, -aryl, -heteroaryl and —(C₁-C₆ alkyl)-aryl;
R¹⁸ and R¹⁹ taken together with the N atom to which they are bonded form a 4-, 5-, 6- or 7-membered ring having 0 to 3 substituents selected from: halogen, —C₁-C₆ alkyl, —C₃-C₈ cycloalkyl and —(C₁-C₆ alkyl)-O—R⁵;
R²⁴, R²⁵ and R²⁶ are each —C₁-C₆ alkyl;
X^{a} and X^{b} are each independently selected from: NH, O and S, and
X^{c} is selected from; O, S and S(O)₂,
with the proviso that the compound is other than (S)-9-amino-11-butyl-4-ethyl-4-hydroxy-1,12-dihydro-14H-pyrano[3′,4′:6,7]indolizino[1,2-b]quinoline-3,14(4H)-dione.

2. The compound according to claim 1, wherein R¹ is NH₂, and R is R³ or R⁴.

3. The compound according to claim 2, wherein R² is other than —H.

4. The compound according to claim 1, wherein R¹ is selected from: —H, —CH₃, —CHF₂, —CF₃, —F, —Br, —Cl, —OH, —OCH₃ and —OCF₃, and R is R⁴.

5. The compound according to claim 1 or 4, wherein R² is selected from: —H, —CH₃, —CF₃, —F, —Cl, —OCH₃ and —OCF₃.

6. The compound according to any one of claims 1 to 4, wherein R⁴ is selected from:

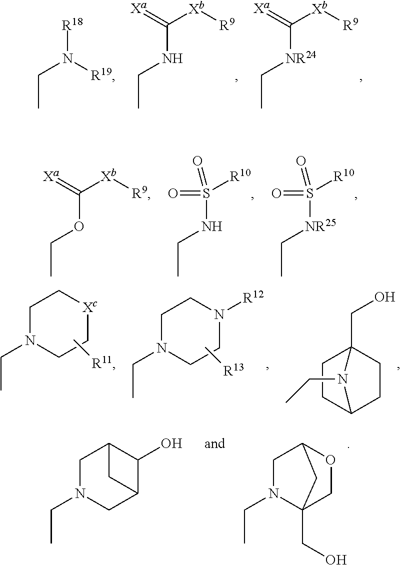

7. The compound according to claim 1 having Formula (II): 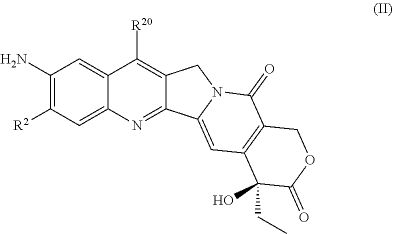
or a pharmaceutically acceptable salt or protected version thereof,
wherein:
R² is selected from: —H, —CH₃, —CF₃, —F, —Br, —Cl, —OH, —OCH₃ and —OCF₃;
R²⁰ is selected from: —H, —C₁-C₆ alkyl, —C₃-C₈ cycloalkyl, —(C₁-C₆ alkyl)-O—R⁵, 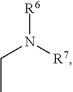
—CO₂R⁸, -aryl, -heteroaryl, —(C₁-C₆ alkyl)-aryl, 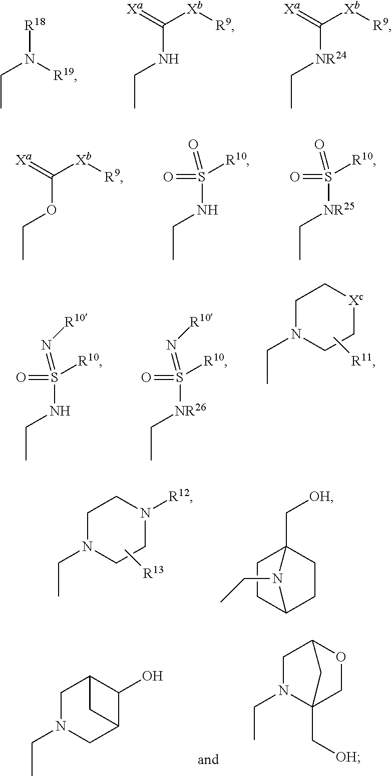
R⁵ is selected from: —H, —C₁-C₆ alkyl, —C₃-C₈ cycloalkyl, -aryl, -heteroaryl and —(C₁-C₆ alkyl)-aryl;
R⁶ and R⁷ are each independently selected from: —H, —C₁-C₆ alkyl, —C₃-C₈ cycloalkyl, —(C₁-C₆ alkyl)-O—R⁵, —C₃-C₈ heterocycloalkyl and —C(O)R¹⁷;
R⁸ is selected from: —H, —C₁-C₆ alkyl, —C₃-C₈ cycloalkyl and —C₃-C₈ heterocycloalkyl;
each R⁹ is independently selected from: —H, —C₁-C₆ alkyl, —C₃-C₈ cycloalkyl, -aryl, -heteroaryl and —(C₁-C₆ alkyl)-aryl;
each R¹⁰ is independently selected from: —C₁-C₆ alkyl, —C₃-C₈ cycloalkyl, —NR¹⁴R^{14′}, -aryl, -heteroaryl and —(C₁-C₆ alkyl)-aryl;
each R^{10′} is independently selected from: —H, —C₁-C₆ alkyl, —C₃-C₈ cycloalkyl, -aryl, -heteroaryl and —(C₁-C₆ alkyl)-aryl;
R¹¹ is selected from: —H and —C₁-C₆ alkyl;
R¹² is selected from: —H, —C₁-C₆ alkyl, —CO₂R⁸, -aryl, -heteroaryl, —(C₁-C₆ alkyl)-aryl, —S(O)₂R¹⁶ and 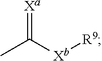
R¹³ is selected from: —H and —C₁-C₆ alkyl;
R¹⁴ and R^{14′} are each independently selected from: —H, C₁-C₆ alkyl, —C₃-C₈ cycloalkyl and —C₃-C₈ heterocycloalkyl;
R¹⁶ is selected from: —C₁-C₆ alkyl, —C₃-C₈ cycloalkyl, -aryl, -heteroaryl and —(C₁-C₆ alkyl)-aryl;
R¹⁷ is selected from: —C₁-C₆ alkyl, —C₃-C₈ cycloalkyl, —C₃-C₈ heterocycloalkyl, —(C₁-C₆ alkyl)-C₃-C₈ heterocycloalkyl, -aryl, -heteroaryl and —(C₁-C₆ alkyl)-aryl;
R¹⁸ and R¹⁹ taken together with the N atom to which they are bonded form a 4-, 5-, 6-, or 7-membered ring having 0 to 3 substituents selected from: halogen, —C₁-C₆ alkyl, —C₃-C₈ cycloalkyl and —(C₁-C₆ alkyl)-O—R⁵;
R²⁴, R²⁵ and R²⁶ are each —C₁-C₆ alkyl;
X^{a} and X^{b} are each independently selected from: NH, O and S, and
X^{c} is selected from: O, S and S(O)₂.

8. The compound according to claim 7, wherein R² is selected from: —CH₃, —CF₃, —F, —Br, —Cl, —OH, —OCH₃ and —OCF₃.

9. The compound according to claim 7, wherein R² is selected from: —CH₃, —CF₃, —F, —Cl, —OCH₃ and —OCF₃.

10. The compound according to any one of claims 7 to 9, wherein R²⁰ is selected from: —H, —C₁-C₆ alkyl, —(C₁-C₆ alkyl)-O—R⁵, 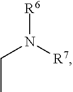
—(C₁-C₆ alkyl)-aryl,

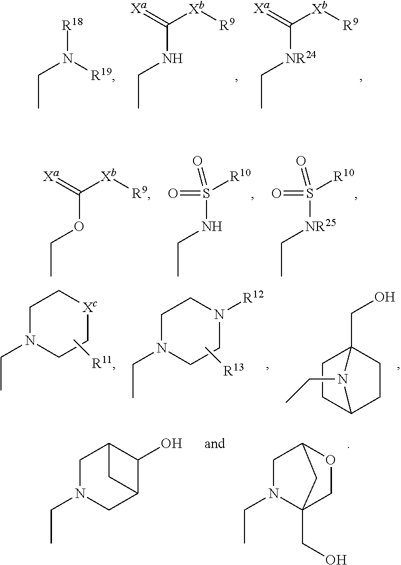

11. The compound according to any one of claims 7 to 9, wherein R²⁰ is selected from: —H, —C₁-C₆ alkyl, —(C₁-C₆ alkyl)-O—R⁵, 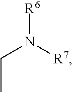
—(C₁-C₆ alkyl)-aryl,

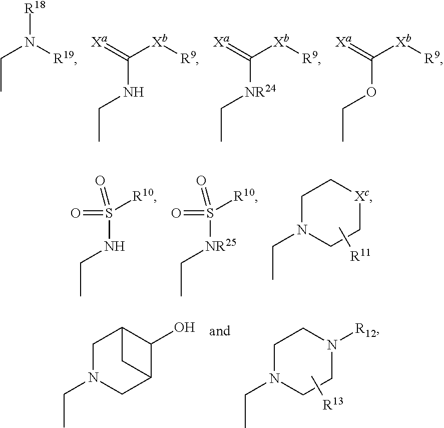

12. The compound according to any one of claims 7 to 9, wherein R²⁰ is selected from: —H, —C₁-C₆ alkyl, —(C₁-C₆ alkyl)-O—R⁵,

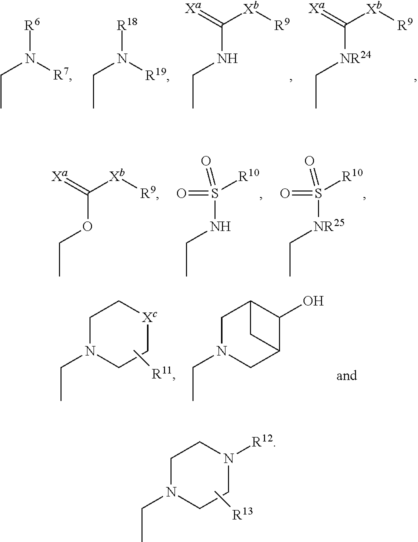

13. The compound according to claim 7 having Formula (IIa): 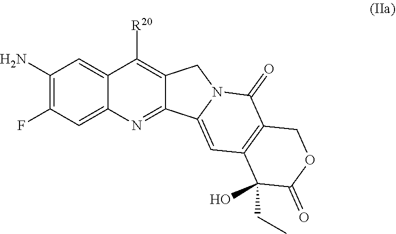
wherein: R²⁰, R⁵, R⁶, R⁷, R⁸, R⁹, R¹⁰, R^{10′}, R¹¹, R¹², R¹³, R¹⁴, R^{14′}, R¹⁶, R¹⁷, R¹⁸, R¹⁹, X^{a}, X^{b} and X^{c} are as defined in claim 7.

14. The compound according to claim 13, wherein R²⁰ is selected from: —H, —C₁-C₆ alkyl, —(C₁-C₆ alkyl)-O—R⁵,

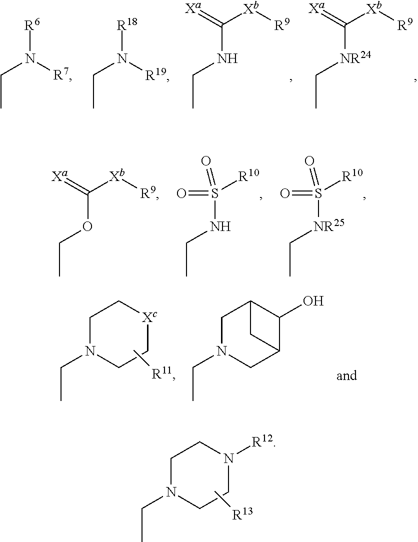

15. The compound according to claim 1 having Formula (III): 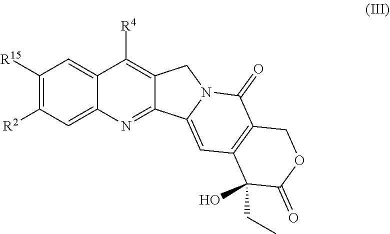
or a pharmaceutically acceptable salt or protected version thereof,
wherein:
R² is selected from: —H, —CH₃, —CF₃, —F, —Br, —Cl, —OH, —OCH₃ and —OCF₃;
R¹⁵ is selected from: —H, —CH₃, —CHF₂, —CF₃, —F, —Br, —Cl, —OH, —OCH₃ and —OCF₃;
R⁴ is selected from: 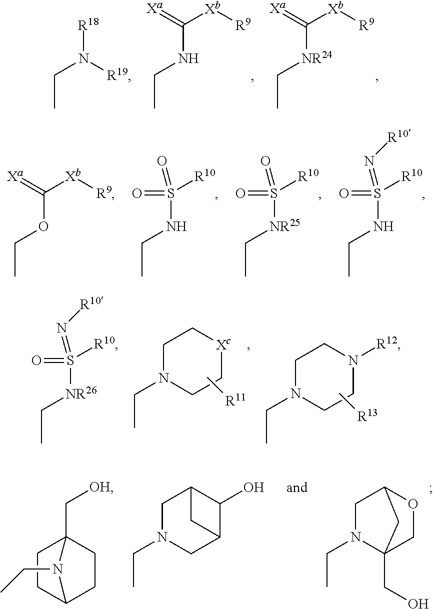
R⁵ is selected from: —H, —C₁-C₆ alkyl, —C₃-C₈ cycloalkyl, -aryl, -heteroaryl and —(C₁-C₆ alkyl)-aryl;
R⁸ is selected from: —H, —C₁-C₆ alkyl, —C₃-C₈ cycloalkyl and —C₃-C₈ heterocycloalkyl;
each R⁹ is independently selected from: —H, —C₁-C₆ alkyl, —C₃-C₈ cycloalkyl, -aryl, -heteroaryl and —(C₁-C₆ alkyl)-aryl;
each R¹⁰ is independently selected from: —C₁-C₆ alkyl, —C₃-C₈ cycloalkyl, —NR¹⁴R^{14′}, -aryl, -heteroaryl and —(C₁-C₆ alkyl)-aryl;
each R^{10′} is independently selected from: —H, —C₁-C₆ alkyl, —C₃-C₈ cycloalkyl, -aryl, -heteroaryl and —(C₁-C₆ alkyl)-aryl;
R¹¹ is selected from: —H and —C₁-C₆ alkyl;
R¹² is selected from: —H, —C₁-C₆ alkyl, —CO₂R⁸, -aryl, -heteroaryl, —(C₁-C₆ alkyl)-aryl, —S(O)₂R¹⁶ and 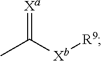
R¹³ is selected from: —H and —C₁-C₆ alkyl;
R¹⁴ and R^{14′} are each independently selected from: —H, C₁-C₆ alkyl, —C₃-C₈ cycloalkyl and —C₃-C₈ heterocycloalkyl;
R¹⁶ is selected from: —C₁-C₆ alkyl, —C₃-C₈ cycloalkyl, -aryl, -heteroaryl and —(C₁-C₆ alkyl)-aryl;
R¹⁸ and R¹⁹ taken together with the N atom to which they are bonded form a 4-, 5-, 6- or 7-membered ring having 0 to 3 substituents selected from: halogen, —C₁-C₆ alkyl, —C₃-C₈ cycloalkyl and —(C₁-C₆ alkyl)-O—R⁵;
R²⁴, R²⁵ and R²⁶ are each —C₁-C₆ alkyl;
X^{a} and X^{b} are each independently selected from: NH, O and S, and
X^{c} is selected from: O, S and S(O)₂.

16. The compound according to claim 15, wherein R² is selected from: —H, —F, —Br and —Cl.

17. The compound according to claim 15, wherein R¹⁵ is selected from: —CH₃, —CF₃, —OCH₃ and —OCF₃.

18. The compound according to claim 15, wherein R¹⁵ is —CH₃ or —OCH₃.

19. The compound according to any one of claims 15 to 18, wherein R⁴ is selected from:

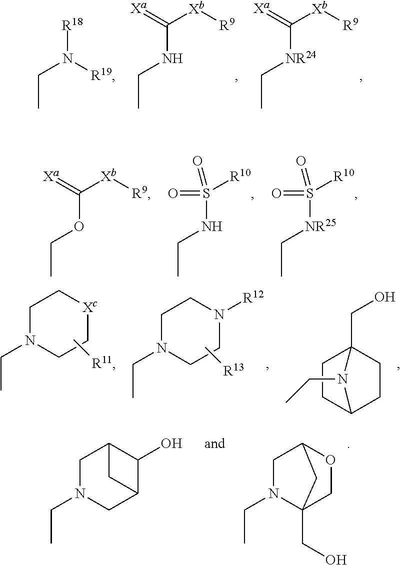

20. The compound according to claim 15 having Formula (IIIa) or (IIIb): 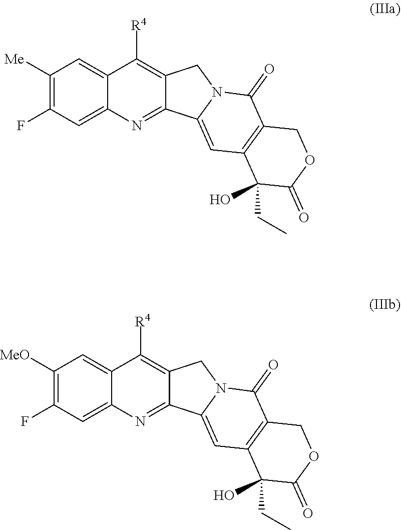
wherein: R⁴, R⁵, R⁸, R⁹, R¹⁰, R^{10′}, R¹¹, R¹², R¹³, R¹⁴, R¹⁴, R¹⁶, R¹⁸, R¹⁹, X^{a}, X^{b} and X^{c} are as defined in claim 15.

21. The compound according to claim 20, wherein R⁴ is selected from:

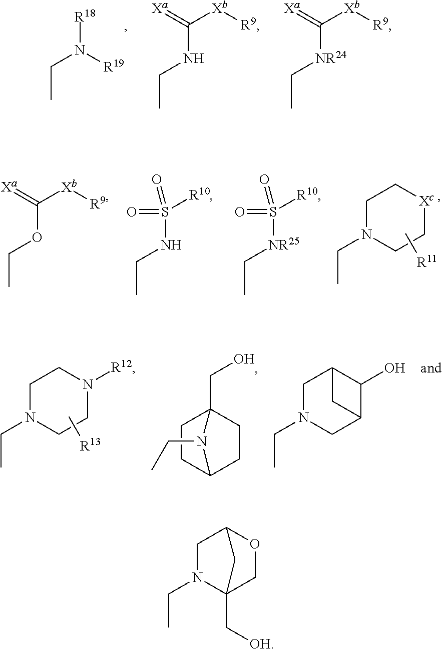

22. The compound according to any one of claims 1 to 21, wherein each alkyl, cycloalkyl, heterocycloalkyl, aryl and heteroaryl group is optionally substituted with one or more substituents selected from: halogen, acyl, acyloxy, alkoxy, carboxy, hydroxy, amino, amido, nitro, cyano, azido, alkylthio, thio, sulfonyl, sulfonamido, alkyl, cycloalkyl, heterocycloalkyl, aryl and heteroaryl.

23. The compound according to any one of claims 1 to 21, wherein each alkyl, cycloalkyl, heterocycloalkyl, aryl and heteroaryl group is optionally substituted with one or more substituents selected from: halogen, acyl, acyloxy, alkoxy, carboxy, hydroxy, amino, amido, nitro, cyano, azido, alkylthio, thio, sulfonyl and sulfonamido.

24. The compound according to claim 1, wherein the compound is selected from compounds 100 to 168 as set forth in Table 1.

25. A pharmaceutical composition comprising a compound according to any one of claims 1 to 24, and a pharmaceutically acceptable carrier or diluent.

26. A conjugate having Formula (X): 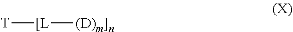
wherein:
T is a targeting moiety;
L is a linker;
D is a compound according to any one of claims 1 to 24;
m is an integer between 1 and 4, and
n is an integer between 1 and 10.

27. A conjugate having Formula (X): 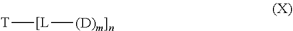
wherein:
T is a targeting moiety;
L is a linker;
m is an integer between 1 and 4;
n is an integer between 1 and 10, and
D is a compound of Formula (IV): 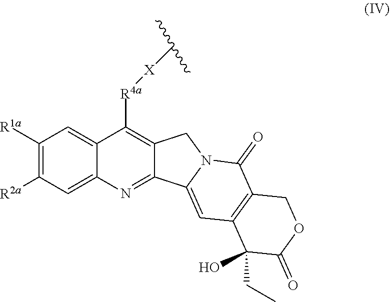
wherein:
R^{1a} is selected from: —H, —CH₃, —CHF₂, —CF₃, —F, —Br, —Cl, —OH, —OCH₃, —OCF₃ and —NH₂;
R^{2a} is selected from: —H, —CH₃, —CF₃, —F, —Br, —Cl, —OH, —OCH₃ and —OCF₃;
X is —O—, —S— or —NH—, and R^{4a} is selected from: 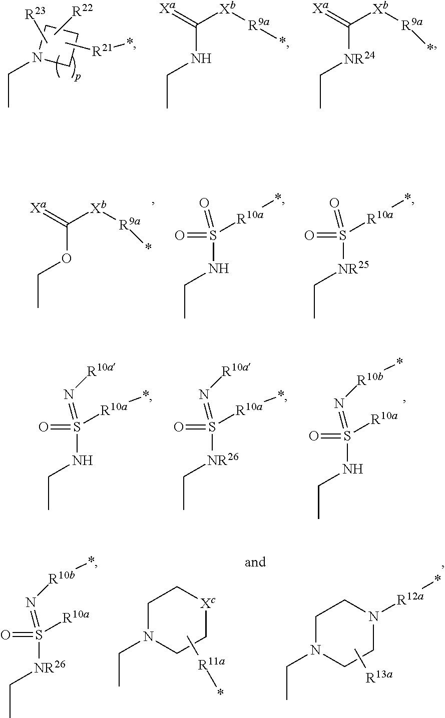
wherein * is the point of attachment to X, and wherein p is 1, 2, 3 or 4; or
X is O, and R^{4a}—X— is selected from: 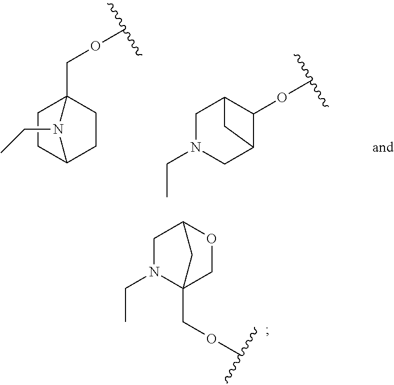
R^{5a} is selected from: —C₁-C₆ alkyl, —C₃-C₈ cycloalkyl, -aryl, -heteroaryl and —(C₁-C₆ alkyl)-aryl;
R^{8a} is selected from: —C₁-C₆ alkyl, —C₃-C₈ cycloalkyl and —C₃-C₈ heterocycloalkyl;
each R^{9a} is independently selected from: —C₁-C₆ alkyl, —C₃-C₈ cycloalkyl, -aryl, -heteroaryl and —(C₁-C₆ alkyl)-aryl; or R^{9a} is absent and X^{b}═X;
each R^{10a} is independently selected from: —C₁-C₆ alkyl, —C₃-C₈ cycloalkyl, -aryl, -heteroaryl, —(C₁-C₆ alkyl)-aryl and 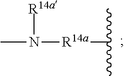
each R^{10a′} is independently selected from: —H, —C₁-C₆ alkyl, —C₃-C₈ cycloalkyl, -aryl, -heteroaryl and —(C₁-C₆ alkyl)-aryl;
each R^{10b} is independently selected from: —C₁-C₆ alkyl, —C₃-C₈ cycloalkyl, -aryl, -heteroaryl and —(C₁-C₆ alkyl)-aryl;
R^{11a} is absent or is —C₁-C₆ alkyl;
R^{12a} is selected from: —C₁-C₆ alkyl, —CO₂R^{8a}, -aryl, -heteroaryl, —(C₁-C₆ alkyl)-aryl, —S(O)₂R^{16a} and 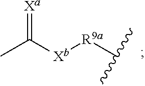
R^{13a} is selected from: —H and —C₁-C₆ alkyl;
R^{14a} is selected from: —C₁-C₆ alkyl, —C₃-C₈ cycloalkyl and —C₃-C₈ heterocycloalkyl;
R^{14a′} is selected from: H, —C₁-C₆ alkyl, —C₃-C₈ cycloalkyl and —C₃-C₈ heterocycloalkyl;
R^{16a} is selected from: —C₁-C₆ alkyl, —C₃-C₈ cycloalkyl, -aryl, -heteroaryl and —(C₁-C₆ alkyl)-aryl;
R²¹ is selected from: —C₁-C₆ alkyl, —C₃-C₈ cycloalkyl and —(C₁-C₆ alkyl)-O—R^{5a};
R²² and R²³ are each independently selected from: —H, -halogen, —C₁-C₆ alkyl and —C₃-C₈ cycloalkyl;
R²⁴, R²⁵ and R²⁶ are each —C₁-C₆ alkyl;
X^{a} and X^{b} are each independently selected from: NH, O and S;
X^{c} is selected from: O, S and S(O)₂, and [symbol]
denotes the point of attachment to linker, L.

28. The conjugate according to claim 27, wherein R^{1a} is selected from: —CH₃, —CF₃, —OCH₃, —OCF₃ and —NH₂.

29. The conjugate according to claim 27, wherein R^{1a} is selected from: —CH₃, —OCH₃ and NH₂.

30. The conjugate according to claim 27, wherein R^{2a} is selected from: —H, —F, —Br and —Cl.

31. The conjugate according to any one of claims 27 to 30, wherein X is —O—, —S— or —NH—, and R^{4a} is selected from:

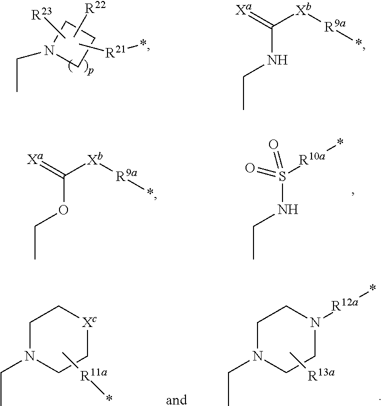

32. A conjugate having Formula (X): 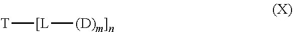
wherein:
T is a targeting moiety;
L is a linker;
m is an integer between 1 and 4;
n is an integer between 1 and 10, and
D is a compound of Formula (V): 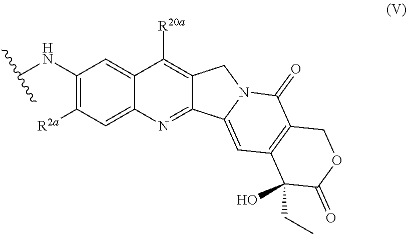
wherein:
R^{2a} is selected from: —CH₃, —CF₃, —F, —Br, —Cl, —OH, —OCH₃ and —OCF₃;
R^{20a} is selected from: —H, —C₁-C₆ alkyl, —C₃-C₈ cycloalkyl, —(C₁-C₆ alkyl)-O—R⁵, 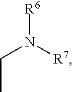
—CO₂R⁸, -aryl, -heteroaryl, —(C₁-C₆ alkyl)-aryl, 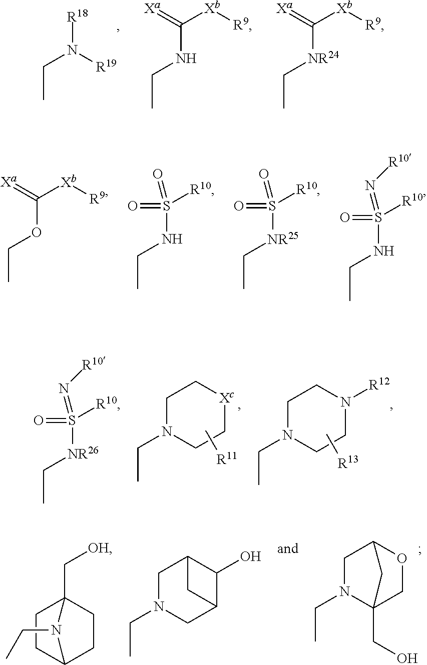
R⁵ is selected from: —H, —C₁-C₆ alkyl, —C₃-C₈ cycloalkyl, -aryl, -heteroaryl and —(C₁-C₆ alkyl)-aryl;
R⁶ and R⁷ are each independently selected from: —H, —C₁-C₆ alkyl, —C₃-C₈ cycloalkyl, —(C₁-C₆ alkyl)-O—R⁵, —C₃-C₈ heterocycloalkyl and —C(O)R¹⁷;
R⁸ is selected from: —H, —C₁-C₆ alkyl, —C₃-C₈ cycloalkyl and —C₃-C₈ heterocycloalkyl;
each R⁹ is independently selected from: —H, —C₁-C₆ alkyl, —C₃-C₈ cycloalkyl, -aryl, -heteroaryl and —(C₁-C₆ alkyl)-aryl;
each R^{10a} is independently selected from: —C₁-C₆ alkyl, —C₃-C₈ cycloalkyl, -aryl, -heteroaryl, —(C₁-C₆ alkyl)-aryl and 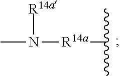
R^{10a′} is selected from: —H, —C₁-C₆ alkyl, —C₃-C₈ cycloalkyl, -aryl, -heteroaryl and —(C₁-C₆ alkyl)-aryl;
R¹¹ is selected from: —H and —C₁-C₆ alkyl;
R¹² is selected from: —H, —C₁-C₆ alkyl, —CO₂R⁸, -aryl, -heteroaryl, —(C₁-C₆ alkyl)-aryl, —S(O)₂R¹⁶ and 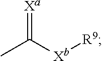
R¹³ is selected from: —H and —C₁-C₆ alkyl;
R¹⁴ and R^{14′} are each independently selected from: —H, C₁-C₆ alkyl, —C₃-C₈ cycloalkyl and —C₃-C₈ heterocycloalkyl;
R¹⁶ is selected from: —C₁-C₆ alkyl, —C₃-C₈ cycloalkyl, -aryl, -heteroaryl and —(C₁-C₆ alkyl)-aryl;
R¹⁷ is selected from: —C₁-C₆ alkyl, —C₃-C₈ cycloalkyl, —C₃-C₈ heterocycloalkyl, —(C₁-C₆ alkyl)-C₃-C₈ heterocycloalkyl, -aryl, -heteroaryl and —(C₁-C₆ alkyl)-aryl;
R¹⁸ and R¹⁹ taken together with the N atom to which they are bonded form a 4-, 5-, 6- or 7-membered ring having 0 to 3 substituents selected from: halogen, —C₁-C₆ alkyl, —C₃-C₈cycloalkyl and —(C₁-C₆ alkyl)-O—R⁵;
R²⁴, R²⁵ and R²⁶ are each —C₁-C₆ alkyl;
X^{a} and X^{b} are each independently selected from: NH, O and S;
X^{c} is selected from: O, S and S(O)₂, and [symbol]
denotes the point of attachment to linker, L.

33. The conjugate according to claim 32, wherein R^{2a} is F.

34. The conjugate according to claim 32 or 33, wherein R^{20a} is selected from: —H, —C₁-C₆ alkyl, —(C₁-C₆ alkyl)-O—R⁵, 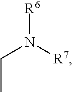
—(C₁-C₆ alkyl)-aryl,

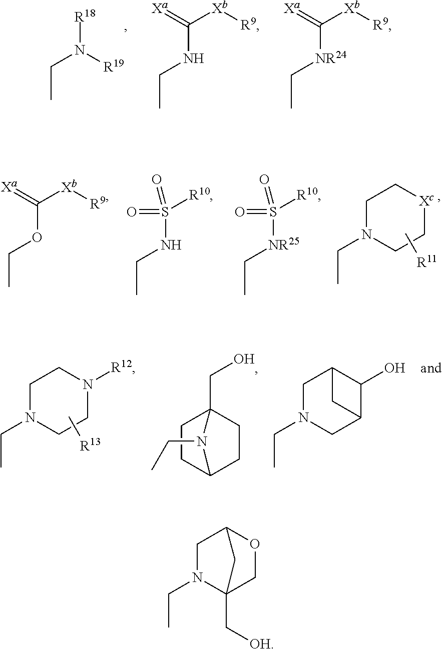

35. A conjugate having Formula (X): 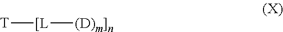
wherein:
T is a targeting moiety;
L is a linker;
m is an integer between 1 and 4;
n is an integer between 1 and 10, and
D is a compound of Formula (VI): 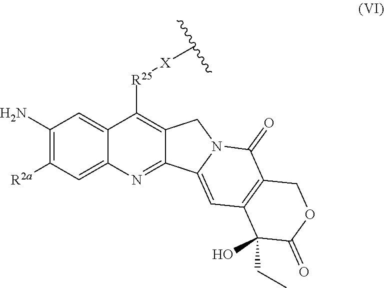
wherein:
R^{2a} is selected from: —H, —CH₃, —CF₃, —F, —Br, —Cl, —OH, —OCH₃ and —OCF₃;
X is —O—, —S— or —NH—, and R²⁵ is selected from: —C₁-C₆ alkyl, —(C₁-C₆ alkyl)-O—R^{5a}, —CO₂R^{8a}, —C(O)—, -aryl, -heteroaryl, —(C₁-C₆ alkyl)-aryl, 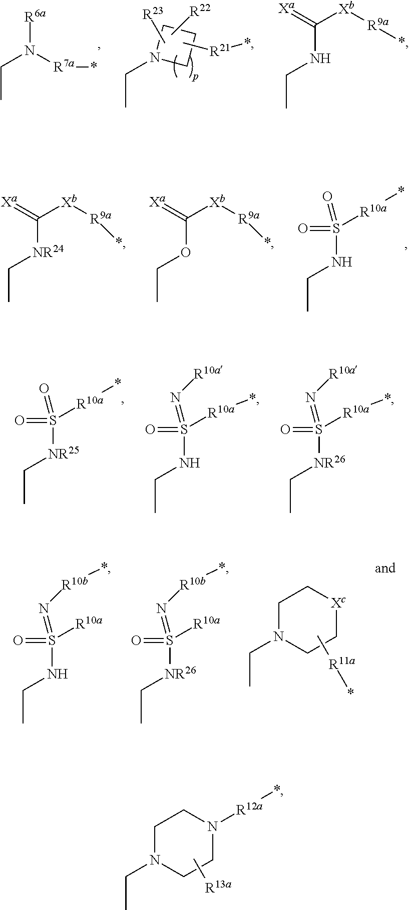
wherein * is the point of attachment to X, and wherein p is 1, 2, 3 or 4; or
X is O, and R²⁵—X— is selected from: 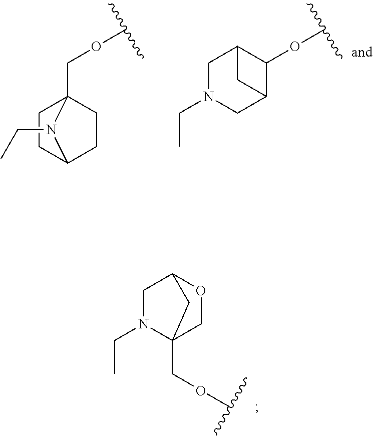 [SMILES: *OCC12CCC(CC1)N2CC ; *OC1C2CC1CN(CC)C2 ; *OCC12COC(CN1CC)C2]
R^{5a} is selected from: —C₁-C₆ alkyl, —C₃-C₈ cycloalkyl, -aryl, -heteroaryl and —(C₁-C₆ alkyl)-aryl;
R^{6a} is selected from: —H, —C₁-C₆ alkyl, —C₃-C₈ cycloalkyl and —C₃-C₈ heterocycloalkyl;
R^{7a} is selected from: —C₁-C₆ alkyl, —C₃-C₈ cycloalkyl, —(C₁-C₆ alkyl)-O—R^{5a}, —C₃-C₈ heterocycloalkyl and —C(O)R^{17a};
R^{8a} is selected from: —C₁-C₆ alkyl, —C₃-C₈ cycloalkyl and —C₃-C₈ heterocycloalkyl;
each R^{9a} is independently selected from: —C₁-C₆ alkyl, —C₃-C₈ cycloalkyl, -aryl, -heteroaryl and —(C₁-C₆ alkyl)-aryl; or R^{9a} is absent and X^{b}═X;
each R^{10a} is independently selected from: —C₁-C₆ alkyl, —C₃-C₈ cycloalkyl, -aryl, -heteroaryl, —(C₁-C₆ alkyl)-aryl and 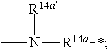
each R^{10a′} is independently selected from: —H, —C₁-C₆ alkyl, —C₃-C₈ cycloalkyl, -aryl, -heteroaryl and —(C₁-C₆ alkyl)-aryl;
each R^{10b} is independently selected from: —C₁-C₆ alkyl, —C₃-C₈ cycloalkyl, -aryl, -heteroaryl and —(C₁-C₆ alkyl)-aryl;
R^{11a} is absent or is —C₁-C₆ alkyl;
R^{12a} is selected from: —C₁-C₆ alkyl, —CO₂R^{8a}, -aryl, -heteroaryl, —(C₁-C₆ alkyl)-aryl, —S(O)₂R^{16a} and 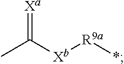
R^{13a} is selected from: —H and —C₁-C₆ alkyl;
R^{14a} is selected from: —C₁-C₆ alkyl, —C₃-C₈ cycloalkyl and —C₃-C₈ heterocycloalkyl;
R^{14a′} is selected from: H, —C₁-C₆ alkyl, —C₃-C₈ cycloalkyl and —C₃-C₈ heterocycloalkyl;
R^{16a} is selected from: —C₁-C₆ alkyl, —C₃-C₈ cycloalkyl, -aryl, -heteroaryl and —(C₁-C₆ alkyl)-aryl;
R^{17a} is selected from: —C₁-C₆ alkyl, —C₃-C₈ cycloalkyl, —C₃-C₈ heterocycloalkyl, —(C₁-C₆ alkyl)-C₃-C₈ heterocycloalkyl, -aryl, -heteroaryl and —(C₁-C₆ alkyl)-aryl;
R²¹ is selected from: —C₁-C₆ alkyl, —C₃-C₈ cycloalkyl and —(C₁-C₆ alkyl)-O—R^{5a};
R²² and R²³ are each independently selected from: —H, -halogen, —C₁-C₆ alkyl and —C₃-C₈ cycloalkyl;
R²⁴, R²⁵ and R²⁶ are each —C₁-C₆ alkyl;
X^{a} and X^{b} are each independently selected from: NH, O and S;
X^{c} is selected from: O, S and S(O)₂, and [symbol]
denotes the point of attachment to linker, L.

36. The conjugate according to claim 35, wherein R^{2a} is selected from: —CH₃, —CF₃, —F, —Br, —Cl, —OH, —OCH₃ and —OCF₃.

37. The conjugate according to claim 35, wherein R^{2a} is F.

38. The conjugate according to any one of claims 35 to 37, wherein X is —O—, —S— or —NH—, and R²⁵ is selected from: —C₁-C₆ alkyl, —(C₁-C₆ alkyl)-O—R^{5a}, —(C₁-C₆ alkyl)-aryl, 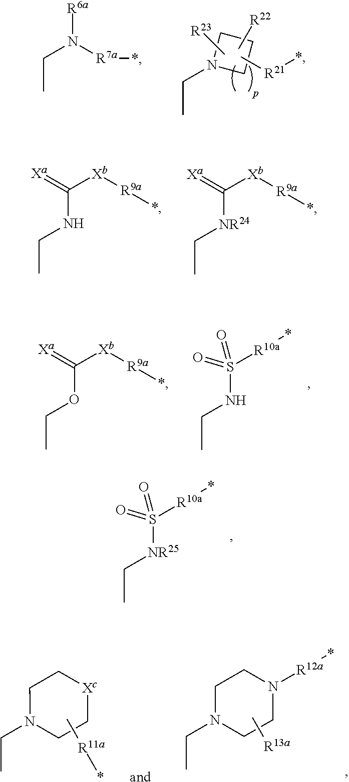
or X is O, and R²⁵—X— is selected from:

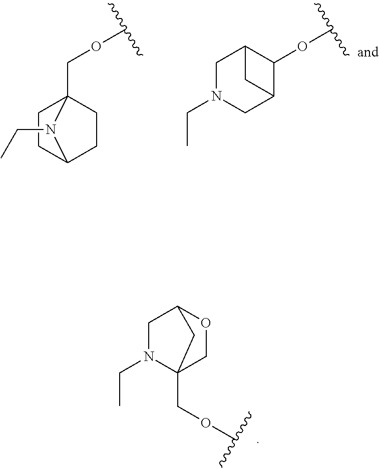 [SMILES: *OCC12CCC(CC1)N2CC ; *OC1C2CC1CN(CC)C2 ; *OCC12COC(CN1CC)C2]

39. The conjugate according to any one of claims 35 to 37, wherein X is —O—, —S— or —NH—, and R²⁵ is selected from: —C₁-C₆ alkyl, —(C₁-C₆ alkyl)-O—R^{5a}, —(C₁-C₆ alkyl)-aryl,

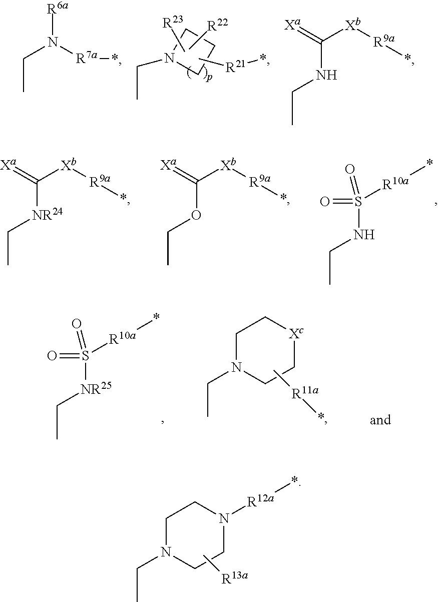

40. The conjugate according to any one of claims 27 to 39, wherein each alkyl, cycloalkyl, heterocycloalkyl, aryl and heteroaryl group is optionally substituted with one or more substituents selected from: halogen, acyl, acyloxy, alkoxy, carboxy, hydroxy, amino, amido, nitro, cyano, azido, alkylthio, thio, sulfonyl, sulfonamido, alkyl, cycloalkyl, heterocycloalkyl, aryl and heteroaryl.

41. The conjugate according to any one of claims 27 to 39, wherein each alkyl, cycloalkyl, heterocycloalkyl, aryl and heteroaryl group is optionally substituted with one or more substituents selected from: halogen, acyl, acyloxy, alkoxy, carboxy, hydroxy, amino, amido, nitro, cyano, azido, alkylthio, thio, sulfonyl and sulfonamido.

42. The conjugate according to any one of claims 26 to 41, wherein m is 1 or 2.

43. The conjugate according to any one of claims 26 to 42, wherein n is between 2 and 8.

44. The conjugate according to any one of claims 26 to 43, wherein L is a cleavable linker.

45. The conjugate according to claim 44, wherein L is a protease cleavable linker.

46. The conjugate according to claim 44 or 45, wherein L comprises a dipeptide, tripeptide or tetrapeptide.

47. The conjugate according to any one of claims 26 to 46, wherein T binds to a tumor associated antigen.

48. The conjugate according to any one of claims 26 to 47, wherein T is an antibody or antigen-binding antibody fragment.

49. The conjugate according to claim 48, wherein the antibody is a bispecific or multispecific antibody.

50. A pharmaceutical composition comprising a conjugate according to any one of claims 26 to 49, and a pharmaceutically acceptable carrier or diluent.

51. A method of inhibiting the proliferation of cancer cells comprising contacting the cells with an effective amount of the compound according to any one of claims 1 to 24, or the conjugate according to any one of claims 26 to 49.

52. A method of killing cancer cells comprising contacting the cells with an effective amount of the compound according to any one of claims 1 to 24, or the conjugate according to any one of claims 26 to 49.

53. A method of treating cancer in a subject in need thereof comprising administering to the subject an effective amount of the compound according to any one of claims 1 to 24, or the conjugate according to any one of claims 26 to 49.

54. A method of treating an autoimmune disease in a subject in need thereof comprising administering to the subject an effective amount of the compound according to any one of claims 1 to 24, or the conjugate according to any one of claims 26 to 49.

55. A method of treating a viral infection in a subject in need thereof comprising administering to the subject an effective amount of the compound according to any one of claims 1 to 24, or the conjugate according to any one of claims 26 to 49.

56. A compound according to any one of claims 1 to 24 or a conjugate according to any one of claims 26 to 49 for use in therapy.

57. A compound according to any one of claims 1 to 24 or a conjugate according to any one of claims 26 to 49 for use in the treatment of cancer, an autoimmune disease or a viral infection.

58. Use of a compound according to any one of claims 1 to 24 or a conjugate according to any one of claims 26 to 49. in the manufacture of a medicament for the treatment of cancer, an autoimmune disease or a viral infection.
